SEQUENCE LISTING (111 sequences)

SEQ ID NO: 1 - DNA, 336 bp, Mus musculus
  CDS (1)..(336)
aagcccctcctctacactgtcatcatgtcgcaaagggtgtgtcaggtgctggtggcaatcccctatttgtactgcacatttgtttctcttctagttaccataaagatttttacattgtctttctgtgggtataatgtcatcagtcatttctactgtgacagtcttcccttgttatctttgatctgttcaaacacaaatgaaattgaaatgattattctggtcttagcagcttttaatttgatatcctcccttctagtggtccttgtttcttacctgttcatccttatagccattctcagaatgaactcggctgagggcagacgcaaggctttctca

SEQ ID NO: 2 - protein, 112 aa, Mus musculus
KPLLYTVIMSQRVCQVLVAIPYLYCTFVSLLVTIKIFTLSFCGYNVISHFYCDSLPLLSLICSNTNEIEMIILVLAAFNLISSLLVVLVSYLFILIAILRMNSAEGRRKAFS

SEQ ID NO: 3 - DNA, 336 bp, Mus musculus
  CDS (1)..(336)
  misc_feature (1)..(1): n is a, c, g, or t
natcctttgcactacactaccattatgaccccatgcctctgcacatctctggtggctttctcttgggtcattgccacttttaatcctctcttgcataccctcatgatggcccgacttcatttctgctctgaaaatattatccaccatttcttctgtgacatcaactctctcctccctctctcctgttctgataccagtctcaatcagttgatggttctgtctgtggtgggcctgatatttgtggtaccatcagtttgtatcttagcttcttatggtcgcattgtctctgctgtgatgaaaattacttcaatggaaggaaagctcaaagcattctca

SEQ ID NO: 4 - protein, 112 aa, Mus musculus
  misc_feature (1)..(1): The ′Xaa′ at location 1 stands for Asn, Asp, His, or Tyr.
XPLHYTTIMTPCLCTSLVAFSWVIATFNPLLHTLMMARLHFCSENIIHHFFCDINSLLPLSCSDTSLNQLMVLSVVGLIFVVPSVCILASYGRIVSAVMKITSMEGKLKAFS

SEQ ID NO: 5 - DNA, 336 bp, Mus musculus
  CDS (1)..(336)
aaccccttactctacacagtttcaatgtctcccaagctgtgtgcattccttgtagctggaacatatatgtggggtgtactctgttccttgacaattacatactctcttttgcaactatcctactgtggacctaacatcatcaaccactttggctgtgagtactctgccattctgtctttgtcctgttctgaccccaccttcagccaagtggtatgtttaaccatttctatattcaatgagacttgcagcctcctcatcatcctggcctcctatgtcttcatagttgtcacaatcattaagatgccttctaagggtggactccaaaaagctttctct

SEQ ID NO: 6 - protein, 112 aa, Mus musculus
NPLLYTVSMSPKLCAFLVAGTYMWGVLCSLTITYSLLQLSYCGPNIINHFGCEYSAILSLSCSDPTFSQVVCLTISIFNETCSLLIILASYVFIVVTIIKMPSKGGLQKAFS

SEQ ID NO: 7 - DNA, 336 bp, Mus musculus
  CDS (1)..(336)
aaccctcttctgtactcatcagtcatgtcacccacactttgcgctcagatggtgatgggaagctacacagcaggaatcatgggttctctatctcaggtgtgtgccttgctgcagctccacttctgtggacctaacgtcatcagacatttcttctgtgacatgccccagctgttaaatctatcctgcactgacactttttttgcacaggtagtgcttctcatattaacaatgttgttttgtatttcaaatgctttagtcatcataatatcctatggctatattgtcttgtccattctgaagatcacttcagctaagggtaggtccaaggcattcaat

SEQ ID NO: 8 - protein, 112 aa, Mus musculus
NPLLYSSVMSPTLCAQMVMGSYTAGIMGSLSQVCALLQLHFCGPNVIRHFFCDMPQLLNLSCTDTFFAQVVLLILTMLFCISNALVIIISYGYIVLSILKITSAKGRSKAFN

SEQ ID NO: 9 - DNA, 666 bp, Mus musculus
  CDS (1)..(666)
ggaaatttggcttttgctgatgcctgtacttcatcctctgtgacaccaaagatgcttatgaaattttcaaataagaatgacatgatatccatgggtgagtgttttgctcaattttattttttttgtttaagtgcaactgcggaatgtttcatcctggtagcgatggcctatgaccgctatgtagccatatgcaaacctctgctctatgtagtggtgatgtccaacagactctgtattcagttcataggtgtatcctatctaattggacttctacatggtctacttcatgtaggattgttatttaggttaacgttttgtagttccaatgtaataaatcatttctactgtgaaatcctgccactttatagaatttcttgcactgacccatctatcaatgtactggtagctttcattatggctattttaatacaagtgagtacctttatgagtattatagtctcctatatcctcatcctctttgccatcctgagaacaaagtctgagaggggcagaaacaaagccttctctacttgtagttcccacctgtcatctgtatctttgttctatggcactctcttcatcatatatgtcctctcttgctctgacaaagataattatcagggtaaaatgtattcactgttctataccattatcattcctctgcta

SEQ ID NO: 10 - protein, 222 aa, Mus musculus
GNLAFADACTSSSVTPKMLMKFSNKNDMISMGECFAQFYFFCLSATAECFILVAMAYDRYVAICKPLLYVVVMSNRLCIQFIGVSYLIGLLHGLLHVGLLFRLTFCSSNVINHFYCEILPLYRISCTDPSINVLVAFIMAILIQVSTFMSIIVSYILILFAILRTKSERGRNKAFSTCSSHLSSVSLFYGTLFIIYVLSCSDKDNYQGKMYSLFYTIIIPLL

SEQ ID NO: 11 - DNA, 666 bp, Mus musculus
  CDS (1)..(666)
agtaacctctctctggtggactgtgtttatgcctcagctgtcacccccaaagtgatagaagggtttctcacagaaaataagatcatatcctacaatgcatgtgctgcccagatgtttttctttgtagcctttgctattactgagtgttttattctggcctcaatggcctatgaccgtcatgcagcagtgtgcaaacccttgcattactccactactatgacaactacgatatgtgtcctgcttcttgctggatcttacttgagtggactcttacaatcttccatccatgtttccttcacattccatctttccttctgtcgttctaatgtagtcaatcactttttctgtgatatccccccactattagctctttcttgctccagtattcacataaatgaaattatactcttcatgttggcaggattcaatgttgttttttccctattggttatattgacctcttacttactaatttctgttgcaattgtgagaatgcgctctgctgagagcaggaagaaggccatttccacctgtgcatcccaccttaccactgtttccatcttctatggcacaattatcttcatgtacttacagcctagctccaatcactccatggacactgacaagatggcatctgttttctacaccatggtcattcccatgctg

SEQ ID NO: 12 - protein, 222 aa, Mus musculus
SNLSLVDCVYASAVTPKVIEGFLTENKIISYNACAAQMFFFVAFAITECFILASMAYDRHAAVCKPLHYSTTMTTTICVLLLAGSYLSGLLQSSIHVSFTFHLSFCRSNVVNHFFCDIPPLLALSCSSIHINEIILFMLAGFNVVFSLLVILTSYLLISVAIVRMRSAESRKKAISTCASHLTTVSIFYGTIIFMYLQPSSNHSMDTDKMASVFYTMVIPML

SEQ ID NO: 13 - DNA, 948 bp, Mus musculus
  CDS (1)..(945)
atgatgcagaatatttcagaactgtcagaatttattcttcttgggttaacagatgcaccattcctgcaaactcctttatttatcatctttactctcatttacttgacaacattgtttgggaatcttggaatgattttgctgattctgctggactccagactccacactcccatgtactttttcctcagtaacctctctctggtggactgtgtttatgcctcagctgtcacccccaaagtgatagaagggtttctcacagaaaataagatcatatcctacaatgcatgtgctgcccagatgtttttctttgtagcctttgctattactgagtgttttattctggcctcaatggcctatgaccgtcatgcagcagtgtgcaaacccttgcattactccactactatgacaactacgatatgtgtcctgcttcttgctggatcttacttgagtggactcttacaatcttccatccatgtttccttcacattccatctttccttctgtcgttctaatgtagtcaatcactttttctgtgatatccccccactattagctctttcttgctccagtattcacataaatgaaattatactcttcatgttggcaggattcaatgttgttttttccctattggttatattgacctcttacttactaatttctgttgcaattgtgagaatgcgctctgctgagagcaggaagaaggccatttccacctgtgcatcccaccttaccactgtttccatcttctatggcacaattatcttcatgtacttacagcctagctccaatcactccatggacactgacaagatggcatctgttttctacaccatggtcattcccatgctgaaccctcttgtatatagcctgagaaataaagaagtcaagaatgcagtcaggaaggttgctggaaaagcattgttttcactgggattagtcaattaa

SEQ ID NO: 14 - protein, 315 aa, Mus musculus
MMQNISELSEFILLGLTDAPFLQTPLFIIFTLIYLTTLFGNLGMILLILLDSRLHTPMYFFLSNLSLVDCVYASAVTPKVIEGFLTENKIISYNACAAQMFFFVAFAITECFILASMAYDRHAAVCKPLHYSTTMTTTICVLLLAGSYLSGLLQSSIHVSFTFHLSFCRSNVVNHFFCDIPPLLALSCSSIHINEIILFMLAGFNVVFSLLVILTSYLLISVAIVRMRSAESRKKAISTCASHLTTVSIFYGTIIFMYLQPSSNHSMDTDKMASVFYTMVIPMLNPLVYSLRNKEVKNAVRKVAGKALFSLGLVN

SEQ ID NO: 15 - DNA, 942 bp, Mus musculus
  CDS (1)..(939)
atggaaaagaacaacctcacagcagtgactcaattcatcctgatgggtattactgagcgccctgaactacaggccccattgtttggattgttcctagtcatctacttgagctcaatgtttggcaacttgggcatgatcattctaaccacagtggactccaaattgcaaacacccatgtactttttcattagacacctggctatcacagaccttggttattctacagctgtgggacctaagatgttggtaaattttgttgtagatttgaacataatctcctataatctttgtgctacacagctagctttttttcttgtgtttataattagtgagctttttattctgtctgcaatgtcctatgaccgctatgtggccatctgtaagcccctcctctacactgtcatcatgtcgcaaagggtgtgtcaggtgctggtggcaatcccctatttgtactgcacatttgtttctcttctagttaccataaagatttttacattgtctttctgtgggtataatgtcatcagtcatttctactgtgacagtcttcccttgttatctttgatctgttcaaacacaaatgaaattgaaatgattattctggtcttagcagcttttaatttgatatcctcccttctagtggtccttgtttcttacctgttcatccttatagccattctcagaatgaactcggctgagggcagacgcaaggctttctcaacctgtggttcccacctgacagtggtcactgtcttctatggtactttaatatttatgtatgtgcagcctcagtccagtcactcttttgacacggataaagtggcttccatcttttataccctgattatacccatgttaaaccccatgatatacagtttgaggaacaaagatgtaaaatatgcacttcaaaggtcattgaaaaagatatacagcatactctcataa

SEQ ID NO: 16 - protein, 313 aa, Mus musculus
MEKNNLTAVTQFILMGITERPELQAPLFGLFLVIYLSSMFGNLGMIILTTVDSKLQTPMYFFIRHLAITDLGYSTAVGPKMLVNFVVDLNIISYNLCATQLAFFLVFIISELFILSAMSYDRYVAICKPLLYTVIMSQRVCQVLVAIPYLYCTFVSLLVTIKIFTLSFCGYNVISHFYCDSLPLLSLICSNTNEIEMIILVLAAFNLISSLLVVLVSYLFILIAILRMNSAEGRRKAFSTCGSHLTVVTVFYGTLIFMYVQPQSSHSFDTDKVASIFYTLIIPMLNPMIYSLRNKDVKYALQRSLKKIYSILS

SEQ ID NO: 17 - DNA, 918 bp, Mus musculus
  CDS (1)..(915)
atggagttgaacaggacccagctgactgaatttgttctcagaggaataacagatcgttcagagctgcaagtccccctgttcctggtgttctttctcatctatgttatcaccatggtgggcaaccttggcttaatctttgtcatctggaaggaccctcatcttcacacacccatgtaccttttccttggaaatttggcttttgctgatgcctgtacttcatcctctgtgacaccaaagatgcttatgaaattttcaaataagaatgacatgatatccatgggtgagtgttttgctcaattttattttttttgtttaagtgcaactgcggaatgtttcatcctggtagcgatggcctatgaccgctatgtagccatatgcaaacctctgctctatgtagtggtgatgtccaacagactctgtattcagttcataggtgtatcctatctaattggacttctacatggtctacttcatgtaggattgttatttaggttaatgttttgtagttccaatgtaataaatcatttctactgtgaaatcctgccactttatagaatttcttgcactgacccatctatcaatgtactggtagttttcattatgggtattttaatacaagtgagtacctttatgagtattatagtctcctatatcctcatcctctttgccatcctgagaacaaagtctgagaggggcagaaacaaagccttctctacttgcagttcccacctgtcatctgtatctttgttctatggcactctcttcatcatatatgtcctctctggctctgacacagataattatcagggtaaaatgtattcactgttccataccattatcattcctctgctgaaccccttcatttacagcctcagaaacaaagaagtcatcggtgccttgagaaaactcagagaatga

SEQ ID NO: 18 - protein, 305 aa, Mus musculus
MELNRTQLTEFVLRGITDRSELQVPLFLVFFLIYVITMVGNLGLIFVIWKDPHLHTPMYLFLGNLAFADACTSSSVTPKMLMKFSNKNDMISMGECFAQFYFFCLSATAECFILVAMAYDRYVAICKPLLYVVVMSNRLCIQFIGVSYLIGLLHGLLHVGLLFRLMFCSSNVINHFYCEILPLYRISCTDPSINVLVVFIMGILIQVSTFMSIIVSYILILFAILRTKSERGRNKAFSTCSSHLSSVSLFYGTLFIIYVLSGSDTDNYQGKMYSLFHTIIIPLLNPFIYSLRNKEVIGALRKLRE

SEQ ID NO: 19 - DNA, 942 bp, Mus musculus
  CDS (1)..(939)
atggagaaggaaaaccaaaccagcgtctctgagtttctcctcctgggtttttcaagttggccagggcatcaagggcttctctttgcactcttcctatgtctctatttaacaggactatttggaaacctcctcatcttgttagccattggttcaaacaatcacctccacacacctatgtatttcttccttgccaatttgtccttggtagacctctgccttccttcagctacagtccccaagatgctattgaacatccaaaccaagtctcagtccatctcctaccctggatgcctggctcagatgtatttctgtatgatgtttgctaacatggacaacttccttctcactgtgatggcatacgaccgctttgtggccatctgccatcctttgcactacactaccattatgaccccatgcctctgcacatctctggtggctttctcttgggtcattgccacttttaatcctctcttgcataccctcatgatggcccgacttcatttctgctctgaaaatattatccaccatttcttctgtgacatcaactctctcctccctctctcctgttctgataccagtctcaatcagttgatggttctgtctgtggtgggcctgatatttgtggtaccatcagtttgtatcttagcttcttatggtcgcattgtctctgctgtgatgaaaattacttcaatggaaggaaagctcaaagcattctcaacctgtggatcccaccttgctttggttattcttttttatggagccattgcaggaatctatatgagcccttcatccaaccattccactgaaaaagattcagctgcatcagtaatttttatggttgtagcccccgtattgaatcctttcatttacagcttaaggaacaatgagctgaaagggactttaaaaaagactcttggccagagcaaaatatgttccaagtga

SEQ ID NO: 20 - protein, 313 aa, Mus musculus
MEKENQTSVSEFLLLGFSSWPGHQGLLFALFLCLYLTGLFGNLLILLAIGSNNHLHTPMYFFLANLSLVDLCLPSATVPKMLLNIQTKSQSISYPGCLAQMYFCMMFANMDNFLLTVMAYDRFVAICHPLHYTTIMTPCLCTSLVAFSWVIATFNPLLHTLMMARLHFCSENIIHHFFCDINSLLPLSCSDTSLNQLMVLSVVGLIFVVPSVCILASYGRIVSAVMKITSMEGKLKAFSTCGSHLALVILFYGAIAGIYMSPSSNHSTEKDSAASVIFMVVAPVLNPFIYSLRNNELKGTLKKTLGQSKICSK

SEQ ID NO: 21 - DNA, 951 bp, Mus musculus
  CDS (1)..(948)
atgatgcttgatttgggaaacgagagttctgtgaccatgttcatcctttcgggattttcagaatatccacatctccatgcacctctttttctcctattttttatgatatatacagttactctaataggtaacctgggcataattgtggtcagaaaggtcaatcccaagctccacacacccatgtacttttttctcagtcatctttcatttttggatatttgttattccagtgtatttacacctaaactgctagaaatcttgattgtggaggacaggacaatctctttcaaaggatgcatgacacaatttttttttatttgtgcatttgtgattacggaaatgttcatgttagcagtgatggcctatgacaggtttgttgctgtgtgtaaccccttactctacacagtttcaatgtctcccaagctgtgtgcattccttgtagctggaacatatatgtggggtgtactctgttccttgacaattacatactctcttttgcaactatcctactgtggacctaacatcatcaaccactttggctgtgagtactctgccattctgtctttgtcctgttctgaccccaccttcagccaagtggtatgtttaaccatttctatattcaatgagacttgcagcctcctcatcatcctggcctcctatgtcttcatagttgtcacaatcattaagatgccttctaagggtggactccaaaaagctttctctacatgttcttcccacctgactgccatcagcatcttccatgggatcattctccttctttactgtgtacccaattccaaaaactcatggcttgtggtcaaagtggcaactgtgcttttcactgtcatgatccccatgcttaaccctctcatctacagccttaggaacaaagatgtaaaggggacagtcagcagactcatgcacttgaaacttcaagctcattcaacatga

SEQ ID NO: 22 - protein, 316 aa, Mus musculus
MMLDLGNESSVTMFILSGFSEYPHLHAPLFLLFFMIYTVTLIGNLGIIVVRKVNPKLHTPMYFFLSHLSFLDICYSSVFTPKLLEILIVEDRTISFKGCMTQFFFICAFVITEMFMLAVMAYDRFVAVCNPLLYTVSMSPKLCAFLVAGTYMWGVLCSLTITYSLLQLSYCGPNIINHFGCEYSAILSLSCSDPTFSQVVCLTISIFNETCSLLIILASYVFIVVTIIKMPSKGGLQKAFSTCSSHLTAISIFHGIILLLYCVPNSKNSWLVVKVATVLFTVMIPMLNPLIYSLRNKDVKGTVSRLMHLKLQAHST

SEQ ID NO: 23 - DNA, 60 bp, artificial sequence
  AL1 primer
attggatccaggccgctctggacaaaatatgaattctttttttttttttttttttttttt

SEQ ID NO: 24 - DNA, 26 bp, artificial sequence
  P26 primer
  modified_base (3)..(3): I
  modified_base (12)..(12): I
  modified_base (18)..(18): I
  modified_base (21)..(21): I
  modified_base (24)..(24): I
gcntaygaycgntaygtngcnatntg

SEQ ID NO: 25 - DNA, 26 bp, artificial sequence
  P27 primer
  modified_base (3)..(3): I
  modified_base (6)..(6): I
  modified_base (9)..(9): I
  modified_base (15)..(15): I
  modified_base (18)..(18): I
  modified_base (24)..(24): I
acnacnganagrtgnganscrcangt

SEQ ID NO: 26 - DNA, 48 bp, artificial sequence
  Anchor T primer
tatagaattcgcggccgctcgcgatttttttttttttttttttttttt

SEQ ID NO: 27 - DNA, 26 bp, artificial sequence
  The primer for Transmembrane domain 2(TM2)
  misc_feature (3)..(3): n is a, c, g, or t
  misc_feature (9)..(9): n is a, c, g, or t
  misc_feature (12)..(12): n is a, c, g, or t
  misc_feature (21)..(21): n is a, c, g, or t
ctncaymmnccnatgtayytnttyyt

SEQ ID NO: 28 - DNA, 26 bp, artificial sequence
  P41 primer
  modified_base (6)..(6): I
  misc_feature (6)..(6): n is a, c, g, or t
  modified_base (9)..(9): I
  misc_feature (9)..(9): n is a, c, g, or t
  modified_base (12)..(12): I
  misc_feature (12)..(12): n is a, c, g, or t
  modified_base (15)..(15): I
  misc_feature (15)..(15): n is a, c, g, or t
  modified_base (21)..(21): I
  misc_feature (21)..(21): n is a, c, g, or t
  modified_base (24)..(24): I
  misc_feature (24)..(24): n is a, c, g, or t
aarkcnttndmnacntgygsntcnca

SEQ ID NO: 29 - DNA, 33 bp, artificial sequence
  P42 primer
  modified_base (6)..(6): I
  misc_feature (6)..(6): n is a, c, g, or t
  modified_base (9)..(9): I
  misc_feature (9)..(9): n is a, c, g, or t
  modified_base (12)..(12): I
  misc_feature (12)..(12): n is a, c, g, or t
  modified_base (15)..(15): I
  misc_feature (15)..(15): n is a, c, g, or t
  modified_base (22)..(22): I
  misc_feature (22)..(22): n is a, c, g, or t
  modified_base (25)..(25): I
  misc_feature (25)..(25): n is a, c, g, or t
  modified_base (28)..(28): I
  misc_feature (28)..(28): n is a, c, g, or t
tcyytngtnytnrynckgatanatnatnggrtt

SEQ ID NO: 30 - DNA, 32 bp, artificial sequence
  W68 primer
  modified_base (3)..(3): I
  misc_feature (3)..(3): n is a, c, g, or t
  modified_base (9)..(9): I
  misc_feature (9)..(9): n is a, c, g, or t
  modified_base (12)..(12): I
  misc_feature (12)..(12): n is a, c, g, or t
  modified_base (15)..(15): I
  misc_feature (15)..(15): n is a, c, g, or t
  modified_base (21)..(21): I
  misc_feature (21)..(21): n is a, c, g, or t
  modified_base (27)..(27): I
  misc_feature (27)..(27): n is a, c, g, or t
tcnytrttncknagngwrtanatraanggrtt

SEQ ID NO: 31 - DNA, 32 bp, artificial sequence
  W69 primer
  modified_base (9)..(9): I
  misc_feature (9)..(9): n is a, c, g, or t
  modified_base (12)..(12): I
  misc_feature (12)..(12): n is a, c, g, or t
  modified_base (15)..(15): I
  misc_feature (15)..(15): n is a, c, g, or t
  modified_base (21)..(21): I
  misc_feature (21)..(21): n is a, c, g, or t
  modified_base (24)..(24): I
  misc_feature (24)..(24): n is a, c, g, or t
  modified_base (27)..(27): I
  misc_feature (27)..(27): n is a, c, g, or t
tcyttrttncknagngwrtanaynasnggrtt

SEQ ID NO: 32 - DNA, 32 bp, artificial sequence
  W 70 primer
  modified_base (3)..(3): I
  misc_feature (3)..(3): n is a, c, g, or t
  modified_base (9)..(9): I
  misc_feature (9)..(9): n is a, c, g, or t
  modified_base (12)..(12): I
  misc_feature (12)..(12): n is a, c, g, or t
  modified_base (15)..(15): I
  misc_feature (15)..(15): n is a, c, g, or t
  modified_base (21)..(21): I
  misc_feature (21)..(21): n is a, c, g, or t
  modified_base (24)..(24): I
  misc_feature (24)..(24): n is a, c, g, or t
  modified_base (27)..(27): I
  misc_feature (27)..(27): n is a, c, g, or t
tcntsrttncknarnsartanatnatnggrtt

SEQ ID NO: 33 - DNA, 27 bp, artificial sequence
  P8 primer
  modified_base (4)..(4): I
  misc_feature (4)..(4): n is a, c, g, or t
  modified_base (7)..(7): I
  misc_feature (7)..(7): n is a, c, g, or t
  modified_base (10)..(10): I
  misc_feature (10)..(10): n is a, c, g, or t
  modified_base (16)..(16): I
  misc_feature (16)..(16): n is a, c, g, or t
  modified_base (22)..(22): I
  misc_feature (22)..(22): n is a, c, g, or t
rttncknarnswrtanatraanggrtt

SEQ ID NO: 34 - DNA, 984 bp, Mus musculus
  CDS (1)..(984)<s300> AF1060071999-02-08(1)..(984)
atggagcgaaggaaccacactgggagagtgagtgaatttgtgttgctgggtttcccagctcctgccccactgcgggcactactatttttcctttctctgttggcctacgtgttggtgctgactgaaaacatactcatcattacagcaattaggaaccaccccaccctccacaaacccatgtattttttcttggctaatatgtcattcctggagatttggtatgtcactgttacgattcctaagatgcttgctggcttcattggttccgaggagaatcatggacagctgatctcctttgaggcatgcatgacacagctctactttttcctaggcttgggttgcacagagtgtgtccttcttgctgtcatggcctatgaccgctatgtggccatctgtcacccactccactatcctgtcattgtcagtagccggctatgtgtgcagatggcagctggatcctgggctggaggttttggtatctccatggttaaagttttcctcatttctcgcctgtcttactgtggccccaacaccatcaaccactttttctgtgatgtttctccattgctcaacttgtcatgcactgacatgtccacagcagagcttacagactttatcctggccatttttattctgctggggccactctctgtcactggggcttcctatatggccatcacaggtgcagtgatgcgcatcccctcagctgctggccgccataaggccttttcaacctgtgcctcccacctcactgttgtgattatcttctatgcagccagtattttcatctatgccaggcctaaggcactctcagcttttgacaccaacaagctggtctctgtactctacgctgtcattgtaccattgctcaatcccatcatctactgcttgcgcaatcaagaagtcaaaaaagccctacgtcgcactctgcacctggcccaaggccaggacgccaataccaagaaatccagcagagatggttag

SEQ ID NO: 35 - protein, 327 aa, Mus musculus
MERRNHTGRVSEFVLLGFPAPAPLRALLFFLSLLAYVLVLTENILIITAIRNHPTLHKPMYFFLANMSFLEIWYVTVTIPKMLAGFIGSEENHGQLISFEACMTQLYFFLGLGCTECVLLAVMAYDRYVAICHPLHYPVIVSSRLCVQMAAGSWAGGFGISMVKVFLISRLSYCGPNTINHFFCDVSPLLNLSCTDMSTAELTDFILAIFILLGPLSVTGASYMAITGAVMRIPSAAGRHKAFSTCASHLTVVIIFYAASIFIYARPKALSAFDTNKLVSVLYAVIVPLLNPIIYCLRNQEVKKALRRTLHLAQGQDANTKKSSRDG

SEQ ID NO: 36 - DNA, 1325 bp, Mus musculus
  CDS (138)..(1112)<s300> AF1219721999-04-25(1)..(1325)
aacacactcaaatcaaaataatattggattggttccatctggtttcagaatactcttgtgtttccttgtagaacttaagtttgacactcataaaaaccttcagacatattgaaagtaagggaattgggattaaactcatgtctctttttccccaaagaaatttagatgccatgaacagatcagcagcacatgtaaccgaatttgttctcttgggatttcctggttcctggaagatacagattttcctcttcgtgttgtttttggtgttttatgtcttgacattgttgggaaatggagccatcatctgtgcagtaagatgtgactcacgtctacatacccccatgtacttcctcctgggaaatttttccttccttgaaatctggtatgtttcctccactattcctaacatactagccaacattctgtctaagaccaaggccatctcattttcagggtgcttcctgcagttctatttcttcttttcactgggtacaactgaatgtctcttcctggcagtaatggcttatgataggtacctggccatttgccgcccattacattaccctactatcatgactaggaggctgtgttgcattctggtatcctcatgctggctcattggatttcttgggtacccaatccctatcttctccatttcccagcttcccttctgtggttctaatatcattgatcacttcctctgtgacatggacccattgatggctttgtcctgtgccccagctcctattactgaatttattttttatgcccaaagttcctttgtcctctttttcactattgcatacattcttcggtcctatattttgttgctcagggctgtttttcaggttccttctgcagctggccgacgaaaagccttctctacctgtggttcccatttagttgtggtgtcactcttctatggtacagtaatggtaatgtatgtgagtcctacatatggcattccaattttgatgcagaagatccttacacttgtatactctgtaatgactcctctctttaatcctctgatttatagccttcgtaacaaggacatgaaacttgctctgagaaatgttttgttaggaatgagaattgtcaaaaatatgtaattcaaagctgtttcatactcacatgttctaataaagaaaaaactggagatgaatcaattcattcagttgtctttaccctttgttctatgtttttgagacactgtctcatgtggccctggctagcctcaaactcattctctagccaaggatgaccttgcaaagatcacttatgtatactctcatatcatctgccaatagtgataccttgacctc

SEQ ID NO: 37 - protein, 324 aa, Mus musculus
MSLFPQRNLDAMNRSAAHVTEFVLLGFPGSWKIQIFLFVLFLVFYVLTLLGNGAIICAVRCDSRLHTPMYFLLGNFSFLEIWYVSSTIPNILANILSKTKAISFSGCFLQFYFFFSLGTTECLFLAVMAYDRYLAICRPLHYPTIMTRRLCCILVSSCWLIGFLGYPIPIFSISQLPFCGSNIIDHFLCDMDPLMALSCAPAPITEFIFYAQSSFVLFFTIAYILRSYILLLRAVFQVPSAAGRRKAFSTCGSHLVVVSLFYGTVMVMYVSPTYGIPILMQKILTLVYSVMTPLFNPLIYSLRNKDMKLALRNVLLGMRIVKNM

SEQ ID NO: 38 - DNA, 1134 bp, Mus musculus
  misc_feature (99)..(99): n is a, c, g, or t
  CDS (106)..(1056)<s300> AF1219801999-04-25(1)..(1134)
ccagtccagcctggtaggctgggcaggtcctacaggtctttcagggactgaacccggcatcctgcccctcccctctccctggagcctccctagccctcnggcgtcatgttgggttggagcaatggcacctacaatgagtcctacaccagcttcctcctcatgggcttcccagggatgcaggaagccagagccctcctggtgctgcccttcctcagcctctacctggtgatcctcttcaccaatgccctggtcatccacacggtggcatcccagcgcagcctgcaccagcccatgtacctgctcattgccctgctcctggctgtcaatatctgtgctgccaccacggtgctgccccccatgctcttcagcttctccacacgcttcaaccgcatctccctccctcgatgcttgggacagatgttctgcatctactttctggtttctatggactgcaacatcctcctggtcatggctctagatcgctatgtggctatctgctaccctctccgctacccagaaatagtgacaggacagttactggctggtctggtggtgttggcagtcaccaggagcacaagcattgttgctccagtggtggtgctggcctcgcgggttcgcttctgccgctcagatgtgatccgccactttgcctgtgagcacatggccctgatgaagctctcctgtggagacatctcgctgaataaaacggcgggactcattattcgaacctttaatagagtcctggatatgctccttctaggcacctcctactcccgcatcatccatgctgccttcaggatctcatcaggtggagcacggtccaaagccctgaacacctgtggttcccacctgctggtcatcttcaccgtctactcctccaccatgtcctcatccattgtctaccgtgtggctcgcactgcctcccaagatgtgcacaacctgctcagtgctttctatctgttgctcccgtgtctggtcaaccccatcatctacggggccagaaccaaggaaatcaggcagcacctggtaaggtcattcctgagtgcaggcccctgactctcctatgatcagtccgtgttggcccctcagtattcctggtgaaactgaggaaggaagaaatggagtcagagggac

SEQ ID NO: 39 - protein, 316 aa, Mus musculus
MLGWSNGTYNESYTSFLLMGFPGMQEARALLVLPFLSLYLVILFTNALVIHTVASQRSLHQPMYLLIALLLAVNICAATTVLPPMLFSFSTRFNRISLPRCLGQMFCIYFLVSMDCNILLVMALDRYVAICYPLRYPEIVTGQLLAGLVVLAVTRSTSIVAPVVVLASRVRFCRSDVIRHFACEHMALMKLSCGDISLNKTAGLIIRTFNRVLDMLLLGTSYSRIIHAAFRISSGGARSKALNTCGSHLLVIFTVYSSTMSSSIVYRVARTASQDVHNLLSAFYLLLPCLVNPIIYGARTKEIRQHLVRSFLSAGP

SEQ ID NO: 40 - DNA, 1421 bp, Mus musculus
  CDS (291)..(1310)<s300> AF1219761999-12-25(1)..(1421)
agaaagatttcaggagtccttaaagacggcacagaaaaccggtacagactgcaccattcagctgaaagccagacgtaacagcaccacggtggtggtgaacacggtgggctcagagaatccggataagcctgcttttttatactaagttggcattataaaaaagcattgcttatcaatttgttgcaacgaacaggtcactatcagtcaaaataaaatcattatttgatttcaattttgtcccactccctgcctctgtcatcacgatactgtgatgccatggtgtccgacttatgcccgagaagatgttgagcaaacttatcgcttatctgcttctcatagagtcttgcagacaaactgcgcaactcgtgaaaggtaggcggatctgggtcgactctaggcctcactggcctaatacgactcactatagggagctcgaggatcagcatgtttggattgctattcccttctgctccatgtacatccttgctctggttggaaatggtaccatcctctatatcattataacagacagggctctccatgagccaatgtacctcttcttgtgtctgctttctatcactgatctggttctctgttcaacaacattgcctaaaatgctggcaatattctggctcagatcccatgtcatttcctaccatggctgcctcactcagatgttttttgtacatgcagtctttgccacagagtcagctgttctgctggccatggcttttgatcgatatgttgctatctgcagaccactccactatacatccatcctcaatgctgttgtaattgggaagattggcctggcatgcgtgactcgtggccttctctttgtcttcccctttgtcattctcattgaacgtttacccttctgtggacatcatataatccctcacacttactgtgagcacatgggcatagccaagctcgcctgtgccagcatcaagcctaacaccatctatggtcttactgtagcactttcagtcactggcatggatgtggtcctcattgcaacctcctacatcctgattctgcaggccgtgctgcgactgccctcaaaggatgcccagttccgagcattcagcacatgtggagcccacatttgtgtaattcttgtcttctatatccccgcattcttttcatttttcactcaccgctttggtcaccacgtgcctcctcaggtacacatcatacttgcaaatctttatctccttgtgcctcctgttctcaaccccctagtctatggcatcaataccaaacaaatccgcctgagaatacttgacttttttgtaaagagaaggtgacaataatctccacatataccaaaggctaatgagttcctggctttagtttgctgcttctgctgatctcagtaagtcagtgtatgtacatttaagattttgagatctagagca

SEQ ID NO: 41 - protein, 339 aa, Mus musculus
MPEKMLSKLIAYLLLIESCRQTAQLVKGRRIWVDSRPHWPNTTHYRELEDQHVWIAIPFCSMYILALVGNGTILYIIITDRALHEPMYLFLCLLSITDLVLCSTTLPKMLAIFWLRSHVISYHGCLTQMFFVHAVFATESAVLLAMAFDRYVAICRPLHYTSILNAVVIGKIGLACVTRGLLFVFPFVILIERLPFCGHHIIPHTYCEHMGIAKLACASIKPNTIYGLTVALSVTGMDVVLIATSYILILQAVLRLPSKDAQFRAFSTCGAHICVILVFYIPAFFSFFTHRFGHHVPPQVHIILANLYLLVPPVLNPLVYGINTKQIRLRILDFFVKRR

SEQ ID NO: 42 - DNA, 930 bp, M.musculus
  CDS (1)..(930)<s300> X929691996-07-01(1)..(930)
atgcagagaaataacttcactgaagtgatagagttcgtcttcctgggattctccagctttggaaagcatcagataaccctctttgtggttttcctaaccatctacattttaactctggctggcaacatcattatagtgacaatcacacacatagaccaccaccttcacactcccatgtacttctttctgagcatgttggcaagctcagagactgtgtacacactggtcattgtcccacgaatgctttccagcctgattttttacaaccttcccatatccttggcaggctgcgcaacccaaatgttcttttttgtcactttggccaccaacaactgctttctgctcacagcaatgggttatgatcgttatgtggctatttgtaatcctctgagatatacaatcatcatgagcaagggaatgtgtgccttgttggtctgtgggtctttaggcactggcctggttatggcagttcttcatgtgccagccatgttccatttgcccttttgtggcacggtggtggagcactttttctgtgacatatacccagtaatgaagctttcttgtgttgataccactgtcaatgagataatcaattatggtgtaagttcatttgtaattcttgtgcccatagggctgatatttatctcctatgtgctcattgtctcttccatccttaaaattgtgtccactgaaggccagaagaaagcctttgccacctgtgcctctcatctcactgtggtcattgtccactatggctgtgcctccattgcctacctcaaacccaaatcagaaagttcagtagaaaaagaccttcttctctctgtgacctacactatcatcactcccttgctgaaccctgttgtctacagcctcaggaacaaagaagtcaaagatgctctatgcagagctgtgggcagaaacacttcttaa

SEQ ID NO: 43 - protein, 309 aa, M.musculus
MQRNNFTEVIEFVFLGFSSFGKHQITLFVVFLTIYILTLAGNIIIVTITHIDHHLHTPMYFFLSMLASSETVYTLVIVPRMLSSLIFYNLPISLAGCATQMFFFVTLATNNCFLLTAMGYDRYVAICNPLRYTIIMSKGMCALLVCGSLGTGLVMAVLHVPAMFHLPFCGTVVEHFFCDIYPVMKLSCVDTTVNEIINYGVSSFVILVPIGLIFISYVLIVSSILKIVSTEGQKKAFATCASHLTVVIVHYGCASIAYLKPKSESSVEKDLLLSVTYTIITPLLNPVVYSLRNKEVKDALCRAVGRNTS

SEQ ID NO: 44 - DNA, 957 bp, Mus musculus
  CDS (1)..(957)<s300> AB0612292001-09-07(1)..(957)
atgatactgtctgaaaaaaacaatagtgggattattttcaccctcttgggcttctcagattatcctgaccttaaagtccctctcttcttggtgtttctcgtcatttacagcatcactgtggtaggaaatattggtatgatcctcgtgatcagaattaatccccaactgcactcccctatgtacttcttcctcagccacctctcctttgtggatttctgctattcttcgatcattgctcccaagatgctggtgaaccttgttgcaaaagacataaccatttcatttgtagaatgcatagtacaatattttttattttgtgtctttgtagtaactgaagcctttttattagtggttatggcatatgaccgatttgtggctatctgtaaccctctgctctacacagtagccatgtcccagaaactctgtatcacactggtggtgggatcctacgcatgggggttcacatgttccttgacactgacgtgttctactgtgcaattatcttttcatggtgtcaataggatcgatcacttcttctgtgaactctcttcactgctagccctttcttcctctgatactctcatcagtcaattactgctgtttgtctttgccacatttaatgctgtcagcacattactccttattctgttgtcttacctgttcattgttgtcactgttcttaagatgcgttcagccagtgggcgtcgtaaggctttctccacctgtgcatcccatctggcagccatcactatcttccatggtaccattttattccttttttgtgttcccaactctaagaattccaggctcacagtcaaagtgggctctgtgttttacacagtggtgatccccatgcttaaccccataatctatagtctgagaaataaggatgtccaagatactattagaaaaataatgacccttatctcatgtgttaagaatgatagacacaattaa

SEQ ID NO: 45 - protein, 318 aa, Mus musculus
MILSEKNNSGIIFTLLGFSDYPDLKVPLFLVFLVIYSITVVGNIGMILVIRINPQLHSPMYFFLSHLSFVDFCYSSIIAPKMLVNLVAKDITISFVECIVQYFLFCVFVVTEAFLLVVMAYDRFVAICNPLLYTVAMSQKLCITLVVGSYAWGFTCSLTLTCSTVQLSFHGVNRIDHFFCELSSLLALSSSDTLISQLLLFVFATFNAVSTLLLILLSYLFIVVTVLKMRSASGRRKAFSTCASHLAAITIFHGTILFLFCVPNSKNSRLTVKVGSVFYTVVIPMLNPIIYSLRNKDVQDTIRKIMTLISCVKNDRHN

SEQ ID NO: 46 - DNA, 1344 bp, Mus musculus
  CDS (61)..(1020)<s300> AJ1334242003-02-01(1)..(1344)
ggaggaagacaatgttgatgctgattgctgagttcctgcaggtttcaaaccgaatgtaccatggacagatccaatgagaccgcccccctgtccggcttcattctcctgggcctctctgcccacccaaagctggagaaaaccttcttcgtgctcatcctgatgatgtacctggtgatcctgctgggcaacggcgtcctcatcctggtgagcatcctcgactcccacctgcacacgcccatgtacttcttcctggggaacctctccttcctggacatctgctacactacctcctctgtccccctcattctggacagctttctgactcccaggaagaccatctccttctcgggctgtgccgtgcagatgtttctctccttcgccatgggagccacggagtgtgtgctcctgagtatgatggcgtttgatcgttatgtggccatctgcaacccccttagatatcctgtggtcatgaacaaggctgcctatgtgcccatggctgccagttcctgggcaggtggtatcactaattctgtagtgcagacatctttggcaatgcggctgcccttctgtggggacaatgtcatcaatcacttcacctgtgagatcctggcagtcctgaaactggcctgtgctgacatctccatcaatgtcatcagcatggttgtggccaacatgatcttcttggcagtcccagtcctcttcatctttgtctcctatgtcttcatccttgtgacaatcctgaggatcccctctgctgaggggaggaagaaggccttctccacctgctctgcccacctcaccgtggtacttgtcttctatggaaccatcctcttcatgtacgggaagcccaagtccaaggacccactgggggcagacaagcaggaccttgcagacaagctcatctccctcttctatggagtggtgacccccatgctaaaccccatcatctacagcttgagaaacaaggacgtgagggctgctgtgaggaacctggtgggccagaaacacctaactgagtgactgtcacagtgcagaacttccaacctcttcattgtgtttgtgagggaagagtggtgcaatgaagaggagccacttccccaaggtccaagtaatgaactcagaactaagactataaacaaactatcaacgttccttaagcaccaatgcttctagttaacaggctggaaggacaagcctttacacctttggagagaatggctggttgtcagctttgtgttcaaccttagtggcgtcgtagaactactctttcatgaccagaggctggcacagatctctggaaagatgctgacatgcataactaggagacagatgcaaagcctggtt

SEQ ID NO: 47 - protein, 319 aa, Mus musculus
MDRSNETAPLSGFILLGLSAHPKLEKTFFVLILMMYLVILLGNGVLILVSILDSHLHTPMYFFLGNLSFLDICYTTSSVPLILDSFLTPRKTISFSGCAVQMFLSFAMGATECVLLSMMAFDRYVAICNPLRYPVVMNKAAYVPMAASSWAGGITNSVVQTSLAMRLPFCGDNVINHFTCEILAVLKLACADISINVISMVVANMIFLAVPVLFIFVSYVFILVTILRIPSAEGRKKAFSTCSAHLTVVLVFYGTILFMYGKPKSKDPLGADKQDLADKLISLFYGVVTPMLNPIIYSLRNKDVRAAVRNLVGQKHLTE

SEQ ID NO: 48 - DNA, 942 bp, Mus musculus
  CDS (1)..(942)<s300> AF1025231999-02-08(1)..(942)
atggcgaacagcactactgttactgagtttattttgctggggctgtcagatgcctgtgagctgcaggtgctcatattcctgggctttctcctgacctacttcctcattctgctgggaaacttcctcatcatcttcatcacccttgtggacaggcgcctttacacccccatgtattacttcctccgcaactttgccatgctggagatctggttcacctctgtcatcttccccaagatgctaaccaacatcatcacaggacataagaccatctccctactaggttgtttcctccaagcattcctctatttcttccttggcaccactgagttctttctactggcagtgatgtcctttgacaggtatgtggccatttgtaaccctttgcgttatgccaccattatgagcaaaagagtctgtgtccagcttgtgttttgctcatggatgtctggattgcttctcatcatagttcctagttcaattgtatttcagcagccattctgtggcccaaacatcattaatcatttcttctgtgacaactttccacttatggaactcatatgtgcagatactagcctggtagagttcctgggttttgttattgccaatttcagcctcctgggcactctggctgtgactgccacctgctatggccacattctctataccattctacacattccttcagccaaggagaggaagaaagccttctcaacttgctcctctcatattattgtggtgtctctcttctacggcagctgtatcttcatgtatgtccggtctggcaagaatggacagggggaggatcataacaaggtggtggcattgctcaacactgtagtgacacccacactcaaccccttcatctacactctgaggaacaagcaggtgaagcaggtatttagggaacacgtaagcaagttccaaaagttcagccagacgtga

SEQ ID NO: 49 - protein, 313 aa, Mus musculus
MANSTTVTEFILLGLSDACELQVLIFLGFLLTYFLILLGNFLIIFITLVDRRLYTPMYYFLRNFAMLEIWFTSVIFPKMLTNIITGHKTISLLGCFLQAFLYFFLGTTEFFLLAVMSFDRYVAICNPLRYATIMSKRVCVQLVFCSWMSGLLLIIVPSSIVFQQPFCGPNIINHFFCDNFPLMELICADTSLVEFLGFVIANFSLLGTLAVTATCYGHILYTILHIPSAKERKKAFSTCSSHIIVVSLFYGSCIFMYVRSGKNGQGEDHNKVVALLNTVVTPTLNPFIYTLRNKQVKQVFREHVSKFQKFSQT

SEQ ID NO: 50 - DNA, 669 bp, Mus musculus
  CDS (1)..(669)<s300> AF1025311999-02-08(1)..(669)
tgcaacttagcgaccatggatattatctgcacctcctctgtactgcccaaggcgctggttggtctactgtctgaggaaaacaccacctccttcaaagggtgcatgactcagctcttctttcttgtgtggtctggatcctctgagctgctgctgctcacagtcatggcctatgaccgctatgtggccatctgtttgcccctgcattacagctctaggatgagtccacagctctgtgggacctttgccgtgggtgtatggtccatctgcgcactaaatgcatctatcaacactggtctgatgacacggctgtcattctgtggccccaaggtcatcacccacttcttctgtgagattcccccactcctcctgctctcctgtagtcctacatatataaatagcgttatgactcttgtggcagatgccttttatggaggcatcaattttttacttaccttgctatcctatggctgcatcattgccagcatcctgcgcatgcgttctgctgagggcaagaggaaggccttttctacctgctcatcccacctcattgtggtctctgtgtactactcatctgtgttctgtgcctatgtcagccctgcttctagctacagcccagaaagaagcaaagtttcctcagtgctgtactcagtcctcagcccaaccctcaac

SEQ ID NO: 51 - protein, 223 aa, Mus musculus
CNLATMDIICTSSVLPKALVGLLSEENTTSFKGCMTQLFFLVWSGSSELLLLTVMAYDRYVAICLPLHYSSRMSPQLCGTFAVGVWSICALNASINTGLMTRLSFCGPKVITHFFCEIPPLLLLSCSPTYINSVMTLVADAFYGGINFLLTLLSYGCIIASILRMRSAEGKRKAFSTCSSHLIVVSVYYSSVFCAYVSPASSYSPERSKVSSVLYSVLSPTLN

SEQ ID NO: 52 - DNA, 1661 bp, Mus musculus
  misc_feature (3)..(3): n is a, c, g, or t
  CDS (303)..(1307)<s300> AF1219741999-04-25(1)..(1661)
gtntacatagtgagttcgaggccagccagggctacacagacaaaccctgtctcgaaaaaccaaaaaaaaaaaaaaaaaaaagaattcattaatgaaaaagaagggggaaaatggagggccatggaaagtagctacttctaacatacaactcttcatttcctccatagaaatgctgtagttaatgtctacacccagtccagcctggtgaggctggggcaggtcctagcagggcctttcagggactgaaccccggcatcctgcccctcccctctccctggagcctccccaagccctcaggcgtcatgtcagggtggagcaatggcacctacaatgagtcctacaccagcttcctcctcatgggcttcccagggatgcaggaagccagagccctcctggtgctgcccttcctcagcctctacctggtgatcctcttcaccaatgccctggtcatccacacggtggcatcccagcgcagcctgcaccagcccatgtacctgctcattgccctgctcctggctgtcaatatctgcgctgccaccaccgtggtgccccccatgctcttcagcttctccacacgcttcaaccgcatctccctccctcgatgcttgggacaaatgttctgcatctacttccttattgtctttgactgcaacatcctcctggtcatggctctagatcgctatgtggctatctgctaccctctccgctacccagaaatagtgacaggacagttactggctggtctggtggtgctggcagtcaccaggagcacaagcattgttgctccagtggtggtgctggcctcgcgggttcgcttctgtcgctcagatgtgatccgccactttgcctgtgagcacatggccctgatgaagctttcctgtggggacatctcgctgaataagacggtgggactcactgttcgcatcttcaaccgagtcctggatatgctcctgttaggtgcctcctactcccgcatcatccatgctgccttcaggatctcatcaggtggagcacggtccaaagccctgaacacctgtggctcccacctgctggtcatcttcaccgtctactcctccaccatgtcctcatccattgtctaccgtgtggcacgcactgcctcccaagatgtgcacaacttgcttagtgctttctatctgttgctcccctgtctggtcaaccccatcatctacggggccagaaccaaggaaatcaggcagcacctggtagctctgttccaaaggactcagcaacaggtcttcactgagaagccccagtccctgccctcgaatagagagcttcctggatgattgtccagaatttgtgggtctcaaaatcactttcactattcagtgaaggaggggcattcaagtgggcattcgtctctggtatattttgtctcggctattttagttcagcatcctatttatgagaagggtctattctatatctccagctgtctagaactccttaagtggcccaggatgacctggaacccaaacaattctcctttcttagtttgccaaatgctagcattagaggcatgagtcacagtgcctggcttatctgcactcatactggagagcctcatgtctgctttccaaaaagcacctactcactctgaactagcaactgaaagcaagctctaaccctggcttgaagtt

SEQ ID NO: 53 - protein, 334 aa, Mus musculus
MSGWSNGTYNESYTSFLLMGFPGMQEARALLVLPFLSLYLVILFTNALVIHTVASQRSLHQPMYLLIALLLAVNICAATTVVPPMLFSFSTRFNRISLPRCLGQMFCIYFLIVFDCNILLVMALDRYVAICYPLRYPEIVTGQLLAGLVVLAVTRSTSIVAPVVVLASRVRFCRSDVIRHFACEHMALMKLSCGDISLNKTVGLTVRIFNRVLDMLLLGASYSRIIHAAFRISSGGARSKALNTCGSHLLVIFTVYSSTMSSSIVYRVARTASQDVHNLLSAFYLLLPCLVNPIIYGARTKEIRQHLVALFQRTQQQVFTEKPQSLPSNRELPG

SEQ ID NO: 54 - DNA, 1116 bp, Mus musculus
  misc_feature (15)..(15): n is a, c, g, or t
  CDS (50)..(1015)<s300> AF1219751999-04-25(1)..(1116)
caagctggctcttcntactgtctctccattagttttagtcgtcacgggaatgaattcaaaagcaagcatgcttggaactaacttcactatcatccatccaactgtgttcatcctgcttggaatcccagggctggagcagtaccacacctggctttctattcctttttgtcttatgtacattgctgcagtcttggggaacggagccctcatccttgttgtcctgagtgaacgcaccctccatgagcccatgtatgtctttctgtccatgctggctggcactgatattctcctgtcaaccaccactgtgcctaagaccttggctatcttttggttccatgctggggagatcccctttgatgcctgcattgctcagatgtttttcatccacgttgcttttgtggctgagtcgggaatccttctggccatggcatttgaccgatatgtggctatttgtactcctctgagatactcagccgtcttaacacctatggcaattggaaaaatgaccctggccatctggggacggagcattgggacaattttccctatcatatttctgctgaagaggctgtcatactgcaggaccaatgtcatcccacactcatattgtgagcatattggtgtagccagattggcttgtgctgacatcactgtcaatatctggtatggcttctcggtgccaatggcttcagttttggtagatgttgcactcattggtatttcttatacgttgatcctccaggctgtgtttagacttccttcccaggatgctaggcacaaggccctcaatacctgtggttctcacattggggtcattctcctctttttcataccatcattttttactttccttactcatcgctttggcaagaacatcccccaccatgtgcacattcttctggcaaatctctatgtgttggttccccccatgcttaaccctatcatctatggtgctaagaccaagcaaattagggacagcatgactcgcatgttgtctgttgtgtggaagtcttgagagcagtcacagttcacaaagctgtcttagtttctcttacaaacaggagagagagagagagagagagagagagagagagagagagagagagagagagagag

SEQ ID NO: 55 - protein, 321 aa, Mus musculus
MNSKASMLGTNFTIIHPTVFILLGIPGLEQYHTWLSIPFCLMYIAAVLGNGALILVVLSERTLHEPMYVFLSMLAGTDILLSTTTVPKTLAIFWFHAGEIPFDACIAQMFFIHVAFVAESGILLAMAFDRYVAICTPLRYSAVLTPMAIGKMTLAIWGRSIGTIFPIIFLLKRLSYCRTNVIPHSYCEHIGVARLACADITVNIWYGFSVPMASVLVDVALIGISYTLILQAVFRLPSQDARHKALNTCGSHIGVILLFFIPSFFTFLTHRFGKNIPHHVHILLANLYVLVPPMLNPIIYGAKTKQIRDSMTRMLSVVWKS

SEQ ID NO: 56 - DNA, 1267 bp, Mus musculus
  misc_feature (108)..(108): n is a, c, g, or t
  CDS (172)..(1200)<s300> AF1219771999-04-25(1)..(1267)
tctattgctcactgaaatataaactagcaacatgaagaacatatgattgaactatatcaaagaaacaaatttttctaatcataaatgaccatgaatcattgaatttcntaagctgaagttctttcatgaggtaccacacaacagcatgttcctgtacacatgtaactacctatgttttgtcatttatataatgagaacaatatgcaagtggcaatcctggattccattctaataccttcttatttttctttcctgacagagatggagcctggaaactacacagttgtaacagaattcattcttttagggttaacagatgatattacagtcagtgtcattttatttgttatgtttctaatcgtctattctgttactttaatgggtaacttgaacataattgtgctaatcagaaccagccctcagcttcacacccccatgtaccttttccttagccatttggcctttctagacattgggtactccagctcagttacacccatcatgctgaggggctttctcagaaagggaacatttatccctgtggctggctgtgtggctcaactctgtattgtggtggcatttgggacatctgaatctttcttgctagcttccatggcctatgaccgctatgtggccatctgctcacctttgctctactcaacacagatgtcctccacagtctgcatcctcctagttggaacttcctacctaggtggatgggtgaatgcttggatatttactggttgctccttaaatctgtcattttgtgggccaaataaaattaatcactttttctgtgactattcaccactattgaagctttcttgttctcatgacttttcttttgaagtcattccagcaatctcttcgggatccatcattgtggtcactgtgtttatcattgctctgtcttatgtctacatccttgtgtcaatcctgaagatgcgctctactgaaggtcgccagaaggccttctccacctgcacttcccacctcactgcagtcactctgttctttgggaccatcacattcatttatgtgatgccccagtccagctactccacagaccagaacaaagtggtgtctgtgttttacacagtggtgatccccatgttgaatcccctcatctacagtttcagaaacaaagaggttaaagaagccatgaaaaaactgattgctaaaacacattggtggtcctgaaatatttgaatttacaaacagtaaattctgctcttacaggtaaatggcagtatactaagtaaattac

SEQ ID NO: 57 - protein, 342 aa, Mus musculus
MFCHLYNENNMQVAILDSILIPSYFSFLTEMEPGNYTVVTEFILLGLTDDITVSVILFVMFLIVYSVTLMGNLNIIVLIRTSPQLHTPMYLFLSHLAFLDIGYSSSVTPIMLRGFLRKGTFIPVAGCVAQLCIVVAFGTSESFLLASMAYDRYVAICSPLLYSTQMSSTVCILLVGTSYLGGWVNAWIFTGCSLNLSFCGPNKINHFFCDYSPLLKLSCSHDFSFEVIPAISSGSIIVVTVFIIALSYVYILVSILKMRSTEGRQKAFSTCTSHLTAVTLFFGTITFIYVMPQSSYSTDQNKVVSVFYTVVIPMLNPLIYSFRNKEVKEAMKKLIAKTHWWS

SEQ ID NO: 58 - DNA, 1120 bp, Mus musculus
  CDS (84)..(1040)
  misc_feature (940)..(940): n is a, c, g, or t
  misc_feature (1083)..(1083): n is a, c, g, or t <s300> AF121979 1999-04-25 (1)..(1120)
tgtcattattagtgctgataaagtgttgtcaagtcctgtgagattccttcaaatgaatatgtccatcagaggctcctgacaacatgtcaccaggcaacagctcatggattcatccttcttccttcctgctcttgggaatcccaggactggaagagttgcagttctggcttggtttgccatttggaacagtctatcttattgctgtcctagggaatgtcatcattctctttgtaatctatctagagcacagccttcaccaacctatgttctacttactggccatactggctgttactgacttgggtctgtctacagcaactgttcccagagcactcggtatattctggtttggcttccataagattgcctttagggactgtgtagctcaaatgtttttcatacatctgtttacaggcatcgaaacattcatgcttgtagctatggcctttgatcgctacattgccatctgtaaccctctccgatataacactatcctcaccaacagaacaatctgcattattgttggagttggactatttaaaaatttcattttggtttttccacttatatttctcattctaaggctttcattctgtggacacaatatcataccacacacatactgtgagcacatgggcattgctcgactggcatgcgtcagcatcaaggttaatgtattatttggattaatactcatatctatgatacttctggatgttgttttgagtgctctgtcctatgcgaaaattcttcatgctgtatttaaactcccatcctgggaagccagactcaaagctcttaatacctgtggttcccatgtgtgtgtgatcttggctttcttcactccagcctttttctccttcttgactcatcgatttggacacaatattccacgatatatccacatcctccttgctaacttatatgtgatcattccccnggctcttaaccctattatttatggggtgagaaccaaacagatacaagatcgtgcggtgacaatattgtgcaacgaggttggacagctggcagacgactagtatgtcttctaatagtctctttccttcctaagaggactactgntttgtaagcttgcatacgtggaacacattacacaatg

SEQ ID NO: 59 - protein, 318 aa, Mus musculus
  misc_feature (286)..(286): The ′Xaa′ at location 286 stands for Gln, Arg, Pro, or Leu.
MSPGNSSWIHPSSFLLLGIPGLEELQFWLGLPFGTVYLIAVLGNVIILFVIYLEHSLHQPMFYLLAILAVTDLGLSTATVPRALGIFWFGFHKIAFRDCVAQMFFIHLFTGIETFMLVAMAFDRYIAICNPLRYNTILTNRTICIIVGVGLFKNFILVFPLIFLILRLSFCGHNIIPHTYCEHMGIARLACVSIKVNVLFGLILISMILLDVVLSALSYAKILHAVFKLPSWEARLKALNTCGSHVCVILAFFTPAFFSFLTHRFGHNIPRYIHILLANLYVIIPXALNPIIYGVRTKQIQDRAVTILCNEVGQLADD

SEQ ID NO: 60 - DNA, 2532 bp, Mus musculus
  CDS (1)..(2532)<s300> AY0326232002-02-05(1)..(2532)
atgggaccccaggcgaggacactccatttgctgtttctcctgctgcatgctctgcctaagccagtcatgctggtagggaactccgactttcacctggctggggactacctcctgggtggcctctttaccctccatgccaacgtgaagagtgtctctcacctcagctacctgcaggtgcccaagtgcaatgagtacaacatgaaggtgttgggctacaacctcatgcaggccatgcgattcgccgtggaggaaatcaacaactgtagctctttgctgcccggcgtgctgctcggctacgagatggtggatgtctgctacctctccaacaatatccagcctgggctctacttcctgtcacagatagatgacttcctgcccatcctcaaagactacagccagtacaggccccaagtggtggctgttattggcccagacaactctgagtctgccatcaccgtgtccaacattctctcctacttcctcgtgccacaggtcacatatagcgccatcaccgacaagctgcaagacaagcggcgcttccctgccatgctgcgcactgtgcccagcgccacccaccacatcgaggccatggtgcaactgatggttcacttccagtggaactggatcgtggtgctggtgagcgatgacgattatggccgagagaacagccacctgctgagccagcgtctgaccaacactggcgacatctgcattgccttccaggaggttctgcccgtaccagaacccaaccaggctgtgaggcctgaggagcaggaccaactggacaacatcctggacaagctgcggcggacttcggcgcgtgtggtggtgatattctcgccggagctgagcctgcacaacttcttccgtgaggtgctgcgctggaacttcacgggctttgtgtggattgcctctgagtcctgggccatcgaccctgttctacacaacctcacagagctgcgccacacgggcactttcctgggtgtcaccatccagagggtgtccatccctggcttcagccagttccgagtgcgccatgacaagccagggtatcgcatgcctaacgagaccagcctgcggactacctgtaaccaggactgcgacgcctgcatgaacatcactgagtccttcaacaacgttctcatgctttcgggggagcgtgtggtctacagcgtgtactcggccgtctacgcggtggcccacaccctccacagactcctccactgcaatcaggtccgctgcaccaagcaaatcgtctatccatggcagctactcagggagatctggcatgtcaacttcacgctcctgggcaaccagctcttcttcgacgaacaaggggacatgccgatgctcctggacatcatccagtggcagtggggcctgagccagaaccccttccaaagcatcgcctcctactcccccaccgagacgaggctgacctacattagcaatgtgtcctggtacacccccaacaacacggtccccatatccatgtgttctaagagttgccagcctgggcaaatgaaaaaacccataggcctccacccatgctgcttcgagtgtgtggactgtccgccggacacctacctcaaccgatcagtagatgagtttaactgtctgtcctgcccgggttccatgtggtcttacaagaacaacatcgcttgcttcaagcggcggctggccttcctggagtggcacgaagtgcccactatcgtggtgaccatcctggccgccctgggcttcatcagtacgctggccattctgctcatcttctggagacatttccagacgcccatggtgcgctcggcgggcggccccatgtgcttcctgatgctggtgcccctgctgctggcgttcgggatggtccccgtgtatgtgggcccccccacggtcttctcctgtttctgccgccaggctttcttcaccgtttgcttctccgtctgcctctcctgcatcacggtgcgctccttccagattgtgtgcgtcttcaagatggccagacgcctgccaagcgcctacggtttctggatgcgttaccacgggccctacgtcttcgtggccttcatcacggccgtcaaggtggccctggtggcgggcaacatgctggccaccaccatcaaccccattggccggaccgaccccgatgaccccaatatcataatcctctcctgccaccctaactaccgcaacgggctactcttcaacaccagcatggacttgctgctgtccgtgctgggtttcagcttcgcgtacgtgggcaaggaactgcccaccaactacaacgaagccaagttcatcaccctcagcatgaccttctccttcacctcctccatctccctctgcacgttcatgtctgtccacgatggcgtgctggtcaccatcatggatctcctggtcactgtgctcaactttctggccatcggcttggggtactttggccccaaatgttacatgatccttttctacccggagcgcaacacttcagcttatttcaatagcatgattcagggctacacgatgaggaagagctag

SEQ ID NO: 61 - protein, 843 aa, Mus musculus
MGPQARTLHLLFLLLHALPKPVMLVGNSDFHLAGDYLLGGLFTLHANVKSVSHLSYLQVPKCNEYNMKVLGYNLMQAMRFAVEEINNCSSLLPGVLLGYEMVDVCYLSNNIQPGLYFLSQIDDFLPILKDYSQYRPQVVAVIGPDNSESAITVSNILSYFLVPQVTYSAITDKLQDKRRFPAMLRTVPSATHHIEAMVQLMVHFQWNWIVVLVSDDDYGRENSHLLSQRLTNTGDICIAFQEVLPVPEPNQAVRPEEQDQLDNILDKLRRTSARVVVIFSPELSLHNFFREVLRWNFTGFVWIASESWAIDPVLHNLTELRHTGTFLGVTIQRVSIPGFSQFRVRHDKPGYRMPNETSLRTTCNQDCDACMNITESFNNVLMLSGERVVYSVYSAVYAVAHTLHRLLHCNQVRCTKQIVYPWQLLREIWHVNFTLLGNQLFFDEQGDMPMLLDIIQWQWGLSQNPFQSIASYSPTETRLTYISNVSWYTPNNTVPISMCSKSCQPGQMKKPIGLHPCCFECVDCPPDTYLNRSVDEFNCLSCPGSMWSYKNNIACFKRRLAFLEWHEVPTIVVTILAALGFISTLAILLIFWRHFQTPMVRSAGGPMCFLMLVPLLLAFGMVPVYVGPPTVFSCFCRQAFFTVCFSVCLSCITVRSFQIVCVFKMARRLPSAYGFWMRYHGPYVFVAFITAVKVALVAGNMLATTINPIGRTDPDDPNIIILSCHPNYRNGLLFNTSMDLLLSVLGFSFAYVGKELPTNYNEAKFITLSMTFSFTSSISLCTFMSVHDGVLVTIMDLLVTVLNFLAIGLGYFGPKCYMILFYPERNTSAYFNSMIQGYTMRKS

SEQ ID NO: 62 - DNA, 2529 bp, Mus musculus
  CDS (1)..(2529)<s300> AY0326222002-02-05(1)..(2529)
atgcttttctgggcagctcacctgctgctcagcctgcagctggccgttgcttactgctgggctttcagctgccaaaggacagaatcctctccaggtttcagcctccctggggacttcctcctggcaggcctgttctccctccatgctgactgtctgcaggtgagacacagacctctggtgacaagttgtgacaggtctgacagcttcaacggccatggctatcacctcttccaagccatgcggttcaccgttgaggagataaacaactccacagctctgcttcccaacatcaccctggggtatgaactgtatgacgtgtgctcagagtcttccaatgtctatgccaccctgagggtgcccgcccagcaagggacaggccacctagagatgcagagagatcttcgcaaccactcctccaaggtggtggcactcattgggcctgataacactgaccacgctgtcaccactgctgccctgctgagcccttttctgatgcccctggtcagctatgaggcgagcagcgtgatcctcagtgggaagcgcaagttcccgtccttcttgcgcaccatccccagcgataagtaccaggtggaagtcatagtgcggctgctgcagagcttcggctgggtctggatctcgctcgttggcagctatggtgactacgggcagctgggcgtacaggcgctggaggagctggccactccacggggcatctgcgtcgccttcaaggacgtggtgcctctctccgcccaggcgggtgacccaaggatgcagcgcatgatgctgcgtctggctcgagccaggaccaccgtggtcgtggtcttctctaaccggcacctggctggagtgttcttcaggtctgtggtgctggccaacctgactggcaaagtgtggatcgcctccgaagactgggccatctccacgtacatcaccaatgtgcccgggatccagggcattgggacggtgctgggggtggccatccagcagagacaagtccctggcctgaaggagtttgaagagtcctatgtccaggcagtgatgggtgctcccagaacttgcccagaggggtcctggtgcggcactaaccagctgtgcagggagtgtcacgctttcacgacatggaacatgcccgagcttggagccttctccatgagcgctgcctacaatgtgtatgaggctgtgtatgctgtggcccacggcctccaccagctcctgggatgtacctctgggacctgtgccagaggcccagtctacccctggcagcttcttcagcagatctacaaggtgaatttccttctacataagaagactgtagcattcgatgacaagggggaccctctaggttattatgacatcatcgcctgggactggaatggacctgaatggacctttgaggtcattggttctgcctcactgtctccagttcatctagacataaataagacaaaaatccagtggcacgggaagaacaatcaggtgcctgtgtcagtgtgtaccagggactgtctcgaagggcaccacaggttggtcatgggttcccaccactgctgcttcgagtgcatgccctgtgaagctgggacatttctcaacacgagtgagcttcacacctgccagccttgtggaacagaagaatgggcccctgaggggagctcagcctgcttctcacgcaccgtggagttcttggggtggcatgaacccatctctttggtgctattagcagctaacacgctattgctgctgctgctgattgggactgctggcctgtttgcctggcgtcttcacacgcctgttgtgaggtcagctgggggtaggctgtgcttcctcatgctgggttccttggtagctgggagttgcagcctctacagcttcttcgggaagcccacggtgcccgcgtgcttgctgcgtcagcccctcttttctctcgggtttgccattttcctctcctgtctgacaatccgctccttccaactggtcatcatcttcaagttttctaccaaggtacccacattctaccacacttgggcccaaaaccatggtgccggaatattcgtcattgtcagctccacggtccatttgttcctctgtctcacgtggcttgcaatgtggaccccacggcccaccagggagtaccagcgcttcccccatctggtgattcttgagtgcacagaggtcaactctgtgggcttcctggtggctttcgcacacaacatcctcctctccatcagcacctttgtctgcagctacctgggtaaggaactgccggagaactataacgaagccaaatgtgtcaccttcagcctgctcctccacttcgtatcctggatcgctttcttcaccatgtccagcatttaccagggcagctacctacccgcggtcaatgtgctggcagggctggccactctgagtggcggcttcagcggctatttcctccctaaatgctacgtgattctctgccgtccagaactcaacaacacagaacactttcaggcctccatccaggactacacgaggcgctgcggcactacctga

SEQ ID NO: 63 - protein, 842 aa, Mus musculus
MLFWAAHLLLSLQLAVAYCWAFSCQRTESSPGFSLPGDFLLAGLFSLHADCLQVRHRPLVTSCDRSDSFNGHGYHLFQAMRFTVEEINNSTALLPNITLGYELYDVCSESSNVYATLRVPAQQGTGHLEMQRDLRNHSSKVVALIGPDNTDHAVTTAALLSPFLMPLVSYEASSVILSGKRKFPSFLRTIPSDKYQVEVIVRLLQSFGWVWISLVGSYGDYGQLGVQALEELATPRGICVAFKDVVPLSAQAGDPRMQRMMLRLARARTTVVVVFSNRHLAGVFFRSVVLANLTGKVWIASEDWAISTYITNVPGIQGIGTVLGVAIQQRQVPGLKEFEESYVQAVMGAPRTCPEGSWCGTNQLCRECHAFTTWNMPELGAFSMSAAYNVYEAVYAVAHGLHQLLGCTSGTCARGPVYPWQLLQQIYKVNFLLHKKTVAFDDKGDPLGYYDIIAWDWNGPEWTFEVIGSASLSPVHLDINKTKIQWHGKNNQVPVSVCTRDCLEGHHRLVMGSHHCCFECMPCEAGTFLNTSELHTCQPCGTEEWAPEGSSACFSRTVEFLGWHEPISLVLLAANTLLLLLLIGTAGLFAWRLHTPVVRSAGGRLCFLMLGSLVAGSCSLYSFFGKPTVPACLLRQPLFSLGFAIFLSCLTIRSFQLVIIFKFSTKVPTFYHTWAQNHGAGIFVIVSSTVHLFLCLTWLAMWTPRPTREYQRFPHLVILECTEVNSVGFLVAFAHNILLSISTFVCSYLGKELPENYNEAKCVTFSLLLHFVSWIAFFTMSSIYQGSYLPAVNVLAGLATLSGGFSGYFLPKCYVILCRPELNNTEHFQASIQDYTRRCGTT

SEQ ID NO: 64 - DNA, 2526 bp, Homo sapiens
  CDS (1)..(2526)<s300> BK0001532002-05-17(1)..(2526)
atgctgctctgcacggctcgcctggtcggcctgcagcttctcatttcctgctgctgggcctttgcctgccatagcacggagtcttctcctgacttcaccctccccggagattacctcctggcaggcctgttccctctccattctggctgtctgcaggtgaggcacagacccgaggtgaccctgtgtgacaggtcttgtagcttcaatgagcatggctaccacctcttccaggctatgcggcttggggttgaggagataaacaactccacggccctgctgcccaacatcaccctggggtaccagctgtatgatgtgtgttctgactctgccaatgtgtatgccacgctgagagtgctctccctgccagggcaacaccacatagagctccaaggagaccttctccactattcccctacggtgctggcagtgattgggcctgacagcaccaaccgtgctgccaccacagccgccctgctgagccctttcctggtgcccatgattagctatgcggccagcagcgagacgctcagcgtgaagcggcagtatccctctttcctgcgcaccatccccaatgacaagtaccaggtggagaccatggtgctgctgctgcagaagttcgggtggacctggatctctctggttggcagcagtgacgactatgggcagctaggggtgcaggcactggagaaccaggccactggtcaggggatctgcattgctttcaaggacatcatgcccttctctgcccaggtgggcgatgagaggatgcagtgcctcatgcgccacctggcccaggccggggccaccgtcgtggttgttttttccagccggcagttggccagggtgtttttcgagtccgtggtgctgaccaacctgactggcaaggtgtgggtcgcctcagaagcctgggccctctccaggcacatcactggggtgcccgggatccagcgcattgggatggtgctgggcgtggccatccagaagagggctgtccctggcctgaaggcgtttgaagaagcctatgcccgggcagacaagaaggcccctaggccttgccacaagggctcctggtgcagcagcaatcagctctgcagagaatgccaagctttcatggcacacacgatgcccaagctcaaagccttctccatgagttctgcctacaacgcataccgggctgtgtatgcggtggcccatggcctccaccagctcctgggctgtgcctctggagcttgttccaggggccgagtctacccctggcagcttttggagcagatccacaaggtgcatttccttctacacaaggacactgtggcgtttaatgacaacagagatcccctcagtagctataacataattgcctgggactggaatggacccaagtggaccttcacggtcctcggttcctccacatggtctccagttcagctaaacataaatgagaccaaaatccagtggcacggaaaggacaaccaggtgcctaagtctgtgtgttccagcgactgtcttgaagggcaccagcgagtggttacgggtttccatcactgctgctttgagtgtgtgccctgtggggctgggaccttcctcaacaagagtgacctctacagatgccagccttgtgggaaagaagagtgggcacctgagggaagccagacctgcttcccgcgcactgtggtgtttttggctttgcgtgagcacacctcttgggtgctgctggcagctaacacgctgctgctgctgctgctgcttgggactgctggcctgtttgcctggcacctagacacccctgtggtgaggtcagcagggggccgcctgtgctttcttatgctgggctccctggcagcaggtagtggcagcctctatggcttctttggggaacccacaaggcctgcgtgcttgctacgccaggccctctttgcccttggtttcaccatcttcctgtcctgcctgacagttcgctcattccaactaatcatcatcttcaagttttccaccaaggtacctacattctaccacgcctgggtccaaaaccacggtgctggcctgtttgtgatgatcagctcagcggcccagctgcttatctgtctaacttggctggtggtgtggaccccactgcctgctagggaataccagcgcttcccccatctggtgatgcttgagtgcacagagaccaactccctgggcttcatactggccttcctctacaatggcctcctctccatcagtgcctttgcctgcagctacctgggtaaggacttgccagagaactacaacgaggccaaatgtgtcaccttcagcctgctcttcaacttcgtgtcctggatcgccttcttcaccacggccagcgtctacgacggcaagtacctgcctgcggccaacatgatggctgggctgagcagcctgagcagcggcttcggtgggtattttctgcctaagtgctacgtgatcctctgccgcccagacctcaacagcacagagcacttccaggcctccattcaggactacacgaggcgctgcggctccacctga

SEQ ID NO: 65 - protein, 841 aa, Homo sapiens
MLLCTARLVGLQLLISCCWAFACHSTESSPDFTLPGDYLLAGLFPLHSGCLQVRHRPEVTLCDRSCSFNEHGYHLFQAMRLGVEEINNSTALLPNITLGYQLYDVCSDSANVYATLRVLSLPGQHHIELQGDLLHYSPTVLAVIGPDSTNRAATTAALLSPFLVPMISYAASSETLSVKRQYPSFLRTIPNDKYQVETMVLLLQKFGWTWISLVGSSDDYGQLGVQALENQATGQGICIAFKDIMPFSAQVGDERMQCLMRHLAQAGATVVVVFSSRQLARVFFESVVLTNLTGKVWVASEAWALSRHITGVPGIQRIGMVLGVAIQKRAVPGLKAFEEAYARADKKAPRPCHKGSWCSSNQLCRECQAFMAHTMPKLKAFSMSSAYNAYRAVYAVAHGLHQLLGCASGACSRGRVYPWQLLEQIHKVHFLLHKDTVAFNDNRDPLSSYNIIAWDWNGPKWTFTVLGSSTWSPVQLNINETKIQWHGKDNQVPKSVCSSDCLEGHQRVVTGFHHCCFECVPCGAGTFLNKSDLYRCQPCGKEEWAPEGSQTCFPRTVVFLALREHTSWVLLAANTLLLLLLLGTAGLFAWHLDTPVVRSAGGRLCFLMLGSLAAGSGSLYGFFGEPTRPACLLRQALFALGFTIFLSCLTVRSFQLIIIFKFSTKVPTFYHAWVQNHGAGLFVMISSAAQLLICLTWLVVWTPLPAREYQRFPHLVMLECTETNSLGFILAFLYNGLLSISAFACSYLGKDLPENYNEAKCVTFSLLFNFVSWIAFFTTASVYDGKYLPAANMMAGLSSLSSGFGGYFLPKCYVILCRPDLNSTEHFQASIQDYTRRCGST

SEQ ID NO: 66 - DNA, 1002 bp, Homo sapiens
  CDS (1)..(1002)<s300> AY2585972003-04-29(1)..(1002)
atgttgactctaactcgcatccgcactgtgtcctatgaagtcaggagtacatttctgttcatttcagtcctggagtttgcagtggggtttctgaccaatgccttcgttttcttggtgaatttttgggatgtagtgaagaggcagccactgagcaacagtgattgtgtgctgctgtgtctcagcatcagccggcttttcctgcatggactgctgttcctgagtgctatccagcttacccacttccagaagttgagtgaaccactgaaccacagctaccaagccatcatcatgctatggatgattgcaaaccaagccaacctctggcttgctgcctgcctcagcctgctttactgctccaagctcatccgtttctctcacaccttcctgatctgcttggcaagctgggtctccaggaagatctcccagatgctcctgggtattattctttgctcctgcatctgcactgtcctctgtgtttggtgcttttttagcagacctcacttcacagtcacaactgtgctattcatgaataacaatacaaggctcaactggcagattaaagatctcaatttattttattcctttctcttctgctatctgtggtctgtgcctcctttcctattgtttctggtttcttctgggatgctgactgtctccctgggaaggcacatgaggacaatgaaggtctataccagaaactctcgtgaccccagcctggaggcccacattaaagccctcaagtctcttgtctcctttttctgcttctttgtgatatcatcctgtgctgccttcatctctgtgcccctactgattctgtggcgcgacaaaataggggtgatggtttgtgttgggataatggcagcttgtccctctgggcatgcagccgtcctgatctcaggcaatgccaagttgaggagagctgtgatgaccattctgctctgggctcagagcagcctgaaggtaagagccgaccacaaggcagattcccggacactgtgctga

SEQ ID NO: 67 - protein, 333 aa, Homo sapiens [sequence omitted]

SEQ ID NO: 68 - DNA, 2333 bp, Mus musculus
  CDS (24)..(1088)<s300> M367781995-08-22(1)..(2333)
gctgtggcagggaaggggccaccatgggatgtacgctgagcgcagaggagagagccgccctcgagcggagcaaggcgattgagaaaaacctcaaagaagatggcatcagcgccgccaaagacgtgaaattactcctgctgggggctggagaatcaggaaaaagcaccattgtgaagcagatgaagatcatccatgaagatggcttctctggggaagacgtgaagcagtacaagcctgtggtctacagcaacaccatccagtctctggcggccattgtccgggccatggacactttgggcgtggagtatggtgacaaggagaggaagacggactccaagatggtgtgtgacgtggtgagtcgtatggaagacactgaaccgttctctgcagaacttctttctgccatgatgcgactctggggcgactcggggatccaggagtgcttcaaccgatctcgggagtatcagctcaatgactctgccaaatactacctggacagcctggatcggattggagccggtgactaccagcccactgagcaggacatcctccgaaccagagtcaaaacaactggcatcgtagaaacccacttcaccttcaagaacctccacttcaggctgtttgacgtcgggggccagcgatctgaacgcaagaagtggatccactgctttgaggatgtcacggccatcatcttctgtgtcgcactcagcggctatgaccaggtgctccacgaggacgaaaccacgaaccgcatgcacgaatccctgaagctcttcgacagcatctgcaacaacaagtggttcacagacacatctattatcctgtttctcaacaagaaggacatatttgaggagaagatcaagaagtccccactcaccatctgctttcctgaatacacaggccccagtgccttcacagaagctgtggctcacatccaagggcagtatgagagtaagaataagtcagctcacaaggaagtctacagccatgtcacctgtgccacggacaccaacaacatccaattcgtctttgatgccgtgacagatgtcatcatcgccaaaaacctacggggctgtggactctactgagccctggcctcctacccagcctgccactcactcctcccctggacccagagctctgtcactgctcagatgccctgttaactgaagaaaacctggaggctagccttgggggcaggaggaggcatcctttgagcatccccaccccacccaacttcagcctcgtgacacgtgggaacagggttgggcagaggtgtggaacagcacaaggccagagaccacggcatgccacttgggtgctgctcactggtcagctgtgtgtcttacacagaggccgagtgggcaacactgccatctgattcagaatgggcatgccctgtcctctgtacctcttgttcagtgtcctggtttctcttccaccttggtgataggatggctggcaggaaggccccatggaaggtgctgcttgattaggggatagtcgatggcatctctcagcagtcctcagggtctgtttggtagagggtggtttcgtcgacaaaagccaacatggaatcaggccacttttggggcgcaaagactcagactttggggacgggttccctcctccttcactttggatcttggcccctctctggtcatcttcccttgcccttgggctccccaggatactcagccctgactcccatggggttgggaatattccttaagactggctgactgcaaaggtcaccgatggagaaacatccctgtgctacagaattgggggtgggacagctgagggggcaggcggctctttcctgatagttgatgacaagccctgagaatgccatctgctggctccactcacacgggctcaactgtcctgggtgatagtgacttgccaggccacaggctgcaggtcacagacagagcaggcaagcagccttgcaactgcagattacttagggagaagcatcggggcctcgtgagccaggccccgtagccagtgccctgctttactccagccttggtcaggaagtcgaaagcccttggtgtattcctggtctcggagcaaataatgagccagcaccctgaagggtgggctccaactcagacatgcagccagccccctaggtgggtaaacgccctagggacctagggagagcctttgctgcagagattcctaagcaaaacggcgtggtggagctttggcaaccctagccccagctaactttggacagtcagcatatgtccctgccatccctagacatctccagtcagctggtatcacagccagtggttcagacaggtttgaatgctcatgtggca

SEQ ID NO: 69 - protein, 354 aa, Mus musculus
MGCTLSAEERAALERSKAIEKNLKEDGISAAKDVKLLLLGAGESGKSTIVKQMKIIHEDGFSGEDVKQYKPVVYSNTIQSLAAIVRAMDTLGVEYGDKERKTDSKMVCDVVSRMEDTEPFSAELLSAMMRLWGDSGIQECFNRSREYQLNDSAKYYLDSLDRIGAGDYQPTEQDILRTRVKTTGIVETHFTFKNLHFRLFDVGGQRSERKKWIHCFEDVTAIIFCVALSGYDQVLHEDETTNRMHESLKLFDSICNNKWFTDTSIILFLNKKDIFEEKIKKSPLTICFPEYTGPSAFTEAVAHIQGQYESKNKSAHKEVYSHVTCATDTNNIQFVFDAVTDVIIAKNLRGCGLY

SEQ ID NO: 70 - DNA, 1135 bp, Mus musculus
  CDS (41)..(1063)<s300> M872861993-04-27(1)..(1135)
gctcttcacttgagacgcctgagggaaaccaccaggcaggatgagcgagctggagcagctgaggcaggaggctgaacagcttcggaatcagatccaggatgctcggaaggcctgcaacgatgccacgctggttcagatcacgtctaatatggactccgtgggccgaatacaaatgcgaacaaggcgcacgctgcgtggccacctcgctaagatctacgccatgcactggggatatgattccaggctactagtcagtgcttcgcaagatggaaaattaattatttgggatagctatacgacaaataagatgcacgccatccctctgaggtcctcctgggtgatgacctgtgcctacgccccgtccgggaactacgttgcctgtggaggcttggataacatctgctccatatacaacctaaagacccgagagggagatgtgcgggtgagccgagaattggcaggacacacgggctacttgtcctgctgccgattcttagatgatggacaaatcattacaagttcgggagacacgacttgtgctttgtgggacattgagaccggacagcagactacgaccttcacaggacactcgggtgacgtgatgagcctctcactgagtcctgacttgaagacgtttgtgtctggtgcttgtgatgcatcctcaaagctgtgggatatccgagatgggatgtgtagacagtctttcaccggacacatctcagacatcaacgctgtcagtttcttcccgagtggatatgcctttgccactggttctgatgatgccacatgccgactctttgacctccgtgcagaccaggagctcctgctatactctcatgacaatatcatctgtggcattacttctgtggccttctcaaagagtgggcgcctcctgttagccggctatgacgacttcaactgcagtgtgtgggacgctctgaaaggaggccggtcaggtgtccttgctggtcatgacaaccgtgttagctgcttaggtgtgactgatgacggcatggctgtggccactggctcctgggacagttttcttagaatctggaattgagtgccatattttctgttctccaatgatacctggagaaatccgtgttacagcctatagctgtgaggaaaaaaa

SEQ ID NO: 71 - protein, 340 aa, Mus musculus
MSELEQLRQEAEQLRNQIQDARKACNDATLVQITSNMDSVGRIQMRTRRTLRGHLAKIYAMHWGYDSRLLVSASQDGKLIIWDSYTTNKMHAIPLRSSWVMTCAYAPSGNYVACGGLDNICSIYNLKTREGDVRVSRELAGHTGYLSCCRFLDDGQIITSSGDTTCALWDIETGQQTTTFTGHSGDVMSLSLSPDLKTFVSGACDASSKLWDIRDGMCRQSFTGHISDINAVSFFPSGYAFATGSDDATCRLFDLRADQELLLYSHDNIICGITSVAFSKSGRLLLAGYDDFNCSVWDALKGGRSGVLAGHDNRVSCLGVTDDGMAVATGSWDSFLRIWN

SEQ ID NO: 72 - DNA, 307 bp, Mus musculus
  CDS (40)..(267)<s300> U375271997-12-30(1)..(307)
tccaagctgctgtaccacctctcagcagggagtgcaggaatgaaggaaggcatgtctaataacagcaccaccagcatctcccaggccaggaaagccgtggagcagctgaagatggaagcctgcatggacagggtgaaggtctcccaggctgcctcagacctcctggcctactgtgaagcccacgtgcgggaggaccccctcatcatcccagtgcctgcctcagaaaaccccttccgggagaagaagttcttctgcaccatcctctaacacccatggcgatgaagcgggccctttcctgctgtaacag

SEQ ID NO: 73 - protein, 75 aa, Mus musculus
MKEGMSNNSTTSISQARKAVEQLKMEACMDRVKVSQAASDLLAYCEAHVREDPLIIPVPASENPFREKKFFCTIL

SEQ ID NO: 74 - DNA, 2666 bp, Mus musculus
  CDS (252)..(2219)<s300> BC0237292003-04-16(1)..(2666)
ccacgcgtccggccccagcgcaacgcgcagcagcctccctcctcttcttcccgcactgtgcgctcctcctgggctagggcgtctggatcgagtcccggaggctaccgcctcccagacagacgacaggtcacctggacgcgagcctgtgtccgggtctcgtcgttgccggcgcagtcactgggcacaaccgtgggactccgtctgtctcggattaatcccggagagccagagccaacctctcccggtcagagatgcgaccctcagggaccgcgagaaccacactgctggtgttgctgaccgcgctctgcgccgcaggtggggcgttggaggaaaagaaagtctgccaaggcacaagtaacaggctcacccaactgggcacttttgaagaccactttctgagcctgcagaggatgtacaacaactgtgaagtggtccttgggaacttggaaattacctatgtgcaaaggaattacgacctttccttcttaaagaccatccaggaggtggccggctatgtcctcattgccctcaacaccgtggagagaatccctttggagaacctgcagatcatcaggggaaatgctctttatgaaaacacctatgccttagccatcctgtccaactatgggacaaacagaactgggcttagggaactgcccatgcggaacttacaggaaatcctgattggtgctgtgcgattcagcaacaaccccatcctctgcaatatggatactatccagtggagggacatcgtccaaaacgtctttatgagcaacatgtcaatggacttacagagccatccgagcagttgccccaaatgtgatccaagctgtcccaatggaagctgctggggaggaggagaggagaactgccagaaattgaccaaaatcatctgtgcccagcaatgttcccatcgctgtcgtggcaggtcccccagtgactgctgccacaaccaatgtgctgcggggtgtacagggccccgagagagtgactgtctggtctgccaaaagttccaagatgaggccacatgcaaagacacctgcccaccactcatgctgtacaaccccaccacctatcagatggatgtcaaccctgaagggaagtacagctttggtgccacctgtgtgaagaagtgcccccgaaactacgtggtgacagatcatggctcatgtgtccgagcctgtgggcctgactactacgaagtggaagaagatggcatccgcaagtgtaaaaaatgtgatgggccctgtcgcaaagtttgtaatggcataggcattggtgaatttaaagacacactctccataaatgctacaaacatcaaacacttcaaatactgcactgccatcagcggggaccttcacatcctgccagtggcctttaagggggattctttcacgcgcactcctcctctagacccacgagaactagaaattctaaaaaccgtaaaggaaataacaggctttttgctgattcaggcttggcctgataactggactgacctccatgctttcgagaacctagaaataatacgtggcagaacaaagcaacatggtcagttttctttggcggtcgttggcctgaacatcacatcactggggctgcgttccctcaaggagatcagtgatggggatgtgatcatttctggaaaccgaaatttgtgctacgcaaacacaataaactggaaaaaactcttcgggacacccaatcagaaaaccaaaatcatgaacaacagagctgagaaagactgcaaggccgtgaaccacgtctgcaatcctttatgctcctcggaaggctgctggggccctgagcccagggactgtgtctcctgccagaatgtgagcagaggcagggagtgcgtggagaaatgcaacatcctggagggggaaccaagggagtttgtggaaaattctgaatgcatccagtgccatccagaatgtctgccccaggccatgaacatcacctgtacaggcaggggaccagacaactgcatccagtgtgcccactacattgatggcccacactgtgtcaagacctgcccagctggcatcatgggagagaacaacactctggtctggaagtatgcagatgccaataatgtctgccacctatgccacgccaactgtacctatggatgtgctgggccaggtcttcaaggatgtgaagtgtggccatctgggtacgttcaatggcagtggatcttaaagaccttttggatctaagaccagaagccatctctgactcccctctcaccttccagtttcttccaaatcctctgggccagccagaggtctcagattctgccctcttgccctgtgcccaccttgttgaccactggacagcatatgtgatggctactgctagtgccagcttcacaagaggttaacactacggactagccattcttcctatgtatctgtttctgcaaatacagccgctttacttaagtctcagcacttcttagtctcctcttttcctctcagtagcccaaggggtcatgtcacaaacatggtgtgaagggctactttgtcaaatgaaaaggtctatcttggggggcatttttttcttttctttttttcttgaaacacattgcccagcaaagccaataaatttctctcatcattttgtttctgataaattcttactattgataaaaaaaaaaaaaaaaa

SEQ ID NO: 75 - protein, 655 aa, Mus musculus
MRPSGTARTTLLVLLTALCAAGGALEEKKVCQGTSNRLTQLGTFEDHFLSLQRMYNNCEVVLGNLEITYVQRNYDLSFLKTIQEVAGYVLIALNTVERIPLENLQIIRGNALYENTYALAILSNYGTNRTGLRELPMRNLQEILIGAVRFSNNPILCNMDTIQWRDIVQNVFMSNMSMDLQSHPSSCPKCDPSCPNGSCWGGGEENCQKLTKIICAQQCSHRCRGRSPSDCCHNQCAAGCTGPRESDCLVCQKFQDEATCKDTCPPLMLYNPTTYQMDVNPEGKYSFGATCVKKCPRNYVVTDHGSCVRACGPDYYEVEEDGIRKCKKCDGPCRKVCNGIGIGEFKDTLSINATNIKHFKYCTAISGDLHILPVAFKGDSFTRTPPLDPRELEILKTVKEITGFLLIQAWPDNWTDLHAFENLEIIRGRTKQHGQFSLAVVGLNITSLGLRSLKEISDGDVIISGNRNLCYANTINWKKLFGTPNQKTKIMNNRAEKDCKAVNHVCNPLCSSEGCWGPEPRDCVSCQNVSRGRECVEKCNILEGEPREFVENSECIQCHPECLPQAMNITCTGRGPDNCIQCAHYIDGPHCVKTCPAGIMGENNTLVWKYADANNVCHLCHANCTYGCAGPGLQGCEVWPSGYVQWQWILKTFWI

SEQ ID NO: 76 - DNA, 669 bp, Mus musculus
  CDS (1)..(669)<s300> AF1025371999-02-08(1)..(669)
agtcagctctccctcatggacctcatgctggtctgtaacattgtgccaaagatggcagtcaacttcctgtctggcaggaagtccatctcttttgccggctgtggcatacaaatcggattttttgtctctcttgtgggatcagagggtctcttgttaggactcatggcttatgatcgctatgtggccattagccacccacttcactatcccattctcatgagccaaaaggtctgtctccagattgctggaagttcctgggcttttgggatccttgatggaataattcagatggtggcagccatgagcctgccctactgtggctcacggtatatagatcacttcttctgtgaagtgccggctttactgaagctggcctgtgcagacacctcccttttcgacaccctgctctttgcttgctgtgtctttatgctgcttcttcctttctcgatcattgtgacttcctatgctcgcatcttgggggctgtgctccgtatgcactctgcccagtcccgaaaaaaggccctggccacttgttcctcccacctgacagctgtctctctcttctacggggcagcaatgttcatctacctgaggccaaggcgatatcgcgctcctagccatgacaaagttgtctcaatcttctacacagttcttactcctatgctcaac

SEQ ID NO: 77 - protein, 223 aa, Mus musculus
SQLSLMDLMLVCNIVPKMAVNFLSGRKSISFAGCGIQIGFFVSLVGSEGLLLGLMAYDRYVAISHPLHYPILMSQKVCLQIAGSSWAFGILDGIIQMVAAMSLPYCGSRYIDHFFCEVPALLKLACADTSLFDTLLFACCVFMLLLPFSIIVTSYARILGAVLRMHSAQSRKKALATCSSHLTAVSLFYGAAMFIYLRPRRYRAPSHDKVVSIFYTVLTPMLN

SEQ ID NO: 78 - DNA, 1140 bp, Rattus norvegicus;
  CDS (1)..(1140)<s300> XM_2239842003-01-28(1)..(1140)
atgaaagccctcagcaactccagcaccatcactggcttcatcctcctgggcttcccctgccccagggaggggcaaatcctcctctttgtgcttttcttcatggtctacctccttaccctcatgggcaatgcttccatcatctgtgctgtgtgttgtgattcgagactccagacccccatgtacctcctgctggccaacttctccttcctggagatctggtatgtcacctccacagtccccaacatgttggccaacttcctgtctgaaaccaaagtcatctctttctctggatgcttcctgcagttctatttcttcttctcctttggttctacagaatgctttttcctggcagtcatggcatttgatcgctaccttgccatctgcaggcctctacattaccctgccctcatgaccgggcgcctctgcaacatccttgtgatcagttgctgggtgcttggtttcctctggttccctgttcccatcatcttcatctcccagatgtccttctgtgggtccagaattatagaccacttcctgtgtgacccaggtcctctgctagcactgacttgcacaaaatctcccctaatagagttggccttctccatcttaagtcctctgcctctcattattccttttgttttcatcatgggatcgtatactctggtcttagcagctgtattgaaggtcccttcagcctctggaaaaagaaaggctttctcaacctgtgggtctcatctggcagtggttgcattattctatggctcagtactggtcatgtatgggagcccaacatctgaacatgaatctgggatgcagaagattgtgactctgttttactctgtcttgaccccgctcctcaatcctgtgatatatagtctcaggaacaaacatatgaagcaaccaggcatccccacagccaatattcctgaacaagtagtagacaatcttacagctgatgtgtccactggcatctattatggctgggccagcgttgggagtggagaggaccataaaatggtggtgagcataggatggaatccatactacaagaatgtgaaaaagtccatggaaacccacgtcatccataccttcaaagaggacttctatgggggaattctcagtgtggccattgttggctaa

SEQ ID NO: 79 - protein, 379 aa, Rattus norvegicus;
MKALSNSSTITGFILLGFPCPREGQILLFVLFFMVYLLTLMGNASIICAVCCDSRLQTPMYLLLANFSFLEIWYVTSTVPNMLANFLSETKVISFSGCFLQFYFFFSFGSTECFFLAVMAFDRYLAICRPLHYPALMTGRLCNILVISCWVLGFLWFPVPIIFISQMSFCGSRIIDHFLCDPGPLLALTCTKSPLIELAFSILSPLPLIIPFVFIMGSYTLVLAAVLKVPSASGKRKAFSTCGSHLAVVALFYGSVLVMYGSPTSEHESGMQKIVTLFYSVLTPLLNPVIYSLRNKHMKQPGIPTANIPEQVVDNLTADVSTGIYYGWASVGSGEDHKMVVSIGWNPYYKNVKKSMETHVIHTFKEDFYGGILSVAIVG

SEQ ID NO: 80 - DNA, 954 bp, Mus musculus
  CDS (1)..(954)<s300> NM_1471112003-04-07(1)..(954)
atgcatactggtatgtccattttcaacctctcagttactgaagtttctacgttccatttggttgggttcccagggatggaatctgcccacatttggatcttcatccccatatgtctcctgtacactgttgccatcctgggaaactgcactatcctctttttcataaaatcggagccttctctgcatgaacccatgtactatttcctctccatgttggctctctctgacctgggactgtccatttcctctcttcctaccatgcttaaggttttcctgttcaattctccagacatttctcctaacgagtgttttgcccaggagttttttattcatgaattctcagctatggagtcatctgtgcttctcattatgtcctttgatcgctatattgccatctgtaaccctctgagatacacttccatccttaccagtgccagggtccttcaaatcgggcttgctttttctctcaagaatgtgctgttgatcctgcctttccctgtaactctagtgcgtctaagatactgcaagaagaatctcttgtcacattcctattgccttcaccaggatgtcatgaaactggcctgctcagacaacaagttcaatgtcatctatggcttatttgtggctctcacaggcatccttgacataaccttcattttcatgtcatatgcactcatcctgagagcagtgctgggcattgcatcccaaagggaaagactcaaggtcctcagtacttgtgtctctcacatctgtgctgtgctcattttctatgtccctgtcatctcccttgctgtcatctatcgccttgcgagtcgtagttcccccatatctaagattctcatggctgacattttcttgcttgtgcctcctgtaatgaatcctatcatatactgtgtgaagagccaacagataagaaatgtgattctagagaaactgtgtcagaaacaaagctga

SEQ ID NO: 81 - protein, 317 aa, Mus musculus
MHTGMSIFNLSVTEVSTFHLVGFPGMESAHIWIFIPICLLYTVAILGNCTILFFIKSEPSLHEPMYYFLSMLALSDLGLSISSLPTMLKVFLFNSPDISPNECFAQEFFIHEFSAMESSVLLIMSFDRYIAICNPLRYTSILTSARVLQIGLAFSLKNVLLILPFPVTLVRLRYCKKNLLSHSYCLHQDVMKLACSDNKFNVIYGLFVALTGILDITFIFMSYALILRAVLGIASQRERLKVLSTCVSHICAVLIFYVPVISLAVIYRLASRSSPISKILMADIFLLVPPVMNPIIYCVKSQQIRNVILEKLCQKQS

SEQ ID NO: 82 - DNA, 921 bp, Mus musculus
  CDS (1)..(921)<s300> AF2823002000-12-13(1)..(921)
atggaaaaaggcaatcaatccacagtgaacaaattcttcctttctggcttaacagaacaaccagaactgcaactgccactcttcctcctctttcttggaatctacctgctcacagtgctggggaacctgggcatgatcatcctgatcctgctcagctcgtacctgcacacccctatgtacttcttcctcagtagtctgtccttcattgacttctgccaatctactgtcattaccccaaaaatgctggtgaaatttgtgagggagaagaatgaaatctcctaccctgagtgcataactcaactttgcttctttgtcatttttgctgtttcagaaagttacatgctggctgcaatggcatatgatcgctatgttgccatctgtagccccttactttacagtagcatcatgtctcaacataagtgcctttctctcgttttaggagtttacattctaggcatagtttgtgcatcagctcatgtaggatgtatatttaggattgatttctgcaaatctgatttgatcaaccattatttctgtgaccttatttctattcttaatctctcatgctctaatatttttgtgaatgatctcgtaattctaatttttagtctaattaataccattttcccaaccctgaccatcctcagttcttatgctttcatcattatcagcatcctacgcattaaatccactgagggaagatctaaagccttcagtacctgcagctcccacatctcagctgttgctatcttttatatctctgctggatttacgtatctgaacccatcatcttcccattcgatggatgaaggaaaagtgtcttctatattttacaccatcattgttcccatgctcaaccccctgatttacagtcttaggaataaggacgtgaaaattgctctgaagaaaatgattgaatga

SEQ ID NO: 83 - protein, 306 aa, Mus musculus
MEKGNQSTVNKFFLSGLTEQPELQLPLFLLFLGIYLLTVLGNLGMIILILLSSYLHTPMYFFLSSLSFIDFCQSTVITPKMLVKFVREKNEISYPECITQLCFFVIFAVSESYMLAAMAYDRYVAICSPLLYSSIMSQHKCLSLVLGVYILGIVCASAHVGCIFRIDFCKSDLINHYFCDLISILNLSCSNIFVNDLVILIFSLINTIFPTLTILSSYAFIIISILRIKSTEGRSKAFSTCSSHISAVAIFYISAGFTYLNPSSSHSMDEGKVSSIFYTIIVPMLNPLIYSLRNKDVKIALKKMIE

SEQ ID NO: 84 - DNA, 933 bp, Mus musculus
  CDS (1)..(933)<s300> AF2822792000-12-13(1)..(933)
atggccactgaaaatgcctctgtgccagagttcatcctcgcaggcctgacagaccagccaggactccgcatgcccctcttctttctgtttctaggtttctacatggtcaccatggtgggaaacctgggcttgatcaccctgattggactgaactctcacctgcacacccctatgtactttttcctcttcaacttgtcccttatagatttctgctactccactgttattactcccaaaatgctggtgagctttgtctcaaagaaaaacatcatctcctactctggatgcatgacccagctcttcttttttcttttctttgttgtctctgagtccttcatcctatcagcaatggcatatgacaggtatgttgccatctgtaatcctctgatgtacaccgtgaccatgtctccccaggtgtgcttactccttttgctaggtgtgtatgtgatgggatttgctggagccatggcccacacagcattcatggtgaaactgaccttctgtgctgacaagcttgtcaatcactacatgtgtgacattctccctcttctggaacgctcttgcaccagcacctatgtaaatgagctggtagtcttcattgttgtgggcattgatataggggtacccacagtcaccatctttatttcttatgccctcatcctctccagtatccttcgcatcagttccacagagggcaggtccaaggccttcagcacctgcagctcccacataattgcagtttctctcttttttggatcaggggcattcatgtatctcaaaccttcttcccttttgcctatgaaccaggggaaagtgtcgtccttgttttatacaattgtggtgcccatgctcaacccattaatctatagcttaaggaataaggatgtcaaagttgctctgagaaaaacattgagcagaagttcattttcctaa

SEQ ID NO: 85 - protein, 310 aa, Mus musculus
MATENASVPEFILAGLTDQPGLRMPLFFLFLGFYMVTMVGNLGLITLIGLNSHLHTPMYFFLFNLSLIDFCYSTVITPKMLVSFVSKKNIISYSGCMTQLFFFLFFVVSESFILSAMAYDRYVAICNPLMYTVTMSPQVCLLLLLGVYVMGFAGAMAHTAFMVKLTFCADKLVNHYMCDILPLLERSCTSTYVNELVVFIVVGIDIGVPTVTIFISYALILSSILRISSTEGRSKAFSTCSSHIIAVSLFFGSGAFMYLKPSSLLPMNQGKVSSLFYTIVVPMLNPLIYSLRNKDVKVALRKTLSRSSFS

SEQ ID NO: 86 - DNA, 930 bp, Mus musculus
  CDS (1)..(930)<s300> AF2835582000-04-02(1)..(930)
atggagttgaaaaatgacacacaaatttcaaaatttattctcctgggaatttcagaggatcctctatggcaacctttcctttttggactatttttgttcatgtacttggtcactctgcttgggaaccttctcattatcattgccaccattacagattcccatcttcacacacccatgtacttcttcctttccaacctgtcctttgcagacatctgctttacttctgctagcatcccaaagatgctagtgaatatacagacaaagaacaaggtgataacctatgaaggttgcatttctcaagtattctttttcatactatttggagttttagataactttcttctagctgtgatggcctatgaccgatatgtggcaatctgtcaccctctgcactatatggtcatcatgaactgccgcctctgtggatttttagttttggggtcttgggtcacaacagcattgaattccttgctgcagagttcaatggcactgcggctgtccttttgtacagacttgaaaattccccactttgtttgtgagcttaatcaactggtactacttgcctgtaatgacacctttcctaatgacatggtgatgtactttgcagctatactgctgggtggtggtcctcttgctggcatcctttactcttattctaagatagtttcctccatacgtgcaatctcatcatcacaggggaagtacaaagcattctccacctgtgcatcccacctctcagttgtttcattattctattctacactcttgggtgtgtatcttagttcttcttttacacaaaactcacactcaactgcacgagcatctgttatgtacagtgtggtcacccccatgttgaacccattcatctatagtttgaggaataaggacctaatgggagctctgagaagactcttaagaaggaagtcatga

SEQ ID NO: 87 - protein, 309 aa, Mus musculus
MELKNDTQISKFILLGISEDPLWQPFLFGLFLFMYLVTLLGNLLIIIATITDSHLHTPMYFFLSNLSFADICFTSASIPKMLVNIQTKNKVITYEGCISQVFFFILFGVLDNFLLAVMAYDRYVAICHPLHYMVIMNCRLCGFLVLGSWVTTALNSLLQSSMALRLSFCTDLKIPHFVCELNQLVLLACNDTFPNDMVMYFAAILLGGGPLAGILYSYSKIVSSIRAISSSQGKYKAFSTCASHLSVVSLFYSTLLGVYLSSSFTQNSHSTARASVMYSVVTPMLNPFIYSLRNKDLMGALRRLLRRKS

SEQ ID NO: 88 - DNA, 660 bp, Mus musculus
  CDS (1)..(660)<s300> AF1025221999-02-08(1)..(660)
tctttcctgtcgctgatagatggctgctgctcctcgtccatgactcctaagatgctggctgactctctctctgtgcggaaaaccatttctttcagtggctgcatgactcaagtctttgccgagcattttttcggggctgctgagattattcttctcacagtgatggcctacgaccgctatgtggccatctgtaaaccgctgcgctacacgatcattatgaacagatttgtgtgtggcctcttggtgggggtggcctgggctgggggttttattcatgcgaccattcagatcctcttcacagtctggctgcctttctgtggtcccaatgtcatagaccactttatgtgtgacctgacccctttgctgaaacttgtttgtatggacacacacaacctgggtctctttgttgctgccaacagtggctttatctgcctgttaaactttctacttctgatgatctcttatatagtcatcctcgatgctctaaaatctcacagcaaggagggaaggcgcaaagccctctccacctgtgtctctcacattactgtggtcatcttattctttgtgccttgcatatttgtgtatcttcgcccagtaattaccttctctattgataaagcggtggctgtattttataccatgattactcctatgttaaac

SEQ ID NO: 89 - protein, 220 aa, Mus musculus
SFLSLIDGCCSSSMTPKMLADSLSVRKTISFSGCMTQVFAEHFFGAAEIILLTVMAYDRYVAICKPLRYTIIMNRFVCGLLVGVAWAGGFIHATIQILFTVWLPFCGPNVIDHFMCDLTPLLKLVCMDTHNLGLFVAANSGFICLLNFLLLMISYIVILDALKSHSKEGRRKALSTCVSHITVVILFFVPCIFVYLRPVITFSIDKAVAVFYTMITPMLN

SEQ ID NO: 90 - DNA, 948 bp, Mus musculus
  CDS (1)..(948)<s300> NM_0136202003-04-06(1)..(948)
atgatcaagttcaatggctcagtcttcatgccttctgtgttaacactagtggggatccctggcctggagtcagtgcagtgctggattggaattccattctgtgtcatgtacatcattgctatgattgggaactccctaattttagttataatcaaaagtgaaaagagcctccacatacccatgtacattttcttggctattttggcagtcacagacattgcccttagcacatgcattcttcccaaaatgttgggcatcttctggtttcacatgccacagatttcctttgatgcctgcctgctccagatggaactcatccactcattccaggccacagaatcaggcatcctcctggccatggctctggatcgctatgtggctatctgcaaccccctcagacatgctactatcttctctccacaactcaccacttgccttggagctggtgcattactcagggctttcattcttgtatccccatccatactactcatcaaatgtcgccttaagtactttagaactaccattatctcccactcgtactgtgaacacatggccattgtgaaactggcagctcaagatatcagaatcaacaagatatgtggtctccttgttgcctttgccatcttagggtttgacatagtcttcattaccttctcctatgtgcgaatcttcatcacggtcttccagctgccccagaaggaggctcgattcaaagccttcaatacctgcattgctcacatctgtgtcttcctgcagttctaccttctggccttcttctctttcttcacacacaggtttggggctcacatacccccctatgtgcacatcctcctgtcagacctttacctgttagtcccaccttttctgaaccccattgtttatggtgttaaaactaaacaaatccgtgaccaagtcctgaaaatgctattctccaagaaacattag

SEQ ID NO: 91 - protein, 315 aa, Mus musculus
MIKFNGSVFMPSVLTLVGIPGLESVQCWIGIPFCVMYIIAMIGNSLILVIIKSEKSLHIPMYIFLAILAVTDIALSTCILPKMLGIFWFHMPQISFDACLLQMELIHSFQATESGILLAMALDRYVAICNPLRHATIFSPQLTTCLGAGALLRAFILVSPSILLIKCRLKYFRTTIISHSYCEHMAIVKLAAQDIRINKICGLLVAFAILGFDIVFITFSYVRIFITVFQLPQKEARFKAFNTCIAHICVFLQFYLLAFFSFFTHRFGAHIPPYVHILLSDLYLLVPPFLNPIVYGVKTKQIRDQVLKMLFSKKH

SEQ ID NO: 92 - DNA, 954 bp, Mus musculus
  CDS (1)..(954)<s300> XM_1388992002-03-16(1)..(354)
atggaaggggacaacacatcatccacggacttcactttcatggggctgttcaacacagaggaaacctcaggtcttgtatttgccacaatctccgtcatcttcctcactgcactggtggccaatggaattatgatctttctgatccacacagacgctcacctccacacccctatgtactttctcctcagtcacttgtccttcattgacatgatgtacatctcaaccattgtgcccaagatgctagttgattatcttctagggcaaaggactatttcctttgtgggatgcacagctcaacactttctatacctcaccctggtgggagccgagttctttcttctgggcctcatggcttatgatcgttatgtggccatctgcaacccactgaggtaccctgtcctcatgagccgccggatctgttggattatcatagcaggctcctggtttgggggatctttggatggcttcctcctcactccaatcaccatgagttttcctttctgtagatcacgagagattaaccacttcttctgtgaggcacctgctgtgctgaagttggcatgtgcagacacagccctctatgagacggtgatgtatgtgtgctgcgttctgatgctgttgattcctttctctgtggttatctcatcctatgcgcggattctggccactgtctaccatatgagctctgtggaaggaaggaagaaagcgtttgctacctgctcgtctcacatgactgtggtaaccttgttttatggggctgccatatacacctatatggtaccacactcttaccattccccatcccaagacaaaattttttctgtgttctataccattctcacacccatgctgaaccccctcatttacagcatgaggaacaaagatgtgtctggaggtctgaggagggcactggggaagatcgggagttctcagcgagtgtccaaagacttttga

SEQ ID NO: 93 - protein, 317 aa, Mus musculus
MEGDNTSSTDFTFMGLFNTEETSGLVFATISVIFLTALVANGIMIFLIHTDAHLHTPMYFLLSHLSFIDMMYISTIVPKMLVDYLLGQRTISFVGCTAQHFLYLTLVGAEFFLLGLMAYDRYVAICNPLRYPVLMSRRICWIIIAGSWFGGSLDGFLLTPITMSFPFCRSREINHFFCEAPAVLKLACADTALYETVMYVCCVLMLLIPFSVVISSYARILATVYHMSSVEGRKKAFATCSSHMTVVTLFYGAAIYTYMVPHSYHSPSQDKIFSVFYTILTPMLNPLIYSMRNKDVSGGLRRALGKIGSSQRVSKDF

SEQ ID NO: 94 - DNA, 936 bp, Mus musculus
  CDS (1)..(936)<s300> NM_1469552000-04-07(1)..(936)
atgatgtcacgactcaaccagacagtagtgacagagtttatactgcaaggtttctcagagcaccctagtctaagactgttcctgacaggctgcttcttgtccctctatgtaatggctctaatgggcaacattttgatcattgctttggtcaccttcagcactgggctccacagtcccatgtactttttcctgtgcaacctggccaccatggatattatctgcacctcctctgtgctgcccaaggcgctggttggtctactatctgaggaaaacaccatctcctttaaagggtgcatggcccagctcttcttccttgtgtggtccttgtcttcagagctgctgctgctcacagtcatggcctatgaccgctatgtggccatctgctttcccctgcactacagctctagaatgagcccacagttgtgtggggctctggccatgggtgtatggtccatctgtgctctgaatgcatctatcaacactggtctgatgacacggctgtcattctgtggacccaaggtcatcacccacttcttctgtgagattcccccactccttctgctctcctgtagccccacatacgtaaacagcattatgactctaatagcagatgtcttctatggaggcatcaattttgtgcttaccttactatcctatggctgcatcattgccagcatcctgcgcatgcgttctgctgagggcaagaggaaggccttttctacctgctcatcccacctcatcgtggtctctgtgtactactcatctgtgttctgtgcctatgtcagccctgcatccagctatagcccagaaagaagcaaagttacctctgtgttgtactcattcctcagcccaaccctgaaccccctcatttacacattgaggaacaaagatgtcaagctcgccattggcaggctcttgccctctttctcacattaa

SEQ ID NO: 95 - protein, 311 aa, Mus musculus
MMSRLNQTVVTEFILQGFSEHPSLRLFLTGCFLSLYVMALMGNILIIALVTFSTGLHSPMYFFLCNLATMDIICTSSVLPKALVGLLSEENTISFKGCMAQLFFLVWSLSSELLLLTVMAYDRYVAICFPLHYSSRMSPQLCGALAMGVWSICALNASINTGLMTRLSFCGPKVITHFFCEIPPLLLLSCSPTYVNSIMTLIADVFYGGINFVLTLLSYGCIIASILRMRSAEGKRKAFSTCSSHLIVVSVYYSSVFCAYVSPASSYSPERSKVTSVLYSFLSPTLNPLIYTLRNKDVKLAIGRLLPSFSH

SEQ ID NO: 96 - DNA, 954 bp, Mus musculus
  CDS (1)..(954)<s300> XM_1384562003-02-24(1)..(954)
atggacccaagcaattacagtactttgcatgtttttatcctgcttggattctctgaccaccctcacctggaaatgatcctctctggagttgtcaccttcttttacattattacactggtgggtaacactgctataattcttgcatccctcctggatccccatctccatacaccaatgtactttttcctcaggaatctatctttcctggatttgtgtttcacaacaagcattgtccctcagatgctggttaacttgtggggacccgaaaagaccattagctctgtgggctgtattgttcagctctatgtgtatatgtggctgggctccattgagtgtctgctcctagctgtcatgtcctatgatcgtttcacagctatttgtaagcccttgcattactttgtaatcatgaatccacgtctatgcgtcaagatgatagtcatggtctggggtattagtttggccaactctgtaatattatgcacactcactgtgaatttgcctcgatgtggacacaacatcctggaccacttcctgtgtgagttgccagccatggtcaggatagcttgtgtagacacgacaacagttgaattgtctgtttttgctctaggcattgtcattgtccttacacctctcatccttattcttatttcctatggctacatagccaaaactgtgctcaacatgaagtcaaaggcagggcaacaaaaagcaatgaatacctgtggatcccatctcactgtggtctccatattctatggaactatcatctacatgtatctacaacccggtaacagggcctctaaggaccagggcaagttcctcaccctcttttacaccatcatcactccaagtctcaaccccctcatttacaccctaaggaacagagacatgaaagatgcactgaaaaaactcatgaggttttaccacagatttgccgaagtaaggagaaactag

SEQ ID NO: 97 - protein, 317 aa, Mus musculus
MDPSNYSTLHVFILLGFSDHPHLEMILSGVVTFFYIITLVGNTAIILASLLDPHLHTPMYFFLRNLSFLDLCFTTSIVPQMLVNLWGPEKTISSVGCIVQLYVYMWLGSIECLLLAVMSYDRFTAICKPLHYFVIMNPRLCVKMIVMVWGISLANSVILCTLTVNLPRCGHNILDHFLCELPAMVRIACVDTTTVELSVFALGIVIVLTPLILILISYGYIAKTVLNMKSKAGQQKAMNTCGSHLTVVSIFYGTIIYMYLQPGNRASKDQGKFLTLFYTIITPSLNPLIYTLRNRDMKDALKKLMRFYHRFAEVRRN

SEQ ID NO: 98 - DNA, 472 bp, Artificial sequence
  IHS Expression Vector(pBK-CMV-IHS-M4nc)
  CDS (89)..(319)
  misc_feature (320)..(328): Dammy sequence
  CDS (328)..(396)
  misc_feature (430)..(430): n is a, c, g, t or u
caaatgggcggtaggcgtgtacggtgggaggtctatataagcagagctggtttagtgaaccgtcagatccgctagtggatccgccaccatgaagacgatcatcgccctgagctacatcttctgcctggttaacgcggacatcgaattcatgccggggcaaaattactcgaccatatcagaatttatcctctttggtttctcagccttcccacaccagatgctccctgctctgttcctgctctacttgctgatgtatttgttcactcttctggggaacctggtcatcatggctgccatctggaccgaacaccgcctgcagcgctggtgctccggaacaaggaattgaagaacgctataattaaaagcttccacaggaatgtctgccaacaatccatctaagtgtcagttctgtgctctagagcggccgcgngcccatcgattttccacccgggtggggtaccaggtaagtgta

SEQ ID NO: 99 - protein, 100 aa, Artificial sequence
  IHS Expression Vector(pBK-CMV-IHS-M4nc)
MKTIIALSYIFCLVNADIEFMPGQNYSTISEFILFGFSAFPHQMLPALFLLYLLMYLFTLLGNLVIMAAIWTEHRLQLRNKELKNAIIKSFHRNVCQQSI

SEQ ID NO: 100 - DNA, 472 bp, Artificial sequence
  Rho Expression Vector (pBK-CMV-Rho-M4nc)
  CDS (125)..(361)
  misc_feature (362)..(440): Dammy sequence
  CDS (370)..(438)
caaatgggcggtaggcgtgtacggtgggaggtctatataagcagagctggtttagtgaaccgtcagatccgctagtggatccccgggttcgcgccgccggcgggcagccgcaagggccgcagccatgaacgggaccgagggcccaaacttctacgtgcctttctccaacaagacgggcgtggtggaattcatgccggggcaaaattactcgaccatatcagaatttatcctctttggtttctcagccttcccacaccagatgctccctgctctgttcctgctctacttgctgatgtatttgttcactcttctggggaacctggtcatcatggctgccatctggaccgaacaccgcctgcagcgctggtgctccggaacaaggaattgaagaacgctataattaaaagcttccacaggaatgtctgccaacaatccatctaagtgtcagttctgtgctctagagcgccgcggg

SEQ ID NO: 101 - protein, 102 aa, Artificial sequence
  Rho Expression Vector (pBK-CMV-Rho-M4nc)
MNGTEGPNFYVPFSNKTGVVEFMPGQNYSTISEFILFGFSAFPHQMLPALFLLYLLMYLFTLLGNLVIMAAIWTEHRLQLRNKELKNAIIKSFHRNVCQQSI

SEQ ID NO: 102 - DNA, 18 bp, artificial sequence
  The sequence containing the Pst I restriction site CTGCAG
accgaacaccgcctgcag

SEQ ID NO: 103 - DNA, 18 bp, artificial sequence
  the sequence containing the Bsp EI restriction site TCCGGA
tccggaacaaggaattga

SEQ ID NO: 104 - DNA, 384 bp, artificial sequence
  Rho-M4 chimera cassette for pBK-CMV vector without a dummy sequence
  CDS (55)..(360)
gctagtggatccccgggttcgcgccgccggcgggcagccgcaagggccgcagccatgaacgggaccgagggcccaaacttctacgtgcctttctccaacaagacgggcgtggtggaattcatgccggggcaaaattactcgaccatatcagaatttatcctctttggtttctcagccttcccacaccagatgctccctgctctgttcctgctctacttgctgatgtatttgttcactcttctggggaacctggtcatcatggctgccatctggaccgaacaccgcctgcagctccggaacaaggaattgaagaacgctataattaaaagcttccacaggaatgtctgccaacaatccatctaagtgtcagttctgtgctctaga

SEQ ID NO: 105 - protein, 102 aa, artificial sequence; identical to SEQ ID NO: 101
  Rho-M4 chimera cassette for pBK-CMV vector without a dummy sequence

SEQ ID NO: 106 - DNA, 392 bp, artificial sequence
  Rho-M4 chimera cassette for pBK-CMV vector with a dummy sequence
  CDS (55)..(291)
  CDS (300)..(368)
gctagtggatccccgggttcgcgccgccggcgggcagccgcaagggccgcagccatgaacgggaccgagggcccaaacttctacgtgcctttctccaacaagacgggcgtggtggaattcatgccggggcaaaattactcgaccatatcagaatttatcctctttggtttctcagccttcccacaccagatgctccctgctctgttcctgctctacttgctgatgtatttgttcactcttctggggaacctggtcatcatggctgccatctggaccgaacaccgcctgcagcgctggtgctccggaacaaggaattgaagaacgctataattaaaagcttccacaggaatgtctgccaacaatccatctaagtgtcagttctgtgctctaga

SEQ ID NO: 107 - protein, 102 aa, artificial sequence; identical to SEQ ID NO: 101
  Rho-M4 chimera cassette for pBK-CMV vector with a dummy sequence

SEQ ID NO: 108 - DNA, 342 bp, artificial sequence
  IHS-M4 chimera cassette for pBK-CMV vector without a dummy sequence
  CDS (19)..(318)
gctagtggatccgccaccatgaagacgatcatcgccctgagctacatcttctgcctggttaacgcggacatcgaattcatgccggggcaaaattactcgaccatatcagaatttatcctctttggtttctcagccttcccacaccagatgctccctgctctgttcctgctctacttgctgatgtatttgttcactcttctggggaacctggtcatcatggctgccatctggaccgaacaccgcctgcagctccggaacaaggaattgaagaacgctataattaaaagcttccacaggaatgtctgccaacaatccatctaagtgtcagttctgtgctctaga

SEQ ID NO: 109 - protein, 100 aa, artificial sequence; identical to SEQ ID NO: 99
  IHS-M4 chimera cassette for pBK-CMV vector without a dummy sequence

SEQ ID NO: 110 - DNA, 350 bp, artificial sequence
  IHS-M4 chimera cassette for pBK-CMV vector with a dummy sequence
  CDS (19)..(249)
  CDS (258)..(326)
gctagtggatccgccaccatgaagacgatcatcgccctgagctacatcttctgcctggttaacgcggacatcgaattcatgccggggcaaaattactcgaccatatcagaatttatcctctttggtttctcagccttcccacaccagatgctccctgctctgttcctgctctacttgctgatgtatttgttcactcttctggggaacctggtcatcatggctgccatctggaccgaacaccgcctgcagcgctggtgctccggaacaaggaattgaagaacgctataattaaaagcttccacaggaatgtctgccaacaatccatctaagtgtcagttctgtgctctaga

SEQ ID NO: 111 - protein, 100 aa, artificial sequence; identical to SEQ ID NO: 99
  IHS-M4 chimera cassette for pBK-CMV vector with a dummy sequence

(19) United States
(12) Patent Application Publication
(10) Pub. No.: US 2007/0054266 A1
(43) Pub. Date: Mar. 8, 2007

(54) Chemical sensor system

(75) Inventors: Takaaki Sato, Amagasaki-shi (JP); Junzo Hirono, Amagasaki-shi (JP); Hiroshi Hamana, Amagasaki-shi (JP); Masato Miyake, Amagasaki-shi (JP); Tomohiro Yoshikawa, Amagasaki-shi (JP); Jun Miyake, Amagasaki-shi (JP)

Correspondence Address: SEED INTELLECTUAL PROPERTY LAW GROUP PLLC, 701 FIFTH AVE, SUITE 5400, SEATTLE, WA, 98104, US

(73) Assignee: NATIONAL INSTITUTE of ADVANCED INDUSTRIAL SCIENCE and TECHNOLOGY, CHIYODA-KU (JP)

(21) Appl. No.: 10/516,210

(22) PCT Filed: May 28, 2003
(86) PCT No.: PCT/JP03/06719

§ 371 Date: Apr. 17, 2006

(30) Foreign Application Priority Data
May 28, 2002 (JP) 2002-154239
Jun. 13, 2002 (JP) 2002-172412
Jan. 14, 2003 (JP) 2003-005175

(51) Int. Cl. C12Q 1/68; C12M 1/34

(52) U.S. Cl. 435/6; 435/287.2

(57) ABSTRACT

A chemical sensor utilizing a chemical receptor (for example, one stimulating the sense of taste or smell) is provided. More specifically speaking, such a receptor is introduced into cells and the cells are immobilized on a support to form a chip. This chip is then employed as a component of a sensor. This sensor shows a reaction almost the same as the body's perception of the taste or smell or sense, thereby enabling analysis. Thus, it is also usable as an artificial sensory organ. Moreover, this sensor is usable in diagnosis, which imparts a high industrial usefulness to it.

### TECHNICAL FIELD

The present invention is in the field of analysis of chemical substances. More specifically, the present invention is related to a method for analyzing a chemical using a cell transfected with a chemical receptor such as an olfactory receptor and a system used thereof. The present invention is further related to a system for evaluating information in accordance with the stimulant responsiveness of a sensory receptor cell, a method for quantifying sensations, and a method for blending a stimulant inducing the sense of interest. The present invention is further related to genes for chemical receptors, chemical receptor proteins and the use thereof for developing odor sensors. The olfactory receptor genes and proteins of the present invention can be used for a sensor system measuring components of odor quality and intensity which are similar to those perceived by humans, animals and the like, and for measuring the intensity thereof, a sensor system extrapolating stimulant elements or a composition thereof from an arisen odor, and a system for automatically blending odor solution/gas having the odor presented.

### BACKGROUND ART

Sensors capable of sensing stimulus from outside have been developed for a long time, and remarkable advances have been seen for those using physicochemical methods. In particular, extremely high-performance sensors substituting for vision and audition have been developed. On the other hand, most of the sensors capable of sensing chemical substances developed, have been based on physicochemical methods; their application is limited and their tuning sensitivity and reproducibility are low.

Conventionally, neither sensor material with high relative sensitivity to specific odor sources such as typical chemical substances, nor odor sensors capable of evaluating the quality and intensity of odor as perceived in the brain have been developed.

The subject to which humans perceive odor is due to molecular chemical substances present in the air. Humans can perceive different odors having slightly different molecular structures owing to the response of olfactory receptor neurons distinguishing odor molecules (molecules producing odor) based on differences in molecular structure.

Olfactory receptor neurons (olfactory cells) are bipolar nerve cells that densely line the olfactory membrane in the recess of the nose, wherein odor receptor proteins that respond to odor molecules, called olfactory receptors, are expressed at high density. In olfactory cells, the chemical substances diffusing in the air from the stimulus source are detected by olfactory receptors and converted to neural signals. These neural signals are transmitted to the brain through the olfactory bulb (mitral cells or tufted cells) and the olfactory cortex such as the piriform cortex (pyramidal cells) and allow humans to sense odors.

The term called “odor quality” is used to express the odor perceived by humans, which are, for example, aromatic, camphoraceous, citrus fruit, herbal, drug, sweet, heavy, and the like.

For example, the optical isomers R(−)carvone and S(+)carvone induce different odor qualities as well as common odor qualities such as sweet, herbal and fresh. R(−)carvone induces the odor quality characteristic of spearmint and S(+)carvone induces that of caraway.

In odor detection devices, with established technology, multiple kinds of small sensors with low identification-specificity to odor substances in the air and small differences in their properties have been lined up and placed (hereinafter, also referred as “exhibited”) in the environment of sweet substances, to evaluate the difference in stimulus composition by comparing the output value between each sensor at the time when sensor output increases by some degree with time or by comparatively analyzing different stimulus-dependencies at the start of the initial response (e.g. for odor substances, Nature, Vol. 299, 352-355, 1982; Nikkei Science, 68-76, October 1991; T.IEE Japan, Vol. 113, C, 621-626, 1993; Japanese Patent No. 2647798, etc.).

The survival of living entities depends on their ability to recognize extracellular signals including gustation and olfaction, and respond to the extracellular signals. At the molecular level, the signals, which cooperatively interact to maintain cellular homeostasis, are recognized and transmitted via a network of interacting proteins which regulate activities such as multiplication, division and differentiation. Information transmission through biological signal transmission network is primarily mediated by protein-protein interactions capable of being dynamically assembled and decomposed in response to signals, thus a transient circuit is formed, linking external events to specific results such as the change in gene expression. However, there is no example using such a network.

With advancement of array technology, the use of this technology in various assays will be developed. The use of this promising technology, as an alternative assay method, using array analysis of high-density transfected cells (cellular array), is advocated. To date, however, array analysis of cells is limitedly applied, and has not been applied to the development of cell-based sensors at all.

Therefore, the object of the present invention is to provide a method, system, composition, devices and components for detecting and discriminating chemical substances, and programs, control methods and genes required for them. In particular, it is an object to efficiently provide information on chemical substances with high reproducibility by establishing a system, wherein cells express chemical receptors and measuring and/or analyzing the resultant signals.

During the evaluation of chemical substances such as odor substances using conventional methods, it was possible to predict from the difference in the profile of detected signals during stimulation (e.g. output signal waveform to odor substance) that there were differences in odor molecule compositions, but impossible to specify the olfactory sensation of a human using the profile. That is because the signal given by the sensor in the device does not correspond to the odor elemental information used to identify odor by the human olfactory system.

Regarding the blending of odor solution/gas, for example, the method, devices and vehicles for creating mimic odors are disclosed in Japanese Patent No. 2741749, but traditionally, it is impossible due to the above failure of detection systems to automatically blend specific odors whilst maintaining consistency with olfactory functions.

As with other sensations including gustation, a method for estimating the sensation in the brain using sensor outputs has not yet been developed.

Another object of the present invention is to provide a sensation-evaluation system capable of qualitative and quantitative evaluation of sensory elemental information in human senses such as olfaction and gustation using sensor output signals, a sensation-evaluation method, and a stimulant blending method capable of reproducing the desired quality of sensation.

### SUMMARY OF THE INVENTION

The above object was resolved by introducing a nucleic acid molecule encoding a chemical receptor into a cell and incorporating an expressable system into a sensor.

Chemical receptors such as the olfactory receptor have excellent detection properties and are capable of distinguishing between odor molecules different by only one carbon atom in a concentration-dependent manner with relatively high selectivity. The present invention thus tries to solve the problem of sensor material not having the same high-identification properties, by directly using the olfactory receptor. In the present invention, aims to selectively identify odors such as spearmint odor, caraway odor, mint odor and sweet odor by using the olfactory receptor showing the highest sensitivity to a specific odor quality.

Data from extensive studies on the mechanisms by which the olfactory system identifies odor, enabled us to identify the principle by which the olfactory system identifies an odor and eventually develop the present invention based on our observations. The qualitative and quantitative signal processing of odors, in analogy to the information processing of the olfactory neural system performed in a living organism, allows us to represent olfactory information as perceived by humans, animals, and the like. As analogous, similar examples may be found in the relationship between vision and video cameras/television, it is considered that a method imitating the mechanism of living entities is appropriate to identify and assay the sensory information.

The basic underlying concept of this aspect of the present invention is described below, using the olfactory system as an example. Odor molecules form the group with the lowest molecular weight amongst the molecules identified by living entities, making identification hard. In mice, processing of the signaling groups triggered by the identification of odor substances by approximately 1,000 kinds of olfactory receptors whose amino acid sequences vary in order to allow the identification of various odors. In humans, it is currently believed that odor is identified by the response profile arising at about 347 kinds of olfactory receptors which are considered to function similar to the olfactory receptors of mice.

At the olfactory receptor, it is thought that odor molecules are identified by differences in the three-dimensional configurations at a plurality of sites (hereinafter, also called “intermolecular interaction sites”) within the structure of an odor molecule. Therefore, for example, odor molecule A and odor molecule B, which partially share molecular structures, can be identified by one olfactory receptor, but cannot be identified by another olfactory receptor.

Even if an olfactory receptor usually responds to any one of odor molecule A and odor molecule B at a low concentration, the receptor may respond to both molecules at a higher concentration. There are many cases where an olfactory receptor can discriminate between two types of odor molecules having extremely similar structures, with a specificity ranging between 1 to 2 orders of magnitude by the presence or the absence of response.

In the olfactory system, we have found that, in order to process signals that clearly distinguish between the specificities of olfactory receptors with minor differences, the piriform cortex, which is one of the secondary sites in olfactory pathway in the brain, functions as a filter and a selective signal integrator by adding olfactory receptor signals sent with relatively high signals from the olfactory system, whilst suppressing relatively weak signals of olfactory receptors. Results obtained from the experiments described herein, show that signals summated amongst single or multiple types of olfactory receptors firstly activate the single neuron responsible for the odor quality at piriform cortex, suppresses excitatory activities in response to subsequently arriving signals at the piriform cortex, thereby reducing the contribution of such subsequent signals to the entire signal. Whilst it is described in Japanese Patent No. 2647798 that the olfactory receptor signal with high sensitivity is first input to the central nervous system, the present invention first discovered that the signals summated amongst other olfactory receptors, including single or multiple types of olfactory receptors most sensitive to a given stimulant also work. According to this observation, it is desirable that signals from a low-sensitivity sensor (olfactory receptor) which sends signals to the olfactory pathway in the brain after the first excitation, or after first exciting the neurons responsible for representing the odor quality at the piriform cortex with its respective signals added to signals from a plurality of sensors, are added to the desired signals after having been decreased by multiplication by an appropriate coefficient of in a range of about ½- 1/10, depending on the signal intensity at that time from the high-sensitivity sensor which had already begun sending signals. This addition of signals is conducted for each odor quality using sensors with sensitivity higher than a certain level to the odor molecules sharing a common odor quality. In many cases, signals from a single type of sensor are added to a plurality of odor qualities at different attributable fractions. Furthermore, the newly excited neurons responsible for representing the odor quality at the piriform cortex always increase the range of decrease in signals input by other sensors to other neurons.

Accordingly, purpose of the present invention is achieved by the following means:
(1) A chemical sensor comprising:

a) a nucleic acid comprising a sequence encoding a chemical receptor gene;

b) a support with a cell located thereon, wherein the cell has the nucleic acid introduced therein;

c) means for measuring a signal caused by the chemical receptor; and

d) means for providing information relating to a chemical by calculating the extent of activation of the chemical receptor from the intensity of the measured signal.
(2) The sensor according to item 1, wherein said nucleic acid further comprises a sequence encoding a marker gene.
(3) The sensor according to item 1, wherein said chemical receptor comprises a receptor selected from the group consisting of nuclear receptors, cytoplasmic receptors and cellular membrane receptors.
(4) The sensor according to Item 1, wherein said chemical receptor is selected from the group consisting of G protein coupled receptors, kinase type receptors, ion-channel type receptors, nuclear receptors, hormone receptors, chemokine receptors, and cytokine receptors.
(5) The sensor according to Item 1, wherein said chemical receptor comprises an olfactory receptor.
(6) The sensor according to Item 1, wherein said chemical receptor gene is selected from the group consisting of retinoic acid receptors, EGF receptors, interleukin receptors, and CSF receptors.
(7) The sensor according to Item 1, wherein said chemical receptor gene comprises a nucleic acid sequence or a variant or fragment thereof, selected from the group consisting of SEQ ID NO: 1, 3, 5, 7, 9, 11, 13, 15, 17, 19, 21, 34, 36, 38, 40, 42, 44, 46, 48, 50, 52, 54, 56, 58, 76, 78, 80, 82, 84, 86, 88, 90, 92, 94 and 96.
(8) The sensor according to Item 1, wherein said chemical receptor gene comprises at least about two species.
(9) The sensor according to Item 1, wherein said chemical receptor gene comprises at least about ten species.
(10) The sensor according to Item 1, wherein said chemical receptor gene comprises at least about twenty species.
(11) The sensor according to Item 1, wherein said chemical receptor gene comprises at least about three hundred species.
(12) The sensor according to Item 1, wherein said chemical receptor gene comprises substantially all species which are possessed by an animal selected from the group consisting of mice, humans, rats, dogs and cats.
(13) The sensor according to Item 1, wherein said chemical receptor gene comprises substantially all species of olfactory receptor genes which are possessed by an animal selected from the group consisting of mice, humans, rats, dogs and cats.
(14) The sensor according to Item 2, wherein said marker gene comprises G protein, said chemical receptor itself, or arrestin.
(15) The sensor according to Item 2, wherein said marker gene comprises Gα gene.
(16) The sensor according to Item 2, wherein said marker gene comprises Gα15, Gαq or Gαolf gene.
(17) The sensor according to Item 2, wherein said marker gene comprises Gα gene, Gβ gene and Gγ gene.
(18) The sensor according to Item 1, wherein said support comprises solid support.
(19) The sensor according to Item 1, wherein the support comprises a material selected from the group consisting of glass, silicon, silica, polystyrene and polymer films, which is coated or non-coated.
(20) The sensor according to Item 1, wherein the signal comprises an agent selected from the group consisting of intracellular calcium concentration, inositol triphosphate, cyclic AMP, diacyl glycerol, cyclic GMP and cellular membrane potential.
(21) The sensor according to Item 1, wherein the signal is intracellular calcium concentration, and said means for measuring signal comprises a means for electronically, chemically or biologically measuring calcium concentration.
(22) The sensor according to Item 2, wherein the marker gene is different from the genes which originally exist in said cell.
(23) The sensor according to Item 1, wherein the cell comprises a cell selected from the group consisting of HEK 293 cell, CHO cell, COS-7 cell, neuroblastoma and NG108-15.
(24) The sensor according to Item 1, wherein the cell substantially comprises one type of cell.
(25) The sensor according to Item 1, wherein the olfactory receptor gene is different from the genes which originally exist in said cell.
(26) The sensor according to Item 1, wherein the support comprises a cellular adhesion molecule.
(27) The sensor according to Item 16, wherein the cellular adhesion molecule comprises fibronectin, vitronectin, or laminin, or a fragment or variant thereof.
(28) The sensor according to Item 1, wherein the nucleic acid molecule or cell on said support is arranged thereon in an array format.
(29) The sensor according to Item 28, wherein the arrayed region has an area of about 200 mm² or less.
(30) The sensor according to Item 28, wherein the arrayed region has about 15 mm or less in length in the longitudinal direction.
(31) The sensor according to Item 1 further comprising liquid sufficient for covering the cell.
(32) The sensor according to Item 1 further comprising a medium for maintaining a cell.
(33) The sensor according to Item 32, wherein the medium is a liquid medium.
(34) The sensor according to Item 1, wherein said d) means for providing information comprises:

d-1) signal processing member for using a stimulus species categorizing method based on a stimulus element tuning specificity of a cell having a chemical receptor to add a first signal output by predetermined plurality of said sensors, to calculate a value of sensory elemental information expressing a sensation, and outputting a calculation result as a second signal; and

d-2) evaluation member for effecting qualitative and/or quantitative evaluation using the second signal output by the signal processing member.
(35) The sensor according to Item 34, wherein the subqualities of sensation to the stimulus categorizing method uses classification according to species of the chemical receptor.
(36) The sensor according to Item 34, wherein said signal processing member reduces, when one of first signals output by the plurality of sensors exceeds a predetermined value, the first signal output by a sensor different from the sensor and uses the reduced signal for producing the second signal.
(37) The sensor according to Item 34, wherein the signal processing member comprises:

a plurality of selection members and addition members corresponding to sensory elemental information;

a plurality of amplification members corresponding to each of the sensors;

a coefficient calculation member for controlling the amplification member, wherein the selection members multiply a plurality of the first signal with the coefficient designated by each of the sensors to produce a plurality of third signals;

the addition members add the plurality of third signals output by the corresponding selection member to produce a plurality of fourth signals;

the coefficient calculation member detects the maximum value among the plurality of fourth signals and normalizes each of the fourth signals using the maximum value to calculate control signals;

the amplification members use the corresponding control signals to produce the second signals corresponding to the intensity of sensory elemental information.
(38) The sensor according to Item 37, wherein when a stimulus is presented, the first signal output by the sensor, is transiently produced directed to a predetermined value corresponding to the intensity or concentration of the stimulus from zero level, wherein the zero level is set as a status where no response is found in response to no stimuli;

the third signal is transiently produced associated therewith directing to a predetermined value corresponding to the intensity or concentration of a stimulus from zero level:

the coefficient calculation member determines a sensor response starting at base time when one of the first signals is determined to be the signal output in response to a stimulus by the sensor for the first time, and

calculates at a predetermined time as an elapsed time from the base time, the control signal for controlling the amplification member using the third signal at the predetermined time;

controls the amplification member using the control signal which was calculated at the last time until a control signal is calculated at the predetermined time.
(39) The sensor according to Item 34, wherein when a stimulus is presented, the first signal output by the sensor, is transiently produced directed to a predetermined value corresponding to the intensity or concentration of the stimulus from zero level, wherein the zero level is set as a status where no response is found in response tono stimuli;

the third signal is transiently produced associated therewith directing to a predetermined value corresponding to the intensity or concentration of a stimulus from zero level;

the coefficient calculation member determines a sensor response starting at base time when one of the first signals is determined to be the signal output in response to a stimulus by the sensor for the first time;

during a period of time when the predetermined number of the plurality of third signals changes from augmentation to reduction,

calculates, at each time when the third signal is determined to start occurring significant output as a corresponding sense element, and when the third signal is determined to have achieved a plurality of boundary values which divide the section between the significant output value and the maximum value preset to the third signal into a plurality of segments;

controls the amplification member using the control signal which was calculated for the last time until the control signal is calculated.
(40) A chip for use in a chemical sensor comprising:
a) a nucleic acid molecule comprising a base sequence encoding a chemical receptor gene; and
b) a support having a cell arranged with the nucleic acid molecule introduced therein.
(41) The chip according to Item 40, wherein the nucleic acid molecule further comprises a sequence encoding a marker gene.
(42) The chip according to Item 40, further comprising means for transmitting a signal derived from the chemical receptor gene.
(43) The chip according to Item 40, wherein the chemical receptor comprises a receptor selected from the group consisting of intranuclear receptors, cytoplasmic receptors and cellular membrane receptors.
(44) The chip according to Item 40, wherein the chemical receptor comprises a receptor selected from the group consisting of G-protein coupled receptors, kinase-type receptors, ion-channel type receptors, intranuclear receptors, hormone receptors, chemokine receptors, and cytokine receptors.
(45) The chip according to Item 40 wherein the chemical receptor comprises an olfactory receptor.
(46) The chip according to Item 40, wherein the chemical receptor gene comprises a receptor selected from the group consisting of retinoic acid receptors, EGF receptors, interleukin receptors and CSF receptors.
(47) The chip according to Item 40, wherein the chemical receptor gene comprises a nucleic acid sequence set forth in SEQ ID NO. selected from the group consisting of SEQ ID NO: 1, 3, 5, 7, 9, 11, 13, 15, 17, 19, 21, 34, 36, 38, 40, 42, 44, 46, 48, 50, 52, 54, 56, 58, 76, 78, 80, 82, 84, 86, 88, 90, 92, 94 and 96, or a variant or fragment thereof.
(48) The chip according to Item 40, wherein the chemical receptor gene comprises at least about two species.
(49) The chip according to Item 40, wherein the chemical receptor gene comprises at least about ten species.
(50) The chip according to Item 40, wherein the chemical receptor gene comprises at least about twenty species.
(51) The chip according to Item 40, wherein the chemical receptor gene comprises at least about three hundred species.
(52) The chip according to Item 40, wherein the chemical receptor gene comprises all species of chemical receptor genes which are possessed by an animal selected from the group consisting of mice, humans, rats, dogs and cats.
(53) The chip according to Item 40, wherein the chemical receptor gene comprises substantially all species of olfactory receptor genes which are possessed by an animal selected from the group consisting of mice, humans, rats, dogs and cats.
(54) The chip according to Item 41, wherein the marker gene comprises Gα gene, the chemical receptor itself, or arrestin.
(55) The chip according to Item 41, wherein the marker gene comprises a Gα gene.
(56) The chip according to Item 41, wherein the marker gene comprises Gα15, Gαq or Gαolf.
(57) The chip according to Item 41, wherein the marker gene comprises Gα gene, Gβ gene and Gγ gene.
(58) The chip according to Item 40, wherein the support comprises a solid support.
(59) The chip according to Item 40, wherein the support comprises material selected from the group consisting of coated or non-coated glass, silicon, silica, polystyrene and polymer film.
(60) The chip according to Item 41, wherein a signal produced by the marker gene comprises an agent selected from the group consisting of intracellular calcium concentration, inositol triphosphate, cyclic AMP, diacylglycerol, cyclic GMP and cell membrane potential.
(61) The chip according to Item 41, wherein the marker gene is different from any genes already present in the cell.
(62) The chip according to Item 40, wherein the cell comprises a cell selected from the group consisting of HEK293 cell, CHO cell, COS-7 cell, neuroblastoma and NG108-15 cell.
(63) The chip according to Item 41, wherein the support comprises a connecting point for transmitting a signal derived from the marker. (64) The chip according to Item 40, wherein the support comprises a connecting point capable of transmitting at least one signal selected from the group consisting of an electrical signal, chemical signal and biological signal.
(65) The chip according to Item 40, wherein the olfactory receptor gene is different from any genes already present in the cell.
(66) The chip according to Item 40, wherein the support comprises a cellular adhesion molecule.
(67) The chip according to Item 66, wherein the cellular adhesion molecule comprises fibronectin, vitronectin or laminin, or a fragment or a variant thereof.
(68) The chip according to Item 40, wherein the nucleic acid molecule or cell on said support is arranged thereon in an array format.
(69) The chip according to Item 68, wherein the arrayed region has an area of about 200 mm² or less.
(70) The chip according to Item 68, wherein the arrayed region has a length of about 15 mm or less in the longitudinal direction.
(71) The chip according to Item 40, further comprising a liquid-sufficient for covering the cell.
(72) The chip according to Item 40, further comprising a medium for maintaining the cell.
(73) The chip according to Item 72, wherein the medium is a liquid medium.
(74) A method for obtaining information relating to a chemical in a sample, comprising the steps of:

A) providing a cell having a nucleic acid molecule introduced therein, wherein the nucleic acid molecule comprises a sequence encoding a chemical receptor gene;

B) providing the cell with a sample comprising or suspected to comprise a chemical of interest;

C) measuring a change induced by the chemical in a signal derived from the chemical receptor gene in the cell; and

D) calculating a level of activation of the chemical receptor from the change in intensity of the measured signal to provide information on the chemical.
(75) The method according to Item 74, wherein the nucleic acid molecule further comprises a sequence encoding a marker gene.
(76) The method according to Item 74, wherein the chemical is a source of olfaction.
(77) The method according to Item 74, wherein the chemical receptor gene comprises an olfactory receptor.
(78) The method according to Item 74, wherein the chemical receptor gene comprises at least about two species.
(79) The method according to Item 74, wherein the chemical receptor gene comprises at least about ten species.
(80) The method according to Item 74, wherein the chemical receptor gene comprises at least about twenty species.
(81) The method according to Item 74, wherein the chemical receptor gene comprises at least about three hundred species.
(82) The method according to Item 74, wherein the chemical receptor gene comprises all species of chemical receptor genes which are possessed by an animal selected from the group consisting of mice, humans, rats, dogs and cats.
(83) The method according to Item 75, wherein the marker gene comprises G protein-coupled protein.
(84) The method according to Item 75, wherein the marker gene comprises Gα gene, the chemical receptor itself, or arrestin.
(85) The method according to Item 75, wherein the marker gene comprises Gα15, Gαq or Gαolf.
(86) The method according to Item 75, wherein the marker gene comprises Gα gene, Gβ gene and Gγ gene.
(87) The method according of Item 74 wherein the cell is fixed on a support.
(88) The method according to Item 87, wherein the support comprises a solid support.
(89) The method according to Item 87, wherein the support comprises material selected from the group consisting of coated or non-coated glass, silicon, silica, polystyrene and polymer film.
(90) The method according to Item 75, wherein a signal produced by the marker gene comprises an agent selected from the group consisting of intracellular calcium concentration, inositol triphosphate, cyclic AMP, diacylglycerol, cyclic GMP and cell membrane potential.
(91) The method according to Item 75, wherein the marker gene is different from any genes already present in the cell.
(92) The method according to Item 74, wherein the cell comprises a cell selected from the group consisting of HEK293 cell, CHO cell, COS-7 cell, neuroblastoma and NG108-15 cell.
(93) The method according to Item 77, wherein the olfactory receptor gene is different from any genes already present in the cell.
(94) The method according to Item 74, wherein the support comprises a cellular adhesion molecule.
(95) The method according to Item 94, wherein the cellular adhesion molecule comprises fibronectin, vitronectin or laminin, or a fragment or a variant thereof.
(96) The method according to Item74, wherein the information regarding the signal comprises change in level of an agent selected from the group consisting of intracellular calcium concentration, inositol triphosphate, cyclic AMP, diacylglycerol, cyclic GMP and cell membrane potential.
(97) The method according to Item 96, wherein the information is presented at real time.
(98) The method according to Item 74, wherein the step B) comprises a step of providing the cell with the sample at a flow rate of about 1-4 mm/second.
(99) The method according to Item 74, wherein the step B) comprises a step of providing the cell with the sample at a flow rate of about 2-3 mm/second.
(100) The method according to Item 74, wherein the cell is provided with a liquid sufficient for covering the cell, and the step of B) comprises a step of providing the sample into the liquid.
(101) The method according to Item 74, wherein further the cell is provided with a medium for maintaining the cell.
(102) The method according to Item 101, wherein said medium comprises a liquid medium.
(103) The method according to Item 87, wherein the nucleic acid molecule or cell on said support is arranged thereon in an array format.
(104) The method according to Item 74, further comprising the step of correlating the information on the chemical with the information on the sample comprising or suspected to comprise the chemical of interest
(105) A sensation-evaluation system for evaluating sensation arising from a stimulant using output signal of a sensor comprising:

A) a plurality of sensors having different response characteristics from each other against stimuli from outside;

B) a signal processing member for using a stimulus species categorizing method based on a stimulus element tuning specificity of a cell having a chemical receptor to add a first signal output by predetermined plurality of said sensors, to calculate a value of sensory elemental information expressing a sensation, and outputting a calculation result as a second signal; and

C) an evaluation member for effecting qualitative and/or quantitative evaluation using the second signal output by the signal processing member.
(106) The sensation-evaluation system according to Item105, wherein the cell is transfected with a nucleic acid molecule comprising a nucleic acid sequence encoding said chemical receptor.
(107) The sensation-evaluation system according to Item105, wherein said signal processing member reduces, when one of first signals output by the plurality of sensors exceeds a predetermined value, the first signal output by a sensor different from the sensor and uses the reduced signal for producing the second signal.
(108) The sensation-evaluation system according to Item105, wherein the signal processing member comprises:

a plurality of selection members and addition members corresponding to sensory elemental information;

a plurality of amplification members corresponding to each of the sensors;

a coefficient calculation member for controlling the amplification member, and wherein

the selection members multiply a plurality of the first signal with the coefficient designated by each of the sensors to produce a plurality of third signals;

the addition members add the plurality of third signals output by the corresponding selection member to produce a plurality of fourth signal;

the coefficient calculation member detects the maximum value among the plurality of fourth signals and normalizes each of the fourth signals using the maximum value to calculate control signals; and

the amplification members use the corresponding control signals to produce the second signals corresponding to the intensity of sensory elemental information.
(109) The sensation-evaluation system according to Item105, wherein when a stimulus is presented, the first signal output by the sensor, is transiently produced directed to a predetermined value corresponding to the intensity or concentration of the stimulus from zero level, wherein the zero level is set as a status where no response is found in response to no stimuli;

the third signal is transiently produced associated therewith directing to a predetermined value corresponding to the intensity or concentration of a stimulus from zero level:

the coefficient calculation member determines a sensor response starting at base time when one of the first signals is determined to be the signal output in response to a stimulus by the sensor for the first time; and

calculates at a predetermined time as an elapsed time from the base time, the control signal for controlling the amplification member using the third signal at the predetermined time;

controls the amplification member using the control signal which was calculated at the last time until a control signal is calculated at the predetermined time.
(110) The sensation-evaluation system according to Item 108, wherein when a stimulus is presented, the first signal output by the sensor is transiently produced directed to a predetermined value corresponding to the intensity or concentration of the stimulus from zero level, wherein the zero level is set as a status where no response is found in response to no stimuli;

the third signal is transiently produced associated therewith directing to a predetermined value corresponding to the intensity or concentration of a stimulus from zero level;

the coefficient calculation member determines a sensor response starting base time when one of the first signals is determined to be the signal output in response to a stimulus by the sensor for the first time;

during a period of time when the predetermined number of the plurality of third signals change from augmentation to reduction,

calculates, at each time when the third signal is determined to start occurring significant output as a corresponding sense element, and when the third signal is determined to have achieved a plurality of boundary values which divide the section between the significant output value and the maximum value preset to the third signal into a plurality of segments;

controls the amplification member using the control signal which was calculated for the last time until the control signal is calculated, wherein when a stimulus is presented, the first signal output by the sensor, is transiently produced directed to a predetermined value corresponding to the intensity or concentration of the stimulus from zero level, wherein the zero level is set as a status where no response is found in response to no stimuli;

the third signal is transiently produced associated therewith directing to a predetermined value corresponding to the intensity or concentration of a stimulus from zero level;

the coefficient calculation member determines a sensor response starting base time when one of the first signals is determined to be the signal output in response to a stimulus by the sensor for the first time;

during a period of time when the predetermined number of the plurality of third signals change from augmentation to reduction,

calculates, at each time when the third signal is determined to start occurring significant output as a corresponding sense element, and when the third signal is determined to have achieved a plurality of boundary values which divide the section between the significant output value and the maximum value preset to the third signal into a plurality of segments;

controls the amplification member using the control signal which was calculated for the last time until the control signal is calculated.
(111) The sensation-evaluation system according to Item 105, wherein the chemical receptor is an olfactory receptor; and

the sensor is a sensor responding to an olfactory stimulus.
(112) A method for evaluating a sense in a sensation-evaluation system for evaluating sensation arising from a stimulant using an output signal of a sensor comprising a plurality of sensors having different response characteristics from each other against stimuli from outside and a signal processing member for processing an output signal from the sensors, comprising the steps of:

the first step wherein the signal processing member for using a stimulus species categorizing method based on a stimulus element tuning specificity of a cell having a chemical receptor to add a first signal output by predetermined plurality of said sensors, to calculate a value of sensory elemental information expressing a sensation, and outputting a calculation result as a second signal;

the second step wherein an evaluation member for effecting qualitative and/or quantitative evaluation using the second signal output by the signal processing member.
(113) The method according to Item 112, wherein the cell is transfected with a nucleic acid molecule comprising a nucleic acid sequence encoding said chemical receptor.
(114) The method according to Item 112, further comprising the third step wherein in the first step, said signal processing member reduces, when one of first signals output by the plurality of sensors exceeds a predetermined value, the first signal output by a sensor different from the sensor and uses the reduced signal for producing the second signal.
(115) The method according to Item 112, wherein the signal processing member comprises:

a plurality of selection members and addition members corresponding to sensory elemental information;

a plurality of amplification members corresponding to each of the sensor;

coefficient calculation member for controlling the amplification member, the first step further comprising

the fourth step wherein the selection members multiplies a plurality of the first signal with the coefficient designated by each of the sensors to produce a plurality of third signals;

the fifth step wherein the addition members add the plurality of third signals output by the corresponding selection member to produce a plurality of fourth signal;

the sixth step wherein the coefficient calculation member detects the maximum value among the plurality of fourth signals and normalizes each of the fourth signals using the maximum value to calculate control signals; and

the seventh step wherein the amplification members use the corresponding control signals to produce the second signals corresponding to the intensity of sensory elemental information.
(116) The method according to Item 114, wherein when a stimulus is presented, the first signal output by the sensor, is transiently produced directed to a predetermined value corresponding to the intensity or concentration of the stimulus from zero level, wherein the zero level is set as a status where no response is found in response to no stimuli and the signal produced by the fourth step is transiently produced associated therewith directing to a predetermined value corresponding to the intensity or concentration of a stimulus from zero level;

the method further comprises the eighth step wherein in the sixth step, the coefficient calculation member determines a sensor response starting base time when one of the first signals is determined to be the signal output in response to a stimulus by the sensor for the first time, and

calculates at a predetermined time as an elapsed time from the base time, the control signal for controlling the amplification member using the signal produced by the fifth step at the predetermined time, and controls the amplification member using the control signal which was calculated at the last time until a control signal is calculated at the predetermined time.
(117) The method according to Item 114, wherein when a stimulus is presented, the first signal output by the sensor, is transiently produced directed to a predetermined value corresponding to the intensity or concentration of the stimulus from zero level, wherein the zero level is set as a status where no response is found in response to no stimuli and the signal produced by the fourth step is transiently produced associated therewith directing to a predetermined value corresponding to the intensity or concentration of a stimulus from zero level;

the method further comprising the eighth step wherein in the sixth step, the coefficient calculation member determines a sensor response starting at base time when one of the first signals is determined to be the signal output in response to a stimulus by the sensor for the first time;

during a period of time when the predetermined number of the plurality of signals produced by the fifth step change from augmentation to reduction,

calculates, at each time when the signal produced by the fifth step is determined to start occurring significant output as a corresponding sense element, and when the signal produced by the fifth step is determined to have achieved a plurality of boundary values which divide the section between the significant output value and the maximum value preset to the signal produced by the fifth step into a plurality of segments;

controls the amplification member using the control signal which was calculated for the last time until the control signal is calculated.
(118) The method according to Item 112, wherein the chemical receptor is an olfactory receptor; and

the sensor is a sensor responding to an olfactory stimulus.
(119) A method for formulating a stimulant, comprising the steps of:

the first step of evaluating a predetermined stimulant using a sensation-evaluation system for evaluating sensation arising from a stimulant using the output signal of a sensor comprising:
A-1) a plurality of sensors having different response characteristics from each other against stimuli from outside;
A-2) a signal processing member for using a stimulus species categorizing method based on a stimulus element tuning specificity of a cell having a chemical receptor to add a first signal output by predetermined plurality of said sensors, to calculate a value of sensory elemental information expressing a sensation, and outputting a calculation result as a second signal; and
A-3) an evaluation member for effecting qualitative and/or quantitative evaluation using the second signal output by the signal processing member;

B) the second step of determining a ratio of stimulant elements to be mixed corresponding thereto using a result of evaluation corresponding to the stimulant elements obtained by the evaluation result of the first step and the sensation-evaluation system; and

C) the third step of mixing the determined stimulant elements at the determined ratio.
(120) The method according to Item 119, further comprising:

the fourth step of evaluating the mixed stimulant in the third step using the sensation-evaluation system; and

the fifth the step of comparing the evaluation step of fourth step and the evaluation result of the first step to determine the ratio to be newly mixed corresponding the stimulant element.
(121) A computer readable recording medium having a computer program recorded thereon for implementing a process in a computer in a sensation-evaluation system for evaluating sensation arising from a stimulant using an output signal of a sensor comprising a plurality of sensors having different response characteristics from each other against stimuli from outside and a signal processing member for processing an output signal from the sensors, the process comprising the procedures of:

the first procedure wherein the signal processing member using a stimulus species categorizing method based on a stimulus element tuning specificity of a cell having a chemical receptor to add a first signal output by predetermined plurality of said sensors, to calculate a value of sensory elemental information expressing a sensation, and outputting a calculation result as a second signal;

the second procedure wherein an evaluation member for effecting qualitative and/or quantitative evaluation using the second signal output by the signal processing member.
(122) A computer program for implementing a process in a computer in a sensation-evaluation system for evaluating sensation arising from a stimulant using output signal of a sensor comprising a plurality of sensors having different response characteristics from each other against stimuli from outside and a signal processing member for processing an output signal from the sensors, the process comprising the procedures of:

the first procedure wherein the signal processing member using a stimulus species categorizing method based on a stimulus element tuning specificity of a cell having a chemical receptor to add a first signal output by predetermined plurality of said sensors, to calculate a value of sensory elemental information expressing a sensation, and outputting a calculation result as a second signal;

the second procedure wherein an evaluation member for effecting qualitative and/or quantitative evaluation using the second signal output by the signal processing member.
(123) A nucleic acid molecule comprising:

(a) a polynucleotide having a base sequence set forth in SEQ ID NO. selected from the group consisting of SEQ ID NO: 1, 3, 5, 7, 9, 11, 13, 15, 17, 19 and 21, or a sequence fragment thereof;

(b) a polynucleotide encoding a polypeptide consisting of an amino acid sequence set forth in SEQ ID NO. selected from the group consisting of SEQ ID NO: 2, 4, 6, 8, 10, 12, 14, 16, 18, 20 and 22, or a fragment thereof;

(c) a polynucleotide encoding a variant polypeptide having an amino acid sequence set forth in SEQ ID NO. selected from the group consisting of SEQ ID NO: 2, 4, 6, 8, 10, 12, 14, 16, 18, 20 and 22, having at least one mutation selected from the group consisting of at least one amino acid substitution, addition and deletion, and having biological activity;

(d) a polynucleotide which is an allelic variant of DNA consisting of a base sequence set forth in SEQ ID NO. selected from the group consisting of SEQ ID NO: 1, 3, 5, 7, 9, 11, 13, 15, 17, 19 and 21;

(e) a polynucleotide encoding a species homolog of a polypeptide consisting of an amino acid sequence set forth in SEQ ID NO. selected from the group consisting of SEQ ID NO: 2, 4, 6, 8, 10, 12, 14, 16, 18, 20 and 22;

(f) a polynucleotide encoding a polypeptide hybridizable to any one of the polynucleotides (a) to (e) under stringent conditions, and having biological activity; or

(g) a polynucleotide consisting of a base sequence having at least 70% identity to any one of the polynucleotides (a) to (e) or a complementary sequence thereof, and having biological activity.
(124) The nucleic acid molecule according to Item 123,wherein the biological activity comprises a signal transduction activity of a chemical.
(125) A polypeptide comprising:

(a) a polypeptide encoded by polynucleotide of a nucleic acid sequence set forth in SEQ ID NO. selected from the group consisting of SEQ ID NO: 1, 3, 5, 7, 9, 11, 13, 15, 17, 19 and 21, or a fragment thereof

(b) a polypeptide consisting of an amino acid sequence set forth in SEQ ID NO. selected from the group consisting of SEQ ID NO: 2, 4, 6, 8, 10, 12, 14, 16, 18, 20 and 22, or a fragment thereof;

(c) a polypeptide comprising an amino acid sequence set forth in SEQ ID NO. selected from the group consisting of SEQ ID NO: 2, 4, 6, 8, 10, 12, 14, 16, 18, 20 and 22, having at least one mutation selected from at least one amino acid substitution, addition and deletion, and having biological activity;

(d) a polypeptide encoded by an allelic variant of a base sequence set forth in SEQ ID NO. selected from the group consisting of SEQ ID NO: 1, 3, 5, 7, 9, 11, 13, 15, 17, 19 and 21;

(e) a polypeptide which is a species homolog of an amino acid sequence set forth in SEQ ID NO. selected from the group consisting of SEQ ID NO: 2, 4, 6, 8, 10, 12, 14, 16, 18, 20 and 22; or

(f) a polypeptide having an amino acid sequence having at least 70% identity to any one of the polypeptides (a) to (e), and having biological activity.
(126) The polypeptide according to Item 125, wherein said biological activity comprises signal transduction activity of a chemical.
(127) Use of the nucleic acid molecule according to Item 123 or the polypeptide according to Item 125 for detecting a chemical.

It is understood that the usefulness, merit, and the like of the present invention would be obvious for those skilled in the art by referring to the following embodiments.

### BRIEF DESCRIPTION OF THE DRAWINGS

FIG. 1 shows a block diagram illustrating a general construction for a sensation evaluation system according to an embodiment of the present invention.

FIG. 2 shows a block diagram illustrating the general construction of the preprocessing member as shown in FIG. 1.

FIG. 3 shows a block diagram illustrating a general construction of a coefficient calculation member and an augmentation member as shown in FIG. 1.

FIG. 4 shows a block diagram illustrating a general construction of a sensation evaluation system of an embodiment of the present invention further comprising an additional addition member.

FIG. 5 shows a block diagram illustrating a general construction of a signal processing member replaceable with the signal processing member as shown in FIG. 1.

FIG. 6 shows tabular form data showing classification data for two olfactory substances S(+)carvone and R(−)carvone, from which response intensity of olfactory cells was derived.

FIG. 7 shows tabular form data showing the response ratio of an olfactory cell having responded two specific olfactory substances, S(+)carvone and R(−)carvone, against other olfactory molecules.

FIG. 8 shows tabular form data showing the numbers of responding cells identified by determining the existence ratio of olfactory receptors sensitive to S(+)carvone and R(−)carvone.

FIG. 9 shows tabular form data showing relative intensities of elements of odors induced by olfactory molecules.

FIG. 10 shows a diagram schematically illustrating a method for manufacturing a solid phase system transfection array (SPTA). This Figure shows the methodologies of solid phase transfection. A1 indicates a transfection array used in an exemplary embodiment of the present invention. 10 indicates the tip of an ink jet printer, a composition comprising an expression vector is included in a tube indicated by A2. 14 indicates a dish. In A2, a liquid cell culture medium is poured into the dish. In A3, the dish containing cell culture medium, is incubated under an appropriate condition, and thereby a transfection assay is completed as shows in A4.

FIG. 11 shows a detailed description of an example of a transfection method according to the present invention.

FIG. 12 shows an exemplary embodiment in which a method according to the present invention is carried out on a computer.

FIG. 13 shows an exemplary embodiment of a sensor according to the present invention. 1302 indicates tubing for aspirating overflow culture liquid. 1304 indicates a cover slip. 1306 indicates a wall for preventing a culture liquid from seeping. 1308 indicates tubing for feeding sample gas. 1310 indicates tubing for introducing culture liquid. 1316 indicates measurement chamber assembly substrate. 1318 indicates a cover slip. 1320 indicates cultured cells. 1322 indicates culture liquid. 1324 indicates a base for supporting the upper glass cover slip. 1330 indicates a connection to measurement apparatus such as fluorescence change measuring apparatus or the like.

FIG. 14 indicates exemplary data from studies in which the response of expressed olfactory receptors in olfactory receptor neurons was investigated by measuring change in fluorescence intensity of a calcium sensitive fluorescence stain. A to F and E′ are a sequential data sets measured, respectively. Between different data sets (“′” indicates the temporal division between sequential continuous measurements), the cells were washed for a couple of minutes with odorless culture liquid. Measurements methodology was as follows: olfactory receptor cell group in which fura-2 had been incorporated, was measured by microscopic image processing apparatus equipped with an SIT video camera to detect fluorescence images at the wavelength of 520 nm caused by radiating a fixed intensity of excitation light at 380 nm wavelength to the olfactory receptor cell groups with fura-2 incorporated therein. Subsequently, fluorescence intensity data corresponding to fluorescence images with eight video frames integrated therein, was recovered on a hard-disk every one third second, and the change in intracellular calcium concentration of individual cells was calculated using the recorded fluorescence images, by adding the detected fluorescence signals detected at 5×5=25 pixels per letter. Thereby, responses per each cell in response to 23 kinds of odor molecules at one or more concentrations are represented. Alphabetical order indicates the order of measurements. Three step traces per letter added correspond to each response of three different olfactory receptor cells. An olfactory receptor cell expressing the olfactory receptor cad-b153, and an olfactory receptor cell expressing the olfactory receptor cad-b158 were simultaneously measured; An olfactory receptor cell expressing the olfactory receptor cad-b86 was measured in a separate experiment. Further, ibmx was measured as a control response for response properties via cAMP, and hk was measured as a standard response for Ca²⁺ concentration increase via membrane potential dependent Ca²⁺ channel. Decrease in fluorescence intensity (change in the downward direction) corresponds to a response to an olfactory receptor. Stimulus was added to the culture liquid at the concentration for which the abbreviation of the odor molecules are set forth thereon, and administered to the cell for the period of time shown by the bar (i.e. 4 or 2 seconds).

Odor molecules and abbreviations are as follows: S(+)-carvone (sCa); R(−)-carvone (rCa); (−)menthone (mn); R(+)-pulegone (pu); isopulegol (ip); menthol (me); R(+)-limonene (lim); isoamyl acetate (am); vanillin (va); o-vanillin (ova); geraniol (ge); nerol (ne); hexanoic acid (mc6); heptanoic acid (mc7); octanoic acid (mc8); nonanoic acid (mc9); 1-hexanol (mh6); 1-heptanol (mh7); 1-octanol (mh8); 1-nonanol (mh9); indole (in); triethylamine (ta); isovaleric acid (iv); KCl (hk), (potassium chloride); isobutyl-1-methylxanthine (ibmx).

FIG. 15 shows the response thresholds corresponding to olfactory responses to each odor molecule, measured by an olfactory receptor cell.

FIG. 16 shows a response of olfactory receptor c257. A) shows the case where the receptor is expressed on an olfactory receptor cell, B) shows the case where the receptor is expressed on an established culture cell CHO. Olfactory receptor c257 response is also shown; B-1 shows the response of a cell expressing a functional receptor; whereas B-2 shows the response of a cell not expressing no such the receptor.

FIG. 17 shows an exemplary sensor of the present invention prepared by transfecting a cell on a chip with an olfactory receptor gene.

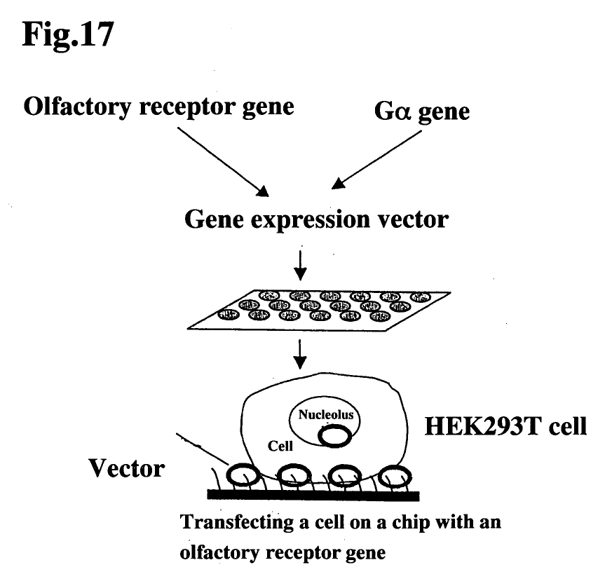

FIG. 18 shows a schematic diagram of a signal response to an olfactory stimulus. Calcium ion flux can be measured by the change in pigment fluorescence such as fura-2, fura-4 and the like, or change in fluorescence of a fluorescence protein sensitive to calcium concentration. 1800 indicates a cell, 1802 indicates an inactivated cell membrane receptor protein. 1804 indicates a signal. 1806 indicates the endoplasmic reticulum. 1808 indicates the nucleus. 1810refers to an extracellular signaling molecule. 1812 refers to an activated receptor protein. 1814 refers to inositol triphosphate.

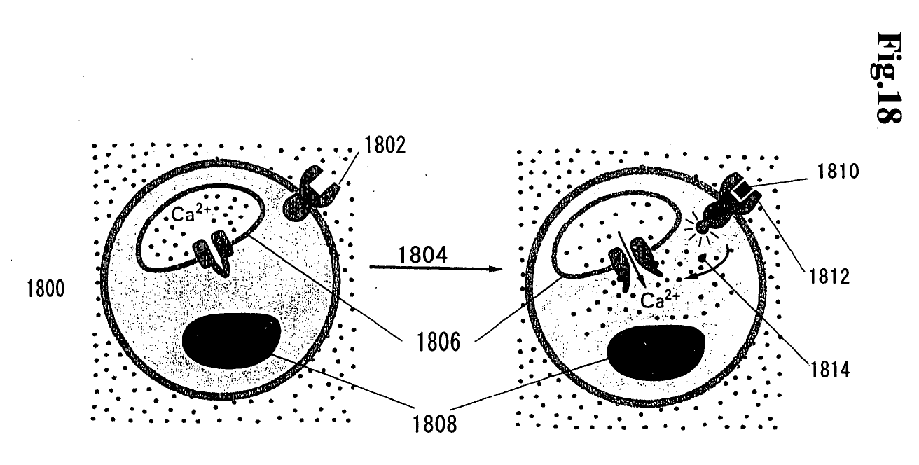

FIG. 19 refers to the data from an experiment using chemical (r-carvone and s-carvone) measured by using an exemplary sensor of the present invention.

FIG. 20 shows an exemplary configuration of the present invention. FIGS. 20A and 20B show an exemplary sensor system according to the present invention. FIG. 20C indicates an exemplary inner measurement member.

FIG. 21 shows an exemplary diagram of the Mercury Pathway Profiling system.

FIG. 22 shows a list of vectors used in the Mercury Pathway Profiling system.

FIG. 23A shows the signal transduction pathway used in the Mercury Pathway Profiling system.

FIG. 23B shows the effect of the activation on the signal transduction pathway used in the Mercury Pathway Profiling system.

FIG. 24 is an exemplary diagram showing the change in expression profile in Mercury Pathway Profiling system.

FIG. 25 shows data from an experiment confirming that a response is induced by a stimulus, using the Mercury Pathway Profiling system.

FIG. 26 shows an exemplary sensor prepared by using an actual cell using the Mercury Pathway Profiling system.

### DESCRIPTION OF SYMBOLS

Od1-Odn: sensors
P0: signal processing member
P1: pre processing member
P2: coefficient calculation member
P3: augmentation member
EV: evaluation member
SA1-SAm: selection member
J1-Jm, JC, AD1-AD3: addition member
RE: relative value determination member
MX: maximum value detection member
NOR: normalization member
M1-Mm: multiplier member

### DESCRIPTION OF SEQUENCES

SEQ ID NO: 1: nucleic acid encoding the transmembrane domains 3-6 of car-n272
SEQ ID NO: 2: protein encoded by the nucleic acid set forth in SEQ ID NO: 1
SEQ ID NO: 3: nucleic acid encoding the transmembrane domains 3-6 of car-c5
SEQ ID NO: 4: protein encoded by the nucleic acid set forth in SEQ ID NO: 3
SEQ ID NO: 5: nucleic acid encoding the transmembrane domains 3-6 of car-b161
SEQ ID NO: 6: protein encoded by the nucleic acid set forth in SEQ ID NO: 5
SEQ ID NO: 7: nucleic acid encoding the transmembrane domains 3-6 of car-b153
SEQ ID NO: 8: protein encoded by the nucleic acid set forth in SEQ ID NO: 7
SEQ ID NO: 9: nucleic acid encoding the transmembrane domains 3-6 of car-b85
SEQ ID NO: 10: protein encoded by the nucleic acid set forth in SEQ ID NO: 9
SEQ ID NO: 11: nucleic acid encoding the transmembrane domains 2-7 of car-n266
SEQ ID NO: 12: protein encoded by the nucleic acid set forth in SEQ ID NO: 11
SEQ ID NO: 13: nucleic acid consisting of the full coding region of car-n266
SEQ ID NO: 14: protein encoded by the nucleic acid set forth in SEQ ID NO: 13
SEQ ID NO: 15: nucleic acid encoding the full coding region of car-n272
SEQ ID NO: 16: protein encoded by the nucleic acid set forth in SEQ ID NO: 15
SEQ ID NO: 17: nucleic acid encoding the full coding region of car-b85
SEQ ID NO: 18: protein encoded by the nucleic acid set forth in SEQ ID NO: 17
SEQ ID NO: 19: nucleic acid encoding the full coding region of car-c5
SEQ ID NO: 20: protein encoded by the nucleic acid set forth in SEQ ID NO: 19
SEQ ID NO: 21: nucleic acid encoding the full coding region of car-b161
SEQ ID NO: 22: protein encoded by the nucleic acid set forth in SEQ ID NO: 21
SEQ ID NO: 23:AL1 primer
SEQ ID NO: 24:P26 primer
SEQ ID NO: 25:P27 primer
SEQ ID NO: 26: Anchor T primer
SEQ ID NO: 27: primer against transmembrane domain 2 (TM2)
SEQ ID NO: 28: P41 primer
SEQ ID NO: 29: P42 primer
SEQ ID NO: 30: W68 primer
SEQ ID NO: 31: W69 primer
SEQ ID NO: 32: W70 primer
SEQ ID NO: 33: P8 primer
SEQ ID NO: 34: nucleic acid encoding the murine olfactory receptor I7 (heptanal-sensitive) (Genbank Accession Number AF106007)
SEQ ID NO: 35: protein encoded by the nucleic acid set forth in SEQ ID NO: 34
SEQ ID NO: 36: nucleic acid encoding the murine olfactory receptor S1 (mc9/bc9-equi-sensitive) (Genbank Accession Number AF121972)
SEQ ID NO: 37: protein encoded by the nucleic acid set forth in SEQ ID NO: 36
SEQ ID NO: 38: nucleic acid encoding the murine olfactory receptor S50 (cc9-sensitive) (Genbank Accession Number AF121980)
SEQ ID NO: 39: protein encoded by the nucleic acid set forth in SEQ ID NO: 38
SEQ ID NO: 40: nucleic acid encoding the murine olfactory receptor S19 (mc9/mh9/bc9-equi-sensitive) (Genbank Accession Number AF121976)
SEQ ID NO: 41: protein encoded by the nucleic acid set forth in SEQ ID NO: 40
SEQ ID NO: 42: nucleic acid encoding the murine OR23 (lyral-sensitive) (only coding region of Genbank Accession Number X92969)
SEQ ID NO: 43: protein encoded by the nucleic acid set forth in SEQ ID NO: 42
SEQ ID NO: 44: nucleic acid encoding the murine olfactory receptor mOR-EV (vanillin-sensitive) (Genbank Accession Number AB061229)
SEQ ID NO: 45: protein encoded by the nucleic acid set forth in SEQ ID NO: 44
SEQ ID NO: 46: nucleic acid encoding the murine olfactory receptor or37a (Genbank Accession Number AJ133424)
SEQ ID NO: 47: protein encoded by the nucleic acid set forth in SEQ ID NO: 46
SEQ ID NO: 48: nucleic acid encoding the murine olfactory receptor C6 (Genbank Accession Number AF102523)
SEQ ID NO: 49: protein encoded by the nucleic acid set forth in SEQ ID NO: 48
SEQ ID NO: 50: nucleic acid encoding the murine olfactory receptor F5 (Genbank Accession Number AF102531)
SEQ ID NO: 51: protein encoded by the nucleic acid set forth in SEQ ID NO: 50
SEQ ID NO: 52: nucleic acid encoding the murine olfactory receptor S6 (Genbank Accession Number AF121974)
SEQ ID NO: 53: protein encoded by the nucleic acid set forth in SEQ ID NO: 52
SEQ ID NO: 54: nucleic acid encoding the murine olfactory receptor S18 (Genbank Accession Number AF121975)
SEQ ID NO: 55: protein encoded by the nucleic acid set forth in SEQ ID NO: 54
SEQ ID NO: 56: nucleic acid encoding the murine olfactory receptor S25 (Genbank Accession Number AF121977)
SEQ ID NO: 57: protein encoded by the nucleic acid set forth in SEQ ID NO: 56
SEQ ID NO: 58: nucleic acid encoding the murine olfactory receptor S46 (Genbank Accession Number AF121979)
SEQ ID NO: 59: protein encoded by the nucleic acid set forth in SEQ ID NO: 58
SEQ ID NO: 60: nucleic acid encoding the murine gustatory receptor T1R2 (Genbank Accession Number AY032623)
SEQ ID NO: 61: protein encoded by the nucleic acid set forth in SEQ ID NO: 60
SEQ ID NO: 62: nucleic acid encoding the murine gustatory receptor T1R1 (Genbank Accession Number AY032622)
SEQ ID NO: 63: protein encoded by the nucleic acid set forth in SEQ ID NO: 62
SEQ ID NO: 64:TPA: nucleic acid encoding the human gustatory receptor (TAS1R1) (Genbank Accession Number BK000153)
SEQ ID NO: 65: protein encoded by the nucleic acid set forth in SEQ ID NO: 64
SEQ ID NO: 66: nucleic acid encoding the human gustatory receptor (PTC) (Genbank Accession Number AY258597)
SEQ ID NO: 67: protein encoded by the nucleic acid set forth in SEQ ID NO: 66
SEQ ID NO: 68: nucleic acid encoding the α subunit of murine G-coupled protein (Genbank Accession Number M36778)
SEQ ID NO: 69: protein encoded by the nucleic acid set forth in SEQ ID NO: 68
SEQ ID NO: 70: nucleic acid encoding the β subunit of murine G-coupled protein (Genbank Accession Number M87286)
SEQ ID NO: 71: protein encoded by the nucleic acid set forth in SEQ ID NO: 70
SEQ ID NO: 72: nucleic acid encoding the γ subunit of murine G-coupled protein (Genbank Accession Number U37527)
SEQ ID NO: 73: protein encoded by the nucleic acid set forth in SEQ ID NO: 72
SEQ ID NO: 74: nucleic acid encoding the epidermal growth factor receptor (Genbank Accession Number BC023729)
SEQ ID NO: 75: protein encoded by the nucleic acid set forth in SEQ ID NO: 74
SEQ ID NO: 76: nucleic acid encoding the murine olfactory receptor G7 (Genbank Accession Number AF102537)
SEQ ID NO: 77: protein encoded by the nucleic acid set forth in SEQ ID NO: 76
SEQ ID NO: 78: nucleic acid encoding the Rattus norvegicus olfactory receptor MOR106-2 (Genbank Accession Number XM_{—}223984)
SEQ ID NO: 79: protein encoded by the nucleic acid set forth in SEQ ID NO: 78
SEQ ID NO: 80: nucleic acid encoding the murine olfactory receptor MOR8-2 (Genbank Accession Number NM_{—}147111)
SEQ ID NO: 81: protein encoded by the nucleic acid set forth in SEQ ID NO: 80
SEQ ID NO: 82: nucleic acid encoding the murine olfactory receptor M15(Genbank Accession Number AF282300)
SEQ ID NO: 83: protein encoded by the nucleic acid set forth in SEQ ID NO: 82
SEQ ID NO: 84: nucleic acid encoding the murine olfactory receptor K21 (Genbank Accession Number AF282279)
SEQ ID NO: 85: protein encoded by the nucleic acid set forth in SEQ ID NO: 84
SEQ ID NO: 86: nucleic acid encoding the murine olfactory receptor M12 (Genbank Accession Number AF283558)
SEQ ID NO: 87: protein encoded by the nucleic acid set forth in SEQ ID NO: 86
SEQ ID NO: 88: nucleic acid encoding the murine olfactory receptor C3 (Genbank Accession Number AF102522)
SEQ ID NO: 89: protein encoded by the nucleic acid set forth in SEQ ID NO: 88
SEQ ID NO: 90: nucleic acid encoding the murine olfactory receptor 68(Olfr68) (Genbank Accession Number NM_{—}013620)
SEQ ID NO: 91: protein encoded by the nucleic acid set forth in SEQ ID NO: 90
SEQ ID NO: 92: nucleic acid encoding the murine olfactory receptor 31(Olfr31) (Genbank Accession Number XM_{—}138899)
SEQ ID NO: 93: protein encoded by the nucleic acid set forth in SEQ ID NO: 92
SEQ ID NO: 94: nucleic acid encoding the murine olfactory receptor 60(Olfr60) (Genbank Accession Number NM_{—}146955)
SEQ ID NO: 95: protein encoded by the nucleic acid set forth in SEQ ID NO: 94
SEQ ID NO: 96: nucleic acid encoding the murine olfactory receptor 42 (Olfr42) (Genbank Accession Number XM_{—}138456)
SEQ ID NO: 97: protein encoded by the nucleic acid set forth in SEQ ID NO: 96
SEQ ID NO: 98: IHS expression vector (pBK-CMV-IHS-M4nc)
SEQ ID NO: 99: protein encoded by the nucleic acid set forth in SEQ ID NO: 98
SEQ ID NO: 100: Rho expression vector (pBK-CMV-Rho-M4nc)
SEQ ID NO: 101: protein encoded by the nucleic acid set forth in SEQ ID NO: 100
SEQ ID NO: 102: an exemplary sequence comprising the Pst I restriction site CTGCAG
SEQ ID NO: 103: an exemplary sequence comprising the Bsp EI restriction site TCCGGA
SEQ ID NO: 104: Rho-M4 chimeric cassette for pBK-CMV vector without the dummy sequence used in the Examples
SEQ ID NO: 105: protein encoded by the nucleic acid set forth in SEQ ID NO: 104
SEQ ID NO: 106: Rho-M4 chimeric cassette for pBK-CMV vector with the dummy sequence used in the Examples
SEQ ID NO: 107: protein encoded by the nucleic acid set forth in SEQ ID NO: 106
SEQ ID NO: 108: IHS-M4 chimeric cassette for pBK-CMV vector without the dummy sequence used in the Examples
SEQ ID NO: 109: protein encoded by the nucleic acid set forth in SEQ ID NO: 108
SEQ ID NO: 110: IHS-M4 chimeric cassette for PBK-CMV vector with the dummy sequence used in the Examples
SEQ ID NO: 111: protein encoded by the nucleic acid set forth in SEQ ID NO: 110

### BEST MODE FOR CARRYING OUT THE INVENTION

Hereinafter, the present invention will be described. It should be understood throughout the present specification that articles for a singular form (e.g., “a”, “an”, “the”, etc. in English; “ein”, “der”, “das”, “die”, etc. and their inflections in German; “un”, “une”, “le”, “la”, etc. in French; “un”, “una”, “el”, “la”, etc. in Spanish, and articles, adjectives, etc. in other languages) include the concept of their plurality unless otherwise mentioned. It should be also understood that the terms as used herein have definitions typically used in the art unless otherwise mentioned. Accordingly, unless otherwise defined, all technical and scientific terms used herein shall have the same meaning as generally understood by those skilled in the art to which the present invention pertains. If there is any inconsistency, the present specification precedes, including definitions.

### DEFINITION OF TERMS

Terms particularly used herein are defined as follows.

(Chemical Substance and Sense)

As used herein, “chemical substance” or “chemical” is used in the broadest sense of the term in the art and refers to the substance having a specific molecular structure. Examples of such a chemical substance include, but are not limited to, olfactory source as the origin of odor, gustatory source as the origin of taste, pheromone, intracellular information transmitter, cytokine, hormone, toxic substance, vitamin, nutritional factor, genetic control signal factor, and toxic gas. Useful chemical substances, being the subjects in the present invention include, but are not limited to, olfactory source, gustatory source, sample, biopsy specimen, chemical substance library, and drug.

As used herein, the term “sense” refers to the single sense unifying the corresponding sense element components formed by processing sensate information such as odor and taste, which are perceived by a living entity/organism (e.g. mammals such as humans, monkeys, dogs, mice, and the like), and “sense element” refers to different quality component consisting of sense, for example, individual olfactory odor qualities (sweet, herbal, and the like). “Sense” includes, but is not limited to, the five senses of gustation, olfaction, vision, audition and taction.

As used herein the terms “odor”, “smell”, “scent”, “fragrance” and “aroma” are interchangeably used herein and all of them refer to the sense transmitted in stimulation of the olfactory system of a living organism (typically, the nose) by chemical substances. Odor includes pheromones in insects and animals. As used herein, “olfaction” or “olfactory” refers to the sense of odor, and may be used herein to refer to same meaning as “odor” herein.

As used herein the terms “olfactory source”, “olfactory substance” and “odor substance” are interchangeably used herein and refer to the original substance causing odor. If the olfactory source consists of molecules herein, the molecules refer to odor molecules or olfactory molecules. Therefore, any chemical substances fall within the definition of olfactory source as long as they can be perceived by olfaction. Olfaction is usually transmitted through the air and it is desirable that an olfactory source has at least volatility.

As used herein, “taste” refers to the sense transmitted in stimulation of the gustatory system of a living organism (typically, the tongue) by chemical substances. If the gustatory source consists of molecules herein, the molecules refer to taste molecules or gustatory molecules. “Gustation” refers to the sense of taste and may be used in the same meaning as “taste” herein. Therefore, “gustatory source” and “gustatory substance” are interchangeably used herein and refer to the original substance causing taste. Examples of such a gustatory substance include, but are not limited to, sweet, sour, bitter, salty and tasty substances. Herein, gustation may also include “hot” in the broad sense of the term.

The gustatory organs responsible for gustation in an organism comprise gustatory buds including dozens of taste cells. The gustatory bud is connected to the taste nerve, which in turn, leads to the brain. The taste information obtained through taste cells is transmitted to the brain via the chorda tympani nerve, glossopharyngeal nerve, rhinopharyngeal nerve and the petrosus superficialis major nerve.

It is currently thought that a taste cell has about −40 to −50 mV of cellular potential. If chemical substances are attached to the microvillar membrane at the tip of taste cell, the taste cell depolarizes, a potential change is transmitted, voltage-dependent Ca channel opens and Ca²⁺ flows into the cell. As a result, the information in the nerve is transmitted by the release of transmitter (norepinephrine and the like) to the end of taste nerve fiber, wherein, it is thought that cyclic AMP, inositol trisphosphate, and the like, function as secondary messengers.

The gustatory receptor is expressed on taste cells. Examples of gustatory receptors include proton channel type receptors (H⁺ channel), sodium channel type receptors (Na⁺ channel), amino acids (glutamic acid, lysine, and the like) receptors, cellular membrane receptors of bitter substance, taste receptor membrane of bitter substance, sugar receptor, sugar receptor specific to artificial sweetener, and potassium channel type receptors (K⁺ channel). Therefore, the use of single or multiple receptor(s) in the present invention allows the detection of taste information with high reproducibility.

It is known that the receptor mechanism for bitter taste acts through various pathways including those mediated through G protein. Bitter substances like denatonium are transmitted by increases in Ca²⁺ concentration, presence of IP₃ receptors. A mudpuppy and the like possess depolarization closing K⁺ channel. It is thought that depolarization directly through the membrane is triggered by these bitter substances. Therefore, the present invention allows for detection of bitter substances.

It is known that the receptor mechanism for sweet taste is transmitted via various pathways including those mediated through G protein. Therefore, using various G proteins as markers, it is possible to detect transmission of information by using cAMP, IP₃ and Ca²⁺ as signal-transduction factors. The present invention allows the detection of sweet substances.

It is known that there are various receptor mechanisms for taste also and specific receptor mechanisms exist for glutamic acid, inosinic acid, guanylic acid, and the like. The glutamic acid receptor is coupled with G protein, thus allowing detection of Therefore, it is possible to detect the information by measuring cAMP concentration and the like. The present invention allows detection of gustation substances.

Ion channels and receptors are involved in gustation of salty, sour, and the like, and the use of these channels and/or receptors makes it possible to detect information, which may then be the subject in the present invention.

As used herein, “receptor” refers to the molecule which is present on the cell or in the cell nucleus and has the capability of binding to factors from either outside or within the cell, and that signal transduction is triggered by the binding. The receptor usually takes the form of a protein. The binding partner of the receptor is usually called a ligand.

As used herein, “agonist” refers to an agent which binds to the receptor of a biologically active substance (ligand) and shows the same (or similar) action as the substance.

As used herein, “antagonist” refers to an agent which antagonistically acts upon the binding to the receptor of a biologically active substance (ligand) and does not show the physiological action mediated by the receptor by itself, which comprises blockers, inhibitors, and the like.

As used herein, “chemical receptor” refers to the receptor using chemical substances as ligands. Examples of such a chemical receptor include, but are not limited to, the receptors selected from the group consisting of nuclear receptors, cytoplasmic receptors and cell membrane receptors.

Preferably, said chemical receptor comprises the receptor selected from the group consisting of G-protein coupled receptors, kinase type receptors, ion-channel type receptors, nuclear receptors, chemokine receptors and cytokine receptors.

As used herein, “olfactory receptor” refers to the receptor involved in olfactory signal transduction. Examples of such an olfactory receptor include, but are not limited to, the receptor encoded by the nucleic acid sequence indicated by, for example, SEQ ID NO. 1, 3, 5, 7, 9, 11, 13, 15, 17, 19, 21, 34, 36, 38, 40, 42, 44, 46, 48, 50, 52, 54, 56, 58, 76, 78, 80, 82, 84, 86, 88, 90, 92, 94, 96, or the like. Examples of an amino acid sequence of the olfactory receptor include, but are not limited to, the amino acid sequence indicated by, for example, SEQ ID NO. 2, 4, 6, 8, 10, 12, 14, 16, 18, 20, 22, 35, 37, 39, 41, 43, 45, 47, 49, 51, 53, 55, 57, 59, 77, 79, 81, 83, 85, 87, 89, 91, 93, 95, 97, or the like. In the present invention, the variant or fragment of such a sequence also can be used as long as the function (i.e. the function as a chemical receptor, including interaction with ligand and downstream signaling sequence transmitting action) is retained. It is known that an olfactory receptor is 7-transmembrane-type protein wherein the amino acid sequence interacting is found in the intracellular portion of transmembrane domain 3. The characteristic sequence to distinguish such a sequence from other G-protein coupled receptors can be typically identified by sequence LHTPMY at the start of transmembrane domain 2, sequence MAYDRYVAIC, which includes the amino acid sequence interacting with G protein of transmembrane domain 3, and sequence PMLNPF of transmembrane domain 7. The encoding region of a gene of an olfactory receptor usually has a characteristic of not including introns. It is also known that the second extracellular loop tends to be relatively long and the intracellular loop tends to be shorter than other receptor groups.

As used herein, “gustatory receptor” refers to the receptor involved in gustatory signal transduction, which is encoded by the nucleic acid sequence indicated by the nucleic acid sequence selected from the group consisting of SEQ ID NO. 60, 62, 64 and 66. Examples of an amino acid sequence of the gustatory receptor include, for example, the amino acid sequence selected from the group consisting of SEQ ID NO. 61, 63, 65 and 67. In the present invention, the variant or fragment of such a sequence can also be used as long as the function (the function as a chemical receptor, including i.e. interaction with the ligand and downstream signaling sequence transmitting action) is retained.

“Kinase-type cytokine receptor” also can be used herein. Examples of such a kinase-type cytokine receptor include, for example, EGF (epithelial growth factor) receptor. Examples of such a sequence include, but are not limited to, the nucleic acid sequence having the sequence indicated by SEQ ID NO. 74 (the amino acid sequence is indicated by SEQ ID NO. 75). In the present invention, the variant or fragment of such a sequence also can be used as long as the function (i.e. function as a chemical receptor, including interaction with the ligand and downstream signaling sequence transmitting action) is retained.

As used herein, “signal” refers to an agent transmitting information. As used herein, a signal especially refers to an agent transmitted by intracellular signal transduction. In the context of a cellular receptor, it refers to serial transduction of information from the initial signal produced in response to a stimulus to the function or expression of a gene product. A lipid(fat)-soluble substance such as a steroid hormone is transmitted by a nuclear receptor, whereas a water-soluble substance (e.g. hormone, cytokine, neurotransmitter, and the like) has a specific receptor on cell membrane which transmits signals within a cell. After signals are transmitted, the cell induces various responses. Examples of such a signal include, but are not limited to, intracellular calcium concentration (Ca²⁺), IP₃, diacyl glycerol, cAMP, cGMP and cellular membrane potential.

Signal transduction is classified by receptor form as the following examples.

1) G-protein coupled receptors: a cell-membrane 7-transmembrane-type receptor coupled with trimeric G-protein. This type further falls into the cAMP system producing cAMP as the second messenger and the inositol phospholipid transmitter system producing inositol-1,4,5-triphosphate (IP₃) or diacyl glycerol (DG) as the second messenger. cAMP can activate some pathways in single or parallel. In some of nerve cells, such as olfactory-receptor nerve cells, cAMP-dependent ion-channel are opened, the cellular membrane is depolarized, and Ca²⁺ enter the cell through the channel, transiently increasing intracellular Ca²⁺ concentration. cAMP activates cAMP-dependent kinase (A kinase), phosphorylates serine and/or threonine residues of function-protein, and modifies its activity. On the other hand, IP₃ binds to IP₃ receptor on the endoplasmic reticulum and accelerates the release of Ca²⁺ into a cell. Diacyl glycerol promotes the action of hormones and the like by activating C kinase.

2) Ion-channel type receptors: the receptor itself forms an ion channel which opens by binding to an agonist such as a neurotransmitter. As a result, ions flow in/out, modifying various bioactivities.

3) Tyrosine-kinase type receptors: a type used by cytokines. Cytokine receptors, many of which have tyrosine-kinase activity, enhances tyrosine-kinase activity by binding to an agonist, thus inducing the phosphorylation of tyrosine residues. As a result, the protein has a Src homology region 2 (SH₂), allowing binding to tyrosine phosphorylation sites, and thus downstream signaling events and promotion of action.

4) Guanylic-acid cyclase type receptors: the receptor itself has guanylic-acid cyclase activity and produces cGMP by receptor stimulation. Within the cell, G protein is responsible for signal transduction. G protein has a 3 subunit structure of αβγ wherein the a subunit usually binds to GDP. The coupled receptor binds to GTP when stimulated and the trimer is then separated into α subunit and βγ subunit. Thus, enzyme activity is accelerated or inhibited, resulting in signal transduction.

The activation of acceleratory G-protein called Gs activates adenylate cyclase and increases cAMP level. The activation of inhibitory G-protein called Gi decreases cAMP wherein adenylate cyclase is inhibited. The activation of transducin of visual cells, which is a Gi sub-type, activates phosphodiesterase of cGMP catabolic enzyme and decreases cGMP level. The activation of a G protein called Gq activates phopholipase C and produces IP₃. All of these pathways can be used in the present invention.

In taste cells, G proteins such as Gs, Gi and Gq, and also, specific G proteins such as gastducin and transducin are present. Therefore, these proteins can be used as marker proteins in the present invention. These proteins are reported by L. Buck et al. Cell, 65, 175 (1991), Abe K., et al. J. Biol. Chem. 268, 12033 (1993), and the like. In the present invention, these G proteins specific to gustation can be used as markers and can also be used for the transmission of chemical substances other than gustatory substance.

As used herein, the term “marker” refers to an agent for generating an agent or a label which is measurable from outside. Accordingly, markers can reflect the level or frequency of a substance or state of interest. Examples of such a marker include, but are not limited to, G-coupled protein, nucleic acids encoding a gene, gene products, metabolic products, receptors, ligands, antibodies, and the like.

As used herein, the “agent” may usually be any substance orotheragent (e.g., energy, such as light, radiation, heat, electricity, or the like) as long as the intended purpose can be achieved. Examples of such a substance include, but are not limited to, proteins, polypeptides, oligopeptides, peptides, polynucleotides, oligonucleotides, nucleotides, nucleic acids (e.g., DNA such as cDNA, genomic DNA, or the like, and RNA such as mRNA), polysaccharides, oligosaccharides, lipids, low molecular weight organic molecules (e.g., hormones, ligands, information transduction substances, fluorophores, luminophores and the like), fluorescent proteins, luminescence proteins and combinations of these molecules.

Therefore, as used herein, examples of a marker include, but are not limited to, G protein as well as the factor (e.g. such as complementary nucleic acid, ligands and antibodies) interacting with the intracellular agent showing the state of cells (e.g. nucleic acid encoding a gene, gene products (e.g. mRNA, protein, post-translationally modified protein), metabolites, receptors, and the like). Such a marker can be, preferably, advantageous to specifically interact with the agent of interest. Such a specific interaction can be achieved by coupling a specific marker such as G protein to a specific chemical receptor. Such specificity refers to, for example, the property wherewith the degree of interaction with the molecule of interest is significantly higher than that with similar molecules. In the present invention, preferably, such a marker is intracellular, but can also be extracellular.

As used herein, “marker gene” refers to a gene capable of producing markers. Usually, a marker gene encodes the marker as a protein, or includes, but not limited to, a protein capable of producing a marker or the protein capable of ultimately producing a marker. Examples of such a typical marker gene include, but are not limited to, G protein.

As used herein, “G protein” refers to guanine nucleotide binding regulatory protein, which is the GTP binding protein specifically binding to GTP (guanosine 5′ triphosphate) or GDP (guanosine 5′ diphosphates) and showing the enzymatic activity of decomposing bound DTP into GDP and phosphate. Typically, G protein functions as a factor in converting and transmitting information in the intracellular signal transduction pathway mediated by receptors such as neurotransmitters. Trimeric G protein consists of three types of subunits, α(Gα), β(Gβ) and γ(Gγ). G proteins are widely present across the eukaryotes from yeast to human, mouse, and the like, thus such G proteins can also be used in the present invention. Examples of such G proteins include, but not are limited to, those described in Gα (SEQ ID NO: 68 (nucleic acid sequence), 69 (amino acid sequence)), Gβ (SEQ ID NO: 70), 71 (amino acid sequence), Gγ (SEQ ID NO: 72 (nucleic acid sequence), SEQ ID NO: 73 (amino acid sequence)). G protein is usually present as the complex of αβγ (Gαβγ) and is activated by a receptor which has a 7 transmembrane structure (G protein coupled receptor). G protein coupled receptor is activated by an extracellular first messenger which exchanges GDP for GTP by binding to Gα. Gα bound to GTP is dissociates from Gβγ and Gα and Gβγ independently or mutually regulates the enzyme, thereby changing the concentration of intracellular second messengers such as adenylate cyclase, the activity of ion channels, and the like. If GTP is decomposed to GDP by the enzymic activity of Gα itself, Gα re-binding to Gβγ returns to inactive trimeric Gαβγ. In the present invention, Gα protein can be used as a marker because Gα proteins are directly coupled with receptor genes. More preferably, it is adgantageous to use all subunits of Gα, Gβ and Gγ in the present invention. Preferably, it is advantageous to use these G-coupled proteins in a coupled manner.

As used herein, “signal intensity” refers to the physical level of signal, which can be measured, corresponding to signal character, using the technique well-known in the art. Examples of such a signal intensity include, but are not limited to: electrical signals such as electrical current and potential, the intensity of electrical signals such as electrical current and potential measured with ammeter, the concentration of calcium, IP₃, and the like, the amount capable of being evaluated by an assay or device which can measure the relative value of the concentration value or change which reflects the concentration or concentration change of calcium, IP₃, and the like. These signals can be measured using patch-clamp method and the like. Preferably, calcium concentration can be qualitatively and quantitatively measured by measuring fluorescence with fura-2. For such a reagent, reagents or kits are commercially available from Sigma, Funakoshi, Dojinkagaku, and the like.

Patch-clamp method records the activities of single or multiple ion channel molecule(s) on cell membranes simultaneously. Examples of such a patch-clamp method include giga-seal method applicable to many cell lines.

During the patch-clamp method, a microelectrode also called glass patch electrode, is touched on the cell surface, negative pressure is applied. This increases the electric resistance between the patch electrode and cells and the adherence without leakage can be obtained, thus forming a giga-seal where usually 10 Go or higher seal is attained. With such high resistance, all the electric current passing through patch membrane is sent to the electrode without leakage into the external solution and can be measured as recorded electric current from the patch electrode. This measurement method is called cell-attached recording.

After forming a giga-seal, by applying further higher negative pressure to patch electrode, the patch membrane is destroyed and the direct coupling of the patch electrode internal solution and the interview of the cell is made, allowing the measurement of the electric current passing through all ion channels present on the cell membrane under the conditions whereon cell membrane potential is fixed. This measurement method is called whole-cell recording.

As used herein, “activation” of receptor means to be in the state wherein a receptor can transmit signals downstream ligand binding.

As used herein, “activation level” of receptor refers to the degree of receptor activation, which can be expressed, for example, using the magnitude of signal.

(Cellular Biology)

The term “cell” is herein used in its broadest sense in the art, referring to a structural unit of tissue of a multicellular organism, which is capable of self replicating, has genetic information and a mechanism for expressing it, and is surrounded by a membrane structure which isolates the cell from the outside. Cells used herein may be either naturally-occurring cells or artificially modified cells (e.g., fusion cells, genetically modified cells, etc.), as long as the cell has a chemical receptor or is capable of having such a chemical receptor introduced therein. Examples of cell sources include, but are not limited to, a single-cell culture; the embryo, blood, or body tissue of normally-grown transgenic animals; a mixture of cells derived from normally-grown cell lines; and the like. Preferably, a cell which is easily transformed or transfected is used. Cells used in the present invention are preferably cells which are easily cultured and/or maintained on a support.

Cells used herein may be derived from any organism (e.g., any unicellular organisms (e.g., bacteria and yeast) or any multicellular organisms (e.g., animals (e.g., vertebrates and invertebrates), plants (e.g., monocotyledons and dicotyledons, etc.)). For example, cells used herein are derived from a vertebrate (e.g., Myxiniformes, Petronyzoniformes, Chondrichthyes, Osteichthyes, amphibian, reptilian, avian, mammalian, etc.), more preferably mammalian (e.g., monotremata, marsupialia, edentate, dermoptera, chiroptera, carnivore, insectivore, proboscidea, perissodactyla, artiodactyla, tubulidentata, pholidota, sirenia, cetacean, primates, rodentia, lagomorpha, etc.). In one embodiment, cells derived from Primates (e.g., chimpanzee, Japanese monkey, human) are used. Particularly, without limitation, cells derived from a human are used. The above-described cells may be either stem cells or somatic cells. Also, the cells may be adherent cells, suspended cells, tissue forming cells, and mixtures thereof. The cells may be used for transplantation.

Any organ may be targeted by the present invention. A tissue or cell targeted by the present invention may be derived from any organ. As used herein, the term “organ” refers to a morphologically independent structure localized at a particular portion of an individual organism in which a certain function is performed. In multicellular organisms (e.g., animals, plants), an organ consists of several tissues spatially arranged in a particular manner, each tissue being composed of a number of cells. An example of such an organ includes an organ relating to the vascular system. In one embodiment, organs targeted by the present invention include, but are not limited to, skin, blood vessel, cornea, kidney, heart, liver, umbilical cord, intestine, nerve, lung, placenta, pancreas, brain, peripheral limbs, retina, and the like.

As used herein, the term “tissue” refers to an aggregate of cells having substantially the same function and/or form in a multicellular organism. “Tissue” is typically an aggregate of cells of the same origin, but may be an aggregate of cells of different origins as long as the cells have the same function and/or form. Therefore, when stem cells of the present invention are used to regenerate tissue, the tissue may be composed of an aggregate of cells of two or more different origins. Typically, a tissue constitutes apart of an organ. Animal tissues are separated into epithelial tissue, connective tissue, muscular tissue, nervous tissue, and the like, on a morphological, functional, or developmental basis. Plant tissues are roughly separated into meristematic tissue and permanent tissue according to the developmental stage of the cells constituting the tissue. Alternatively, tissues may be separated into single tissues and composite tissues according to the type of cells constituting the tissue. Thus, tissues are separated into various categories. In the present invention, sensors or chips can be consitututed using a tissue.

As used herein, the term “stem cell” refers to a cell capable of self replication and pluripotency. Typically, stem cells can regenerate an injured tissue. Stem cells used herein may be, but are not limited to, embryonic stem (ES) cells or tissue stem cells (also called tissular stem cell, tissue-specific stem cell, or somatic stem cell). Accordingly, a stem cell may be used in the present invention.

As used herein, the term “somatic cell” refers to any cell other than a germ cell, such as an egg, a sperm, or the like, which does not transfer its DNA to the next generation. Typically, somatic cells have limited or no pluripotency. Somatic cells used herein may be naturally-occurring or genetically modified.

As used herein, the term “isolated” means that naturally accompanying material is at least reduced, or preferably substantially completely eliminated, in normal circumstances. Therefore, the term “isolated cell” refers to a cell substantially free from other accompanying substances (e.g., other cells, proteins, nucleic acids, etc.) in natural circumstances. The term “isolated” in relation to nucleic acids or polypeptides means that, for example, the nucleic acids or the polypeptides are substantially free from cellular substances or culture media when they are produced by recombinant DNA techniques; or precursory chemical substances or other chemical substances when they are chemically synthesized. Isolated nucleic acids are preferably free from sequences naturally flanking the nucleic acid within an organism from which the nucleic acid is derived (i.e., sequences positioned at the 5′ terminus and the 3′ terminus of the nucleic acid).

As used herein, the term “established” in relation to cells refers to a state of a cell in which a particular property (pluripotency) of the cell is maintained and the cell undergoes stable proliferation under culture conditions. Therefore, established stem cells maintain pluripotency. In the present invention, such an established cell is preferably used since such a cell provides a stablized result.

As used herein, the term “differentiated cell” refers to a cell having a specialized function and form (e.g., muscle cells, neurons, etc.). Unlike stem cells, differentiated cells have no or little pluripotency. Examples of differentiated cells include epidermial cells, pancreatic parenchymal cells, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, cartilage cells, and the like.

As used herein, the term “state” refers to a condition concerning various parameters of a cell (e.g., cell cycle, response to an external factor, signal transduction, gene expression, gene transcription, etc.). Examples of such a state include, but are not limited to, differentiated states, undifferentiated states, responses to external factors, cell cycles, growth states, and the like.

(Biochemistiry and Molecular Biology)

As used herein, the term “gene” refers to an element defining a genetic trait. A gene is typically arranged in a given sequence on a chromosome. A gene which defines the primary structure of a protein is called a structural gene. A gene which regulates the expression of a structural gene is called a regulatory gene (e.g., promoter). Genes herein include structural genes and regulatory genes unless otherwise specified. Therefore, the term “cyclin gene” typically includes the structural gene of cyclin and the promoter of cyclin. As used herein, “gene” may refer to “polynucleotide”, “oligonucleotide”, “nucleic acid”, and “nucleic acid molecule” and/or “protein”, “polypeptide”, “oligopeptide” and “peptide”. As used herein, “gene product” includes “polynucleotide”, “oligonucleotide”, “nucleic acid” and “nucleic acid molecule” and/or “protein”, “polypeptide”, “oligopeptide” and “peptide”, which are expressed by a gene. Those skilled in the art understand what a gene product is, according to the context. Accordingly, gene used herein usually includes not only double-stranded DNA but also each single-stranded DNA such as sense chain and antisense chain constituting thereof. Therefore, the genes of the present invention include any of double-stranded DNA including human genome DNA, and single-stranded DNA (sense chain) including cDNA, as well as a single stranded DNA (antisense) having a sequence complementary to the sense chain, as well as fragments thereof.

As used herein, the term “homology” in relation to a sequence (e.g., a nucleic acid sequence, an amino acid sequence, etc.) refers to the proportion of identity between two or more gene sequences. Therefore, the greater the homology between two given genes is, the greater is the identity or similarity between their sequences. Whether or not two genes have homology is determined by comparing their sequences directly or by a hybridization method under stringent conditions. When two gene sequences are directly compared with each other, these genes have homology if the DNA sequences of the genes have representatively at least 50% identity, preferably at least 70% identity, more preferably at least 80%, 90%, 95%, 96%, 97%, 98%, or 99% identity with each other. As used herein, the term “similarity” in relation to a sequence (e.g., a nucleic acid sequence, an amino acid sequence, or the like) refers to the proportion of identity between two or more sequences when conservative substitution is regarded as positive (identical) in the above-described homology. Therefore, homology and similarity differ from each other in the presence of conservative substitutions. If no conservative substitutions are present, homology and similarity have the same value.

The similarity, identity and homology of amino acid sequences and base sequences are herein compared using FASTA with the default parameters.

The terms “protein”, “polypeptide”, “oligopeptide” and “peptide” as used herein have the same meaning and refer to an amino acid polymer having any length. This polymer may be a straight, branched or cyclic chain. An amino acid may be a naturally-occurring or non-naturally-occurring amino acid, or a variant amino acid. The term may include those assembled into a composite of a plurality of polypeptide chains. The term also includes a naturally-occurring or artificially modified amino acid polymer. Such modification includes, for example, disulfide bond formation, glycosylation, lipidation (acylation), acetylation, phosphorylation, or any other manipulation or modification (e.g., conjugation with a labeling moiety). This definition encompasses a polypeptide containing at least one amino acid analog (e.g., non-naturally-occurring amino acid, etc.), a peptide-like compound (e.g., peptoid), and other variants known in the art. Gene products, such as extracellular matrix proteins (e.g., fibronectin, etc.), are usually in the form of polypeptide, however, there may be a form of a polypeptide variant as long as it has the same function. Polypeptides having specific amino acid sequences include fragments, cognates, derivatives and variants thereof.

The terms “polynucleotide”, “oligonucleotide”, “nucleic acid molecule” and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having linkages between nucleotides different from typical linkages, which are interchangeably used. Examples of such an oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond in an oligonucleotide are converted to a peptide-nucleic acid bond, an oligonucleotide derivative in which uracil in an oligonucleotide is substituted with C-5 propynyl uracil, an oligonucleotide derivative in which uracil in an oligonucleotide is substituted with C-5 thiazole uracil, an oligonucleotide derivative in which cytosine in an oligonucleotide is substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, and an oligonucleotide derivative in which ribose in an oligonucleotide is substituted with 2′-methoxyethoxy ribose. Unless otherwise indicated, a particular nucleic acid sequence also implicitly encompasses conservatively-modified variants thereof (e.g. degenerate codon substitutions) and complementary sequences as well as the sequence explicitly indicated. Specifically, degenerate codon substitutions may be produced by generating sequences in which the third position of one or more selected (or all) codons is substituted with mixed-base and/or deoxyinosine residues (Batzer et al., Nucleic Acid Res. 19:5081(1991); Ohtsuka et al., J. Biol. Chem. 260:2605-2608 (1985); Rossolini et al., Mol. Cell. Probes 8:91-98(1994)). A gene encoding an extracellular matrix protein (e.g., fibronectin, etc.) or the like is usually in the form of polynucleotide. A molecule to be transfected is in the form of polynucleotide.

As used herein the term “nucleotide” refers to a nucleoside in which the sugar moiety is phosphate ester, and includes DNA, RNA and the like, and may be naturally occurring or non-naturally occurring. Nucleoside refers to a compound in which a base and a sugar are bound via N-glycoside bonding. “Nucleotide derivative” or “nucleotide analog” are interchangeably used herein to refer to a derivative or an analog which is different from a naturally occurring nucleotide but has a similar function as that of such a nucleotide. Such a nucleotide derivative and nucleotide analog is well known in the art. Examples of such a nucleotide derivative and nucleotide analog include, for example, but are not limited to phosphorothioate, phosphoramidate, methyl phosphonate, chiral methyl phosphonate, 2-O-methyl. ribonucleotide, peptide-nucleic acid (PNA). DNA includes cDNA, genomic DNA, and synthetic DNA.

In an embodiment, the variant refers to a naturally occurring allelic variant, non-naturally occurring variant, a variant having deletion, substitution, addition and addition, a polynucleotide sequence which does not substantially alter the function of the encoded polypeptide.

In an embodiment, variation such as mutation of such amino acid sequences may occur in nature such as natural mutation, post-translational modification and the like, but also may artificially made using a naturally occurring gene such as specific genes of the present invention.

In an embodiment, the polypeptide comprises the allelic variants, homologs, natural variants, having at least 70%, preferably at least 80%, more preferably at least 95%, still more preferably at least 97% homology with the naturally occurring polypeptide.

As used herein, the term “corresponding” amino acid or nucleic acid refers to an amino acid or nucleotide in a given polypeptide or polynucleotide molecule, which has, or is anticipated to have, a function similar to that of a predetermined amino acid or nucleotide in a polypeptide or polynucleotide as a reference for comparison. Particularly, in the case of enzyme molecules, the term refers to an amino acid which is present at a similar position in an active site and similarly contributes to catalytic activity. For example, in the case of the transcription controlling activity sequence for a certain polynucleotide, the term refers to a similar portion in an ortholog corresponding to a particular portion of the transcription controlling activity sequence.

As used herein, the term “corresponding” gene (e.g., a polypeptide or polynucleotide molecule) refers to a gene in a given species, which has, or is anticipated to have, a function similar to that of a predetermined gene in a species as a reference for comparison. When there are a plurality of genes having such a function, the term refers to a gene having the same evolutionary origin. Therefore, a gene corresponding to a given gene may be an ortholog of the given gene. Therefore, genes corresponding to chemical receptors such as murine olfactory receptors and murine gustatory receptors can be found in other animals. Such a corresponding gene can be identified by techniques well known in the art. Therefore, for example, a corresponding gene in a given animal can be found by searching a sequence database of the animal (e.g., human, rat, dog, cat) using the sequence of a reference gene (e.g., chemical receptors such as murine olfactory receptors, murine gustatory receptors, etc.) as a query sequence. Such corresponding genes can be readily obtained by those skilled in the art using genome databases. Methods for obtaining such genome sequences are well known in the art and described herein elsewhere. In the present invention, sequences obtained by such search can also be used.

As used herein, the term “fragment” with respect to a polypeptide or polynucleotide refer to a polypeptide or polynucleotide having a sequence length ranging from 1 to n-1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more nucleotides. Lengths represented by integers which are not herein specified (e.g., 11 and the like) may be appropriate as a lower limit. For example, in the case of polynucleotides, the lower limit of the length of the fragment includes 5, 6, 7, 8, 9, 10, 15, 20, 25, 30, 40, 50, 75, 100or more nucleotides. Lengths represented by integers which are not herein specified (e.g., 11 and the like) may be appropriate as a lower limit. As used herein, the length of polypeptides or polynucleotides can be represented by the number of amino acids or nucleic acids, respectively. However, the above-described numbers are not absolute. The above-described numbers as the upper or lower limit are intended to include some greater or smaller numbers (e.g., ±10%), as long as the same function is maintained. For this purpose, “about” may be herein put ahead of the numbers. However, it should be understood that the interpretation of numbers is not affected by the presence or absence of “about” in the present specification. In the present invention, it should be understood that any fragment can be used, as long as the fragment functions as a chemical receptor, i.e., is capable of binding to a ligand (a chemical) thereby transducing the bound information downstream.

As used herein, the term “biological molecule” refers to a molecule relating to an organism and an aggregation thereof. As used herein, the term “biological” or “organism” refers to a biological organism, including, but being not limited to, an animal, a plant, a fungus, a virus, and the like. A biological molecule includes a molecule extracted from an organism and an aggregation thereof, though the present invention is not limited to this. Any molecule capable of affecting an organism and an aggregation thereof fall within the definition of a biological molecule. Therefore, low molecular weight molecules (e.g., low molecular weight molecule ligands, etc.) capable of being used as medicaments fall within the definition of biological molecule as long as an effect on an organism is intended. Examples of such a biological molecule include, but are not limited to, a protein, a polypeptide, an oligopeptide, a peptide, a polynucleotide, an oligonucleotide, a nucleotide, a nucleic acid (e.g., DNA such as cDNA and genomic DNA; RNA such as mRNA), a polysaccharide, an oligosaccharide, a lipid, a low molecular weight molecule (e.g., a hormone, a ligand, an information transmitting substance, a low molecular weight organic molecule, etc.), and a composite molecule thereof (glycolipids, glycoproteins, lipoproteins, etc.), and the like. A biological molecule may include a cell itself or a portion of tissue as long as it is intended to be introduced into a cell. Preferably, a biological molecule may include a nucleic acid (DNA or RNA) or a protein. In another preferred embodiment, a biological molecule is a nucleic acid (e.g., genomic DNA or cDNA, or DNA synthesized by PCR or the like). In another preferred embodiment, a biological molecule may be a protein. Preferably, such a biological molecule may be a hormone or cytokine. Accordingly, the sensor and system of the present invention can measure such a biological molecule. Measurement of such a biological molecule allows it use in diagnosis or the like. Application to diagnosis cannot be achieved by the prior art technology, and therefore it should be noted that such an effect is significant.

As used herein “chemical synthesized substance” refers to any substance which may be synthesized using an ordinary chemical technology. Accordingly, the chemical synthesized substance” are within chemical substances. Substantially all chemical substances may be synthesized. Such synthetic technology is well known in the art, and those skilled in the art can produce chemical synthesized substance appropriately combining such technology.

The term “cytokine” is used herein in the broadest sense in the art and refers to a physiologically active substance which is produced by a cell and acts on the same or different cell. Cytokines are generally proteins or polypeptides having a function of controlling an immune response, regulating the endocrine system, regulating the nervous system, acting against a tumor, acting against a virus, regulating cell growth, regulating cell differentiation, or the like. Cytokines are used herein in the form of a protein or a nucleic acid or in other forms. In actual practice, cytokines are typically proteins. The terms “growth factor” refers to a substance which promotes or controls cell growth. Growth factors are also called “proliferation factors” or “development factors”. Growth factors may be added to cell or tissue culture medium, substituting for serum macromolecules. It has been revealed that a number of growth factors have a function of controlling differentiation in addition to a function of promoting cell growth. Examples of cytokines representatively include, but are not limited to, interleukins, chemokines, hematopoietic factors (e.g., colony stimulating factors), tumor necrosis factor, and interferons. Representative examples of growth factors include, but are not limited to, platelet-derived growth factor (PDGF), epidermal growth factor (EGF), fibroblast growth factor (FGF), hepatocyte growth factor (HGF), endothelial cell growth factor (VEGF), cardiotrophin, and the like, which have proliferative activity.

As used herein “cytokine receptor” refers to a receptor to which a ligand responds to the above-mentioned cytokine. There is at least one such receptor corresponding to respective agent as described above. For example, an EGF receptor is present against the EGF.

As used herein “hormone” is used in the broadest sense as usually used in the art, and refers to a physiological organic compound having a physiologic action specific to an organ which is separated from the site of production, normally at a specific organ or cell of an animal or a plant. Such hormones include but are not limited to growth hormones, sex hormones, thyroid hormones. Such hormones may partially overlap the concept of the above-mentioned cytokines.

As used herein, the term “biological activity” refers to activity possessed by an agent (e.g., a polynucleotide, a protein, etc.) within an organism, including activities exhibiting various functions (e.g., transcription promoting activity, etc.). For example, when an agent is an antisense molecule, the biological activity thereof includes binding to a targeted nucleic acid molecule, suppression of expression thereby and the like. For example, when an agent is an enzyme, the biological activity thereof includes the enzymatic activity thereof. As for another example, when an agent is a ligand or a receptor, binding to the receptor or the ligand corresponding to the ligand or receptor, respectively, is included in the biological activity thereof. When the biological activity is transcriptional regulation activity, the activity refers to an activity for regulating transcriptional level or the variation thereof. Accordingly, the biological activity of a chemical receptor as used herein, is that the chemical receptor responds to a chemical known to be a target, and signal corresponding thereto is transduced. Such biological activity can be determined by well known technology in the art. Accordingly, the biological activity of a chemical receptor as used herein may be determined by measuring a response to a chemical using an indicator capable of measuring signal transduction such as physical signals, intracellular calcium concentration, downstream gene expression levels and the like, using a system for measuring a signal transduction of the chemical receptor such as a system operably linked to a marker encoding a G-coupled protein. For example, when the signal is calcium concentration, the calcium concentration can be measured in order to measure the indicator of interest.

As used herein, “polynucleotides hybridizing under stringent conditions” refers to conditions commonly used and well known in the art. Such a polynucleotide can be obtained by conducting colony hybridization, plaque hybridization, Southern blot hybridization, or the like using a polynucleotide selected from the polynucleotides of the present invention. Specifically, a filter on which DNA derived from a colony or plaque is immobilized is used to conduct hybridization at 65° C. in the presence of 0.7 to 1.0 M NaCl. Thereafter, a 0.1 to 2-fold concentration SSC (saline-sodium citrate) solution (1-fold concentration SSC solution composed of 150 mM sodium chloride and 15 mM sodium citrate) is used to wash the filter at 65° C. Polynucleotides identified by this method are referred to as “polynucleotides hybridizing under stringent conditions”. Hybridization can be conducted in accordance with a method described in, for example, Molecular Cloning 2nd ed., Current Protocols in Molecular Biology, Supplement 1-38, DNA Cloning 1: Core Techniques, A Practical Approach, Second Edition, Oxford University Press (1995), and the like. Here, sequences hybridizing under stringent conditions exclude, preferably, sequences containing only A (adenine) or T (thymine).

As used herein, “hybridizable polynucleotide” refers to a polynucleotide which can hybridize to other polynucleotides under the above-described hybridization conditions. Specifically, the hybridizable polynucleotide includes at least a polynucleotide having a homology of at least 60% to the base sequence of DNA encoding a polypeptide having an amino acid sequence as specifically set forth herein, preferably a polynucleotide having a homology of at least 80%, and more preferably a polynucleotide having a homology of at least 95%.

As used herein, the term “probe” refers to a substance for use in searching, which is used in a biological experiment, such as in vitro and/or in vivo screening or the like, including, but not being limited to, for example, a nucleic acid molecule having a specific base sequence or a peptide containing a specific amino acid sequence.

Examples of a nucleic acid molecule as a common probe include one having a nucleic acid sequence having a length of at least 8 contiguous nucleotides, which is homologous or complementary to the nucleic acid sequence of a gene of interest. Such a nucleic acid sequence may be preferably a nucleic acid sequence having a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 25 contiguous nucleotides, a length of at least 30 contiguous nucleotides, a length of at least 40 contiguous nucleotides, or a length of at least 50 contiguous nucleotides. A nucleic acid sequence used as a probe includes a nucleic acid sequence having at least 70% homology to the above-described sequence, more preferably at least 80%, and even more preferably at least 90% or at least 95%.

As used herein, the term “search” indicates that a given nucleic acid sequence is utilized to find other nucleic acid base sequences having a specific function and/or property either electronically or biologically, or using other methods. Examples of an electronic search include, but are not limited to, BLAST (Altschul et al., J. Mol. Biol. 215:403-410 (1990)), FASTA (Pearson & Lipman, Proc. Natl. Acad. Sci., USA 85:2444-2448 (1988)), Smith and Waterman method (Smith and Waterman, J. Mol. Biol. 147:195-197 (1981)), and Needleman and Wunsch method (Needleman and Wunsch, J. Mol. Biol. 48:443-453 (1970)), and the like. Examples of a biological search include, but are not limited to, a macro array in which genomic DNA is attached to a nylon membrane or the like or a microarray (microassay) in which genomic DNA is attached to a glass plate under stringent hybridization, PCR and in situ hybridization, and the like.

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.0015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual (2nd ed., Cold Spring Harbor Laboratory, N.Y., 1989); Anderson et al., Nucleic Acid Hybridization: A Practical Approach Ch. 4 (IRL Press Limited) (Oxford Express). More stringent conditions (such as higher temperature, lower ionic strength, higher formamide, or other denaturing agents) may be optionally used. Other agents may be included in the hybridization and washing buffers for the purpose of reducing non-specific and/or background hybridization. Examples are 0.1% bovine serum albumin, 0.1% polyvinylpyrrolidone, 0.1% sodium pyrophosphate, 0.1% sodium dodecylsulfate (NaDodSO₄ or SDS), Ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are ordinarily carried out at pH 6.8-7.4; however, at typical ionic strength conditions, the rate of hybridization is nearly independent of pH. See Anderson et al., Nucleic Acid Hybridization: A Practical Approach Ch. 4 (IRL Press Limited, Oxford UK).

Agents affecting the stability of DNA duplex include base composition, length, and degree of base pair mismatch. Hybridization conditions can be adjusted by those skilled in the art in order to accommodate these variables and allow DNAs of different sequence relatedness to form hybrids. The melting temperature of a perfectly matched DNA duplex can be estimated by the following equation:
Tm(° C.)=81.5+16.6(log [Na⁺])+0.41(% G+C)−600/N−0.72(% formamide)
where N is the length of the duplex formed, [Na⁺] is the molar concentration of the sodium ion in the hybridization or washing solution, % G+C is the percentage of (guanine+cytosine) bases in the hybrid. For imperfectly matched hybrids, the melting temperature is reduced by approximately 1° C. for each 1% mismatch.

The term “moderately stringent conditions” refers to conditions under which a DNA duplex with a greater degree of base pair mismatching than could occur under “highly stringent conditions” is able to form. Examples of typical “moderately stringent conditions” are 0.015 M sodium chloride, 0.0015 M sodium citrate at 50-65° C. or 0.015 M sodium chloride, 0.0015 M sodium citrate, and 20% formamide at 37-50° C. By way of example, “moderately stringent conditions” of 50° C. in 0.015 M sodium ion will allow about a 21% mismatch.

It will be appreciated by those skilled in the art that there is no absolute distinction between “highly stringent conditions” and “moderately stringent conditions”. For example, at 0.015 M sodium ion (no formamide), the melting temperature of perfectly matched long DNA is about 71° C. With a wash at 65° C. (at the same ionic strength), this would allow for approximately a 6% mismatch. To capture more distantly related sequences, those skilled in the art can simply lower the temperature or raise the ionic strength.

A good estimate of the melting temperature in 1 M NaCl for oligonucleotide probes up to about 20 nucleotides is given by:
Tm=(2° C. per A-T base pair)+(4° C. per G-C base pair).

Note that the sodium ion concentration in 6× salt sodium citrate (SSC) is 1 M. See Suggs et al., Developmental Biology Using Purified Genes 683 (Brown and Fox, eds., 1981).

A naturally-occurring nucleic acid encoding a protein (e.g., chemical receptor, or variants or fragments thereof, or the like) may be readily isolated from a cDNA library having PCR primers and hybridization probes containing part of a nucleic acid sequence indicated by, for example, SEQ ID NO. 1, 3, 5, 7, 9, 11, 13, 15, 17, 19, 21, 34, 36, 38, 40, 42, 44, 46, 48, 50, 52, 54, 56, 58 or the like. A preferable nucleic acid encoding chemical receptors, or variants or fragments thereof, or the like is hybridizable to the whole or part of a sequence as set forth in SEQ ID NO: 1, 3, 5, 7, 9, 11, 13, 15, 17, 19, 21, 34, 36, 38, 40, 42, 44, 46, 48, 50, 52, 54, 56, 58 or the like under low stringency conditions defined by hybridization buffer essentially containing 1% bovine serum albumin (BSA); 500 mM sodium phosphate (NaPO₄); 1 mM EDTA; and 7% SDS at 42° C., and wash buffer essentially containing 2×SSC (600 mM NaCl; 60 mM sodium citrate); and 0.1% SDS at 50° C., more preferably under low stringency conditions defined by hybridization buffer essentially containing 1% bovine serum albumin (BSA); 500 mM sodium phosphate (NaPO₄); 15% formamide; 1 mM EDTA; and 7% SDS at 50° C., and wash buffer essentially containing 1×SSC (300 mM NaCl; 30 mM sodium citrate); and 1% SDS at 50° C., and most preferably under low stringency conditions defined by hybridization buffer essentially containing 1% bovine serum albumin (BSA); 200 mM sodium phosphate (NaPO₄); 15% formamide; 1 mM EDTA; and 7% SDS at 50° C., and wash buffer essentially containing 0.5×SSC (150 mM NaCl; 15 mM sodium citrate); and 0.1% SDS at 65° C.

As used herein, the term “probe” refers to a substance for use in searching, which is used in a biological experiment, such as in vitro and/or in vivo screening or the like, including, but not being limited to, for example, a nucleic acid molecule having a specific base sequence or a peptide containing a specific amino acid sequence.

Examples of a nucleic acid molecule as a common probe include one having a nucleic acid sequence having a length of at least 8 contiguous nucleotides, which is homologous or complementary to the nucleic acid sequence of a gene of interest. Such a nucleic acid sequence may be preferably a nucleic acid sequence having a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 25 contiguous nucleotides, a length of at least 30 contiguous nucleotides, a length of at least 40 contiguous nucleotides, or a length of at least 50 contiguous nucleotides. A nucleic acid sequence used as a probe includes a nucleic acid sequence having at least 70% homology to the above-described sequence, more preferably at least 80%, and even more preferably at least 90% or at least 95%.

As used herein, the term “primer” refers to a substance required for initiation of a reaction of a macromolecule compound to be synthesized, in a macromolecule synthesis enzymatic reaction. In a reaction for synthesizing a nucleic acid molecule, a nucleic acid molecule (e.g., DNA, RNA, or the like) which is complementary to part of a macromolecule compound to be synthesized may be used.

A nucleic acid molecule which is ordinarily used as a primer includes one that has a nucleic acid sequence having a length of at least 8 contiguous nucleotides, which is complementary to the nucleic acid sequence of a gene of interest. Such a nucleic acid sequence preferably has a length of at least 9 contiguous nucleotides, more preferably a length of at least 10 contiguous nucleotides, even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 16 contiguous nucleotides, a length of at least 17 contiguous nucleotides, a length of at least 18 contiguous nucleotides, a length of at least 19 contiguous nucleotides, a length of at least 20 contiguous nucleotides, a length of at least 25 contiguous nucleotides, a length of at least 30 contiguous nucleotides, a length of at least 40 contiguous nucleotides, and a length of at least 50 contiguous nucleotides. A nucleic acid sequence used as a primer includes a nucleic acid sequence having at least 70% homology to the above-described sequence, more preferably at least 80%, even more preferably at least 90%, and most preferably at least 95%. An appropriate sequence as a primer may vary depending on the property of the sequence to be synthesized (amplified). Those skilled in the art can design an appropriate primer depending on the sequence of interest. Such a primer design is well known in the art and may be performed manually or using a computer program (e.g., LASERGENE, Primer Select, DNA Star).

As used herein, the term “epitope” refers to an antigenic determinant whose structure is clear. Therefore, the term “epitope” includes a set of amino acid residues which are involved in recognition by a particular immunoglobulin, or in the context of T cells, those residues necessary for recognition by T cell receptor proteins and/or Major Histocompatibility Complex (MHC) receptors. This term is also used interchangeably with “antigenic determinant” or “antigenic determinant site”. In the field of immunology, in vivo or in vitro, an epitope is the feature of a molecule (e.g., primary, secondary and tertiary peptide structure, and charge) that forms a site recognized by an immunoglobulin, T cell receptor or MHC (e.g. HLA) molecule. An epitope including a peptide comprises 3 or more amino acids in a spatial conformation which is unique to the epitope. Generally, anepitope consists of at least 5 such amino acids, and more ordinarily, consists of at least 6, 7, 8, 9 or 10 such amino acids. The greater the length of an epitope, the more the similarity of the epitope to the original peptide, i.e., longer epitopes are generally preferable. This is not necessarily the case when the conformation is taken into account. Methods of determining the spatial conformation of amino acids are known in the art, and include, for example, X-ray crystallography and two-dimensional nuclear magnetic resonance spectroscopy. Furthermore, the identification of epitopes in a given protein is readily accomplished using techniques well known in the art. See, also, Geysen et al., Proc. Natl. Acad. Sci. USA (1984) 81: 3998 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23: 709 (technique for identifying peptides with high affinity for a given antibody). Antibodies that recognize the same epitope can be identified in a simple immunoassay. Thus, methods for determining an epitope including a peptide are well known in the art. Such an epitope can be determined using a well-known, common technique by those skilled in the art if the primary nucleic acid or amino acid sequence of the epitope is provided.

Therefore, an epitope including a peptide requires a sequence having a length of at least 3 amino acids, preferably at least 4 amino acids, more preferably at least 5 amino acids, at least 6 amino acids, at least 7 amino acids, at least 8 amino acids, at least 9 amino acids, at least 10 amino acids, at least 15 amino acids, at least 20 amino acids, and at least 25 amino acids. Epitopes may be determined by those skilled in the art by using commercially available kit such as PepSet™ (Kurabo). In the present invention, presenting a protein epitope playing a role in signal transduction may be used as a system for measuring signal transduction.

As used herein, the term “agent binding specifically to” a certain nucleic acid molecule or polypeptide refers to an agent which has a level of binding to the nucleic acid molecule or polypeptide equal to or higher than a level of binding to other nucleic acid molecules or polypeptides. Examples of such an agent include, but are not limited to, when a target is a nucleic acid molecule, a nucleic acid molecule having a complementary sequence of a nucleic acid molecule of interest, a polypeptide capable of binding to a nucleic acid sequence of interest (e.g., a transcription agent, etc.), and the like, and when a target is a polypeptide, an antibody, a single chain antibody, either of a pair of a receptor and a ligand, either of a pair of an enzyme and a substrate, and the like. As used herein, such an agent specifically binding to (such as an agent specifically binding to calcium, an antibody against a specific gene product and the like), can be used in measuring signal transduction.

As used herein, the term “antibody” encompasses polyclonal antibodies, monoclonal antibodies, human antibodies, humanized antibodies, polyfunctional antibodies, chimeric antibodies, and anti-idiotype antibodies, and fragments thereof (e.g., F(ab′)2 and Fab fragments), and other recombinant conjugates. These antibodies may be fused with an enzyme (e.g., alkaline phosphatase, horseradish peroxidase, α-galactosidase, and the like) via a covalent bond or by recombination.

As used herein, the term “monoclonal antibody” refers to an antibody composition having a group of homologous antibodies. This term is not limited by the production manner thereof. This term encompasses all immunoglobulin molecules and Fab molecules, F(ab′)2 fragments, Fv fragments, and other molecules having an immunological binding property of the original monoclonal antibody molecule. Methods for producing polyclonal antibodies and monoclonal antibodies are well known in the art, and will be more sufficiently described below.

Monoclonal antibodies are prepared by using the standard technique well known in the art (e.g., Kohler and Milstein, Nature (1975) 256:495) or a modification thereof (e.g., Bucket al. (1982) In Vitro 18:377). Representatively, a mouse or rat is immunized with a protein bound to a protein carrier, and boosted. Subsequently, the spleen (and optionally several large lymph nodes) is removed and dissociated into a single cell suspension. If desired, the spleen cells may be screened (after removal of nonspecifically adherent cells) by applying the cell suspension to a plate or well coated with a protein antigen. B-cells that express membrane-bound immunoglobulin specific for the antigen bind to the plate, and are not rinsed away with the rest of the suspension. Resulting B-cells, or all dissociated spleen cells, are then induced to fuse with myeloma cells to form hybridomas. The hybridomas are used to produce monoclonal antibodies.

As used herein, the term “antigen” refers to any substrate to which an antibody molecule may specifically bind. As used herein, the term “immunogen” refers to an antigen capable of initiating activation of the antigen-specific immune response of a lymphocyte. Accordingly, chemical receptors or products of the downstream thereof may be used as an antigen or immunogen and uses antibody-antigen response to realize the sensor of the present invention.

(Variation of Polypeptides or Polynucleotides)

In the present invention, when using a functional polypeptide such as a chemical receptor and the like, a variant thereof may be used as long as the variant can attain similar functions such as signal transduction and the like.

A given amino acid may be substituted with another amino acid in a protein structure, such as a cationic region or a substrate molecule binding site, without a clear reduction or loss of interactive binding ability. A given biological function of a protein is defined by the interactive ability or other property of the protein. Therefore, a particular amino acid substitution may be performed in an amino acid sequence, or at the DNA code sequence level, to produce a protein which maintains the original property after the substitution. Therefore, various modifications of peptides as disclosed herein and DNA encoding such peptides may be performed without clear losses of biological usefulness.

When the above-described modifications are designed, the hydrophobicity indices of amino acids may be taken into consideration. Hydrophobic amino acid indices play an important role in providing a protein with an interactive biological function, which is generally recognized in the art (Kyte, J. and Doolittle, R. F., J. Mol. Biol. 157(1):105-132, 1982). The hydrophobic property of an amino acid contributes to the secondary structure of a protein and then regulates interactions between the protein and other molecules (e.g., enzymes, substrates, receptors, DNA, antibodies, antigens, etc.). Each amino acid is given a hydrophobicity index based on the hydrophobicity and charge properties thereof as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); lutamicacid (−3.5); glutamine (−3.5); asparticacid (−3.5); asparagine (−3.5); lysine (−3.9); and arginine (−4.5).

It is well known that if a given amino acid is substituted with another amino acid having a similar hydrophobicity index, the resultant protein may still have a biological function similar to that of the original protein (e.g., a protein having an equivalent enzymatic activity). For such an amino acid substitution, the hydrophobicity index is preferably within +2, more preferably within ±1, and even more preferably within ±0.5. It is understood in the art that such an amino acid substitution based on hydrophobicity is efficient.

A hydrophilicity index is also useful for modification of an amino acid sequence of the present invention. As described in U.S. Pat. No. 4,554,101, amino acid residues are given the following hydrophilicity indices: arginine (+3.0); lysine (+3.0); aspartic acid (+3.0+1); glutamic acid (+3.0±1); serine (+0.3); asparagine (+0.2); glutamine (+0.2); glycine (0); threonine (−0.4); proline (−0.5±1) ; alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid may be substituted with another amino acid which has a similar hydrophilicity index and can still provide a biological equivalent. For such an amino acid substitution, the hydrophilicity index is preferably within ±2, more preferably ±1, and even more preferably ±0.5.

(Profile and its Relevant Techniques)

As used herein, the term “profile” in relation to a cell refers to a set of measurements of the biological state of the cell. Particularly, the term “profile of a cell” refers to a set of discrete or continuous values obtained by quantitatively measuring a level of a “cellular component”. A level of a cellular component includes the expression level of a gene, the transcription level of a gene (the activity level of a transcription control sequence), the amount of mRNA encoding a specific gene, and the expression level of a protein in biological systems. The level of each cellular component, such as the expression level of mRNA and/or protein, is known to be altered in response to treatment with drugs or cellular biological perturbation or vibration. Therefore, the measurement of a plurality of “cellular components” generates a large amount of information about the effects of stimuli on the biological states of cells. Therefore, the profile is increasingly important in analysis of cells. Mammalian cells contain about 30,000 or more cellular components. Therefore, the profile of an individual cell is usually complicated. A profile in a predetermined state of a biological system may often be measured after stimulating the biological system. Such stimulation is performed under experimental or environmental conditions associated with the biological system. Examples of a stimulus include exposure of a biological system to a drug candidate, introduction of an exogenous gene, passage of time, deletion of a gene from the system, alteration of culture conditions, and the like. The wide range measurement of cellular components (i.e., profiles of gene replication or transcription, protein expression, and response to stimuli) has a high level of utility including comparison and investigation of the effects of drugs, diagnosis of diseases, and optimization of drug administration to patients as well as investigation of cells. Further, profiles are useful for basic life science research.

As used herein, the term “transcription control sequence” refers to a sequence which can regulate the transcription level of a gene. Such a sequence is at least two nucleotides in length. Examples of such a sequence include, but are not limited to, promoters, enhancers, silencers, terminators, sequences flanking other genome structural genes, genomic sequences other than exons, sequences within exons, and the like. A transcription control sequence used herein is not related to particular types. Rather, important information about a transcription control sequence is time-lapse fluctuation. Such fluctuation is referred to as a process (changes in a state of a cell). Therefore, such a transcription control sequence may be herein arbitrarily selected. Such a transcription control sequence may include those which are not conventionally used as markers. Preferably, a transcription control sequence has the capability of binding to a transcription factor.

As used herein, the term “transcription factor” refers to a factor which regulates the process of transcription of a gene. The term “transcription factor” mainly indicates a factor which regulates a transcription initiating reaction. Transcription factors are roughly divided into the following groups: basic transcription factors required for placing an RNA polymerase into a promoter region on DNA; and transcription regulatory factors which bind to cis-acting elements present upstream or downstream of a transcription region to regulate the synthesis initiation frequency of RNA.

Basic transcription factors are prepared depending on the type of RNA polymerase. A TATA-binding protein is believed to be common to all transcription systems. Although there are a number of types of transcription factors, a typical transcription factor consists of a portion structurally required for binding to DNA and a portion required for activating or suppressing transcription. Factors which have a DNA-binding portion and can bind to cis-acting elements are collectively referred to as trans-acting factors.

A portion required for activating or suppressing transcription is involved in interaction with other transcription factors or basic transcription factors. Such a portion is believed to play a role in regulating transcription via a structural change in DNA or a transcription initiating complex. Transcription regulatory factors are divided into several groups or families according to the structural properties of these portions, including factors which play an important role in the development or differentiation of a cell.

Examples of such a transcription factor include, but are not limited to, STAT1, STAT2, STAT3, GAS, NFAT, Myc, AP1, CREB, NFkB, E2F, Rb, p53, RUNX1, RUNX2, RUNX3, Nkx-2, CF2-II, Skn-1, SRY, HFH-2, Oct-1, Oct-3, Sox-5, HNF-3b, PPARγ, and the like. In the present invention, use of such transcriptional factors allows investigation of activation of signal transduction factors, and thus these transcriptional factors are applicable to the sensors of the present invention.

As used herein, the term “time-lapse” means any action or phenomenon that is related to the passage of time.

As used herein, the term “monitor” refers to measurement of the state of a cell using at least one parameter as measure (e.g., a label signal attributed to transcription, etc.). Preferably, monitoring is performed using a device, such as a detector, a measuring instrument, or the like. More preferably, such a device is connected to a computer for recording and/or processing data. Monitoring may comprise the step of obtaining the image data of a solid phase support (e.g., an array, a plate, etc.). Alternatively, “monitor” may include the step of measuring physical data, chemical data, biological data and the like obtained from signal transduction.

As used herein, the term “real time” means that a certain state is substantially simultaneously displayed in another form (e.g., as an image on a display or a graph with processed data). In such a case, the “real time” lags behind an actual event by the time required for data processing. Such a time lag is included in the scope of “real time” if it is substantially negligible. Such a time lag may be typically within 10 seconds, and preferably within 1 second, without limitation. A time lag exceeding 10 seconds may be included in the scope of “real time” for certain uses.

As used herein, the “determination” of a state of a cell can be performed using various methods. Examples of such methods include, but are not limited to, mathematical processing (e.g., signal processing, multivariate analysis, etc.), empirical processing, phase changes, and the like.

As used herein, the term “difference” refers to a result of mathematical processing in which a value of a control profile (e.g., without a stimulus) is subtracted from a certain profile.

As used herein, the term “phase” in relation to a time-lapse profile refers to the result of determination of whether the profile is positive or negative with respect to a reference point (typically 0), which is expressed with + or −, and also refers to analysis based on such a result.

As used herein, the term “correlate” in relation to obtained information (e.g. profile, etc.) and a chemical substance of measurement interest, such as an olfactory source, gustatory source, and the like, refers to an act of associating the information such as profile or particular information about changes with the information with the chemical substance such as an olfactory source, gustatory source or the like. A relationship between them is referred to as “correlation” or “correlation relationship”.

As used herein, correlation can be performed by associating at least one piece of information (e.g., information, profile or the like, regarding the activation of a receptor and the like, etc.) or changes thereof with information regarding the existence, change, species of the chemical substances and the like. For example, such information is quantitatively or qualitatively associated with at least one parameter indicating a state of a cell. A small number of parameters may be used for correlation as long as correlation can be performed, typically including, without limitation, at least 1, preferably at least 2, and more preferably at least 3. The present invention demonstrated that at least 2, preferably at least 3, parameters are sufficient for specifying substantially all entities. At least one parameter may be subjected to mathematical processing by utilizing a matrix to associate the profile with a state of a cell. In one preferred embodiment, at least 8 profiles (e.g., a time-lapse profile, etc.) may be advantageously used. Alternatively, all chemical receptors such as all olfactory receptors, all gustatory receptors, and the like, possessed by a living organism may be used. This is because use of all chemical receptors possessed by a living organism allows analyses all chemicals which can be perceived by the living organism.

Examples of a specific method for correlation include, but are not limited to, signal processing (e.g., wavelet analysis, etc.), multivariate analysis (e.g., cluster analysis, etc.), and the like.

Correlation may be performed in advance or may be performed at the time of determination of cells using a control, every time a different chip, a different lot of a sensor or different measurement is used.

As used herein, the term “external factor” in relation to a cell refers to a factor which is not usually present in the cell (e.g., a substance, energy, etc.). As used herein, the term “factor” may refer to any substance or element as long as an intended object can be achieved (e.g., energy, such as ionizing radiation, radiation, light, acoustic waves, and the like). Examples of such a substance include, but are not limited to, proteins, polypeptides, oligopeptides, peptides, polynucleotides, oligonucleotides, nucleotides, nucleic acids (e.g., DNA such as cDNA, genomic DNA and the like, or RNA such as mRNA, RNAi and the like), polysaccharides, oligosaccharides, lipids, low molecular weight organic molecules (e.g., hormones, ligands, information transduction substances, low molecular weight organic molecules, molecules synthesized by combinatorial chemistry, low molecular weight molecules usable as medicaments (e.g., low molecular weight molecule ligands, etc.), etc.), and composite molecules thereof. External factors may be used singly or in combination. Examples of an external factor as used herein include, but are not limited to, temperature changes, humidity changes, electromagnetic waves, potential difference, visible light, infrared light, ultraviolet light, X-rays, chemical substances, pressure, gravity changes, gas partial pressure, osmotic pressure, and the like. In one preferable embodiment, an external factor may be a biological molecule or a chemically synthesized substance. The sensor of the present invention using chemical receptors is basically directed to chemical substances as a target for measurement, however, those skilled in the art will be able to readily understand that in principle, all such external factors can be measured using the present invention.

(Fixation to Support/Substrate)

As used herein, the terms “cell adhesion agent”, “cell adhesion molecule”, “adhesion agent” and “adhesion molecule” are used interchangeably to refer to a molecule capable of mediating the joining of two or more cells (cell adhesion) or adhesion between a substrate and a cell. In general, cell adhesion molecules are divided into two groups: molecules involved in cell-cell adhesion (intercellular adhesion) (cell-cell adhesion molecules) and molecules involved in cell-extracellular matrix adhesion (cell-substrate adhesion) (cell-substrate adhesion molecules). For a method of the present invention, either type of molecule is useful and can be effectively used. Therefore, cell adhesion molecules herein include a substrate protein and a cellular protein (e.g., integrin, etc.) involved in cell-substrate adhesion. A molecule other than a protein can fall within the concept of cell adhesion molecule as long as it can mediate cell adhesion.

For cell-cell adhesion, collagen, fibronectin, laminin, vitronectin, cadherin, a number of molecules belonging in an immunoglobulin superfamily (NCAM, L1, ICAM, fasciclin II, III, etc.), selectin, and the like are known, each of which is known to connect cell membranes via a specific molecular interaction.

On the other hand, a major cell adhesion molecule functioning for cell-substrate adhesion is integrin, which recognizes and binds to various proteins contained in extracellular matrices. These cell adhesion molecules are all located on cell membranes and can be regarded as a type of receptor (cell adhesion receptor). Therefore, receptors present on cell membranes can also be used in a method of the present invention. Examples of such a receptor include, but are not limited to, α-integrin, β-integrin, CD44, syndecan, aggrecan, and the like. Techniques for cell adhesion are well known as described above and as described in, for example, “Saibogaimatorikkusu—Rinsho heno Oyo—[Extracellular matrix—Clinical Applications—], Medical Review.

It can be determined whether or not a certain molecule is a cell adhesion molecule, by an assay, such as biochemical quantification (an SDS-PAGE method, a labeled-collagen method, etc.), immunological quantification (an enzyme antibody method, a fluorescent antibody method, an immunohistological study, etc.), a PDR method, a hybridization method, or the like, in which a positive reaction is detected. Examples of such a cell adhesion molecule include, but are not limited to, collagen, integrin, fibronectin, laminin, vitronectin, fibrinogen, immunoglobulin superfamily members (e.g., CD2, CD4, CD8, ICM1, ICAM2, VCAM1), selectin, cadherin, and the like. Most of these cell adhesion molecules transmit an auxiliary signal for cell activation into a cell due to intercellular interaction as well as cell adhesion. It can be determined whether or not such an auxiliary signal can be transmitted into a cell, by an assay, such as biochemical quantification (an SDS-PAGE method, a labeled-collagen method, etc.), immunological quantification (an enzyme antibody method, a fluorescent antibody method, an immunohistological study, etc.), a PCR method, a hybridization method, or the like, in which a positive reaction is detected.

Examples of cell adhesion molecules include, but are not limited to, fibronectin, vitronectin, laminin, collagens, cadherin, immunoglobulin superfamily molecules (LFA-3, ICAM-1, CD2, CD4, CD8, ICM1, ICAM2, VCAM1, etc.); integrin family molecules (LFA-1, Mac-1, gpIIbIIIa, p150, p95, VLA1, VLA2, VLA3, VLA4, VLA5, VLA6, etc.); selectin family molecules (L-selectin, E-selectin, P-selectin, etc.), and the like.

As used herein, the term “extracellular matrix protein” refers to a protein constituting an “extracellular matrix”. As used herein, the term “extracellular matrix” (ECM) is also called “extracellular substrate” and has the same meaning as commonly used in the art, and refers to a substance existing between somatic cells no matter whether the cells are epithelial cells or non-epithelial cells. Extracellular matrices are involved in supporting tissue as well as in internal environmental structures essential for survival of all somatic cells. Extracellular matrices are generally produced from connective tissue cells. Some extracellular matrices are secreted from cells possessing basal membrane, such as epithelial cells or endothelial cells. Extracellular matrices are roughly divided into fibrous components and matrices filling there between. Fibrous components include collagen fibers and elastic fibers. A basic component of matrices is glycosaminoglycan (acidic mucopolysaccharide), most of which is bound to non-collagenous protein to form a polymer of a proteoglycan (acidic mucopolysaccharide-protein complex). In addition, matrices include glycoproteins, such as laminin of basal membrane, microfibrils around elastic fibers, fibers, fibronectins on cell surfaces, and the like. Particularly differentiated tissue has the same basic structure. For example, in hyaline cartilage, chondroblasts characteristically produce a large amount of cartilage matrices including proteoglycans. In bones, osteoblasts produce bone matrices which cause calcification. Examples of extracellular matrices for use in the present invention include, but are not limited to, collagens, elastin, proteoglycan, glycosaminoglycan, fibronectin, laminin, elastic fiber, collagen fiber, and the like. Such extracellular matrices may be used to fix a cell in the present invention.

(Devices and Solid Phase Supports)

As used herein, the term “device” refers to a part which can constitute the whole or a portion of an apparatus, and comprises a support (preferably, a solid phase support) and a target substance carried thereon. Examples of such a device include, but are not limited to, chips, arrays, microtiter plates, cell culture plates, Petri dishes, films, beads, and the like. Preferably, device which can be applied to a form of sensor is used herein. In the present invention, chi format is preferably used as a device.

As used herein, the term “support” refers to a material which can fix a substance, such as a biological molecule. Such a support may be made from any fixing material which has a capability of binding to a biological molecule as used herein via covalent or noncovalent bonds, or which may be induced to have such a capability.

Examples of materials used for supports include any material capable of forming a solid surface, such as, without limitation, glass, silica, silicon, ceramics, silicon dioxide, plastics, metals (including alloys), naturally-occurring and synthetic polymers (e.g., polystyrene, cellulose, chitosan, dextran, and nylon), and the like. A support may be formed of layers made of a plurality of materials. For example, a support may be made of an inorganic insulating material, such as glass, quartz glass, alumina, sapphire, forsterite, silicon oxide, silicon carbide, silicon nitride, or the like. A support may be made of an organic material, such as polyethylene, ethylene, polypropylene, polyisobutylene, polyethylene terephthalate, unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenol resin, urea resin, epoxy resin, melamine resin, styrene-acrylonitrile co-polymer, acrylonitrile-butadiene-styrene co-polymer, silicone resin, polyphenylene oxide, polysulfone, and the like. Also in the present invention, nitrocellulose film, nylon film, PVDF membrane, or the like, which are used in blotting, may be used as a material for a support. When a material constituting a support is in the solid phase, such as a support is herein particularly referred to as a “solid phase support”. A solid phase support may herein take the form of a plate, a microwell plate, a chip, a glass slide, a film, beads, a metal (surface), or the like. A support may not be coated or may be coated.

As used herein, the term “liquid phase” has the same meanings as commonly understood by those skilled in the art, typically referring a state in solution.

As used herein, the term “solid phase” has the same meanings as commonly understood by those skilled in the art, typically referring to a solid state. As used herein, liquid and solid may be collectively referred to as a “fluid”.

As used herein, the term “substrate” refers to a material (preferably, solid) which is used to construct a chip or array according to the present invention. Therefore, substrates are included in the concept of plates. Such a substrate may be made from any solid material which has a capability of binding to a biological molecule as used herein via covalent or noncovalent bonds, or which may be induced to have such a capability.

Examples of materials used for plates and substrates include any material capable of forming a solid surface, such as, without limitation, glass, silica, silicon, ceramics, silicon dioxide, plastics, metals (including alloys), naturally-occurring and synthetic polymers (e.g., polystyrene, cellulose, chitosan, dextran, and nylon), and the like. A support maybe formed of layers made of a plurality of materials. For example, a support may be made of an inorganic insulating material, such as glass, quartz glass, alumina, sapphire, forsterite, silicon oxide, silicon carbide, silicon nitride, or the like. A support may be made of an organic material, such as polyethylene, ethylene, polypropylene, polyisobutylene, polyethylene terephthalate, unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenol resin, urea resin, epoxy resin, melamine resin, styrene-acrylonitrile co-polymer, acrylonitrile-butadiene-styrene co-polymer, silicone resin, polyphenylene oxide, polysulfone, and the like. A material preferable as a substrate varies depending on various parameters such as the measuring device, and can be selected from the above-described various materials as appropriate by those skilled in the art. For transfection arrays, glass slides are preferable. Preferably, such a substrate may have a coating of the same or a different material.

As used herein, the term “coating” in relation to a solid phase support or substrate refers to an act of forming a film of a material on a surface of the solid phase support or substrate, and also refers to a film itself. Coating is performed for various purposes, such as, for example, improvement in the quality of a solid phase support and substrate (e.g., elongation of life span, improvement in resistance to hostile environment, such as resistance to acids, etc.), an improvement in affinity to a substance integrated with a solid phase support or substrate, and the like. Various materials may be used for such coating, including, without limitation, biological substances (e.g., DNA, RNA, protein, lipid, etc.), polymers (e.g., poly-L-lysine, MAS (available from Matsunami Glass, Kishiwada, Japan), and hydrophobic fluorine resin), silane (APS (e.g., γ-aminopropyl silane, etc.)), metals (e.g., gold, etc.), in addition to the above-described solid phase support and substrate. The selection of such materials is within the technical scope of those skilled in the art and thus can be performed using techniques well known in the art. In one preferred embodiment, such a coating may be advantageously made of poly-L-lysine, silane (e.g., epoxy silane or mercapto silane, APS (γ-aminopropyl silane), etc.), MAS, hydrophobic fluorine resin, a metal (e.g., gold, etc.). Such a material may be preferably a substance suitable for cells or objects containing cells (e.g., organisms, organs, etc.).

As used herein, the terms “chip” or “microchip” are used interchangeably to refer to a micro integrated circuit which has versatile functions and constitutes a portion of a system. Examples of a chip include, but are not limited to, DNA chips, protein chips, and the like. Chips may comprise tubing for supplying a solution. Such tubings may be made of any material as long as no adverse effect is given to the substance of interest in a sample of interest. When administering the stimulus as a solution, a flow rate may be about 1-4 mm/second, preferably about 2.5 mm/second so that cells are not affected by mechanical stimulation by hydraulic pressure, and the entire cell can receive the stimulus in a short period of time. When administering a stimulus in a gaseous form, a gas of interest is introduced into the center of the sensor in an array format. Such a method may be any known method in the art. As an example, the sensor member is enclosed, the exhaust pump is subjected to a weak negative pressure by connecting thereto, the opening of a tubing for introducing a gas of interest from outside is fixed to the vicinity of the sensor member, and when introducing a gas into a solution in which a cell is soaked, in order that a physical fluctuation arisen by wind to the water interface, does not give effects on a signal of interest, the liquid interface is maintained at place by subjecting glass cover onto the site of measure on a sensor array. The introduced gas may be equally distributed onto the sensor member by spraying the gas above the cell in a solution having no odor which soaks the cell and flows at a determined flow rate. Further, in another embodiment, it is possible that no glass cover is arranged onto the above of the sensor member and the introduced gas is directly subjected to the sensor array at a flow rate so that signals have no effects of water interface fluctuation by wind. In this instance, the water depth of the sensor member is about 1-2 mm and the sensor member may be in a condition where a solution should not repel from the sensor member by subjecting stimulus hydrophobic components to the sensor member. In order to maintain the cleanliness in the vicinity of a cell, twenty to thirty seconds after the measurement, it may be necessary to replace the solution in the vicinity with a solution without the chemical of interest.

As used herein, the term “array” refers to a substrate (e.g., a chip, etc.) which has a pattern of composition containing at least one (e.g., 1000 or more, etc.) target substances (e.g., DNA, proteins, transfection mixtures, etc.), which are arrayed. Among arrays, patterned substrates having a small size (e.g., 10×10 mm, etc.) are particularly referred to as microarrays. The terms “microarray” and “array” are used interchangeably. Therefore, a patterned substrate having a larger size than that which is described above may be referred to as a microarray. For example, an array comprises a set of desired transfection mixtures fixed to a solid phase surface or a film thereof. An array preferably comprises at least 10² antibodies of the same or different types, more preferably at least 10³, even more preferably at least 10⁴, and still even more preferably at least 10⁵. These antibodies are placed on a surface of up to 125×80 mm, more preferably 10×10 mm. An array includes, but is not limited to, a 96-well microtiter plate, a 384-well microtiter plate, a microtiter plate the size of a glass slide, and the like. A composition to be fixed may contain one or a plurality of types of target substances. Such a number of target substance types may be in the range of from one to the number of spots, including, without limitation, about 10, about 100, about 500, and about 1,000.

As described above, any number of target substances (e.g., proteins, such as antibodies) may be provided on a solid phase surface or film, typically including no more than 10⁸ biological molecules per substrate, in another embodiment no more than 10⁷ biological molecules, no more than 10⁶ biological molecules, no more than 10⁵ biological molecules, no more than 10⁴ biological molecules, no more than 10³ biological molecules, or no more than 10² biological molecules. A composition containing more than 10⁸ biological molecule target substances may be provided on a substrate. In these cases, the size of a substrate is preferably small. Particularly, the size of a spot of a composition containing target substances (e.g., proteins such as antibodies) may be as small as the size of a single biological molecule (e.g., 1 to 2 nm order). In some cases, the minimum area of a substrate may be determined based on the number of biological molecules on a substrate. A composition containing target substances, which are intended to be introduced into cells, are herein typically arrayed on and fixed via covalent bonds or physical interaction to the substrate in the form of spots having a size of 0.01 mm to 10 mm.

“Spots” of biological molecules may be provided on an array. As used herein, the term “spot” refers to a certain set of compositions containing target substances. As used herein, the term “spotting” refers to an act of preparing a spot of a composition containing a certain target substance on a substrate or plate. Spotting may be performed by any method, for example, pipetting or the like, or alternatively, using an automatic device. These methods are well known in the art.

As used herein, the term “address” refers to a unique position on a substrate, which may be distinguished from other unique positions. Addresses are appropriately associated with spots. Addresses can have any distinguishable shape such that substances at each address maybe distinguished from substances at other addresses (e.g., optically). A shape defining an address may be, for example, without limitation, a circle, an ellipse, a square, a rectangle, or an irregular shape. Therefore, the term “address” is used to indicate an abstract concept, while the term “spot” is used to indicate a specific concept. Unless it is necessary to distinguish them from each other, the terms “address” and “spot” may be herein used interchangeably. As used herein, cells expressing different chemical receptors for each may be located to addresses or spots, and a portion or the whole portion thereof may have cells expressing the same chemical receptors located therein. Additionally or alternatively, a cell expressing a plurality of chemical receptors may be located at at least one address or spot, however, the present invention is not limited to this.

The size of each address particularly depends on the size of the substrate, the number of addresses on the substrate, the amount of a composition containing target substances and/or available reagents, the size of microparticles, and the level of resolution required for any method used for the array. The size of each address may be, for example, in the range of from 1-2 nm to several centimeters, though the address may have any size suited to an array.

When using the present sensor in order to evaluate types, quality, intensity and the like of stimulants of detection target, stimulants are preferably introduced into a sensor chip member in a gaseous or solution form. Accordingly, in a preferable embodiment of the present invention, the portion including a cell is preferably covered by a liquid. In a preferable embodiment, a cell is preferably included in a medium supporting the maintenance of survival or propagation of the cell, and in a more preferable embodiment, the medium may be liquid medium. In this case, stimulants are at least diluted in consideration of surface area of an array to be used, and in order to conduct detection of stimulant with high sensitivity and evaluation of high accuracy, it is preferable to make the size of the arrays of 10 mm×10 mm to a smaller size of array. Further, it is considered that miniaturization allows stimulating with time shift that is substantially simultaneous over the entire array. This is believed to have the effect of minimizing effects of changing noise components over time, which is an inevitable problem for general measurement systems. However, if such time shift is too small, then the system is amenable to the effects of noise. Therefore, in order that all the element sensors have simultaneous and uniform contact with a stimulant, the size of the sensor chip member of the array is preferably appropriately minimized. In this regard, the size of the sensor chip member of the array is preferably 15 mm or less in a longitudinal direction (in a case of circular array, diameter), more preferably, 7.5 mm or less in a longitudinal direction, and if sensitivity and stability is sufficient, it may be preferable to have 1 mm or less in a longitudinal direction, but the present invention is not limited to this. Even if the array has longer than 15 mm in a longitudinal direction, as long as the delay in arrival of the stimulant within the array is caused in the same manner every time when the stimulant is introduced, it may be used by amending detected signals in each element sensor with time and intensity using coefficient or constant number for amendment of reduction of stimulant concentration due to time delay and diffusion to produce signal groups as if obtained by simultaneous and uniform stimulation. Further, in a preferable embodiment wherein a cell portion is soaked into a liquid, advantages of the case where the present sensor is used as an odor sensor, lies also in a method for administering the stimulant to the sensor. Many conventional odor sensors have been designed to measure stimulation under dry conditions, and therefore are susceptible to effects of different moisture depending on the surrounding circumstances. Therefore, the conventional technologies have struggled to maintain sample humidity at a fixed level when introduced to a sensor member by the addition or reduction of moisture of the sample. On the other hand, in a preferable embodiment, the present sensor uses a cell as a sensor soaked in a solution and thus humidity has no effects on signals. Therefore, the present invention has advantages in this respect.

The spatial arrangement and shape which define an address are designed so that the microarray is suited to a particular application. Addresses may be densely arranged or sparsely distributed, or subgrouped into a desired pattern appropriate for a particular type of material to be analyzed.

Microarrays are widely reviewed in, for example, “Genomu Kino Kenkyu Purotokoru [Genomic Function Research Protocol] (Jikken Igaku Bessatsu [Special Issue of Experimental Medicine], Posuto Genomu Jidai no Jikken Koza 1 [Lecture 1 on Experimentation in Post-genome Era), “Genomu Ikagaku to korekarano Genomu Iryo [Genome Medical Science and Futuristic Genome Therapy (Jikken Igaku Zokan [Special Issue of Experimental Medicine]), and the like.

A vast amount of data can be obtained from a microarray. Therefore, data analysis software is important for administration of correspondence between clones and spots, data analysis, and the like. Such software may be attached to various detection systems (e.g., Ermolaeva O. et al., (1998) Nat. Genet., 20: 19-23). The format of database includes, for example, GATC (genetic analysis technology consortium) proposed by Affymetrix.

(Detection)

In a method for obtaining information on a chemical substance using the chemical sensor of the present invention, various detection methods and means can be used as long as they can detect information such as physical signals, chemical signals, or biological signals or the like, attributed to a cell or a substance interacting therewith. Examples of such detection methods and means include, but are not limited to, visual inspection, optical microscopes, cofocal microscopes, reading devices using a laser light source, surface plasmon resonance (SPR) imaging, electric signals, means for measuring intracellular calcium, methods or means for detecting either or a plurality of chemical or biochemical markers, which may be used singly or in combination, which may optically be used in combination with image processing apparatus. Examples of such a detecting device include, but are not limited to, fluorescence analyzing devices, spectrophotometers, scintillation counters, CCD, luminometers, and the like. Any means capable of detecting a biological molecule may be used.

As used herein, the term “label” refers to a factor which distinguishes a molecule or substance of interest from others (e.g., substances, energy, electromagnetic waves, etc.). Examples of labeling methods include, but are not limited to, RI (radioisotope) methods, fluorescence methods, biotinylation methods, chemoluminescence methods, and the like. When the above-described nucleic acid fragments and complementary oligonucleotides are labeled by fluorescence methods, fluorescent substances having different fluorescence emission maximum wavelengths are used for labeling. The difference between each fluorescence emission maximum wavelength may be preferably 10 nm or more. Any fluorescent substance which can bind to a base portion of a nucleic acid may be used, preferably including a cyanine dye (e.g., Cy3 and Cy5 in the Cy Dye™ series, etc.), a rhodamine 6G reagent, N-acetoxy-N2-acetyl amino fluorene (AAF), AAIF (iodine derivative of AAF), and the like. Examples of fluorescent substances having a difference in fluorescence emission maximum wavelength of 10 nm or more include a combination of Cy5 and a rhodamine 6 G reagent, a combination of Cy3 and fluorescein, a combination of a rhodamine 6 G reagent and fluorescein, and the like. In the present invention, such a label can be used to alter a sample of interest so that the sample can be detected by detection means. Such alteration is known in the art. Those skilled in the art can perform such alteration using a method appropriate for a label and a sample of interest.

As used herein, the term “interaction” refers to, without limitation, hydrophobic interactions, hydrophilic interactions, hydrogen bonds, Van der Waals forces, ionic interactions, non-ionic interactions, electrostatic interactions, and the like.

As used herein, the term “interaction level” in relation to interaction between two substances (e.g., cells, etc.) refers to the extent or frequency of interaction between the two substances. Such an interaction level can be measured by methods well known in the art. For example, the number of cells which are fixed and actually perform interaction is counted directly or indirectly (e.g., the intensity of reflected light), for example, without limitation, by using an optical microscope, a fluorescence microscope, a phase-contrast microscope, or the like, or alternatively by staining cells with a marker, an antibody, a fluorescent label or the like specific thereto and measuring the intensity thereof. Such a level can be displayed directly from a marker or indirectly via a label. Based on the measured values of such levels, the number or frequency of genes, which are actually transcribed or expressed in a certain spot, can be calculated.

(Analysis of Gene Expression)

Mathematical processes used herein can be performed by using well-known techniques described in, for example, Kazuyuki Shimizu, “Seimei Sisutemu Kaiseki notameno Sugaku [Mathematics for Analyzing Biological Systems]”, Corona sha, 1999; and the like. Among these techniques, representative analyzing techniques will be described below.

In one embodiment, such a mathematical process maybe regression analysis. Examples of regression analysis include, but are not limited to, linear regression (e.g., simple regression analysis, multiple regression analysis, robust estimation, etc.), nonlinear estimation, and the like.

In simple regression analysis, n sets of data (x₁, y₁) to (xₙ, yₙ) are fitted to yᵢ=ax₁+b+e₁ (i=1, 2, . . . , n) where a and b are model parameters, and e₁ represents a deviation or an error from the straight line. The parameters a and b are typically determined so that the mean of sum of squares of the distance between a data point and the straight line is minimum. In this case, the rms of the distance is partially differentiated to produce simultaneous linear equations. These equations are solved for a and b which minimize the square errors. Such values are called least square estimates.

Next, a regression line is calculated based on the value obtained by subtracting the mean of all data values from each data value. A regression line represented by:
AΣᵢXᵢ+B=ΣYᵢ
is assumed. Further, it is assumed that B=0. The mean (xₐᵥₑ, yₐᵥₑ) of (xᵢ, yᵢ) (i=1, 2, . . . , n) is calculated, and the variance of x (sₓₓ) and the covariance of x and y (s_{xy}) are calculated.
The above-described regression line can be represented by:
y−yₐᵥₑ=(s_{xy}/sₓₓ)(x−xₐᵥₑ).
The correlation coefficient r_{xy} is represented by:
r_{xy}=s_{xy}/√(s_{xy}s_{yy}).

In this case, the relationship Σeᵢ²/n=s_{yy}(1−r_{xy}²) is satisfied. Therefore, as |r_{xy}| approaches 1, the error is decreased, which means that data can be satisfactorily represented by the regression line. As used herein, r_{xy}=s_{xy}/√ (s_{xy}s_{yy}).

In another embodiment, multiple regression analysis is used. In this technique, y is not a single independent variable, and is considered to be a function of two or more variables, e.g., and is represented by:
y=a₀+a₁x₁+a₂x₂+ . . . +aₙxₙ.

This equation is called a multiple regression equation. a₀ and the like are called (partial) regression coefficients. In multiple regression analysis, a least square method is used and normal equations are solved to obtain least square estimates. Evaluation can be performed as with single regression analysis.

In another embodiment, robust estimation is used. The least square method is based on the premise that measurement values are not biased and measurement errors have a normal distribution, and models have no approximation error. In actual situations, however, there may be errors in measurement. In robust estimation, unreliable data is detected and separated as outliers from the great majority of data which are reliable, or is subjected to a statistical process. Such a robust estimation may be utilized herein.

Nonlinear estimation may also be used herein. With nonlinear estimation, it is possible to represent a nonlinear model as vector equations which are in turn solved.

Other mathematical processes used herein include principal component analysis, which utilizes two-dimensional data principal component analysis, multi-dimensional data principal component analysis, singular value decomposition, and generalized inverse matrix. Alternatively, canonical correlation analysis, factor analysis, discrimination analysis, cluster analysis, and the like may be used herein.

(Gene Set Classification by Cluster Analysis)

For a number of applications, it may be desirable to obtain a set of reference transcription control sequences which are cooperatively controlled under a wide range of conditions. An embodiment of identifying such a set of reference transcription control sequences is, for example, a clustering algorithm, which is reviewed in, for example, Fukunaga, 1990, “Statistical Pattern Recognition”, 2nd ed., Academic Press, San Diego; Anderberg, 1973, “Cluster Analysis for Applications”, Academic Press: New York; Everitt, 1974, “Cluster Analysis”, London: Heinemann Educ. Books; Hartigan, 1975, “Clustering Algorithms”, New York: Wiley; and Sneath and Sokal, 1973, “Numerical Taxonomy”, Freeman.

A set of stimulation or sensory elemental information (such as gustatory elements, olfactory elements and the like) in a chemical receptor can also be defined based on a signal transduction mechanism. Such signal transduction mechanisms may be compared and analyzed with one another using multiple alignment analysis (Stormo and Hartzell, 1989, “Identifying protein binding sites from unaligned DNA fragments”, Proc. Natl. Acad. Sci., 86:1183-1187; Hertz and Stormo, 1995, “Identification of consensus patterns in unaligned DNA and protein sequences: a large-deviation statistical basis for penalizing gaps”, Proc. of 3rd Intl. Conf. on Bioinformatics and Genome Research, Lim and Cantor, ed., World Scientific Publishing Co., Ltd. Singapore, pp. 201-216).

In an embodiment using cluster analysis, the transcription levels of a number of chemical receptors can be monitored while applying various sensory stimuli to biological samples. A table of data containing measurements of the chemical receptors is used in cluster analysis. In order for cluster analyses, typically at least two, preferably at least 3, more preferably at least 10, even more preferably more than about 50, even more preferably more than about 20, still more preferably more than about 100, most preferably more than 300 stimuli or conditions are used. Cluster analysis is performed for a table of data having m×k dimensions where m is the total number of conditions or stimuli and k is the number of chemical receptors to be measured.

A number of clustering algorithms are useful for clustering analysis. In clustering algorithms, differences or distances between samples are used to form clusters. In a certain embodiment, a distance used is a Euclidean distance, Manhattan distance and the like.

In an embodiment, various cluster linkage methods are useful in a method of the present invention.

Examples of such a technique include a simple linkage method, a nearest neighbor method, and the like. In these techniques, a distance between the two closest samples is measured. Alternatively, in a complete linkage method, which may be herein used, a maximum distance between two samples in different clusters is measured. This technique is particularly useful when genes or other cellular components naturally form separate “clumps”.

Alternatively, the mean of non-weighted pairs is used to define the mean distance of all sample pairs in two different clusters. This technique is also useful in clustering genes or other cellular components which naturally form separate “clumps”. Finally, a weighted pair mean technique is also available. This technique is the same as a non-weighted pair mean technique, except that in the former, the size of each cluster is used as a weight. This technique is particularly useful in an embodiment in which it is suspected that the size of a cluster of transcription control sequences or the like varies considerably (Sneath and Sokal, 1973, “Numerical taxonomy”, San Francisco: W.H. Freeman & Co.). Other cluster linkage methods, such as, for example, non-weighted and weighted pair group, centroid and Ward's method, are also useful in several embodiments of the present invention. See, for example, Ward, 1963, J. Am. Stat. Assn., 58: 236; and Hartigan, 1975, “Clustering algorithms”, New York: Wiley.

In a certain preferred embodiment, cluster analysis can be performed using the well-known hclust technique (e.g., see a well-known procedure in “hclust” available from Program S-Plus, MathSoft, Inc., Cambridge, Mass.).

According to the present invention, it was found that even if the versatility of stimuli to a clustering set is increased, a state of a cell can be substantially elucidated by analyzing typically at least two, preferably at least 3, profiles using a method of the present invention. Stimulation conditions include treatment with a pharmaceutical agent in different concentrations, different measurement times after treatment, response to genetic mutations in various genes, a combination of treatment of a pharmaceutical agent and mutation, and changes in growth conditions (temperature, density, calcium concentration, etc.).

As used herein, the term “significantly different” in relation to two statistics means that the two statistics are different from each other with a statistical significance. In an embodiment of the present invention, data of a set of experiments concerning the responses of cellular components can be randomized by a Monte Carlo method to define an objective test.

In a preferable embodiment, an objective statistical test can be preferably used to determine the statistical reliability of grouping any clustering methods or algorithms. Preferably, similar tests can be applied to both hierarchical and nonhierarchical clustering methods. The compactness of a cluster is quantitatively defined as, for example, the mean of squares of the distances of elements in the cluster from the “mean of the cluster”, or more preferably, the inverse of the mean of squares of the distances of elements from the mean of the cluster. The mean of a specific cluster is generally defined as the mean of response vectors of all elements in the cluster. However, in a specific embodiment (e.g., the definition of the mean of the cluster is doubtful), for example, the absolute values of normalized or weighted inner products are used to evaluate the distance function of a clustering algorithm (i.e., I=1−|r|). Typically, the above-described definition of the mean may raise a problem in an embodiment in which response vectors have opposing directions so that the mean of the cluster as defined above is zero. Therefore, in such an embodiment, a different definition is preferably selected for the compactness of a cluster, for example, without limitation, the mean of squares of the distances of all pairs of elements in a cluster. Alternatively, the compactness of a cluster may be defined as the mean of distances between each element (e.g., a cellular component) of a cluster and another element of the cluster (or more preferably the inverse of the mean distance).

Other definitions, which may be used in statistical techniques used in the present invention, are obvious to those skilled in the art.

In another embodiment, information obtained according to the present invention can be analyzed using signal processing techniques. In these signal processing techniques, a correlation function is defined, a correlation coefficient is calculated, an autocorrelation function and a cross-correlation function are defined, and these functions are weighted where the sum of the weights is equal to 1. Thereby, moving averages can be obtained.

In signal processing, it is important to consider a time domain and a frequency domain. Rhythm often plays an important role in dynamic characteristic analysis for natural phenomena, particularly living entities. If a certain time function f (t) satisfies the following condition, the function is called a periodic function:
f(t)=f(t+T).

At time 0, the function takes a value of f(0). The function takes a value of f(0) at time T again after taking various values after time 0. Such a function is called a periodic function. Such a function includes a sine wave. T is called a period. The function has one cycle per time T. Alternatively, this feature may be represented by 1/T which means the number of cycles per unit time (cycles/time) without loss of the information. The concept represented by the number of cycles per unit time is called frequency. If the frequency is represented by f, f is represented by:
f=1/T.

Thus, the frequency is an inverse of the time. The time is dealt with in a time domain, while the frequency is dealt with in a frequency domain. The frequency may be represented in an electrical engineering manner. For example, the frequency is represented by angular measure where one period corresponds to 360° or 2π radians. In this case, f (cycles/sec) is converted to 2 πf (radians/sec), which is generally represented by ω (=2 πf) and is called angular frequency.

Now, a sine wave is compared with a cosine wave. The cosine wave is obtained by translating the sine wave by 90° or π/2 radians. The sine wave may be represented by the delayed cosine wave. This time delay is called phase. For example, when a pure cosine wave has a phase of 0, a sine wave has a phase of 90°. When a sine wave is added to a cosine wave, the amplitude of the resultant wave is increased by a factor of √2 and the phase is π/4.

In such analysis, Fourier series and frequency analysis may be available. In addition, Fourier transformation, discrete Fourier transformation, and power spectrum may be available. In Fourier expansion, techniques, such as wavelet transformation and the like, may be available. These techniques are well known in the art and are described in, for example, Yukio Shimizu, “Seimei Sisutemu Kaiseki notameno Sugaku [Mathematics for analyzing life systems]”, Corona sha, (1999); and Yasuhiro Ishikawa, “Rinsho Igaku notameno Ueburetto Kaiseki [Wavelet analysis for clinical medicine]”, Igaku Shuppan.

In an exemplary embodiment, below is explained an analysis method for evaluating the level of response of each receptor species within an array from a change in fluorescence intensity measured as a response of a cell expressing the receptor, using a change of a transient intracellular calcium concentration using fura-2 as an indicator. First, in order to exclude change in intracellular calcium concentration that may occur in the circumstances without stimulation, many cells may employ as an indicator whether or not responses are started at substantially the same time within 0.5-3 seconds from the start of stimulation. In order thereto, the frequency of repeating measurement of fluorescence images is desirably about once in a ⅓ second, however, depending on the nature of a cell used, once per two seconds may be used. When an olfactory receptor cell is directly used or indirectly used after transforming a cultured cell to introduce the receptor for expression, whether or not the cell is responsive to the stimulus, may be evaluated from the time property of the change in fluorescence intensity at 520 nm by 380 nm excitation. As described in a report of Hamana et al., Chem. Senses, 28: 87-104(2003), rise time of a response may be approximated using logistic curve (b/(1+c·exp(−a·t))), and the regression of the response may be approximated by using n·exp(−m·t). When an olfactory receptor cell is used, frequency of repeating fluorescence measurement suitable for the analysis is about once per ⅓ second. Response that cannot be approximated by a logistic curve, or in case where time constant 1/m obtained by approximating curve for regression of a response, shows changes more than differentials shown by each cell or cell population for average values, it can be determined that the response is not normal response to the stimulant. As a borderline for the evaluation, when an olfactory receptor cell is used, more than twice as much as standard deviation may be deemed as being aberrant. Other cells may have similar values for evaluation, it may be possible that some cell may be acceptable for larger variation, and therefore the determination may be conducted depending on the nature of a cell used.

When one measures responses in a time resolution such that one can approximate the rise of the response as mentioned above to the approximating curve, it is also possible to evaluate response intensity from the dynamic behavior of the rise. Specifically, it can be determined that the faster the rise speed of the response is, the larger the response intensity is. More generally, amplitude widths of response peaks may be calculated as a difference between the post- and pre-stimulation signal values, and evaluation may be conducted to calculate the matter, property, concentration, intensity of the stimulant based on the amplitude widths. When calculation is conducted at a time resolution such that peak positions can be specified, the amplitude widths may be desirable to be the peak values thereof, it may also be possible to use one data point measured data at a time when the value reaches at about 80% of the peak, rather than peak per se. In this case, there is a possibility where a response is a signal corresponding to differences of different components of different stimulants of two components having saturated signal intensities, and thus it is expected that evaluation of stimuli that due difficult for evaluation in the period of time showing the peak thereof, will be possible. This is not limited to a time responding to about 80% of response, and it may be applicable to the time showing 50%, 40%, or 30%, and it is also possible to analyze using data measured at a plurality of discrete times of 30%, 50%, 80% or the peak, or the like. Further, responses to each receptor used may be obtained from a single cell expressing the receptor species against each of the receptors, data added for a plurality of cells for responses calculated for each of the cells, or data subjected for addition processing the signals of cell population expressing the same receptor species in total. However, in order to make S/N ratio per receptor to the same level, the number of cells transmitting signals represents the responses of each receptor is made substantially the same. Further, partial extinction of fluorescence may occur due to excitation radiation, degradation or leakage of pigment, or the like. In order to exclude such influence from extinction, it is desirable to conduct normalization of signals using fluorescence intensities before starting a series of measurement presenting each stimulant.

(Presentation and Display)

As used herein, the terms “display” and “presentation” are used interchangeably to refer to an act of providing a profile obtained by a method of the present invention or information derived therefrom directly or indirectly, or in an information-processed form. Examples of such displayed forms include, but are not limited to, various methods, such as graphs, photographs, tables, animations, and the like. Such techniques are described in, for example, METHODS IN CELL BIOLOGY, VOL. 56, ed. 1998, pp: 185-215, A High-Resolution Multimode Digital Microscope System (Sluder & Wolf, Salmon), which discusses application software for automating a microscope and controlling a camera and the design of a hardware device comprising an automated optical microscope, a camera, and a Z-axis focusing device, which can be used herein. Image acquisition by a camera is described in detail in, for example, Inoue and Spring, Video Microscopy, 2d. Edition, 1997, which is herein incorporated by reference.

Real time display can also be performed using techniques well known in the art. For example, after all images are obtained and stored in a semi-permanent memory, or substantially at the same time as when an image is obtained, the image can be processed with appropriate application software to obtain processed data. For example, data may be processed by a method for playing back a sequence of images without interruption, a method for displaying images in real time, or a method for displaying images as a “movie” or “streaming” showing irradiating light as changes or continuation on a focal plane.

In another embodiment, application software for measurement and presentation typically includes software for setting conditions for applying stimuli or conditions for recording detected signals. With such a measurement and presentation application, a computer can have a means for applying a stimulus to cells and a means for processing signals detected from cells, and in addition, can control an optically observing means (a SIT camera and an image filing device) and/or a cell culturing means.

By inputting stimulus variables on a parameter setting screen using a keyboard, a touch panel, a mouse, or the like, it is possible to set desired complicated conditions for stimulation. In addition, various conditions, such as a temperature for cell culture, pH, and the like, can be set using a keyboard, a mouse, or the like. A display screen displays a time-lapse profile detected from a cell or information derived therefrom in real time or after recording. In addition, another recorded profile or information derived from of a cell can be displayed while being superimposed with a microscopic image of the cell. In addition to recorded information, measurement parameters in recording (stimulation conditions, recording conditions, display conditions, process conditions, various conditions for cells, temperature, pH, etc.) can be displayed in real time. The present invention may be equipped with a function of issuing an alarm when a temperature or pH departs from the tolerable range.

On a data analysis screen, it is possible to set conditions for various mathematical analyses, such as Fourier transformation, cluster analysis, FFT analysis, coherence analysis, correlation analysis, and the like. The present invention may be equipped with a function of temporarily displaying a profile, a function of displaying topography, or the like. The results of these analyses can be displayed while being superimposed with microscopic images stored in a recording medium.

(Gene Introduction)

Any technique may be used herein for introduction of a nucleic acid molecule into cells, including, for example, transformation, transduction, transfection, and the like. In the present invention, transfection is preferable.

As used herein, the term “transfection” refers to an act of performing gene introduction or transfection by culturing cells with gene DNA, plasmid DNA, viral DNA, viral RNA or the like in a substantially naked form (excluding viral particles), or adding such genetic material into a cell suspension to allow the cells to take up the genetic material. A gene introduced by transfection. is typically expressed within cells in a temporary manner or may be incorporated into cells in a permanent manner.

Such a nucleic acid molecule introduction technique is well known in the art and commonly used, and is described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed., Cold Spring Harbor Laboratory Press, Cold Spring Harbor, N.Y.; Special issue, Jikken Igaku [Experimental Medicine] “Experimental Methods for Gene introduction & Expression Analysis”, Yodo-sha, 1997; and the like. Gene introduction can be confirmed by methods as described herein, such as Northern blotting analysis and Western blotting analysis, or other well-known, common techniques.

When a gene is mentioned herein, the term “vector” or “recombinant vector” refers to a vector transferring a polynucleotide sequence of interest to a target cell. Such a vector is capable of self-replication or incorporation into a chromosome in a host cell (e.g., a prokaryotic cell, yeast, an animal cell, a plant cell, an insect cell, an individual animal, and an individual plant, etc.), and contains a promoter at a site suitable for transcription of a polynucleotide of the present invention. A vector suitable for performing cloning is referred to as a “cloning vector”. Such a cloning vector ordinarily contains a multiple cloning site containing a plurality of restriction sites. Restriction enzyme sites and multiple cloning sites as described above are well known in the art and can be used as appropriate by those skilled in the art depending on the purpose in accordance with publications described herein (e.g., Sambrook et al., supra).

As used herein, the term “expression vector” refers to a nucleic acid sequence comprising a structural gene and a promoter for regulating expression thereof, and in addition, various regulatory elements in a state that allows them to operate within host cells. The regulatory element may include, preferably, terminators, selectable markers such as drug-resistance genes, and enhancers. It is well known in the art that a type of an expression vector of a living organism such as an animal and a species of a regulatory element used may vary depending on the type of host cell used.

Examples of “recombinant vectors” for prokaryotic cells include, but are not limited to, pcDNA3 (+), pBluescript-SK(+/−), PGEM-T, PEF-BOS, PEGFP, pHAT, pUC18, pFT-DEST™42GATEWAY (Invitrogen), and the like.

Examples of “recombinant vectors” for animal cells include, but are not limited to, pcDNAI/Amp, pcDNAI, pCDM8 (all commercially available from Funakoshi), pAGE107 [Japanese Laid-Open Publication No. 3-229 (Invitrogen), pAGE103 [J. Biochem., 101, 1307(1987)], pAMo, pAMoA [J. Biol. Chem., 268, 22782-22787(1993)], a retrovirus expression vector based on a murine stem cell virus (MSCV), pEF-BOS, pEGFP, and the like.

Examples of recombinant vectors for plant cells include, but are not limited to, pPCVICEn4HPT, pCGN1548, pCGN1549, pBI221, pBI121, and the like.

Any of the above-described methods for introducing DNA into cells can be used as a vector introduction method, including, for example, transfection, transduction, transformation, and the like (e.g., a calcium phosphate method, a liposome method, a DEAE dextran method, an electroporation method, a particle gun (gene gun) method, and the like), a lipofection method, a spheroplast method (Proc. Natl. Acad. Sci. USA, 84, 1929(1978)), a lithium acetate method (J. Bacteriol., 153, 163(1983); and Proc. Natl. Acad. Sci. USA, 75, 1929(1978)), and the like.

As used herein, the term “operably linked” indicates that a desired sequence is located such that expression (operation) thereof is under control of a transcription and translation regulatory sequence (e.g., a promoter, an enhancer, and the like) or a translation regulatory sequence. In order for a promoter to be operably linked to a gene, typically, the promoter is located immediately upstream of the gene. A promoter is not necessarily adjacent to a structural gene.

As used herein, the term “terminator” refers to a sequence which is located downstream of a protein-encoding region of a gene and which is involved in the termination of transcription when DNA is transcribed into mRNA, and the addition of a poly-A sequence. It is known that a terminator contributes to the stability of mRNA, and has an influence on the amount of gene expression.

As used herein, the term “promoter” refers to a base sequence which determines the initiation site of transcription of a gene and is a DNA region which directly regulates the frequency of transcription. Transcription is started by RNA polymerase binding to a promoter. Accordingly, a portion having promoter function of a gene herein refers to “promoter moiety”. A promoter region is usually located within about 2 kbp upstream of the first exon of a putative protein coding region. Therefore, it is possible to estimate a promoter region by predicting a protein coding region in a genomic base sequence using DNA analysis software. A putative promoter region is usually located upstream of a structural gene, but depending on the structural gene, i.e., a putative promoter region may be located downstream of a structural gene. Preferably, a putative promoter region is located within about 2 kbp upstream of the translation initiation site of the first exon. Promoters include, but are not limited to for example, constitutive promoters, specific promoters and inductive promoters.

As used herein, the term “enhancer” refers to a sequence which is used so as to enhance the expression efficiency of a gene of interest. One or more enhancers may be used, or no enhancer may be used.

As used herein, the term “silencer” refers to a sequence which has a function of suppressing and arresting the expression of a gene. Any silencer which has such a function may be herein used. No silencer may be used.

As used herein, the term “operably linked” indicates that a desired sequence is located such that expression (operation) thereof is under control of a transcription and translation regulatory sequence (e.g., a promoter, an enhancer, and the like) or a translation regulatory sequence. In order for a promoter to be operably linked to a gene, typically, the promoter is located immediately upstream of the gene. A promoter is not necessarily adjacent to a structural gene. “Operably linked” also refers, when herein used to refer to signal transduction, to that each signal transduction molecule interacts directly or indirectly via another molecule to contribute to the signal transduction.

As used herein, the term “gene introduction reagent” refers to a reagent which is used in a gene introduction method so as to enhance introduction efficiency. Examples of such a gene introduction reagent include, but are not limited to, cationic polymers, cationic lipids, polyamine-based reagents, polyimine-based reagents, calcium phosphate, and the like. Specific examples of a reagent used in transfection include reagents available from various sources, such as, without limitation, Effectene Transfection Reagent (cat. no. 301425, Qiagen, Calif.), TransFast™ Transfection Reagent (E2431, Promega, Wisc.), Tfx™-20 Reagent (E2391, Promega, Wisc.), SuperFect Transfection Reagent (301305, Qiagen, Calif.), PolyFect Transfection Reagent (301105, Qiagen, Calif.), LipofectAMINE 2000 Reagent (11668-019, Invitrogen corporation, Calif.), JetPEI (×4) conc. (101-30, Polyplus-transfection, France) and ExGen 500 (R0511, Fermentas Inc., Md.), and the like.

Gene expression (e.g., mRNA expression, polypeptide expression) may be “detected” or “quantified” by an appropriate method, including mRNA measurement and immunological measurement. Examples of molecular biological measurement methods include Northern blotting methods, dot blotting methods, PCR methods, and the like. Examples of immunological measurement method include ELISA methods, RIA methods, fluorescent antibody methods, Western blotting methods, immunohistological staining methods, and the like, where a microtiter plate may be used. Examples of quantification methods include ELISA methods, RIA methods, and the like. A gene analysis method using an array (e.g., a DNA array, a protein array, etc.) may be used. The DNA array is widely reviewed in Saibo-Kogaku [Cell Engineering], special issue, “DNA Microarray and Up-to-date PCR Method”, edited by Shujun-sha. The protein array is described in detail in Nat Genet. 2002 December; 32 Suppl: 526-32. Examples of methods for analyzing gene expression include, but are not limited to, RT-PCR methods, RACE methods, SSCP methods, immunoprecipitation methods, two-hybrid systems, in vitro translation methods, and the like in addition to the above-described techniques. Other analysis methods are described in, for example, “Genome Analysis Experimental Method, Yusuke Nakamura's Lab-Manual, edited by Yusuke Nakamura, Yodo-sha (2002), and the like. All of the above-described publications are herein incorporated by reference.

As used herein, the term “expression level” refers to the amount of a polypeptide or mRNA expressed in a subject cell. The term “expression level” includes the level of protein expression of a polypeptide evaluated by any appropriate method using an antibody, including immunological measurement methods (e.g., an ELISA method, an RIA method, a fluorescent antibody method, a Western blotting method, an immunohistological staining method, and the like, or the mRNA level of expression of a polypeptide evaluated by any appropriate method, including molecular biological measurement methods (e.g., a Northern blotting method, a dot blotting method, a PCR method, and the like). The term “change in expression level” indicates that an increase or decrease in the protein or mRNA level of expression of a polypeptide evaluated by an appropriate method including the above-described immunological measurement method or molecular biological measurement method.

(Screening)

As used herein, the term “screening” refers to selection of a target, such as an organism, a substance, or the like, a given specific property of interest from a population containing a number of elements using a specific operation/evaluation method. For screening, a method or system of the present invention may be used. In the present invention, a sensor for a chemical is provided, any chemical substances may be screened.

In the present invention, a library of any chemical such as gustatory sources, olfactory sources and the like may be screened by the use of the sensor, chip, system or method of the present invention. The present invention is also intended to comprise chemicals identified by the screening or the combination thereof.

(Diagnosis)

As used herein, the term “diagnosis” refers to an act of identifying various parameters associated with a disease, a disorder, a condition, or the like of a subject and determining a current state of the disease, the disorder, the condition, or the like. A method, sensor, chip or system of the present invention can be used to analyze a chemical substance. Such information can be used to select parameters, such as a disease, a disorder, a condition, and a prescription or method for treatment or prevention of a subject. Such a chemical is preferably derived from the subject of interest. Samples suspected to contain or containing such chemical may be prepared by a subject of interest. Such preparation is well known in the art and include but is not limited to for example, blood, bodily odor, urine, biopsy sample and the like.

A diagnosis method of the present invention can use, in principle, a sample which is derived from the body of a subject. Therefore, it is possible for some one which is not a medical practitioner, such as a medical doctor, to deal with such a sample. The present invention is thus industrially useful.

(Therapy)

The present invention may also be applied to treat or prevent a subject or patient in a tailor-made manner using the result of the above-mentioned diagnosis. Such tailor-made therapy or prevention is within the present invention.

As used herein, the term “therapy” refers to an act of preventing progression of a disease or a disorder, preferably maintaining the current state of a disease or a disorder, more preferably alleviating a disease or a disorder, and more preferably extinguishing a disease or a disorder.

As used herein, the term “subject” refers to a living organism which is subjected to the treatment of the present invention. A subject is also referred to as a “patient”. A patient or subject may preferably be a human.

As used herein, the term “cause” or “pathogen” in relation to a disease, a disorder or a condition of a subject refers to an agent associated with the disease, the disorder or the condition (also collectively referred to as a “lesion”, or “disease damage” in plants), including, without limitation, a causative or pathogenic substance (pathogenic agent), a disease agent, a disease cell, a pathogenic virus, and the like.

A disease targeted by the present invention may be any disease associated with a pathogenic gene. Examples of such a disease include, but are not limited to, cancer, infectious diseases due to viruses or bacteria, allergy, hypertension, hyperlipemia, diabetes, cardiac diseases, cerebral infarction, dementia, obesity, arteriosclerosis, infertility, mental and nervous diseases, cataract, progeria, hypersensitivity to ultraviolet radiation, and the like.

A disorder targeted by the present invention may be any disorder associated with a pathogenic gene.

Examples of such a disease, disorder or condition include, but are not limited to, circulatory diseases (anemia (e.g., aplastic anemia (particularly, severe aplastic anemia), renal anemia, cancerous anemia, secondary anemia, refractory anemia, etc.), cancer or tumors (e.g., leukemia, multiple myeloma), etc.); neurological diseases (dementia, cerebral stroke and sequelae thereof, cerebral tumor, spinal injury, etc.); immunological diseases (T-cell deficiency syndrome, leukemia, etc.); motor organ and the skeletal system diseases (fracture, osteoporosis, luxation of joints, subluxation, sprain, ligament injury, osteoarthritis, osteosarcoma, Ewing's sarcoma, osteogenesis imperfecta, osteochondrodysplasia, etc.); dermatologic diseases (atrichia, melanoma, cutis malignant lymphoma, hemangiosarcoma, histiocytosis, hydroa, pustulosis, dermatitis, eczema, etc.); endocrinologic diseases (hypothalamus/hypophysis diseases, thyroid gland diseases, accessory thyroid gland (parathyroid) diseases, adrenal cortex/medulla diseases, saccharometabolism abnormality, lipid metabolism abnormality, protein metabolism abnormality, nucleic acid metabolism abnormality, inborn error of metabolism (phenylketonuria, galactosemia, homocystinuria, maple syrup urine disease), analbuminemia, lack of ascorbic acid synthetic ability, hyperbilirubinemia, hyperbilirubinuria, kallikrein deficiency, mast cell deficiency, diabetes insipidus, vasopressin secretion abnormality, dwarfism, Wolman's disease (acid lipase deficiency)), mucopolysaccharidosis VI, etc.); respiratory diseases (pulmonary diseases (e.g., pneumonia, lung cancer, etc.), bronchial diseases, lung cancer, bronchial cancer, etc.); alimentary diseases (esophagial diseases (e.g., esophagial cancer, etc.), stomach/duodenum diseases (e.g., stomach cancer, duodenum cancer, etc.), small intestine diseases/large intestine diseases (e.g., polyps of the colon, colon cancer, rectal cancer, etc.), bile duct diseases, liver diseases (e.g., liver cirrhosis, hepatitis (A, B, C, D, E, etc.), fulminant hepatitis, chronic hepatitis, primary liver cancer, alcoholic liver disorders, drug induced liver disorders, etc.), pancreatic diseases (acute pancreatitis, chronic pancreatitis, pancreatic cancer, cystic pancreas diseases, etc.), peritoneum/abdominal wall/diaphragm diseases (hernia, etc.), Hirschsprung's disease, etc.); urinary diseases (kidney diseases (e.g., renal failure, primary glomerulus diseases, renovascular disorders, tubular function abnormality, interstitial kidney diseases, kidney disorders due to systemic diseases, kidney cancer, etc.), bladder diseases (e.g., cystitis, bladder cancer, etc.); genital diseases (male genital organ diseases (e.g., male sterility, prostatomegaly, prostate cancer, testicular cancer, etc.), female genital organ diseases (e.g., female sterility, ovary function disorders, hysteromyoma, adenomyosis uteri, uterine cancer, endometriosis, ovarian cancer, villosity diseases, etc.), etc); circulatory diseases (heart failure, angina pectoris, myocardial infarct, arrhythmia, valvulitis, cardiac muscle/pericardium diseases, congenital heart diseases (e.g., atrial septal defect, arterial canal patency, tetralogy of Fallot, etc.), artery diseases (e.g., arteriosclerosis, aneurysm), vein diseases (e.g., phlebeurysm, etc.), lymphoduct diseases (e.g., lymphedema, etc.), etc.); and the like.

As used herein, the term “cancer” refers to a malignant tumor which has a high level of atypism, grows faster than normal cells, tends to disruptively invade surrounding tissue or metastasize to new body sites or a condition characterized by the presence of such a malignant tumor. In the present invention, cancer includes, without limitation, solid cancer and hematological cancer. Diagnosis of cancers using the system of the present invention may be conducted using the technlogy described in reports of Yamazaki et al. (Proc. Natl. Acad. Sci. USA, 99: 5612 (2002).

As used herein, the term “solid cancer” refers to a cancer having a solid shape in contrast to hematological cancer, such as leukemia and the like. Examples of such a solid cancer include, but are not limited to, breast cancer, liver cancer, stomach cancer, lung cancer, head and neck cancer, uterocervical cancer, prostate cancer, retinoblastoma, malignant lymphoma, esophagal cancer, brain tumor, osteoncus, and the like.

As used herein, the term “cancer therapy” encompasses administration of an anticancer agent (e.g., a chemotherapeutic agent, radiation therapy, etc.) or surgical therapy, such as surgical excision and the like.

Chemotherapeutic agents used herein are well known in the art and are described in, for example, Shigeru Tsukagoshi et al. editors, “Kogan zai Manyuaru [Manual of Anticanceragents]”, 2nd ed., Chugai Igakusha; Pharmacology; and Lippincott Williams & Wilkins, Inc. Examples of such chemotherapeutic agents are described below: 1) alkylating agents which alkylate cell components, such as DNA, protein, and the like, to produce cytotoxicity (e.g., cyclophosphamide, busulfan, thiotepa, dacarbazine, etc.); 2) antimetabolites which mainly inhibit synthesis of nucleic acids (e.g., antifolics (methotrexate, etc.), antipurines (6-mercaptopurine, etc.), antipyrimidines (fluorourasil (5-FU), etc.); 3) DNA topoisomerase inhibitors (e.g., camptothecin and etoposide, each of which inhibits topoisomerases I and II)); 4) tubulin agents which inhibit formation of microtubules and suppress cell division (vinblastine, vincristine, etc.); 5) platinum compounds which bind to DNA and proteins to exhibit cytotoxicity (cisplatin, carboplatin, etc.); 6) anticancer antibiotics which bind to DNA to inhibit synthesis of DNA and RNA (adriamycin, dactinomycin, mitomycin C, bleomycin, etc.); 7) hormone agents which are applicable to hormone-dependent cancer, such as breast cancer, uterus cancer, prostate cancer, and the like (e.g., tamoxifen, leuprorelin (LH-RH), etc.); 8) biological formulations (asparaginase effective for asparagine requiring malignant tumors blood, interferon exhibiting direct antitumor action and indirect action by immunopotentiation, etc.); 9) immunostimulants which exhibit capability of immune response, indirectly leading to antitumor activity (e.g., rentinan which is a polysaccharide derived from shiitake mushroom, bestatin which is a peptide derived from a microorganism, etc.).

An “anticancer agent” used herein selectively suppresses the growth of cancerous (tumor) cells, and includes both pharmaceutical agents and radiation therapy. Such an anticancer agent is well known in the art and described in, for example, Shigeru Tsukagoshi et al. editors, “Kogan zai Manyuaru [Manual of Anticancer agents]”, 2nd ed., Chugai Igaku sha; Pharmacology; and Lippincott Williams & Wilkins, Inc.

As used herein, the term “radiation therapy” refers to a therapy for diseases using ionizing radiation or radioactive substances. Representative examples of radiation therapy include, but are not limited to, X-ray therapy, γ-ray therapy, electron beam therapy, proton beam therapy, heavy particle beam therapy, neutron capture therapy, and the like. For example, heavy particle beam therapy is preferable. However, heavy particle beam therapy requires a large-size device and is not generally used. The above-described radiation therapies are well known in the art and are described in, for example, Sho Kei Zen, “Hoshasenkensa to Chiryo no Kiso: Hoshasen Chiryo to Shugakuteki Chiryo [Basics of Radiation Examination and Therapies: Radiation Therapy and Incentive Therapy]”, (Shiga Medical School, Radiation): Total digestive system care, Vol. 6, No. 6, Pages 79-89, 6-7 (2002.02). For drug resistance to be identified in the present invention, chemotherapies are typically considered. However, resistance to radiation therapy is also associated with time-lapse profiles. Therefore, radiation therapy is herein encompassed by the concept of pharmaceutical agents.

As used herein, the term “pharmaceutically acceptable carrier” refers to a material for use in production of a medicament, an animal drug or an agricultural chemical, which does not have an adverse effect on an effective component. Examples of such a pharmaceutically acceptable carrier include, but are not limited to, antioxidants, preservatives, colorants, flavoring agents, diluents, emulsifiers, suspending agents, solvents, fillers, bulking agents, buffers, delivery vehicles, excipients, agricultural or pharmaceutical adjuvants, and the like.

The type and amount of a pharmaceutical agent used in a treatment method of the present invention can be easily determined by those skilled in the art based on information obtained by a method of the present invention (e.g., information about the level of drug resistance, etc.) and with reference to the purpose of use, a target disease (type, severity, and the like), the patient's age, weight, sex, and case history, the form or type of the cell, and the like. The frequency of the treatment method of the present invention applied to a subject (or patient) is also determined by those skilled in the art with respect to the purpose of use, target disease (type, severity, and the like), the patient's age, weight, sex, and case history, the progression of the therapy, and the like. Examples of the frequency include once per day to several months (e.g., once per week to once per month). Preferably, administration is performed once per week to month with reference to the progression.

As used herein, the term “instructions” refers to a description of a tailor made therapy of the present invention for a person who performs administration, such as a medical doctor, a patient, or the like. Instructions state when to administer a medicament of the present invention, such as immediately after or before radiation therapy (e.g., within 24 hours, etc.). The instructions are prepared in accordance with a format defined by an authority of a country in which the present invention is practiced (e.g., Health, Labor and Welfare Ministry in Japan, Food and Drug Administration (FDA) in the U.S., and the like), explicitly describing that the instructions are approved by the authority. The instructions are so-called package insert and are typically provided in paper media. The instructions are not so limited and may be provided in the form of electronic media (e.g., web sites, electronic mails, and the like provided on the internet).

In a therapy of the present invention, two or more pharmaceutical agents may be used as required. When two or more pharmaceutical agents are used, these agents may have similar properties or may be derived from similar origins, or alternatively, may have different properties or may be derived from different origins. A method of the present invention can be used to obtain information about the drug resistance level of a method of administering two or more pharmaceutical agents.

Also, in the present invention, gene therapy can be performed based on the resultant information about drug resistance. As used herein, the term “gene therapy” refers to a therapy in which a nucleic acid, which has been expressed or can be expressed, is administered into a subject. In such an embodiment of the present invention, a protein encoded by a nucleic acid is produced to mediate a therapeutic effect.

In the present invention, it will be understood by those skilled in the art that if the result of analysis of a certain specific time-lapse profile is once correlated with a state of a cell in a similar organism (e.g., mouse with respect to human, etc.), the result of analysis of a corresponding time-lapse profile can be correlated with a state of a cell. This feature is supported by, for example, Dobutsu Baiyo Saibo Manuaru [Animal Culture Cell Manual], Seno, ed., Kyoritsu Shuppan, 1993, which is herein incorporated by reference.

Any methods for gene therapy available in the art may be used in accordance with the present invention. Illustrative methods will be described below.

Methods for gene therapy are generally reviewed in, for example, Goldspiel et al., Clinical Pharmacy 12: 488-505(1993); Wu and Wu, Biotherapy 3: 87-95(1991); Tolstoshev, Ann. Rev. Pharmacol. Toxicol., 32:573-596(1993); Mulligan, Science260: 926-932(1993);Morgan and Anderson, Ann. Rev. Biochem., 62: 191-217(1993); and May, TIBTECH 11(5): 155-215(1993). Commonly known recombinant DNA techniques used in gene therapy are described in, for example, Ausubel et al. (ed.), Current Protocols in Molecular Biology, John Wiley & Sons, NY(1993); and Kriegler, Gene Transfer and Expression, A Laboratory Manual, Stockton Press, NY (1990).

(Basic Techniques)

Techniques used herein are within the technical scope of the present invention unless otherwise specified. These techniques are commonly used in the fields of fluidics, micromachining, organic chemistry, biochemistry, genetic engineering, molecular biology, microbiology, genetics, and their relevant fields. The techniques are well described in documents described below and the documents mentioned herein elsewhere.

Micromachining is described in, for example, Campbell, S. A. (1996), “The Science and Engineering of Microelectronic Fabrication”, Oxford University Press; Zaut, P. V. (1996), “Micromicroarray Fabrication: a Practical Guide to Semiconductor Processing”, Semiconductor Services; Madou, M. J. (1997), “Fundamentals of Microfabrication”, CRC1 5 Press; Rai-Choudhury, P. (1997), “Handbook of Microlithography, Micromachining, & Microfabrication: Microlithography”. Relevant portions (or possibly the entirety) of each of these publications are herein incorporated by reference.

Molecular biology techniques, biochemistry techniques, and microbiology techniques used herein are well known and commonly used in the art, and are described in, for example, Sambrook J. et al. (1989), “Molecular Cloning: A Laboratory Manual”, Cold Spring Harbor and its 3rd Ed. (2001); Ausubel, F. M. (1987), “Current Protocols in Molecular Biology”, Greene Pub. Associates and Wiley-Interscience; Ausubel, F. M. (1989), “Short Protocols in Molecular Biology: A Compendium of Methods from Current Protocols in Molecular Biology”, Greene Pub. Associates and Wiley-Interscience; Innis, M. A. (1990), “PCR Protocols: A Guide to Methods and Applications”, Academic Press; Ausubel, F. M. (1992), “Short Protocols in Molecular Biology: A Compendium of Methods from Current Protocols in Molecular Biology”, Greene Pub. Associates; Ausubel, F. M. (1995), “Short Protocols in Molecular Biology: A Compendium of Methods from Current Protocols in Molecular Biology”, Greene Pub. Associates; Innis, M. A. et al. (1995), “PCR Strategies”, Academic Press; Ausubel, F. M. (1999), “Short Protocols in Molecular Biology: A Compendium of Methods from Current Protocols in Molecular Biology”, Wiley, and annual updates; Sninsky, J. J. et al. (1999), “PCR Applications: Protocols for Functional Genomics”, Academic Press; Special issue, Jikken Igaku [Experimental Medicine] “Idenshi Donyu & Hatsugenkaiseki Jikkenho [Experimental Method for Gene introduction & Expression Analysis]”, Yodo-sha, 1997; and the like. Relevant portions (or possibly the entirety) of each of these publications are herein incorporated by reference.

DNA synthesis techniques and nucleic acid chemistry for producing artificially synthesized genes are described in, for example, Gait, M. J. (1985), “Oligonucleotide Synthesis: A Practical Approach”, IRL Press; Gait, M. J. (1990), “Oligonucleotide Synthesis: A Practical Approach”, IRL Press; Eckstein, F. (1991), “Oligonucleotides and Analogues: A Practical Approach”, IRL Press; Adams, R. L. et al. (1992), “The Biochemistry of the Nucleic Acids”, Chapman & Hall; Shabarova, Z. et al. (1994), “Advanced Organic Chemistry of Nucleic Acids”, Weinheim; Blackburn, G. M. et al. (1996), “Nucleic Acids in Chemistry and Biology”, Oxford University Press; Hermanson, G. T. (1996), “Bioconjugate Techniques”, Academic Press; and the like. Relevant portions (or possibly the entirety) of each of these publications are herein incorporated by reference.

(Sense Receptors and use Thereof)

The present invention provides a sensor using chemical receptors, and those inventions related thereto.

According to the present invention, qualitative/quantitative evaluation of odor in accordance with olfactory information processing performed in living entities should lead to the expression of the olfactory sense of human, animals, and the like. Similar examples can be found in the relation between vision and video-camera/television so that the method imitating biological mechanism is considered to be an appropriate method of identifying and assaying sensory variables.

The basic underlying concept of the present invention is described below taking olfaction as an example. Odor molecules form the group with the lowest molecular weight amongst the molecules identified by living entities, making hard. In mouse, odor substances are identified by 873 kinds of olfactory receptors, which are expressed in receptor cells (olfactory cells) and function, of 1,296 kinds of those and the processing of raised signal groups in the brain allow to identify various odors. In humans, odor is identified according to the response profile arising at 347 kinds of olfactory receptors which are considered to function in similar to the olfactory receptors of mouse. Within the olfactory receptor, an odor molecule is identified based on the molecular structures at a plurality of specific sites on the odor molecule so that odor molecule A and odor molecule B, which partially share common molecular structures, can be identified by olfactory receptor 1, but not by olfactory receptor 2. Also, the olfactory receptor responds to either odor molecule A and odor molecule B at low concentration of stimulus, but it largely responds to both of them at an increased concentration of stimulant. In many cases, the stimulant concentration whereat the identifying potential of olfactory receptor is high ranges between 1-2 orders of magnitude. To clearly distinguish between such slight differences in specificity of olfactory receptors, odor should be identified using the sensor group having the same properties as the main olfactory receptor group functioning to identify odor in living entities. Investigation of the response sensitivity of individual olfactory receptors for multiple odor substances concluded that the olfactory receptor with the highest sensitivity to the odor substances contributes the major portion of the characteristic odor element induced by the odor substances. Therefore, it is considered that the use of the olfactory receptor with the highest sensitivity to specific odor qualities or odor sensor materials imitating the 3D structures at the binding sites of the odor molecules allows the identification and detection of specific odor qualities.

The comparison of the outputs of multiple kinds of olfactory receptors with different relative sensitivities to odor molecules with different odor qualities allows the measurement and evaluation of the quality and intensity of presented odors. To measure the response of an olfactory receptor, using olfactory cells expressing one kind of designated olfactory receptor and the proteins related to the receptor response system, or cultured cells, the effect of response on a number of markers, including an increase in intracellular calcium concentration, a change in membrane potential, and the like, are optically/electrically measured. To reduce the expression of the proteins related to the receptor response system, fluctuation in the response due to change in the state of cell, and the like, in individual cells, the mean value of responses of the cells which are expected to present statistically significant numbers of the same kind of responses is used for evaluating individual odor qualities. It is also contemplated herein that the system for response measurement in noncellular system is established by recombining olfactory receptor genes.

The present invention is also directed to olfactory receptors having the following DNA sequences and the olfactory receptors as multiple types of genes whose recombinants do not affect the functions and whose bases are partially different.

Therefore, 11 types of base sequences of the present invention in one aspect are associated with the following;
SEQ ID NO. 1: transmembrane domain 3-6 of car-n272
SEQ ID NO. 3: transmembrane domain 3-6 of car-c5
SEQ ID NO. 5: transmembrane domain 3-6 of car-b161
SEQ ID NO. 7: transmembrane domain 3-6 of car-b153
SEQ ID NO. 9: transmembrane domain 2-7 of car-b85
SEQ ID NO. 11: transmenbrane domain 2-7 of car-n266
SEQ ID NO. 13: all coding regions of car-n266
SEQ ID NO. 15: all coding regions of car-n272
SEQ ID NO. 17: all coding regions of car-b85
SEQ ID NO. 19: all coding regions of car-c5
SEQ ID NO. 21: all coding regions of car-b161.

In the above description, sequences of SEQ ID NOs. 1-11 are partial sequences, but retain the function as olfactory receptor.

The present invention makes use of the detection of olfactory receptor's excellent sensitivity capable of concentration-dependently identifying the length of the odor molecule different only by one carbon atom, and the direct use of this sensitivity is also within the scope of the present invention. The difference in threshold concentration for different odor qualities is extremely small, ranging within about 1 order of magnitude. Therefore, for more precise assays, it is required to compare the relative sensitivities at the start of response. The response intensity is usually low, so it is important to increase the S/N (signal/noise) ratio of measurement and use a system wherein the scattering of response of an individual element sensor is statistically decreased.

(Description of Sensation-Evaluation System)

In the present invention developed based on the above findings, the embodiments defined by the sensation-evaluation system are described below based on attached drawings.

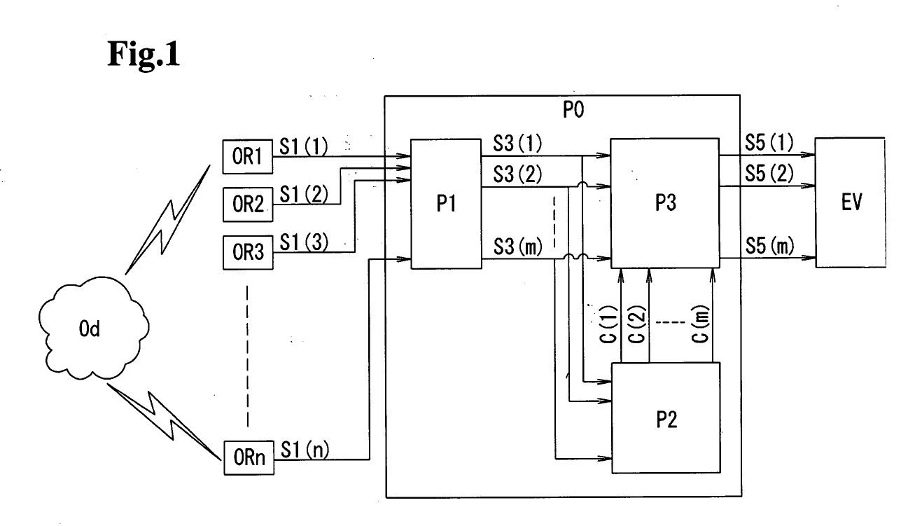

FIG. 1 is a block diagram showing the general structure of the sensation-evaluation system defined by the present invention. The sensation-evaluation system has a plurality of sensors OR1 to ORn, a signal processing member P0 consisting of preprocessing member P1, a coefficient calculation member P2 and an amplification member P3, and an evaluation member EV. Each sensor ORi outputs signal S1(i) depending on presented stimulus Od and the output signal S1(i) is input into preprocessing member P1. Preprocessing member P1 performs given processing on input signal S1(i) (i=1 to n) to output multiple signals S3(j) (j=1 to m), wherein number n of sensors ORi (i=1 to n) is not necessarily the same as number m of signal S3(j) (j=1 to m). Signal S3(j) (j=1 to m) is input to the coefficient calculation member P2 and the amplification member P3, and coefficient calculation member P2 performs given processing on the input signal and outputs control signal C(j) (j=1 to m) to amplification member P2. Amplification member P3, depending on the control signal C(j) (j=1 to m), amplifies the input signal S3(j) (j=1 to m) and outputs it as signal S5(j) (j=1 to m). Evaluation member EV evaluates stimulus Od using signal S5(j) (j=1 to m).

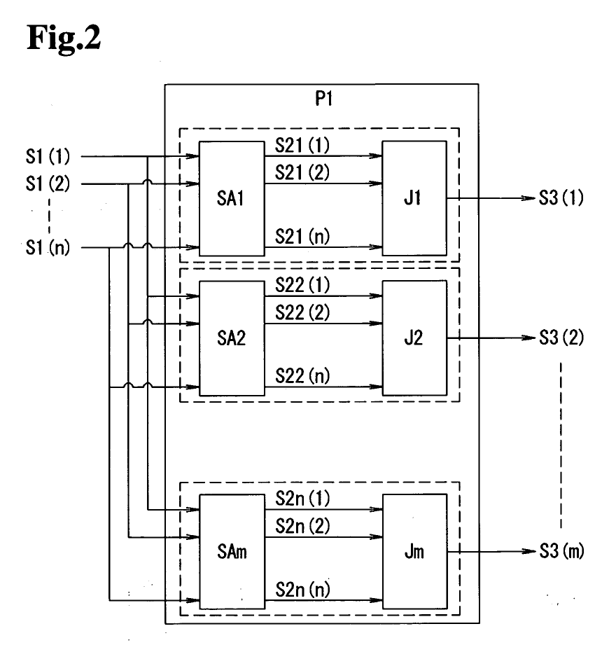

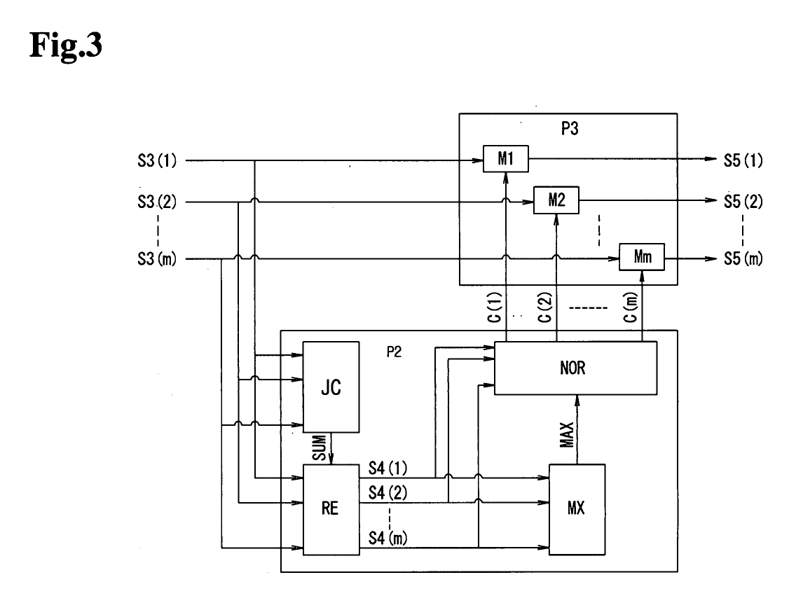

Processings at preprocessing member P1, coefficient calculation member P2 and amplification member P3 are broadly described in FIGS. 2 and 3. FIG. 2 is a block diagram showing the general structure of the preprocessing member P1 and FIG. 3 is a block diagram showing the rough structure of the coefficient calculation member P2 and the amplification member P3.

As shown in FIG. 2, the preprocessing member P1 has a plurality of selection members SA1 to SAm and addition members J1 to Jm, which are structured as pairs. Here, each pair of selection members SAi and Ji corresponds to sensory elemental information (for example, different odor qualities). For example, in the sensation-evaluation system regarding odor substances as the subjects of evaluation, the structure, wherein SA1 and J1, SA2 and J2, and SA3 and J3 correspond to sweet, herbal and fresh, respectively, is possible.

Output signals S1(i) from all the sensors ORi (i=1 to n) are input to selection member SAj. Selection member SAj amplifies each signal S1(i) at given portion aj (i) and outputs it as S2j (i)=αj (i)×S1(i). Here, coefficient aj (i) is the value set by the exterior beforehand for each selection member SAj, which is decided depending on the sense element to be expressed with the property of used sensor ORi, selection member SAi and Ji.

The detailed method of indicating coefficient aj (i) is specifically described for odor below and the overview is described here. Coefficient aj (i) is 1 if the corresponding addition member Jj expresses the sense element specific to a stimulus (e.g. odor molecule). If the corresponding addition member Jj expresses the sense element common to multiple kinds of different stimuli, when the number of kinds of element stimulant (e.g. single substance stimuli such as odor molecules) detected with the highest sensitivity by the sensor is only single, coefficient aj (i) can be the same value as the relative intensity corresponding to the sense element to be expressed at Jj in the relative intensity of multiple sensory elemental information (1 in total) induced by a single kind of the element stimulus. The coefficient is adjusted on system optimization, which becomes a larger number such as 2 when trying to emphasize the peculiar sense element, and, when trying to weakly evaluate the sense element common to multiple element stimuli, the total amount of relative intensity of the common sense element can also be processed using the value below 1 such as 0.7. If a sensor responds to multiple element stimuli with the same degree of sensitivity and the highest intensity, the lowest value among the relative intensities to the sense element of each of corresponding multiple element stimuli is adopted to the coefficient aj(i) as the part common to all the element stimuli.

Addition member Jj adds all the input signals S2j (i) and outputs them as signal S3(j)=Σ_{i=1˜n}S2j (i). Thus, with the setting wherein only the output signal of sensor ORi designated beforehand is multiplied by the coefficient depending on sensor property and passed through each selection member SAj (in the signal transduction line set as being the coefficient to be 0, the signal cannot pass), the addition processing of signals S1(1) to S1(n) input at each addition member Jj, for corresponding sensory elemental information, synthesizes the attributes of each sensor. For example, in the case where odor substances are the evaluation subjects, if it is postulated that addition member J1 corresponds to sweet, the processing at addition member J1 corresponds to the addition of the attribution of each sensor to the intensity perceived as sweet by humans. In humans, after additional given processing is performed on the result of such addition, human senses sweet at certain intensity.

Then, the processing at coefficient calculation member P2and amplification member P3 is described. As shown in FIG. 3, coefficient calculation member P2 consists of addition member JC, relative value determination member RE, maximum value detection member MX and normalization member NOR. Amplification member P3 consists of multiplication members M1 to Mm.

The output signals S3(1) to S3(m) at preprocessing member P1 are input to addition member JC and the relative value determination member RE. Addition member JC adds input signals S3(1) to S3(m) and outputs the result of the addition as SUM=Σ_{i=1˜m}S³(i). Relative value determination member RE, using signal SUM, calculates the proportion of each of input signals S3(1) to S3(m) to the sum total, i.e. S4(j)=S3(j)/SUM. This processing is performed only when any one or more of S3(1) to S3(m) fall within the range of values, by which it is judged that sensors OR1 to ORn respond. Signal S4(i) is input to the maximum value detection member MX and the normalization member NOR. Maximum value detection member MX detects the maximum signal among the input signals S4(1) to S4(m) and outputs it as MAX. Normalization member NOR normalizes input signals S4(1) to S4(m) using MAX and relays control signal C(j)=S4(j)/MAX to the amplification member P3. If the maximum value of S3(i) is S3(X), the result shows C(j)=(S3(j)/SUM)/(S3(X)/SUM)=S3(j)/S3(X). Therefore, it is also acceptable to omit the calculation of S4(j), and directly seek the maximum value S3(X) of S3(i) thus seeking control signal C(j) as C(j)=S3(j)/S3(X). Each multiplication member Mj(j=1 to m) amplifies the corresponding output signal S3(j) from the processing member using the corresponding control signal C(j), and produces S5(j)=C(j)×S3(j).

Evaluation member EV qualitatively and/or quantitatively evaluates the sensory elemental information which would be induced in humans by presented stimulus (e.g. odor quality).

Next, the sensation-evaluation system defined by the present invention wherein odor substances are evaluation subjects is described in a specific manner below.

Each sensor ORi functions in a manner corresponding to olfactory receptors in living entities or olfactory cells as the sensation-evaluation systems shown in FIGS. 1 to 3 function as the systems to evaluate the properties of odor substance Od. Hundreds to thousands types of olfactory receptors respond differently to odor stimuli. Each sensor reacts differently depending on the nature of odor substance Od and outputs a signal depending on the molecular structure of specific sites of the odor molecules constituting the odor substance Od. Each sensor sensitively responds to a different odor stimulus Od. This sensor may use the olfactory receptor cells of a living organism, a product imitating the amino acid sequence of olfactory receptor, and the like.

Selection member SAj and addition member Jj of preprocessing member P1 correspond to, as a pair, different element components of multiple odor qualities to be used determined beforehand. For example, if mint, sweet and fresh are used as element components of the odor qualities, it is possible to make selection member SA1 and addition member J1 correspond to mint, selection member SA2 and addition member J2 correspond to sweet, and selection member SA3 and addition member J3 correspond to fresh. The number of odor qualities which should be specifically evaluate, and thus the number of pairs of selection member SAj and addition member Jj to be employed, depends on the degree to which odor substances are aimed to be classified.

As described above, the coefficient aj(j), designated to each selection member SAj of the preprocessing member P1 is determined according to the properties of the sensor ORi to be used and the sense element to be expressed using selection member SAi and Ji, i.e. odor quality, in consideration of findings 1 and 2.

The mechanism of signal addition for obtaining sufficient information to express odor quality can refer to nerve signal processing by the piriform cortex and, assuming that the data from one kind of olfactory receptor in a known article (Nature, 414: pp. 173-179 (2001)) can extend to that of all of about a thousand types of olfactory receptors as a generalization, it is considered that signals from dozens of kinds of olfactory receptors are added and treated as one signal by nerve cells in piriform cortex. The present inventors conducted experiments based on such an assumption and, as newly obtained data, found that, as described above, the signals from olfactory receptors reaching piriform cortex at the initial stage of response inhibit the signals subsequently reaching there, decreasing the attributable fraction when the latter signals are added, and that the signals showing the odor recognition are conducted some time after the response reaches the piriform cortex (hereinafter called finding 1). In the identification of other odors, the olfactory receptor signals initially input to piriform cortex are generally different, any one of all olfactory receptor signals can reach piriform cortex first and be the leading signal that induces the first output. Therefore, it is considered that the second and later output signals of piriform cortex also try to form the circuit inhibits the input signals, other than the signals attributed to form the output signals. In other words, the circuits inhibiting other input signals are progressively formed depending on the degree to which the nerve cells in the piriform cortex output output signals and that one sensation-evaluation is formed by the combination of multiple different element sense output amounts, based on the addition of these input signals decreased by differing degree.

The present inventors also found experimentally that the olfactory receptors incapable of distinguishing between two different types of odor molecules (i.e. responding to any odor molecules) account for about ½ of the olfactory receptors capable of responding to either of these odor molecules and that of about ¼ of these shows higher sensitivity to one or other of these odor molecules. Considering this finding, and that humans often recognize odor qualities characteristic of two specific odor molecules better than those common to individual shared between different types of odor molecules, it is concluded that the signals from the olfactory receptors incapable of distinguishing two types of odor molecules weakly contribute to the formation of common odor qualities and are responsible for decreasing the addition effects by inhibiting the signals in the part when their responses overlap each other (hereinafter called finding 2).

Based on the above findings 1 and 2, the multiple types of specific odor molecules that make up the element stimuli in odor, commonly responding sensor signals are all used in order to quantitatively evaluate the sense element expressing “odor quality” commonly induced by multiple specific element stimuli. For example, the odor molecules induce the sensory elemental information constituting an odor quality of sweet include many molecules such as R(−)carvone, S(+)carvone, menthone and geraniol, all the signals showing the highest sensitivity to these are used by the quantitative evaluation of sensory elemental information constituting “odor quality” corresponding to sweet. The output signals from these sensors can be used following proper addition. Therefore, it is necessary to investigate in advance to which odor molecule each sensor ORi responds with high sensitivity, i.e. the output signal level from sensor ORi, and, based on such data, the coefficient aj(i) amplifying the output signal of sensor ORi is determined.

Considering the above findings 1 and 2, the method of determining coefficient aj(i) designated to selection member SAj in preprocessing member P1 is further specifically described.

For example, it is postulated that human senses mint, sweet, fresh, herbal and other when smelling pulegone (hereinafter called pu), a kind of odor molecule, (in other words, “the odor element (odor quality) of pu includes mint, sweet, fresh, herbal and others.”), the relative intensities are 0.6, 0.2, 0.1, 0.1 and 0, respectively, (regarding other intensities as 0 for simplicity, expressed using relative intensities adding up to 1), and sensor ORx independently responds to pu with the highest sensitivity. It is also postulated that five pairs of selection member SAi and addition Ji (i=1 to 5) are constituted respectively corresponding to mint, sweet, fresh, herbal and other odor elements. In this case, the output signals S1(x) from sensor ORx are amplified by 0.6-, 0.2-, 0.1-, 0.1- and 0-fold in selection members SA1 to SA5 corresponding to mint, sweet, fresh, herbal and others, respectively, and input to the corresponding addition members J1 to J5. Thus, the output signal S1(x) from sensor ORx, which is input to the signal selection member SA1 corresponding to mint, is amplified by 0.6-fold and input to addition member J1, and the output signal S1(x) from sensor ORx, which is input to the signal selection member SA2 corresponding to sweet, is amplified by 0.2-fold and input to addition member J2. Similarly, the output signals S1(x) input to signal selection members SA3 to SA5 corresponding to fresh, herbal and others are input to addition members J3 to J5, respectively.

The specific sense element amount of Pu is also calculated. If selection member SA6 and addition J6 corresponding to an odor quality specific to pu, which is not found in other odor molecules, are provided, 1 is designated to selection member SA6as the coefficient. Thus, output signal S1(x) from sensor ORx, which is input to selection member SA6, is amplified by 1-hold and input to addition member J6.

Also, it is postulated that humans would perceive mint, sweet, fresh, herbal and other odors when smelling menthone (hereinafter called mn), a kind of odor molecule, to pu compared the relative intensities would be 0.5, 0.3, 0.2, 0 and 0, respectively. Sensor ORy cannot distinguish and responds to pu and mn with the highest sensitivity among odor molecule groups as subjects for detection. Thus, it is assumed that sensor ORy would respond to pu and mn if separately presented with the highest sensitivity among other sensors and the response intensity to pu and that to mn would be the same. In this case, based on finding 2described above, the value in the common part of relative intensities of pu and mn, i.e. the value of lower the relative intensity between the two, is adopted as the coefficient. Using the above relative intensity of pu, if the minimum value is expressed with mark min( ), the output signal S1(y) from sensor ORy, which is input to the selection member SA1 corresponding to mint, is amplified by min(0.6, 0.5)-fold, i.e. 0.5-fold, and input to the addition member J1, and the output signal S1(y) from sensor ORy, which is input to selection member SA2 corresponding to sweet, is amplified by min(0.2, 0.3)-fold, i.e. 0.2-fold, and input to the addition member J2. Similarly, the coefficients to be multiplied by input signals S1(y) to selection members SA2 corresponding to fresh, herbal and others are min(0.1, 0.2), min(0.1, 0) and min(0, 0), i.e. 0.1, 0 and 0, respectively.

It is clear that the determination of coefficient aj (i) as described above is consistent with finding 2. Similarly in the case where the number of types of odor molecules (element stimuli) to which sensor ORy shows maximum sensitivity is three or more, the minimum value of relative intensity corresponding to each odor is used as the coefficient aj (i).

Then, the processing carried out by preprocessing member P1, coefficient calculation member P2 and amplification member P3 to evaluate the properties of odor substances using the signals output by sensors in response to odor substances is the same as the processing described based on FIG. 3. Thus, addition member JC adds output signals S3(1) to S3(m) of input preprocessing member P1 and outputs the addition result as SUM=Σ_{i=1˜m}S3(i). Relative value determination member RE, using SUM, produces the proportion of each of input signals S3(1) to S3(m) to the sum total, i.e. S4(j)=S4(j)/SUM. Signal S4(i) is input to maximum value detection member MX and normalization member NOR, and maximum value detection member MX detects the maximum signal among input signals S4(1) to S4(m) and outputs it as MAX. Normalization member NOR normalizes input signals S4(1) to S3(m) using MAX and relays the control signal C(j)=S4(j)/MAX to amplification member P3. Each multiplication member Mj(j=1 to m) amplifies the corresponding output signal S3(j) from preprocessing member using the corresponding control signal C(j) and produces S5(j)=C(j)×S3(j).

For example, if it is assumed that, after odor substances are presented to sensor ORi, S3(1) among S3(1) to S3(m) of preprocessing member P1 exceeds the given value first but other signals S3(2) to S3(m) show low values, the output signal of the relative value determination member RE would be S4(i)=S3(i)/SUM and the output signal of the maximum value detection member MX would be MAX S4(1). Therefore, control signal C(1)=S4(1)/S4(1)=1, but other control signals C(j) (j≠1)=S4(j)/S4(1)=(S3(j)/SUM)/(S3(1)/SUM)=S3(j)/S3(1), which are less than 1. Thus, the magnitude of signal S3(1) which exceeds the given value first is input to evaluation member EV without influence by multiplication member M1, while other signals S3(2) to S3(m) are inhibited by being multiplied by lower values C(j) than 1 and input to evaluation member EV. Therefore, it is clear that this processing is consistent with finding 1 described above.

As described above, it is possible to realize the sensation-evaluation system wherein odor substances are the subjects of evaluation, which is consistent with findings 1 and 2, and the recording of the odor molecules (element stimuli) evaluation results as database in the recording member (not shown in figures) of the sense possible for evaluation system makes evaluation member EV to qualitatively/quantitatively evaluate the odor substances using the data.

When investigating which element stimuli each sensor responds to with high sensitivity, in evaluation by sense amount (sensual examination), it is difficult to assure that the relative intensities of heterogeneous elements of sense amount, which stimulant elements have, are accurately found, since odor evaluation varies between individuals depending on their experiences. To do this objectively, the following method is used. In olfaction, the responsiveness to various element stimuli is investigated with appropriate intensity (the concentration for appropriate intensity, which is found to suit the odor substance, such as 0.1 mmol in investigation of olfactory cells using solution stimulus, 0.1 to 0.0001 at volume ratio concentration on using satured vapor gas of odor substance solution diluted with odorless innocuous organic solvent, and the like). For all the sensors responding to the element stimulus wherein the relative intensity of presented stimulus quality is to be evaluated, the response rate to each element stimulus is calculated based on the presence or the absence of response. This response rate indicates the ratio of sensory elemental information constituting the common quality on sense amount which the element stimulus. The intensity or level at which the sense element common to multiple element stimuli is duplicated by the typical sense element is as small as possible (ideally, making them orthogonal) leads to the precise evaluation of stimulus quality with less information. Decomposition to orthogonal elements is generally difficult and multivariate analysis may allow the optimization of the intensity or level of sensory elemental information.

As described above, in the classification of odor substances, the type of odor quality to be distinctively treated varies depending on the degree of classification. Therefore, it is possible to be selected and use necessary items among the items exemplified below. If odor substances are roughly classified, a small number of terms are selected from the following odor qualities, if they are to be more narrowly defined into smaller parts, many terms are selected. The classfication of odor substances becomes possible by structuring the sensation-evaluation system having corresponding selection members and addition members and by determining the coefficient α designated to selection members following an initial experiment wherein odor substances (element stimuli) are presented in advance.

### EXAMPLES OF ODOR QUALITY

Examples of odor quality include, but not are limited to, aromatic, camphoraceous, citrus fruit, banana/pineapple, fragrant, immature, sweet, heavy, light, fresh, repulsive, moldy, earthy, acid stimulatory, rancid, drug, herbal, woody, hinoki (Japanese cypress), pine, resinous, overheated meat, raw meat, fishy, garlic, onion, bell pepper, carrot, celery, perilla, sesame, almond, cinnamon, floral, rose, jasmine, lavender, muguet, vanilla, peppermint, spearmint, spicy, cheese, carnivore, herbivore, excrement, sweaty, ammonia, alchohol, organic solvent, ether, oily/fatty, naphthalene, musky, sulfurous, and the like.

In the sensation-evaluation system shown in FIGS. 1 to 3, the function of the coefficient calculation member P2 continues to inhibit the output signals from the sensors which respond to stimulus Od second or later. However, in odor processing by living entities, continuous presentation of odor stimuli leads olfactory receptors that have slowly responded to progressively increase the frequency output signals to be used for odor processing. Therefore, for higher consistency with actual odor processing, the present sensation-evaluation system adds the inhibited signals other than the maximum signal amongst signals S3(1) to S3(m) and, if the addition value becomes higher than given value, it is possible to restructure the system with a new control member (not shown in figures) outputting a control signal to increase the inhibited signal to the corresponding multiplication member Mi. For example, the new control member, when the additional value of inhibited signals is below the given value, outputs control signal C1(i) of zero level to multiplication member Mi and, when the additional value increases to the given value or higher, if it is assumed that the maximum signal among the inhibited signals is S3(k), outputs only control signal C1(k) corresponding to signal S3(k) to multiplication member Mk at given value, keeping other signals C1(i) (i≠k) to zero. Multiplication member Mk to which control signal C1(k) is input amplifies signal S3(k) by the rate determined using control signals C1(k) and C(k), e.g. (C1(k)+C(k))-fold. If the repetition of such procedure increases the output signal S3(i) from inhibited sensor with time while stimulus Od is presented, those inhibited output signals S3(i) are, in order of increasing magnitude, deregulated at the corresponding multiplication members and become amplified, allowing the present sensation-evaluation system to perform processing which is more consistent the odor processing in a living entity.

As described above, the present inventors found that the signals from olfactory receptors incapable of distinguishing between two types of odor molecules that weakly contribute to the formation of common odor qualities are responsible for decreasing the addition effects by being inhibited (see finding 2 described above). While, in combinations of odor stimulus and olfactory receptors, it can be considered that the signals from olfactory receptors incapable of distinguishing between two types between of odor molecules may largely contribute to the formation of common odor quality and may be responsible for increasing the addition effects by being amplified. Therefore, in this case, amongst the relative intensities of odor qualities that humans sense with respect to each of multiple types of odor molecules that are indistinguishable, the maximum value of a specific odor quality is used as coefficient aj(i) designated for selection member SAj.

For example, by the same mechanism as described herein for odor molecules, pu and mn, if sensor ORy cannot distinguish pu and mn, i.e. if it is assumed that sensor ORy responds to presented pu and mn with the highest sensitivity amongst other sensors and the same response intensity, the values in common portion of relative intensities to pu and mn, i.e. the values of higher relative intensity, are used as coefficients. If the maximum value is expressed with mark ( ) using the above relative intensities to pu and mn, the output signal S1(y) from sensor ORy, which is input to selection member SA1 which corresponds to mint, is amplified by max (0.6, 0.5)-fold, i.e. 0.6-fold and input to addition member J1, while the output signal S1(y) from sensor ORy, which is input to selection member SA2 corresponding to sweet, is amplified by max (0.2, 0.3)-fold, i.e. 0.3-fold and input to addition member J2. Similarly, the coefficients to be multiplied by the input signals to fresh, herbal and other selection members are max (0.1, 0.2), max(0.1, 0) and max(0, 0), i.e. 0.2, 0.1 and 0, respectively. Similarly in the case that the number of types of odor molecules (element stimuli) to which sensor ORy shows the highest sensitivity is three or more, the maximum value of relative intensity corresponding to each of the odor molecules is used as the coefficient. In this case, since the sum total of coefficients, Σ_{j=1˜m}αj(i), may be more than 1, the coefficients determined by subtraction as being the sum total to be 1, can also be used.

The transient property of sensor is not considered in the above, however, the output signals of living olfactory receptors increase from zero to the values depending on stimulus concentration for limited time after odor substances are presented. The output signals from olfactory receptors arise faster to odor components with higher intensity, and the output signals from the olfactory receptor responding to specific odor stimuli with higher sensitivity arise faster than the output signals from other olfactory receptors.

Therefore, though the processing of output signals from sensors can be started shortly after odor substances are presented, the output signal S3(i) is on almost zero level or on noise signal level at that time, and subtraction processing does not lead to normal results. For example, since output signal SUM of addition member JC at coefficient calculation member P2 is on almost zero level, there is a high possibility that the subtraction result at relative value determination member RE is not normal. Therefore, after odor substances are presented, it is desirable that the processing is started from the point in time when significant output signal S3(i) arises. For example, evaluation member EV continuously observing output signals from each sensor, in given time t, e.g. t=0.2 seconds (corresponding to about 0.2 seconds of difference between the following about 0.3 and 0.1; it is considered that it takes about 0.3 seconds for the brain to judge the presented odor substance as a significant signal and, in olfactory receptors, 0.1 seconds for the initial signal to reach a significant level since odor substances are presented), from the point of time when judging that the output signals from one sensor or another are higher than the given level, i.e. that one sensor or another clearly sends signals for presented stimuli, outputs triggers to each member, especially, relative value determination member RE and maximum value detection member MX, for the start of the processing.

In the setting of coefficient C(i) to be input to the multiplication member Mi, the value normalized by using the highest value among output signals S3(i) of preprocessing member P1 is used and the coefficient depends on what point in time the value is obtained. Therefore, time t while evaluation member EV outputs triggers is not always 0.2 seconds and, as a parameter optimizing the performance of sensation-evaluation system, can be regulated depending on the property of used sensor.

Therefore, as regards processing within the sensation-evaluation system, signal processing considering the transient nature of the sensor allows it to cope with situations wherein the sensor has the same transient property as the living olfactory receptor used.

As described above, with continuous observation of output signals from each sensor, it becomes unnecessary to know the point in time when the stimulus has actually been presented. Furthermore, the recording of observed values using observation methods can be used for confirmation of motion of each sensor, change in stimulus intensity, the confirmation of the peak value and time of each sensor output, the measurement at each multiplication member of the timing when the coefficient for decreasing the output of signal processing member is set, and the verification of the change in sense element amount sensing each sensor, and the like.

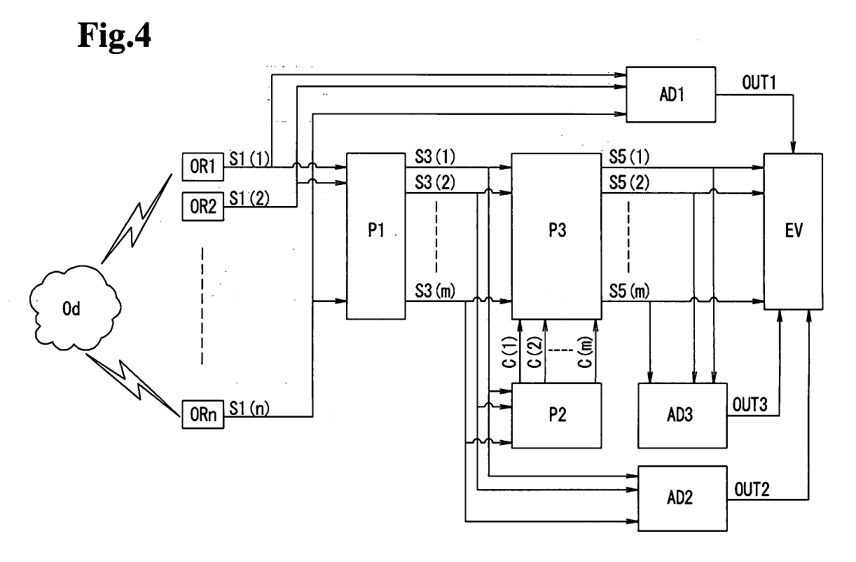

Further, in the sensation-evaluation system defined by the embodiments of the present invention, which is shown in FIG. 1, and in FIG. 4, it is also possible to provide an addition member AD1 adding output signal S1(i) of each sensor ORi, addition member AD2 to add output signal S3(i) of preprocessing member P1, and addition member AD3 adding output signal of amplification member P3.

Evaluation member EV can judge the point in time when the stimulus is presented, the change in stimulus intensity, and the like, by monitering output value OUT1 of the addition result at addition member AD1. This allows, for example, for washing the sensor (i.e. sending odorless air containing no odor molecule across the sensor) at the point in time when the stimulus intensity drops below a certain set value, thus allowing the detection of the next stimulus to be precisely conducted untainted by the previous stimulus.

Also, the evaluation member EV can perform, using output value OUT3 of the addition result at addition member AD3 if necessary, the processing of adjustment to signals S5(1) to S5(m) expressing the intensity of each odor quality (sense element), e.g. the processing of subtraction of OUT3 from each signal of S5(1) to S5(m), and, using the result of processing, facilitate the qualitative/quantitative evaluation of element of the sense which the stimulus may induce in humans.

The evaluation member can also evaluate whether the motion of the sensation-evaluation system is normal, in which part in the identification range the current stimulus intensity is located, and the like, by comparing addition results OUT1 to OUT3 at addition members AD1 to AD3.

Furthermore, by providing a signal transduction line (not shown in figures) to relay input output signal S1(i) of each sensor ORi directly to evaluation member EV, evaluation member EV can judge by monitoring signal S1(i) whether each sensor is normal or not.

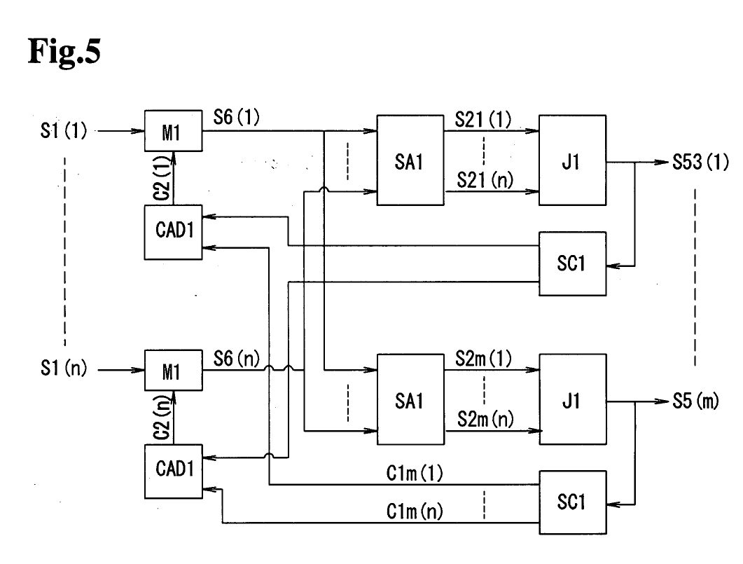

The structure of signal processing member is not limited to the embodiments described above, and various deformations, substitutions, and the like, are possible within the scope consistent with findings 1 and 2 described above. For example, as shown in FIG. 5, a structure wherein multiplication members M1 to Mn are located just behind sensors OR1 to ORn in signal processing member P0 is also possible.

FIG. 5 is a block diagram showing the structure of another signal processing member P0 which can be substituted as signal processing member P0 in the sensation-evaluation system shown in FIG. 1. Signal processing member P0 shown in FIG. 5 is provided with multiplication members M1 to Mn, selection members SA1 to SAm, addition members J1 to Jm, coefficient distribution members SC1 to SCm, and coefficient addition members CAD1 to CADn. N number of sensors is not necessarily the same as m number of selection members SA1 to SAm or addition members J1 to Jm.

Multiplication member Mi produces signal S6(i) by multiplying the output signal S1(i) of the sensor (not shown in figures) by the coefficient depending on control signal C2(i) from the coefficient addition member CADi. Signal S6(i) is input to selection members SA1 to SAm and takes given processing by selection members SA1 to SAm and addition members J1 to Jm and, relays the resultant signal, signal S5 (i) is produced and input to evaluation member EV (not shown in figures). The processing by selection members SA1 to SAm is the same as that by selection members SA1 to SAm in FIG. 2 and the description is omitted here. Also, addition members J1 to Jm perform the processing of multiplying the addition result by given coefficient, besides the addition processing performed by addition members J1 to Jm in FIG. 2.

Output signal S5(j) is output to coefficient distribution member SCj, and outputs predetermined control signals C1j(i) based on a signal S5(j) to signal (signal designated as zero as a coefficient at selection member SA1-SAm) not selected at the corresponding selection member SAj. For example, at selection member SAj, when the coefficient is designated to be 0 in response to output signals from sensors 1 and 3, predetermined values other than 0 for control signals C1j(1) and C1j(3) corresponding to sensors 1 and 3 are output and other control signals C1j(i) (i≠1, 3) are output as 0. Control signals C1j(i) are input to the corresponding coefficient addition member CADi, and the coefficient addition member CADi adds a plurality of control signals C1j(i) (j=1˜m) with optionally multiplication by a predetermined coefficient to output the reciprocal of the results thereof as control signals C2(i). If necessary, the reprocical of the addition results may be multiplied with a coefficient for adjustment in order to produce control signals C2(i). Multiplier member Mi multiplies output signal S1(i) of the sensor with a coefficient depending on the control signal C2(i) to produce signals S6(i).

When the control signal C2(i) exceeds above a predetermined reference value, the predetermined reference value may be used instead of the control signal C2(i). This allows reflection of the fact that there is limitation to signal suppressing effects.

Further, it is also possible to configure addition members AD1-AD3 as in FIG. 5, in a similar manner as in the block diagram shown in FIG. 4.

In the above-described configuration, it was described whilst not making clear as to whether the signals are analog or digital. When the output signals of sensors ORn are analog signals, then it is possible to configure the present sensation-evaluation system to use an analog element to process the signals (for example, a circuit configuration with an analog transistor), or to configure a sensation-evaluation system to comprise an A/D converter within each signal transmission line so that the subsequent signals may be processed using digital elements. When processing as digital signals, for example, supposing that the control signals C(i) from the normalization member NOR are four bit data, then it is possible to control the amplification factor in the multiplier member Mi, in the range of the factor of 1/1 to 1/16 (or 0-15).

Using the sensation-evaluation system of the present invention as exemplified above, it is possible to obtain intensity distribution against the spectrum of odor molecules, i.e. the quality of each odor, by conducting evaluations against each species of odor molecule (stimulus element). Accordingly, the plurality of species of spectrum data obtained may be superimposed, i.e., each of the plurality of species of spectrum data may be multiplied with an appropriate coefficient to obtain a spectrum of the desired odor molecule.

Accordingly, an odor formulation system of the present invention comprising a pluratility of capsules with a plurality of odor molecules stored therein in a sealed manner per species, an opening means for opening the odor molecule from the capsule, and a control measure for controling an amount of odor molecule released by the releasing means, is used to calculate a coefficient for multiplying a spectrum for each odor molecules which can form a desired spectrum of odor or a similar spectrum thereto. Control signals corresponding to the coefficient of the calculation results corresponding to each of the opening means for each capsule from the control means is transmitted to release the desired amount of the odor molecule from the capsule to allow human sensing.

Specifically, an odorant substance inducing the odor of interest (mixture of a plurality of odor molecule species) is evaluated using the sensation-evaluation system of the present invention. The result thereof is used to open a plurality of odorant capsules to release predetermined odor molecules as described above, and the yielded mixed odorant substances are re-evaluated by the sensation-evaluation system of the present invention. The results of the odor of interest and the result of the mixture are compared to allow correction/amendment. As a result of the comparison, any defect in any aspect of odorant quality, the quality of the odorant can be compensated by determining the species of the odor molecule to be added and the amount thereof, and if there is any excess quantity of specific odorant odor molecule, then the species of such odor molecule can be decreased, furthermore the amount to be decreased may be determined in order to decrease the amount of the quality of the odorant. By repeating this procedure, it is possible to determine the necessary odor molecules and the necessary amounts thereof for inducing the desired odor in an accurate manner.

As used herein, it is desired to prepare data bases of the evaluation results obtained by the sensation-evaluation system relating to odor molecules which are element stimulants.

In addition to odor, stimulants such as gustation and the other senses may also be formulated by using the evaluation data obtained by using the sensation-evaluation system of the present invention with respect to the corresponding element stimulant elements.

Further, species and number of the chemical receptors to be arrayed may be varied depending on the target to be measured. When very limited odor species are targeted, the number of species can be reduced. For example, in a case of sensor for identifying spearmint and caraway odors, it is possible to configure a sensor to comprise, for each molecule, two sensitive olfactory receptors distinguishing R(−)carvone and S(+)carvone, which are odor molecules constituting major components of the two odors, those sensitive olfactory receptors not capsule of distinguishing the same, one to two olfactory receptors sensitively distinguishing (−) menthol which is a major component of mentha and peppermint, one or two olfactory receptors sensitively distingushing limonen which is contained in mint and caraway, two or more olfactory receptors not responding to the mentioned components, resulting in the inclusion of ten or more olfactory receptors in total. It is predicted that there are about 70 species of olfactory receptors responding to R(−)carvone and S(+)carvone which are major component for spearmint and caraway odors. Therefore, it is believed that 20 to 30 species of those olfactory receptors not responding to the same may be sufficient to distinguish both odors in an accurate manner. Therefore, a maximum about 100 receptors may be sufficient for preparing such an array. It has been reported that there are 347 species of functional human olfactory receptors, and thus it is believed that preparing array having about 300 species of typical olfactory receptors of human, mice and the like, allows a sensor to have similar distinguishing capabilities to the human olfactory sense.

### DESCRIPTION OF BEST MODE FOR CARRYING OUT THE PRESENT INVENTION

Hereinafter, the present invention will be described by way of embodiments. Embodiments described below are provided for illustrative purposes only. Accordingly, the scope of the present invention is not limited by the preferred embodiment.

In one aspect, the present invention provides a chemical sensor. The present sensor comprises a) a nucleic acid comprising a sequence encoding a chemical receptor gene; b) a support with a cell located thereon wherein the cell is the nucleic acid; c) means for measuring a signal caused by the chemical receptor; and d) means for providing information relating to a chemical by calculating the extent of activation of the chemical receptor from the intensity of the measured signal. Chemicals as used herein may be any substance, and preferably a substance interacting with a living organism. Such a chemical substance interacting with a living organism includes biological signal transduction substances including, but not limited to, cytokines (for example EGF, HGF, FGF and the like), hormones, parakines, midkines and the like; substances reacting with sensory sources (sweet, sour, bitter, salty, umami(savory)) including for example sugars (glucose, sucrose, and the like), acids (for example, citric acid, acetic acid and the like), bitter substances (for example, tannins and the like), salty substances (for example, sodium chloride and the like), umami (for example, sodium glutamate and the like). Pungent taste (pain sensory) is also considered to be a kind of taste, and thus a pungent taste (for example, capsaicin) may also be included in the category of chemical substance. Alternatively, the chemical substances may be olfactory sources. Such olfactory sources include all substances that may be a target of olfactory receptors. Such chemicals may typically be volatile, and include but are not limited to for example, low molecular weight organic molecules such as ethanol or the like. Senses relating to sugars such as glucose, sucrose and the like; salt such as sodium chloride and the like; and umami such as sodium glutamate and the like, are known to be mediated by a receptor coupled with a G-protein. Senses relating to acids such as citric acid, acetic acid and the like, and bitter substances such as tannin and the like, are known to be signaled via a pathway mediated by channel. With respect to odors or olfactory sense, there are a number of basic olfactory sources, including, but not limited to, for example, aromatic, camphoraceous, citrus fruity, banana/pineapple fruity, perfume, immature, sweet, heavy, light (fresh), repulsive, moldy, earthy, acid, rancid, chemical, herbal, woody, retinispora (hinoki), pine, resinous, well-done meat, raw meat, fishy, garlic, onion, pimento, carrot, celery, perilla smell, sesame, almond, cinnamon, floral, rose, jasmine, lavender, muguet, vanilla, peppermint, spearmint, spicy, cheese, carnivore, herbivore, fecal, sweaty, ammonia, alcohol, organic solvent, ether smell, oily/fatty, naphthalene smell, musky, sulfur smell and the like. In an exemplary embodiment, fresh, herbal, sweet, caraway and spearmint are selected as basic olfactory sources, but the present invention is not limited to these. Such molecular recognition is described in detail in Aji to Nioi no Bunshi Ninshiki (Molecular Recognition of taste and odor), ed. The Chemical Society of Japan, Quarterly Review, 40, 1999, which is incorporated herein as reference in its entity.

In another aspect, the present invention provides a chip for use in a chemical sensor. The chip comprises at least a) a nucleic acid molecule comprising a base sequence encoding a chemical substance receptor gene; and b) a substrate with a cell located thereon with the nucleic acid molecule introduced therein.

In another aspect, the present invention is related to a method for obtaining information relating to a chemical substance in a sample. The method comprises the steps of A) providing a cell with a nucleic acid molecule introduced therein, the nucleic acid molecule comprising a sequence encoding a chemical receptor gene; B) providing the cell with a sample containing or suspected to contain the chemical of interest; C) determining a change in signal derived from the chemical receptor gene in the cell, by the chemical; and D) calculating the level of activation of the chemical receptor from the change in intensity of the determined signal to provide information about the chemical.

Following are specific descriptions relating to preferable embodiments is applicable to all categories including the sensor, chip, method, system and the like of the present invention, where applicable unless otherwise specified The nucleic acid molecule used in the present invention preferably further comprises a sequence encoding a marker gene.

The nucleic acid molecule comprising a sequence encoding a chemical receptor gene and an optional sequence encoding a marker gene, may be prepared using genetic engineering technology well known in the art. Such two sequences may be located in a continuous manner, or in a completely separate manner. Such a nucleic acid molecule may comprise a regulatory sequence so that when introduced into a cell, the sequence may be expressed. However, there are cases where similar regulatory sequences may be available in the cell, such regulatory sequences are not necessarily contained in the present invention. Further, such a nucleic acid molecule is preferably contained in a vector to facilitate introduction into a cell. Selection of such a vector is within the ordinary skill in the art, and as described elsewhere herein, those skilled in the art may carry out the same in an appropriate manner.

In the present invention, a substrate with a cell located thereon, the cell having the nucleic acid molecule introduced (for example, by transformation, transduction, transfection and the like) therein, may be prepared by fixing the cell with the nucleic acid molecule introduced therein to a substrate, or introducing (for example, by transformation, transduction, transfection and the like) the nucleic acid molecule to a cell after the cell is fixed to a substrate. Cell as used herein may be any cell as long as the cell may express a nucleic acid introduced therein. Preferably, cells that can be easily maintained on a substrate are desirable. Such a cell include, but is not limited to, for example, HEK293 (HEK293T), CHO, COS-7, neuroblastoma, NG108-15 and the like. Any substrate may be used with any material or form, as long as the substrate can be used as a sensor of the present invention. Preferably, the material is advantageously biocompatible. The sensor of the present invention uses a mechanism using expression sustaining biological activity of a chemical receptor in a cell, and therefore the survival of the cell is preferable. Accordingly, when no biocompatible material is used, it may be desirable to coat such a material with a biocompatible material. Preferable form may be for example, quadrangle such as square, as this form is amenable for normalization.

In the present invention, means for determining signals derived from a marker gene or chemical receptor genes, may be prepared by using a technology well known in the art depending on the signal to be determined. Such means for determination may be located in a position so that signals can be detected from a cell. When a signal is fluorescence, any fluorescence measuring apparatus well known in the art may be used. When signal is an electric signal, means for determining electric signal may be used. When signal is calcium concentration, it may be determined as an electric signal, or otherwise physically determined as for example fluorescence using different means such as a specific reagent including fura-2 and the like, for example. When signal is a chemical signal, then any means for causing such a specific chemical reaction specific to the chemical signal, may be used. When a signal is a biological agent, then means for determining change in cellular morphology, migration of a cell and the like, may be used. Means for detecting physical signal are preferably used, as it is amenable for digitization, comparison in a relative manner and the like.

In the present invention, the means for providing information relating to a chemical by calculating the extent of activation of the chemical receptor from the intensity of the measured signal, may be means for conducting calculation which provides information relating to chemical substances, based on signal information obtained from the means for detecting the signals, and may usually be prepared using a computer. Correlation of signal intensities and information of chemical substances may be conducted using an algorithm known in the art or a combination thereof. Accordingly, a system with a computer program stored therein implementing such an algorithm or a combination thereof, may be used as such means for providing information relating to such information relating to chemical substances. Construction of such a system may be made using well known technology in the art, and those skilled in the art may be able to construct the invention using such technologies. Preferably, such means for providing information relating to chemical substances may conduct measurement by means for measuring such signals.

In the present invention, a nucleic acid molecule to be introduced in a cell, preferably comprises a sequence encoding a marker gene. Inclusion of such a marker gene facilitates transduction of signals from chemical receptor occurred by interacting with the chemical receptor. Accordingly, in a preferable embodiment, such a marker gene is preferably conjugated with a chemical receptor. As used herein “conjugate” refers to conjugation of a chemical receptor and a marker gene to be stimulated using a ligand to the receptor as a stimulant, and such stimulation may be determined by measuring a label presented by the marker gene.

In a preferable embodiment, the chemical receptor comprises a receptor selected from the group consisting of nuclear receptors, cytoplasmic receptors and cellular membrane receptors. Such a receptor may be a single species, or may include a plurality thereof. Alternatively, one kind of a plurality of species of receptors may be included (for example, nuclear receptors and cellular membrane receptors and the like).

In a preferable embodiment, chemical receptors used in the sensor and chips of the present invention, may include a receptor selected from the group consisting of G protein coupled receptors, kinase type receptors, ion-channel type receptors, nuclear receptors, hormone receptors, chemokine receptors, and cytokine receptors. G-protein coupled receptor is preferable, because G-protein can be co-expressed as a marker gene and the conjugation thereof simplify signal transduction and allows identification of signals.

In a more preferable embodiment, the chemical receptor used in the sensor and chip of the present invention, comprise an olfactory or a gustatory receptor. The chemical receptor may include both, as there may be a common receptor for both. In another embodiment, the chemical receptor includes a gustatory receptor, as the use of such a gustatory receptor allows reproduction of a taste. In another embodiment, the chemical receptor includes an olfactory receptor, as the use of an olfactory receptor allows reproduction of an odor sensor.

In another preferable embodiment, in the present invention, the chemical receptor gene is selected from the group consisting of retinoic acid receptors, EGF receptors, hormone receptors, interleukin receptors, interferon receptors, and CSF receptors In another embodiment, said chemical receptor gene used in the present invention comprises at least one, preferably at least two, more preferably at least about 10, still more preferably at least about 20 nucleic acid sequence, selected from the group consisting of SEQ ID NO: 1, 3, 5, 7, 9, 11, 13, 15, 17, 19, 21, 34, 36, 38, 40, 42, 44, 46, 48, 50, 52, 54, 56, 58, 76, 78, 80, 82, 84, 86, 88, 90, 92, 94 and 96, or a variant or fragment thereof. Alternatively, the chemical receptor gene used in the present invention comprises at least one, preferably at least two nucleic acid sequence selected from the group consisting of sequences set forth in SEQ ID NO: 60, 62, 64 and 66, or a variant or fragment thereof. In a preferable embodiment, the chemical receptor gene used in the present invention, comprises at least one, preferably at least two, more preferably at least about 10, still more preferably all of nucleic acid sequence(s), selected from the group consisting of SEQ ID NO: 13, 15, 17, 19, 21, 34, 36, 38, 40, 42, 44, 46, 48, 50, 52, 54, 56, 58, 76, 78, 80, 82, 84, 86, 88, 90, 92, 94 and 96, or a variant or fragment thereof. In a preferable embodiment, the chemical receptor gene used in the present invention, comprises at least one, preferably at least two, more preferably at least about 10, still more preferably all of nucleic acid sequence(s), selected from the group consisting of SEQ ID NO: 13, 15, 17, 19, 21, 34, 36, 38, 40, 42, 44, 46, 48, 50, 52, 54, 56, 58, 76, 78, 80, 82, 84, 86, 88, 90, 92, 94 and 96, or a variant or fragment thereof.

In the present invention, it is sufficient for the number of the chemical receptors used, to be at least one. Preferably, at least about two chemical receptor genes are included in the present invention, more preferably, at least about ten chemical receptor genes are included. Having at least two genes allows one to sufficiently construct a simple odor sensor, and having at least ten receptors allows one to produce a sensor having distinguishing ability that is amenable for routine uses. More preferably, at least about twenty chemical receptor genes are included in the present invention. Having at least about twenty chemical receptors, allow the creation of a sensor capable of substantially distinguishing between all chemical substances. Most preferably, it may be advantageous to use almost all species of human olfactory receptors present, such as at least about 300 species of chemical receptors. As such, the use of substantially all set of chemical receptors (for example, olfactory receptors, gustatory receptors, signal transduction pathways and the like) allow reproduction of the substantially the same sense as possessed by a living entity. In such a case, it may be advantageous to use chemical receptors of a species which has very sensitive senses. For example, substantially all of the set of olfactory receptors of mice, dogs and the like which are believed to be excellent in olfactory sense, may be use to prepare an odor sensor superior to the nose of a human. Such a set of substantially all of the olfactory receptors are available from genome sequences or libraries, or a gene database, and those skilled in the art can readily obtain such information to carry out the present invention.

Accordingly, in a preferable embodiment, chemical receptor genes as used in the present invention, include substantially all species possessed by an animal selected by the group consisting of mice, humans, rats, dogs and cats.

Alternatively, in another preferable embodiment, the chemical receptors used in the present invention, comprise substantially all species of olfactory or gustatory receptor genes which are possessed by an animal selected from the group consisting of mice, humans, rats, dogs and cats.

In a preferable embodiment, a marker gene used in the present invention comprises G protein, said chemical receptor itself, or arrestin.

In a preferable embodiment, the marker gene comprises Gα gene such as Gα15, Gαq or Gαolf gene or the like, and more preferably, it is advantageous that the marker genes comprise all of Gα gene (for example, SEQ ID NO: 68 (nucleic acid sequence), SEQ ID NO: 69 (amino acid sequence) and the like), Gβ gene (for example, SEQ ID NO: 70 (nucleic acid sequence), SEQ ID NO: 71 (amino acid sequence) and the like) and Gγ gene (for example, SEQ ID NO: 73 (nucleic acid sequence)), SEQ ID NO: 73 (amino acid sequence) and the like). The G-protein coupled gene may advantageously use a set of those conjugated in nature, but the present invention is not limited to this.

The support or substrate used in the present invention may be of any material, and preferably solid support is used. More preferably, the substrate used in the present invention may contain coated or non-coated glass, silicon, silica, polystyrene or polymer films, or the like. It is advantageous to use a firm material such as glass, as it is simple to incorporate into a sensor such as a chip, and the possibility to be resistant to reuses, is enhanced. Such solid support is preferably coated. Coated material is preferably biologically compatible with the cell. Such material includes but is not limited to, for example, poly-L-lysine, silane and the like, and preferably silane is used, as the cell is more firmly fixed. When such a substrate is used, it is preferable to use a cellular adhesion molecule. Use of such a cellular adhesion molecule facilitates the introduction of a gene into a cell, and such a cell is more firmly fixed on a substrate. Such a cellular adhesion molecule includes but is not limited to, for example, fibronectin, vitronectin, or laminin, or a fragment or a variant thereof.

In the present invention, signals may comprise an agent selected from the group consisting of intracellular calcium concentration, inositol triphosphate, cyclic AMP, diacyl glycerol, cyclic GMP and cellular membrane potential, MAP kinase, PKA, PKC and the like. It is preferable to use intracellular calcium concentration as a signal. Calcium concentration may be specifically detected using a fluorescent pigment such as fura-2.

Accordingly, in a more preferable embodiment, the signal is intracellular calcium concentration, and a means for measuring a signal may be those for electrically, chemically or biologically measuring calcium concentration, and most preferably, means for measuring by a fluorescence measuring apparatus using fluorescent pigment such as fura-2.

In a preferable embodiment, it may be advantageous that the marker gene is different from the genes which originally exist in said cell, as the use of such a marker gene different from the originally existing genes, may reduce noise signals.

The cell used in the present invention, may comprise HEK293 cell, CHO cell, COS-7 cell, neuroblastoma, NG108-15 cell and the like. More preferably, the cell comprises substantially one species of a cell. Use of substantially one species of a cell allows one to obtain results with more uniform and more reproducible by reducing different noise signals.

In another preferable embodiment, an olfactory receptor gene is advantageously different from the originally existing genes in the cell. Use of such a marker gene different from the originally existing genes, may reduce noise signals.

In a preferable embodiment, spots or addresses of a nucleic acid molecule or cell or the like on a substrate included in the present invention, are preferably arrayed. In such a case, such a sensor or a chip may be called an “array”.

In the case where the sensor or the chip is arrayed, the size of the array region to be used in the present invention, may be any size, and preferably the smaller is better. In a preferable embodiment, it is more appropriate to have an area of about 200 mm² or less for use as a sensor. Such a form may be quadrangle, more preferably rectangle, and sill more preferably square or circle. Triangle or hexagon or the like, may preferably be used for normalization purposes. The length in a longitudinal direction of an array region, may advantageously be about 15 mm or less, and more preferably, the length in a longitudinal direction is preferably about 7.5 mm or less. In another preferable embodiment, if there are no problems of sensitivity and stability, the length of about 1 mm or less in a longitudinal direction may advantageously be used.

In a preferable embodiment, the chip or sensor of the present invention may advantageously further comprise liquid sufficient for covering the cell The present invention preferably comprises a medium for maintaining a cell. Such a medium may be sustained by surface tension on a chip, or alternatively may be held in a container for retention. By having medium for maintaining a cell, such a cell survives longer, and the shelf life of the sensor used is prolonged, and therefore it is significantly advantageous embodiment. Such a medium varies depending on the cell used, and those skilled in the art can appropriately select a suitable medium using well known technologies in the art. Such a medium includes, but is not limited to, for example, DMEM, RPMI1640, Ham's 12 media and the like. It is preferable for the medium to be a liquid medium. Preferably, it is advantageous that the medium does not contain the chemical of interest for measurement, or contains such a chemical at a known concentration.

It is preferable to provide a liquid medium or liquid around a cell, because a small volume of the solution used in a shallow layer (for example, depth so that detachment on the wall is caused by surface tension, such as about 100 μm-1 mm) to use the surroundings of an arrayed sensor, thereby providing the sensor with a wet environment, and avoiding unnecessary reduction of intensity of gaseous stimulants. This alleviates humidity dependency of the sensor and thereby enables enhanced identification of stimulants. Setting conditions for such wet environments was not set forth in conventional sensors with a wet environment. Further, physical or electrical “nose” or “tongue” imitating sensors were not intended to be used in wet environments due to problems such as shelf life, operation and the like. Therefore, the above mentioned identification is unexpected and achieved for the first time in the present invention. Such a wet sensor is to provide the same environment as in the nose or tongue, and thus the range of applications is enormous.

In an embodiment, in the sensor of the present invention, the d) means for providing information comprises d-1) signal processing member for using a stimulus species categorizing method based on the stimulus element tuning specificity of a cell having a chemical receptor to add a first signal output by predetermined plurality of said sensors, to calculate a value of sensory elemental information expressing a sensation, and outputting a calculation result as a second signal; and d-2) evaluation member for effecting qualitative and/or quantitative evaluation using the second signal output by the signal processing member. Such means for providing information is described herein in detail elsewhere in the specification, and is enabled. It is currently possible to conduct quantitative or qualitative evaluation which was not achievable by the conventional methods, by conducting such information analysis.

Preferably, the stimulus species categorizing method used in the aforesaid means for providing information, advantageously uses classification according to the species of the chemical receptor. Classification allows more detailed or specific analysis.

In an embodiment, the signal processing member reduces as used in the present sensor, when one of first signals output by the plurality of sensors exceeds a predetermined value, the first signal output by a sensor different from the sensor and uses the reduced signal for producing the second signal. Such analysis allows more detailed analysis.

In another embodiment, the signal processing member used in the sensor of the present invention comprises: a plurality of selection members and addition members corresponding to sensory elemental information; a plurality of amplification members corresponding to each of the sensors; a coefficient calculation member for controlling the amplification member, wherein the selection members multiplies a plurality of the first signal with the coefficient designated by each of the sensors to produce a plurality of third signals; the addition members add the plurality of third signals output by the corresponding selection member to produce a plurality of fourth signal; the coefficient calculation member detects the maximum value among the plurality of fourth signals and normalizes each of the fourth signals using the maximum value to calculate control signals; the amplification members use the corresponding control signals to produce the second signals corresponding to the intensity of sensory elemental information. Such a step allows normalization of signals for presenting analyzed data such that subsequent analysis can be simplified.

In another embodiment, in the sensor of the present invention, when a stimulus such as a chemical including gustatory source, olfactory source and the like, is presented, the first signal output by the sensor, is transiently produced directed to a predetermined value corresponding to the intensity or concentration of the stimulus from zero level, wherein the zero level is set as a status where no response is found in response to no stimuli; the third signal is transiently produced associated therewith directing to a predetermined value corresponding to the intensity or concentration of a stimulus from zero level: the coefficient calculation member determines a sensor response starting at base time when one of the first signals is determined to be the signal output in response to a stimulus by the sensor for the first time, and calculates at a predetermined time as an elapsed time from the base time, the control signal for controlling the amplification member using the third signal at the predetermined time; controls the amplification member using the control signal which was calculated at the last time until a control signal is calculated at the predetermined time. Such a step allows more detailed analysis.

In another embodiment, in the present invention, when a stimulus such as a chemical including gustatory sources, olfactory sources and the like, is presented, the first signal output by the sensor, is transiently produced directed to predetermined value corresponding to the intensity or concentration of the stimulus from zero level, wherein the zero level is set as a status where no response is found in response to no stimuli; the third signal is transiently produced associated therewith directing to a predetermined value corresponding to the intensity or concentration of a stimulus from zero level; the coefficient calculation member determines a sensor response starting at base time when one of the first signals is determined to be the signal output in response to a stimulus by the sensor for the first time; during a period of time when the predetermined number of the plurality of third signals change from augmentation to reduction, and calculates, at each time when the third signal is determined to start occurring significant output as a corresponding sense element, and when the third signal is determined to have achieved a plurality of boundary values which divide the section between the significant output value and the maximum value preset to the third signal into a plurality of segments; controls the amplification member using the control signal which was calculated for the last time until the control signal is calculated. Such a step allows representation of more detailed analysis in a normalized manner.

In the chip of the present invention, it is preferably to further comprise c) means for transmitting signals derived from the chemical receptor genes, in addition to comprising a) a nucleic acid molecule comprising a base sequence encoding a chemical receptor gene; and b) a support having arranged thereon a cell with the nucleic acid molecule introduced therein. Means for transmitting such a signal may appropriately select such means according to species of the signals. Such means for transmitting signals include but are not limited to, for example, fura-2 which is a substance specifically emitting fluorescence in the presence of calcium, and the like.

In the method for obtaining information relating to a chemical substance in a sample, the step of A) providing a cell with a nucleic acid molecule introduced therein, the nucleic acid molecule comprising a sequence encoding a chemical receptor gene, may be conducted using gene engineering and cellular biological technologies well known in the art by those skilled in the art. The introduction of a nucleic acid molecule in a cell, may be conducted for example, as described herein, using transformation, transduction, transfection, and the like, and preferably using a transfection reagent. Preferably, it is advantageous to add a cellular adhesion molecule thereto for transfection, as the cell is fixed onto a substrate.

In the method for obtaining information relating to a chemical substance in a sample, of the present invention, the step of B) providing the cell with a sample containing or suspected to contain the chemical of interest may also be conducted by those skilled in the art in an appropriate manner. It is understood that the provision of such may be selected by those skilled in the art depending on the sample of target. For example, if the smell is the target, then a sample containing an olfactory source is approximated as much as possible to a cell so that interaction therebetween is possible. If a taste is determined, a sample containing gustatory source is preferably contacted with a cell.

In the method for obtaining information relating to a chemical substance in a sample, the step of C) determining a change in signal derived from the chemical receptor gene in the cell, by the chemical may also be readily carried out by those skilled in the art. Such determination varies depending on the nature and species of a signal, and those skilled in the art can employ an appropriate method for determination considering a variety of conditions of signals. Such determination includes, but is not limited to, for example, fluorimetry, amperometry, electrometery, antigen-antibody interaction measurement, and the like. When a signal is fluorescence, any fluorometer well known in the art may be used. When signal is an electric signal, means for determining electric signal may be used. When signal is calcium concentration, it may be determined as an electric signal, or otherwise physically determined as for example fluorescence using different means such as a specific reagent including fura-2 and the like, for example. When signal is a chemical signal, then any means for causing such a specific chemical reaction specific to the chemical signal, may be used. When a signal is a biological agent, then means for determining change in cellular morphology, migration of a cell and the like, may be used. Means for detecting physical signal are preferably used, as it is amenable for digitization, comparison in a relative manner and the like.

In the method for obtaining information relating to a chemical substance in a sample, the step of D) calculating the level of activation of the chemical receptor from the change in intensity of the determined signal to provide information of the chemical, may also be readily carried out by those skilled in the art. The signal obtained and the level of activation of the chemical receptor is interrelated, and it is possible to obtain information relating to the detected chemical substance based on the correlation of the signal obtained and the level of activation of the chemical receptor. Accordingly, if such correlation is known, it is possible to calculate information based on such correlation. When such a correlation is unknown or more accurate and confirmatory detection is desired, it is preferable to produce standard curve using known amounts of the chemical and the signals obtained.

In the method for obtaining information relating to a chemical substance in a sample, the chemical of target may be olfactory sources, gustatory sources or the like. An olfactory source may be substantially all chemicals of target capable of binding to an olfactory receptor. Accordingly, when an olfactory source is targeted, the chemical receptor used in the present invention, preferably comprises an olfactory receptor. A gustatory source includes but is not limited to, for example, substance having sweet, sour, bitter, salty, umami(savory), glucose, aspartame, acetic acid, citric acid, butyric acid, amino acids such as lysine, glutamic acid, glycine and the like, quinine, caffeine, potassium chloride, sodium chloride, inosinic acid, guanylic acid, and the like. Accordingly, when a gustatory source is targeted, the chemical receptor used in the present method preferably comprises a gustatory receptor.

In a preferable embodiment, information relating to signals, comprise intracellular calcium concentration, inositoltriphosphate level, cyclic AMP level, diacyl glycerol level. Observation of at secondary messenger level, allows extracellular observation of stimulation.

In the present invention, the step of B) providing the cell with a sample comprising or suspected to comprise a chemical of interest comprises a step of providing the cell with the sample at a flow rate of about 1-4 mm/second. The provision of a sample with such a speed, allows minimization of damage to the sensor and cells, and minimization of diffusion of the chemical substances. Accordingly, more preferably, the step B) comprises a step of providing the cell with the sample at a flow rate of about 2-3 mm/second. Most preferably, the speed may be about 2.5 mm/second. The provision of sample may be carried out by adding a sample containing a chemical substance to an extracellular solution (medium or other liquid), by for example, using an aerosol of gas so that the sample is dissolved. Such a method of provision allows avoiding effects of humidity which was problematic in conventional olfactory or odorant sensors.

Methods for obtaining information relating to a chemical in a sample of the present invention, may comprise the step of correlating the information on the chemical with the information on the sample comprising or suspected to comprise the chemical of interest. Such a step of correlation allows determination of an amount contained in a sample in a quantitative manner. Such a correlation may be carried out by using a variety of technologies well known in the art, and exemplary portions thereof are described herein.

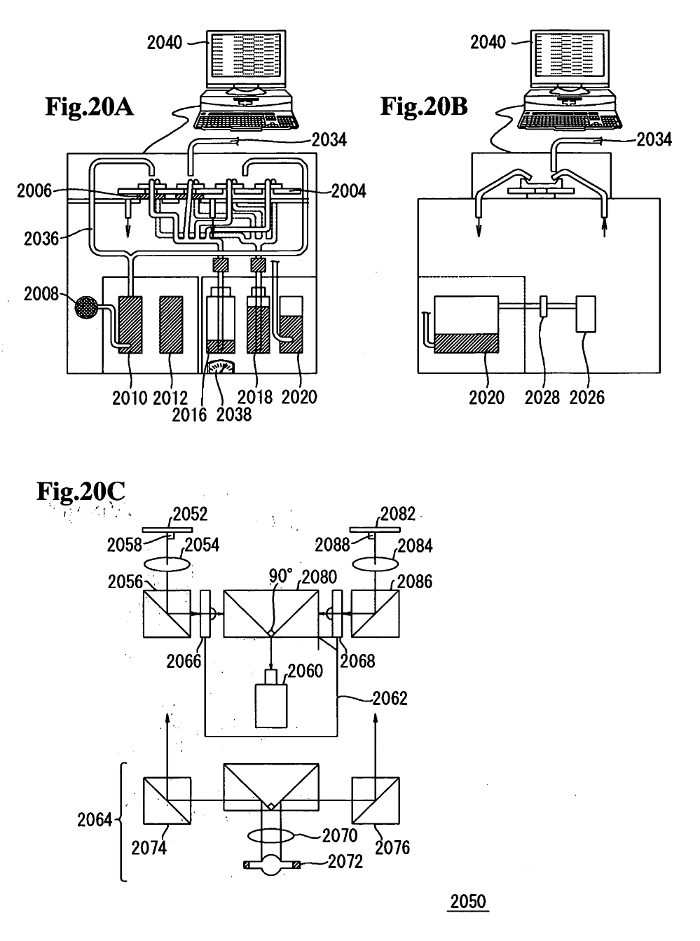

In an exemplary embodiment of the present invention, the present system using the sensor according to the present invention has a configuration as illustrated in FIG. 20. FIGS. 20A and 20B comprise reference odor-free air feed system 2036, and a chemical (gas) sampling system of interest 2034. In the present system, signals such as fluorescence from array-side openings 2004 in four sets of measurement chambers 2032 are transmitted, and the signals are adjusted by opening or closing of movable shutter 2006, to which the chip of the present invention such as an array in case where the chip is provided in an array format, may be mounted. The movable shutter may be elevated at its upper end in order to allow aspiration through emission tubing in case liquid such as culture medium leaks. Reference odor-free air feed system 2036 comprises activated carbon for odor-free air 2010 and additional activated carbon for odor-free air 2012 as a precautionary measure. The system further comprises a culture bottle 2016 for cell culture, and a waste bottle 2018 for collecting waste. These bottles are connected to the measurement chamber via tubing. These bottles and/or tubings comprise aspiration pump 2014 and feed pump 2018 so that liquids may be fed and exhausted and regulated. Such regulation may be conducted via monitor 2038. These pumps are optionally controlled by personal computer 2040, but may also be manually controlled. The system may comprise a negative pressure type air pump 2026 in order to maintain cleanliness therein. Air feed opening (not shown) may be provided with a filter, and may further be provided with feed deodorization cassette 2020 which may be connected to the chamber via tubing. The cassette may be prepared from material resistant in circumstances where activated carbons or liquid are filled to about a half of the cassette.

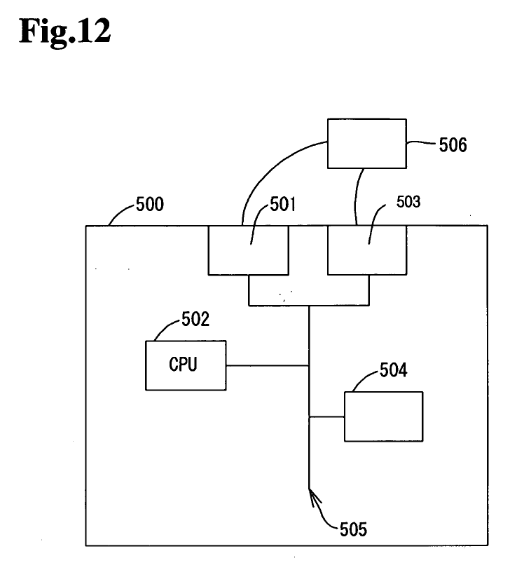

Four sets of measurement chambers for example, consist of two sets of measurement targets, and two sets for preliminary measure, and may be composed so that exchange can be made by sliding the same. One of the two sets of measurement targets may be used for measuring a sample gas, and the other may be used for measuring reference odor-free air, but the present invention is not limited to this, and thus other combination may be used. The bottles may be provided with liquid volume monitor 2038 (for example, optical monitor). Control of each of the elements of the system may be conducted though a monitor, connected to personal computer 2040 operably linked to the system, or may be manually conducted. When manually controled, the system may comprise a button, knob, and the like so that manual control can be easily performed. Personal computer 2040 may use technology well known the art so that data collection, analysis, display and the like can be carried out. Examples of a system of such a computer include, but are not limited, for example, the configuration as shown in FIG. 12.

The inner measurement member may be configured as shown in FIG. 20C. Light enters the camera 2060 via shutter 2052, lens 2054, and beam splitter 2056. In another optical system, signals enters the camera 2060 via shutter 2082, lens 2084, and beam splitter 2086. 2058 and 2088 indicate regions for measurement. In the measurement system, one set of shutter allows the introduction of the light signal to the measurement site via the beam splitter. In a preferable embodiment, as shown in FIG. 20B, respective halves of visual field of the camera are used for capturing both images of sample and control. 2064 is an illumination system for excitation, and exitation light derived from lamp 2072 enters the measurement member through lens 2070 after reflection by beam splitter 2076. Camera 2060 is shaded by a shading device 2062.

In another aspect, the present invention provides a sensation-evaluation system for evaluating sensation arising from a stimulant using output signal of a sensor. The system comprises A) a plurality of sensors having different response characteristics from each other against stimuli from outside; B) a signal processing member for using a stimulus species categorizing method based on a stimulus element tuning specificity of a cell having a chemical receptor to add a first signal output by predetermined plurality of said sensors, to calculate a value of a sense element expressing a sensation, and outputting a calculation result as a second signal; and C) an evaluation member for effecting qualitative and/or quantitative evaluation using the second signal output by the signal processing member. The sensor as used in the present system may be the sensor according to the present invention as described herein, and in addition chemical sensors using conventional physicochemical means may also be used, or both can be combined in the present system. A cell having a chemical receptor may be naturally occurring or artificial. Preferably, it may be advantageous to use cells with a gene introduced using methodologies as described herein, as it allows facilitates normalization of data obtained.

Cells used in the sensation-evaluation system of the present invention may be of any origin, and may be naturally occurring or artificial. Preferably, it may be advantageous that such a cell may be a cell transfected with a nucleic acid molecule comprising a nucleic acid sequence encoding a chemical receptor, as preparation of cells may be possible in a normalized manner using a variety of chemical receptors.

In a preferable embodiment, the signal processing member used in the sensation-evaluation system included in the present invention reduces, when one of first signals output by the plurality of sensors exceeds a predetermined value, the first signal output by a sensor different from the sensor and uses the reduced signal for producing the second signal. By doing so, the present invention allows leveling or smoothing of signals, reduction of noises, and normalization of signals.

The signal processing member as used in the sensation-evaluation system of the present invention, comprises a plurality of selection members and addition members corresponding to sensory elemental information; a plurality of amplification members corresponding to each of the sensors; a coefficient calculation member for controlling the amplification member, and wherein the selection members multiplies a plurality of the first signal with the coefficient designated by each of the sensors to produce a plurality of third signals; the addition members add the plurality of third signals output by the corresponding selection member to produce a plurality of fourth signals; the coefficient calculation member detects the maximum value among the plurality of fourth signals and normalizes each of the fourth signals using the maximum value to calculate control signals; and the amplification members use the corresponding control signals to produce the second signals corresponding to the intensity of sensory elemental information.

In an embodiment, in the sensation-evaluation system of the present invention, when a stimulus is presented, the first signal output by the sensor is transiently produced directed to predetermined value corresponding to the intensity or concentration of the stimulus from zero level, wherein the zero level is set as a status where no response is found in response to no stimuli; the third signal is transiently produced associated therewith directing to a predetermined value corresponding to the intensity or concentration of a stimulus from zero level: the coefficient calculation member determines a sensor response starting at base time when one of the first signals is determined to be the signal output in response to stimulation by the sensor for the first time; and calculates at a predetermined time as an elapsed time from the base time, the control signal for controlling the amplification member using the third signal at the predetermined time; controls the amplification member using the control signal which was calculated at the last time until a control signal is calculated at the predetermined time.

In another embodiment, in the sensation-evaluation system of the present invention, when a stimulus is presented, the first signal output by the sensor, is transiently produced directed to predetermined value corresponding to the intensity or concentration of the stimulus from zero level, wherein the zero level is set as a status where no response is found in response to no stimuli; the third signal is transiently produced associated therewith directing to a predetermined value corresponding to the intensity or concentration of a stimulus from zero level; the coefficient calculation member determines a sensor response starting at base time when one of the first signals is determined to be the signal output in response to a stimulus by the sensor for the first time; during a period of time when the predetermined number of the plurality of third signals change from augmentation to reduction, calculates, at each time when the third signal is determined to start occurring significant output as a corresponding sense element, and when the third signal is determined to have achieved a plurality of boundary values which divide the section between the significant output value and the maximum value preset to the third signal into a plurality of segments; controls the amplification member using the control signal which was calculated for the last time until the control signal is calculated, wherein, when a stimulus is presented, the first signal output by the sensor, is transiently produced directed to a predetermined value corresponding to the intensity or concentration of the stimulus from zero level, wherein the zero level is set as a status where no response is found in response to no stimuli; the third signal is transiently produced associated therewith directing to a predetermined value corresponding to the intensity or concentration of a stimulus from zero level; the coefficient calculation member determines a sensor response starting base time when one of the first signals is determined to be the signal output in response to a stimulus by the sensor for the first time; during a period of time when the predetermined number of the plurality of third signals change from augmentation to reduction, calculates, at each time when the third signal is determined to start occurring significant output as a corresponding sense element, and when the third signal is determined to have achieved a plurality of boundary values which divide the section between the significant output value and the maximum value preset to the third signal into a plurality of segments; controls the amplification member using the control signal which was calculated for the last time until the control signal is calculated.

In an embodiment, the sensation-evaluation system of the present invention evaluates olfactory sense. In this case, the chemical receptor used therein preferably comprises an olfactory receptor, and the sensor may preferably be a sensor reacting to an olfactory stimulus.

In an embodiment, the sensation-evaluation system of the present invention preferably evaluates a gustatory sense. In this case, the chemical receptor used therein preferably comprises a gustatory receptor, and the sensor may preferably be a sensor reacting to a gustation stimulus.

In another aspect, the present invention provides a sensation-evaluation system for evaluating sensation arising from a stimulus using output signal of a sensor comprising a plurality of sensors having different response characteristics from each other against stimuli from outside, a signal processing member for processing output signals from the sensor, and an evaluation member for effecting qualitative and/or quantitative evaluation of a sense from the output signals from the signal processing member. In the present system, the signal processing member comprises a first step in which one analyzes output signals from the sensors, using stimulant species classification method according to the stimulant elements response specificity of a cell having a chemical receptor, and adds the signal output from the plurality of predetermined sensors, calculates a value of sensory elemental information expressing a sensation, and outputs the yielded results as a evaluated signal; and a second step in which the evaluation member uses the evaluated signal to effect a qualitative and/or quantitative evaluation.

The cell used in the sensor evaluation system according to the present invention, may be any cell of any origin, and may be naturally occurring or artificial. Such a cell may be preferably transfected with a nucleic acid molecule comprising a nucleic acid sequence encoding said chemical receptor.

In a preferable embodiment, in the sensation-evaluation method of the present invention further comprises a third step wherein in the first step, said signal processing member reduces, when one of first signals output by the plurality of sensors exceeds a predetermined value, the signal output by a sensor different from the sensor and uses the reduced signal for producing a different signal to be evaluated. By doing so, signal elements corresponding to major stimulatory properties can be extracted, and signal elements corresponding to less important stimulatory properties can be reduced, and it is also possible to digitize stimulatory elements of major stimuli that contribute to overall sensation in a manner so that such major properties are relatively greater than those which are not.

In a preferable embodiment, in the method for evaluating a sense of the present invention, the signal processing member comprises a plurality of selection members and addition members corresponding to sensory elemental information; a plurality of amplification members corresponding to each of the sensor; coefficient calculation member for controlling the amplification member. In an embodiment, the first step of the method for evaluating a sense of the present invention further comprises the fourth step wherein the selection members multiplies a plurality of the signal with the coefficient designated by each of the sensors to produce a plurality of signals; the fifth step wherein the addition members add the plurality of signals output by the corresponding selection member to produce a plurality of the signal; the sixth step wherein the coefficient calculation member detects the maximum value among the plurality of the signals and normalizes each of the fourth signals using the maximum value to calculate control signals; and the seventh step wherein the amplification members use the corresponding control signals to produce the evaluated signals corresponding to the intensity of sensory elemental information.

In an embodiment, in the method for evaluating a sense of the present invention, when a stimulus is presented, the first signal output by the sensor, is transiently produced directed to a predetermined value corresponding to the intensity or concentration of the stimulus from zero level, wherein the zero level is set as a status where no response is found in response to no stimuli and the signal produced by the fourth step is transiently produced associated therewith directing to a predetermined value corresponding to the intensity or concentration of a stimulus from zero level. The present method further comprises the eighth step wherein in the sixth step, the coefficient calculation member determines a sensor response starting base time when one of the first signals is determined to be the signal output in response to a stimulus by the sensor for the first time, and calculates at a predetermined time as an elapsed time from the base time, the control signal for controlling the amplification member using the signal produced by the fifth step at the predetermined time, and controls the amplification member using the control signal which was calculated at the last time until a control signal is calculated at the predetermined time.

In an embodiment, in the method for evaluating a sense of the present invention, when a stimulus is presented, the first signal output by the sensor, is transiently produced directed to a predetermined value corresponding to the intensity or concentration of the stimulus from zero level, wherein the zero level is set as a status where no response is found in response to no stimuli and the signal produced by the fourth step is transiently produced associated therewith directing to a predetermined value corresponding to the intensity or concentration of a stimulus from zero level; the method further comprising the eighth step wherein in the sixth step, the coefficient calculation member determines a sensor response starting at base time when one of the first signals is determined to be the signal output in response to a stimulus by the sensor for the first time; during a period of time when the predetermined number of the plurality of signals produced by the fifth step change from augmentation to reduction, and calculates, at each time when the signal produced by the fifth step is determined to start occurring significant output as a corresponding sense element, and when the signal produced by the fifth step is determined to have achieved a plurality of boundary values which divide the section between the significant output value and the maximum value preset to the signal produced by the fifth step into a plurality of segments; controls the amplification member using the control signal which was calculated for the last time until the control signal is calculated.

In an embodiment, the method for evaluating a sense of the present invention evaluates olfactory sense. In this case, the chemical receptors used preferably include olfactory receptors, and the sensor preferably is a sensor capable of reacting to olfactory sense.

In another embodiment, the method for evaluating a sense of the present invention evaluates gustatory sense. In this case, the chemical receptors used preferably include gustatory receptors, and the sensor preferably is a sensor capable of reacting to gustatory sense.

In another aspect, the present invention provides a method for formulating a stimulant. The method comprises the steps of: the first step of evaluating a predetermined stimulant using a sensation-evaluation system for evaluating sensation arising from a stimulant using the output signal of a sensor comprising: A-1) a plurality of sensors having different response characteristics from each other against stimuli from outside; A-2) a signal processing member for using a stimulus species categorizing method based on a stimulus element tuning specificity of a cell having a chemical receptor to add a first signal output by predetermined plurality of said sensors, calculates the value of sense element expressing a sensation, and outputs the a calculation result as a second signal; and A-3) an evaluation member for effecting qualitative and/or quantitative evaluation using the second signal output by the signal processing member; B) the second step of determining a ratio of stimulant elements to be mixed corresponding thereto using a result of evaluation corresponding to the stimulant elements obtained by the evaluation result of the first step and the sensation-evaluation system; and C) the third step of mixing the determined stimulant elements at the determined ratio.

In a preferable embodiment, the method for formulating a stimulant of the present invention further comprises the steps of: the fourth step of evaluating the mixed stimulant in the third step using the sensation-evaluation system; and the fifth the step of comparing the evaluation step of fourth step and the evaluation result of the first step to determine the ratio to be newly mixed corresponding to the stimulant elements.

In another aspect, the present invention provides a computer readable recording medium having a computer program recorded thereon for implementing a process in a computer in a sensation-evaluation system for evaluating sensation arising from a stimulus using output signal of a sensor comprising a plurality of sensors having different response characteristics from each other against stimuli from outside and a signal processing member for processing an output signal from the sensors. The process herein comprises the procedures of: the first procedure wherein the signal processing member for using a stimulus species categorizing method based on a stimulant element tuning specificity of a cell having a chemical receptor to add a first signal output by predetermined plurality of said sensors, calculates the value of sense element expressing a sensation, and outputs the calculation result as a second signal; the second procedure wherein an evaluation member for effecting qualitative and/or quantitative evaluation using the second signal output by the signal processing member. The recording medium includes but is not limited to, flexible disk, MO, CD-R, CD-RW, CD-ROM, DVD-RAM, DVD-R, DVD-RW, DVD+RW, DVD-ROM, memory card and the like.

In another aspect, the present invention provides a computer program for implementing a process in a computer in a sensation-evaluation system for evaluating sensation arising from a stimulus using the output signal of a sensor comprising a plurality of sensors having different response characteristics from each other against stimuli from outside and a signal processing member for processing an output signal from the sensors. The process comprises the procedures of: the first procedure wherein the signal processing member for using a stimulus species categorizing method based on the stimulus element tuning capability of a cell having a chemical receptor to add a first signal output by predetermined plurality of said sensors, to calculate a value of sensory elemental information expressing a sensation, and outputting a calculation result as a second signal; the second procedure wherein an evaluation member for effecting qualitative and/or quantitative evaluation using the second signal output by the signal processing member.

In another aspect, the present invention provides novel olfactory receptors. The nucleic acid molecules encoding the olfactory receptors comprise: (a) a polynucleotide having a base sequence set forth in SEQ ID NO. selected from the group consisting of SEQ ID NO: 1, 3, 5, 7, 9, 11, 13, 15, 17, 19 and 21, or a sequence fragment thereof; (b) a polynucleotide encoding a polypeptide consisting of an amino acid sequence set forth in SEQ ID NO. selected from the group consisting of SEQ ID NO: 2, 4, 6, 8, 10, 12, 14, 16, 18, 20 and 22, or a fragment thereof; (c) a polynucleotide encoding a variant polypeptide having an amino acid sequence set forth in SEQ ID NO. selected from the group consisting of SEQ ID NO: 2, 4, 6, 8, 10, 12, 14, 16, 18, 20 and 22, having at least one mutation selected from the group consisting of at least one amino acid substitution, addition and deletion, and having biological activity; (d) a polynucleotide which is an allelic variant of DNA consisting of a base sequence set forth in SEQ ID NO. selected from the group consisting of SEQ ID NO: 1, 3, 5, 7, 9, 11, 13, 15, 17, 19 and 21; (e) a polynucleotide encoding a species homolog of a polypeptide consisting of an amino acid sequence set forth in SEQ ID NO. selected from the group consisting of SEQ ID NO: 2, 4, 6, 8, 10, 12, 14, 16, 18, 20 and 22; (f) a polynucleotide encoding a polypeptide hybridizable to any one of the polynucleotides (a) to (e) under stringent conditions, and having biological activity; or (g) a polynucleotide consisting of a base sequence having at least 70% identity to any one of the polynucleotides (a) to (e) or a complementary sequence thereof, and having biological activity.

In a preferred embodiment, the biological activity comprises a signal transduction activity of a chemical. Such signal transduction activity may be determined by directly or indirectly measuring a transduced signal.

The polypeptides of the olfactory receptors of the present invention comprise: (a) a polypeptide encoded by polynucleotide of a nucleic acid sequence set forth in SEQ ID NO. selected from the group consisting of SEQ ID NO: 1, 3, 5, 7, 9, 11, 13, 15, 17, 19 and 21, or a fragment thereof; (b) a polypeptide consisting of an amino acid sequence set forth in SEQ ID NO. selected from the group consisting of SEQ ID NO: 2, 4, 6, 8, 10, 12, 14, 16, 18, 20 and 22, or a fragment thereof; (c) a polypeptide comprising an amino acid sequence set forth in SEQ ID NO. selected from the group consisting of SEQ ID NO: 2, 4, 6, 8, 10, 12, 14, 16, 18, 20 and 22, having at least one mutation selected from at least one amino acid substitution, addition and deletion, and having biological activity; (d) a polypeptide encoded by an allelic variant of a base sequence set forth in SEQ ID NO. selected from the group consisting of SEQ ID NO: 1, 3, 5, 7, 9, 11, 13, 15, 17, 19 and 21; (e) a polypeptide which is a species homolog of an amino acid sequence set forth in SEQ ID NO. selected from the group consisting of SEQ ID NO: 2, 4, 6, 8, 10, 12, 14, 16, 18, 20 and 22; or (f) a polypeptide having an amino acid sequence having at least 70% identity to any one of the polypeptides (a) to (e), and having biological activity.

In a preferable embodiment, the above mentioned biological activity comprises a signal transduction activity of a chemical.

In another aspect, the present invention is related to use of a chemical receptor of the present invention such as those nucleic acid molecules, polypeptides and the like for detecting a chemical. Preferred embodiments for such use may be the same as in the chip, sensor, system, method and the like as described herein.

In another aspect, the present invention provides a method for determining the health condition of a living entity. The method comprises the steps of A) providing a cell with a nucleic acid introduced therein, the nucleic acid sequence comprising a sequence encoding a chemical receptor gene, and a sequence encoding a marker gene; B) approximating or contacting a biological body of interest to the cell to a distance so that at least one chemical can be detected by the cell; C) determining the change of signal derived from the marker gene in the cell by the chemical; D) calculating the level of activation of the chemical receptor from a change in intensity of the signal determined to provide information of the chemical; and E) determining the health condition of the biological body from the information of the chemical. The nucleic acid molecule may comprise a sequence encoding a marker gene. Approximation or contact to a biological body may be conducted using a well known technology in the art. Similarly, measurement of change in signals may be conducted using well known technology in the art as described above. Provision of information and determination health condition may also be conducted by combination of known technologies in the art as described herein.

In a preferable embodiment, the chemical substance may be an olfactory source, and the chemical receptor may comprise an olfactory receptor.

In another aspect, the present invention provides a system for determining the health condition of a biological body. The system comprises A) a cell with a nucleic acid introduced therein, the nucleic acid sequence comprising a sequence encoding a chemical receptor gene, and a sequence encoding a marker gene; B) an opening for a biological body of interest to the cell to a distance so that at least one chemical can be detected by the cell; C) means for determining the change of signal derived from the marker gene in the cell by the chemical; D) means for calculating the level of activation of the chemical receptor from a change in intensity of the signal determined to provide information of the chemical; and E) means for determining the health condition of the biological body from the information of the chemical.

In a preferable embodiment, the chemical substance of target in the system is olfactory source, and the chemical receptor includes an olfactory receptor.

The sensor, chip, system, method, program, recording medium of the present invention will be described more in detail with preferable embodiments thereof, however, it should be understood that the present invention is not limited thereto.

In the present invention, presentation of results may be displayed using any method, for example, may be visually displayed using a display device (e.g., an x axis showing time whilst the y axis shows signal intensity), or alternatively, may be displayed as a table of numerical values. Alternatively, signal intensity may be displayed as optical intensity.

Preferably, cells are fixed to a solid phase support (e.g., an array, a plate, a microtiter plate, etc.) when they are monitored. Such fixation can be carried out using techniques known in the art or techniques as described herein.

In a preferred embodiment, results may be presented in real time. The real time presentation may contain a time lag to some extent if it is performed substantially in real time. A tolerable time lag is, for example, 10 seconds at maximum, and more preferably 1 second at maximum, though the tolerable time lag depends on the required level of real time (simultaneity). For example, in a therapy requiring real time diagnosis, the time lag may be, for example, 30 seconds at maximum.

In a preferable embodiment, the nucleic acid of the present invention, further comprises promoter sequence, enhance sequence, silencer sequence, other flanking sequence in a structural gene sequence in the genome construct, and a genomic sequence other than exon sequences. Promoters may be constitutive, specific, and inducible promoters. Introduction of a promoter allows construction of a system operable as a sensor in a specific case.

In a specific preferable embodiment, conditions to be determined by the present diagnosis method, include, but are not limited to, for example, a response to an anti-cancer agent in a cancer cell, drug resistance, a response to a biological time, a health condition, a response to a treatment, emotion, estrus and the like.

The chip of the present invention may be called an array when cells and chemicals are arrayed in an aligned manner.

In a particularly preferable embodiment, the chemical receptor used in the present invention is preferably transfected within a cell of interest in a form of a nucleic acid molecule containing a sequence encoding a marker gene operably linked to the sequence encoding the chemical receptor.

Such transfection may be performed in solid phase or in liquid phase. For transfection, a method for enhancing introduction efficiency of a substance of target into a cell, may be used. The present invention provides a cell with a substance of target that is rarely introduced into a cell such as DNA, RNA, polypeptide, sugar chain and a complex thereof and the like, together with a cellular adhesion molecule such as fibronectin, preferably by contact to each other, so that the target substance is efficiently introduced into the cell. Accordingly, the transfection method comprises the steps of A) providing a substance of target (for example, a nucleic acid molecule containing the chemical receptor) and B) providing a cellular adhesion molecule, in an arbitrary order, and in addition thereto, further comprises the step of C) subjecting the substance of target and the cellular adhesion molecule in contact with a cell. The substance of target and the cellular adhesion molecule can be provided together, or may be provided separately. Cellular adhesion molecules may be in any form or embodiment described in detail for the composition for enhancing the efficiency of the introduction of a target substance of the present invention into a cell, as described above. Such embodiments may be readily selected by those skilled in the art based on the description of the present application for carrying out the present invention. Accordingly, such cellular adhesion molecules may be any form that is arbitrarily selected by those skilled in the art from those applicable for the composition for enhancing introduction of a substance of target of the present invention into a cell in order to carry out the present invention. Preferably, the cellular adhesion molecule may be extracellular matrix protein such as fibronectin, vitronectin, laminin or the like, or a variant thereof.

In another preferred embodiment, the calculation or determination step of the present invention comprises a mathematical process selected from the group consisting of signal processing and multivariate analysis. Such a mathematical process can be easily carried out by those skilled in the art based on the description of the present specification.

In another aspect, the present invention provides a method for correlating a chemical substance, and a response of a cell having a chemical receptor. The present method may comprise a) exposing the cell to a chemical; b) monitoring the cell in a time-lapsed manner to obtain a profiled of a signal of the cell; and c) correlating the chemical and the profile.

The chemical substance to be correlated in the present invention may be any substance. Such a chemical substance is preferably directly or indirectly applicable to the cell. A method for subjecting a cell to a chemical is well known in the art, and varies depending on the species of the chemical substance. If the substance is a soluble substance, the substance is dissolved in a solvent, and the solution is added to a medium containing the cell in a drop-wise manner to complete the exposure.

In a method for correlation of the present invention, production of a profile may be conducted as described herein above.

Correlation of a chemical substance and a profile in the method for correlation of the present invention, may be provided using a variety of methods. In brief, patterning of profiles is performed in the case where a chemical substance is assessed. In case where there is little difference between the profiles, it can be assumed that the chemical substance is added in a drop-wise manner.

Preferably, a cell may be monitored in a state fixed to a solid phase substrate such as an array, plate, microtiter plate, and the like. Such a method for fixation may be conducted based on a known method in the art or methods described in the present application.

In a preferable embodiment, the method for correlation of the present invention may comprise a step of obtaining a profile corresponding to each chemical substance, using at least two chemical substances. In an embodiment, such chemical substances may be included about at least about three, or at least about four, more preferably at least about 10, but the present invention is not limited to these.

In a specific embodiment, the method of correlation of the present invention comprises a step of categorizing a chemical corresponding to a profile by classifying at least two profiles. Such a classification is readily carried out by those skilled in the art in view of the description of the present application. Such a classification used in the present invention allows correlation and identification of an unknown chemical.

Preferably, the method of the present invention is conducted in an advantageous manner when the cell used is cultured on an array such as a chip. Culturing on an array allows multiple observation of a number of cells at once.

In a preferable embodiment, monitoring may comprise a step of obtaining an image from the array. Provision of such an image allows gross inspection, and thus it is currently possible for a human, in particular those skilled in the art such as a medical practitioner to determine a response by naked eyes.

In a particularly preferable embodiment, chemical substances to be identified by the method of the present invention include bio-molecules, chemically synthesized substances, media and the like.

Examples of such a biological molecule include, but are not limited to, nucleic acids, proteins, lipids, sugars, proteolipids, lipoproteins, glycoproteins, proteoglycans, and the like. Such a biological molecules are known in the art to affect living entities, or if unknown, are considered to be highly likely to have such an effect, and therefore are important as a target of interest.

Yet preferably, such a biological molecule to be evaluated may also be, for example, a hormone, a cytokine, a cell adhesion factor, extracellular matrix, a receptor agonist or antagonist which is expected to affect a cell.

In another aspect, the present invention provides a method for inferring an unidentified chemical substance given to a cell from a profile of a cell. The method comprises the steps of: a) exposing the cell to a plurality of known chemicals; b) obtaining a time-lapse profile of the cell for each known chemicals by time-lapse monitoring the cell; c) correlating the known chemicals with the respective time-lapse profiles; d) exposing the cell to the unidentified chemicals; e) obtaining a time-lapse profile of the unidentified chemicals by time-lapse monitoring the cell; f) determining a profile corresponding to the time-lapse profile obtained in the step of e) from the time-lapse profiles obtained in the step of b); and g) determining that the unidentified chemicals is the known chemicals corresponding to the profile determined in the step of f).

As used herein in the present method, exposure of a chemical substance may use any technology and embodiments described hereinabove, and exemplified in the Examples. As used herein in the present method, production of a profile may use any technology and embodiments described hereinabove, and exemplified in the Examples. As used herein in the present method, correlation may use any technology and embodiments described hereinabove, and exemplified in the Examples. As such, using information relating to known chemicals, and an unidentified chemical monitored in a similar manner, comparison between the known and unidentified chemicals will allow determination whether the unidentified chemical is identical to the known chemical or not. In this case, if the profile is completely identical, then it is of course possible to determine that both chemicals are identical, and when the profiles of both chemicals are substantially the same, then it is also possible to determine that the unidentified chemical is the known chemical. Such determination depends on the amount and quality of information relating to the known chemicals. Such determination is readily carried out by those skilled in the art and may be determined by considering a variety of factors.

In another aspect, the present invention provides a method for inferring an unidentified chemical given to a cell. The method comprises a) providing data relating to a correlation relationship between known chemicals and time-lapse profiles of the cell in response to the known chemicals, in relation to profile of the cell; b) exposing the cell to the unidentified chemical; c) further obtaining a profile of the cell; d) determining a profile corresponding to the profile obtained in the step of c) from the profiles obtained in the step of a); and e) determining that the unidentified chemical is the known chemical corresponding to the profile determined in the step of d).

As used herein, exposure of a chemical, production of a profile, correlation and the like may use any technology and embodiments described hereinabove, and exemplified in the Examples.

### DESCRIPTION OF EMBODIMENTS USING A COMPUTER

A configuration of a computer or system for implementing the method of the present invention is shown in FIG. 12. FIG. 12 shows an exemplary configuration of a computer 500 for executing the method of the present invention.

The computer 500 comprises an input section 501, a CPU 502, an output section 503, a memory 504, and a bus 505. The input section 501, the CPU 502, the output section 503, and the memory 504 are connected via a bus 505. The input section 501 and the output section 503 are connected to an I/O device 506.

An outline of a process executed by the computer 500, is described below.

A program for executing the sense determination method, diagnosis method and the like (hereinafter referred to as a “program”) is stored in, for example, the memory 502. Alternatively, each component of the program may be stored in any type of recording medium, such as a floppy disk, MO, CD-ROM, CD-R, DVD-ROM, or the like separately or together. Alternatively, the program may be stored in an application server. The program stored in such a recording medium is loaded via the I/O device 506 (e.g., a disk drive, a network (e.g., the Internet)) to the memory 504 of the computer 500. The CPU 502 executes the cellular state presenting program, so that the computer 500 functions as a device for performing the process of the present invention.

Information about a chemical substance, a chemical receptor, a cell or the like is input via the input section 501 as well as profile data obtained. Known information may be input as appropriate.

The CPU 502 generates display data based on the information about profile data and cells through the input section 501, and stored the display data into the memory 504. Thereafter, the CPU 502 may store the information in the memory 504. Thereafter, the output section 503 outputs a cellular state selected by the CPU 502 as display data. The output data is output through the I/O device 506.

When the present invention is provided as a program embodiment as described above, each element may be any element in the same manner as when the present invention is provided as a method, including application of each detailed description and preferable embodiment for carrying out the same, and the selection of such preferable embodiments will be readily understood and carried out by those skilled in the art. Those skilled in the art can readily carry out such preferable embodiments of the program of the present invention in view of the description of the present application. The description format of such a program is well known in the art and for example, C+ language may be applied.

As such, the present invention is applicable to tailor-made diagnosis and therapy such as determination of health conditions, determination of condition of excitation state, drug resistance, selection of an appropriate anticancer agent, selection of an appropriate cell type to be transplanted, and the like. Preferably, the diagnosis method of the present invention, is provided as a therapy or prevention method comprising a step of treating a subject the selected therapy or prevention according to the diagnosis result. In another preferable embodiment, the diagnosis system of the present invention may be provided as a therapy or prevention system comprising means for providing a therapy or prevention selected according to the diagnosis results.

A configuration of a computer or system for implementing a method of the present invention for treatment or diagnosis is shown in FIG. 12. FIG. 12 shows an exemplary configuration of a computer 500 for executing a method of the present invention for diagnosis.

The computer 500 comprises an input section 501, a CPU 502, an output section 503, a memory 504, and a bus 505. The input section 501, the CPU 502, the output section 503, and the memory 504 are connected via a bus 505. The input section 501 and the output section 503 are connected to an I/O device 506.

An outline of a process for correlating processing, which is executed by the computer 500, will be described below.

A program for executing the correlation method and/or selection of treatment or prevention (hereinafter referred to as a “correlation program” and “selection program”, respectively) is stored in, for example, the memory 502. Alternatively, each component of the cellular state presenting program may be stored in any type of recording medium, such as a floppy disk, MO, CD-ROM, CD-R, DVD-ROM, or the like separately or together. Alternatively, the program may be stored in an application server. The correlation program and selection program stored in such a recording medium is loaded via the I/O device 506 (e.g., a disk drive, a network (e.g., the internet)) to the memory 504 of the computer 500. The CPU 502 executes the correlation program and selection program, so that the computer 500 functions as a device for performing the correlation program and selection program of the present invention.

Results of analysis of profile (for example, phases) and information about a cell state or the like are input via the input section 501 as well as profile data obtained. Additional information such as secondary information including conditions, disorders or diseases as correlated with the profiles, information on treatments and/or diagnosis may be input as appropriate.

The CPU 502 correlates information about the profile with the state of cells or the condition, disorders or diseases of the subject, and optionally with a method for prevention or treatment, based on the information through the input section 501, and stores the correlation data in the memory 504. Thereafter, the CPU 502 may store the information in the memory 504. Thereafter, the output section 503 outputs information on the cellular state, information about a condition, disorders or diseases of a subject and optionally methods for prevention or diagnosis and the like selected by the CPU 502 as diagnosis data. The output data is output through the I/O device 506.

All the references such as scientific articles, patents, patent application as cited herein are incorporated herein by reference as if they had been specifically described to the same extent for the entirety thereof.

The preferred embodiments of the present invention have heretofore been described to provide a better understanding of the present invention. Hereinafter, the present invention will be described by way of examples. Examples described below are provided only for illustrative purposes. Accordingly, the scope of the present invention is not limited except as by the appended claims.

### EXAMPLES

Hereinafter, the present invention will be described in greater detail by way of examples, though the present invention is not limited to the examples below. Reagents, supports, and the like were commercially available from Sigma (St. Louis, USA), Wako Pure Chemical Industries (Osaka, Japan), Matsunami Glass (Kishiwada, Japan) unless otherwise specified.

### Example 1

### Isolation of an Olfactory Receptor, an Example of a Chemical Receptor

In the present example, the isolation and functional analysis of an olfactory receptor as an exemplary example of chemical receptors was carried out. The procedures are described below. An exemplary chemical sensor was manufactured using such an isolated olfactory receptor as a typical chemical receptor.

(Olfactory Receptor Response Specificity to Odor Molecule)

It is reported that, in mice, the olfactory receptor is expressed is olfactory cells and detects and identifies odor molecules (Cell 96: 713-723 (1999) and the like). It is reported that there are four zones indicated by No. 1 to 4 in order from the dorsum to the ventral olfactory epithelium wherein olfactory cells are distributed and each olfactory receptor is limitedly expressed in any one of the four zones (Science 286 (5440): 706-711 (1999) and the like). As it is estimated by gene analysis estimates that there are about 1,000 types of olfactory receptors, the responsiveness to specific odor molecules of the olfactory cells sampled in statistically significant number from the four zones is equivalent. Therefore, in the present invention, the responsiveness of 2,740 olfactory cells, about 2.7 times as many as 1,000 specific types of cells, was investigated. It cannot be declared that the statistical significance is maintained for all zones, as the number of types of olfactory receptor included in each zone is not clear. However, at least in zone 1 and zone 2, there were three times or more as many of 250 types of cells which are considered to be present in one zone only when 1,000 kinds of cells are distributed equally to each zone were sampled and, even in zone 3 wherein the number of sampled cells is the least amongst the four zones, about 320 specific cell types were sampled, suggesting that almost all types of olfactory receptors were investigated. As a result, 4 olfactory cells sensitively and selectively responding to R(−)carvone of spearmint odor of a typical olfactory source, 18 olfactory cells sensitively and selectively responding to S(+)carvone of caraway odor of another typical olfactory source, 3 olfactory cells responding, with higher sensitivity than others and to the same extent, to both pulegone and (−)menthone commonly having mint odor of another typical olfactory source, and 3 olfactory cells responding, with the highest sensitivity and to the same extent, to both R(−)carvone and S(+)carvone commonly having sweet odor of another typical olfactory source were found. Respectively, 2, 2, 1 and 1 type(s) of genes identified by RT-PCR determined from a single cell among such olfactory cells were found and described in the present invention.

(Identification of Olfactory Receptor Gene)

The olfactory epithelium was quickly excised from a mouse euthanized by decapitation under anesthesia and divided into small pieces. These pieces were trypsinized to cleave intercellular junctions, and a sample of viable multiple mouse olfactory cells isolated from cells adhered to a cover slip by softly putting the treated pieces onto the cover slip taking care not to rupture them. Calcium-sensitive fluorescent dye fura-2 was intracellularly loaded on the sample, a solution of each odor molecule was administered, and the transient increase in intracellular calcium concentration triggered by the olfactory cells responding was detected by measuring and analysing the change in fura-2 fluorescence intensity within the cell using a microscope (Nikon), high-sensitive SIT video camera and image analysis device Argus-50 (Hamamatsu Photonics for both of the latter two), allowing the analysis individual cells response to specific odor molecules. Olfactory cell with a confirmed response were individually sampled with a micropipette containing 4 μl of cell lysis mix (1×MMLV buffer (GIBCO BRL), 0.5% NP-40, 290 U/ml RNA guard (Pharmacia), 300 U/ml Prime RNase inhibitor (Eppendorf), 10 μM each dNTP, 200 ng/ml pd(T)₂₅₋₃₀) under microscopy and moved into a tube for PCR. After the tube was incubated at 65° C. for 1 minute, 0.5 μl of RT mixed solution containing 50 U of MMLV reverse transcriptase (GIBCO BRL) and 0.5 U of AMV reverse transcriptase (GIBCO BRL) was added to each tube, followed by reverse-transcription at 37° C. for 30 minutes and treatment at 65° C. for 10 minutes prior to the termination of reaction. To form polyA tails, 5 μl of 2×TdT buffer (GIBCO BRL), 1.5 mM DATP, and 3 U/μl Terminal deoxynucleotidyl transferase (GIBCO BRL) were added, followed by incubation at 37° C. for 15 minutes and treatment at 65° C. for 10 minutes prior to the termination of reaction.

To amplify cDNA, the mixed solution of 1×PCR buffer II (Perkin-Elmer), 2.5 mM MgCl₂, 1 mM each dNTP, 0.1 mg/ml BSA, 0.05% TritonX-100, 0.1 U/μl AmpliTaq LD polymerase (Perkin-Elmer), and 0.05 μg/μl AL1 primer (ATTggATCCAggCCgCTCTggACAAAATATgAATTC(T)₂₄ (SEQ ID NO: 23)) was added to the resultant product to a final volume of be 100 μl, followed by treatment at 96° C. for 3 minutes, 25 cycles of 96° C. for 1 minute+42° C. for 2 minutes+72° C. (6 minutes+10 seconds prolongation/cycle), and treatment at 72° C. for 10 minutes. To the resultant, 5 U of AmpliTaq LD polymerase (Perkin-Elmer) was added, followed by 25 cycles of treatment.

One microliter of 1/10 dilution of the above treated solution was transferred into a new tube for PCR containing 49 μl of mixed solution of 1×PCR Gold buffer (Perkin-Elmer), 2.5 mM MgCl₂, 2 μM each degenerate primer, 0.2 mM each dNTP, and 0.05 U/μl AmpliTaq Gold polymerase (Perkin-Elmer) to conduct PCR specific to the olfactory receptor. After treatment at 96° C. for 3 minutes, PCR was conducted at 40 cycles of 96° C. for 1 minute+40° C. for 3 minutes+72° C. for 6 minutes, followed by treatment at 72° C. for 10 minutes. The sequences used as degenerate primers were, for TM3-TM6 (transmembrane 3-transmembrane 6) domain, P26 (GCITA(C/T)GA(C/T)CGITA(C/T)GTIGCIATITG (SEQ ID NO: 24)) and P27 (ACIACIGAIAG(G/A)TGIGAI(G/C)C(G/A)CAIGT (SEQ ID NO: 25)). A PCR product of the size corresponding to olfactory receptor was isolated by agarose gel electrophoresis, followed by subcloning to pCR 2.1 or pCR II-TOPO vector (Invitrogen) and sequence determination with DNA sequencer of plate gel (Shimazu).

SuperScript II (GIBCO BRL) was also used as a reverse transcriptase. When using this reverse transcriptase, the experiment was performed with changes in the following points. Instead of RT mixed solution of 4.5 μl of cell lysis mix (10 mM Tris-HCl [pH8.3], 50 mM KCl, 0.05% NP-40, 600 U/ml RNAguard (Pharmacia), 600 U/ml Prime RNase inhibitor (Eppendorf), 50 μM each dNTP, 200 ng/ml Anchor T primer (TATAgAATTCgCggCCgCTCgCgA(T)₂₄ (SEQ ID NO: 26)), 50 U of MMLV reverse transcriptase (GIBCO BRL) and 0.5 U of AMV reverse transcriptase (GIBCO BRL), 0.5 μl of RT mixed solution (171 U/μl SuperScript II(GIBCO BRL), 2 U/μl Prime RNase inhibitor and 2 U/μl RNAguard) was used, followed by reaction at 37° C. for 120 minutes (0.5 μl of RT mixed solution was added every 40 minutes). To form polyA tails, 5 μl of 3 mM DATP, 10 mM Tris-HCl[pH8.3], 1.5 mM MgCl₂, 50 mM KCl, 2.5 U/μl Terminal deoxynucleotidyl transferase (Roche), and 1 U/μl RNaseH(Roche) were added to the resultant product, followed by reaction at 37° C. for 20 minutes and treatment at 65° C. for 10 minutes prior to the termination of reaction. To amplify cDNA, 2.5 μl of the solution treated to form poly dA tail was added to 25 μl of mixed solution (1×LA PCR Buffer II (TaKaRa), 250 mM each dNTP, 2.5 mM MgCl₂, 20 ng/μl Anchor T primer, 0.05 U/μl TaKaRa LA Taq (TaKaRa)), followed by 1 cycle of 95° C. for 2 minutes+37° C. for 5 minutes+72° C. for 20 minutes, 35 cycles of 95° C. for 30 seconds+67° C. for 1 minute+72° C. (6minutes+6 seconds prolongation/cycle) and treatment at 72° C. for 10 minutes.

As the primer for transmembrane domain 2 (TM2), CT(ATgC)CA(TC)(AC)(AC)(ATgC)CC(ATgC)ATgTA(TC)(TC)T(ATgC)TT(TC) (TC)T (SEQ ID NO: 27) was used and, as the primers for transmembrane domain 7 (TM7), P41: AA(gA) (Tg)CITTI(AgT) (AC) IACITg(CT)g(gC) ITCICA (SEQ ID NO: 28 ), P42: TC(TC)(TC)TIgTI(TC)TI(Ag)(TC)IC(Tg)gATAIATIATIgg(gA)TT (SEQ ID NO: 29), W68: TCI(TC)T(gA)TTIC(Tg)IAgIg(TA)(gA)TAIAT(gA)AAIgg(gA)TT (SEQ ID NO: 30), W69: TC(TC)TT(gA)TTIC(Tg)IAgIg(TA)(gA)TAIA(TC)IA(gC)Igg(gA)TT (SEQ ID NO: 31), W70: TCIT(gC)(gA)TTIC(Tg)IA(gA)I(gC)A(gA)TAIATIATIgg(gA)TT (SEQ ID NO: 32), and P8: (gA)TTIC(Tg)IA(Ag)I(gC)(TA)(gA)TAIAT(Ag)AAIgg(gA)TT (SEQ ID NO: 33) were used.

Regarding the olfactory receptor in the present invention, using genomic DNA in mouse as a template, by using primers regions containing both ends with adequate length of the specific sequences in the domain including required portions of target genes among the above SEQ ID NOs: 1, 3, 5, 7, 9, 11, 13, 15, 17, 19 and 21 as the subjects, followed by PCR, the corresponding regions of gene of the olfactory receptor of interest can be obtained.

The olfactory receptors with high sensitivity to specific odor qualities are shown as follows.

| Odor quality | | Response at 1 μM | Response at 10 μM |
|---|---|---|---|
| car-n272 | Spearmint | rCa | rCa, sCa |
| car-c5 | Spearmint | rCa | rCa, sCa |
| car-b161 | Caraway | None | sCa |
| car-b153 | Caraway | None | sCa |
| car-b85 | Mint | pu, mn > sCa, rCa pu, | mn, sCa, rCa, ip |
| car-n266 | Sweet odor | rCa, sCa | rCa, sCa |

rCa: R (−)—carvone, sCa: S (+)—carvone, pu: pulegone, mn: (−)—menthone, ip: isopulegol

The rising of output OUT (olfactory receptor type) reflecting the olfactory receptor responses are compared and the corresponding odor intensity S can be estimated if they meet the conditions of magnitude correlation of responses shown below.

Spearmint Odor:
OUT(car-n266)=OUT(car-n272)=OUT(car-ZAc5)>OUT(car-b85)>OUT(car-b161)=OUT(car-b153), [Number 1]
Response intensity S=OUT(car-n266)+OUT(car-n272)+OUT(car-c5)
Caraway Odor:
OUT(car-n266)>OUT(car-b85)>OUT(car-b161)=OUT(car-b153)>OUT(car-n272)=OUT(car-c5), [Number 2]
Response intensity S=OUT(car-n266)+OUT(car-b161)+OUT(car-b153)
Mint Odor:
OUT(car-b85)>OUT(car-n266),OUT(car-b161),OUT(car-b153),OUT(car-n272),OUT(car-c5), [Number 3]
Response intensity S=OUT(car-b85)
Sweet Odor:
OUT(car-n266)=OUT(car-n272)>OUT(car-b85),OUT(car-b161),OUT(car-b153),OUT(car-c5), [Number 4]
(including the magnitude conditions of the response to spearmint odor)
Response intensity S=OUT(car-n266)

As an example of response measurement, by making each group of multiple olfactory cells form six different six clusters expressing a type of the above olfactory receptors, incorporating calcium-sensitive fluorescent dye into the cells, and measuring the change in fluorescence intensity around 510 nm induced by excitation with 380 nm of light when the sample odorant gas was administered equally to these cell groups, by-cluster decrease rates of fluorescence intensity are compared. If the results meet the above conditions, the corresponding response intensity can be obtained as an optional unit.

### Example 2

### Olfactory System as a Sensation-Evaluation System

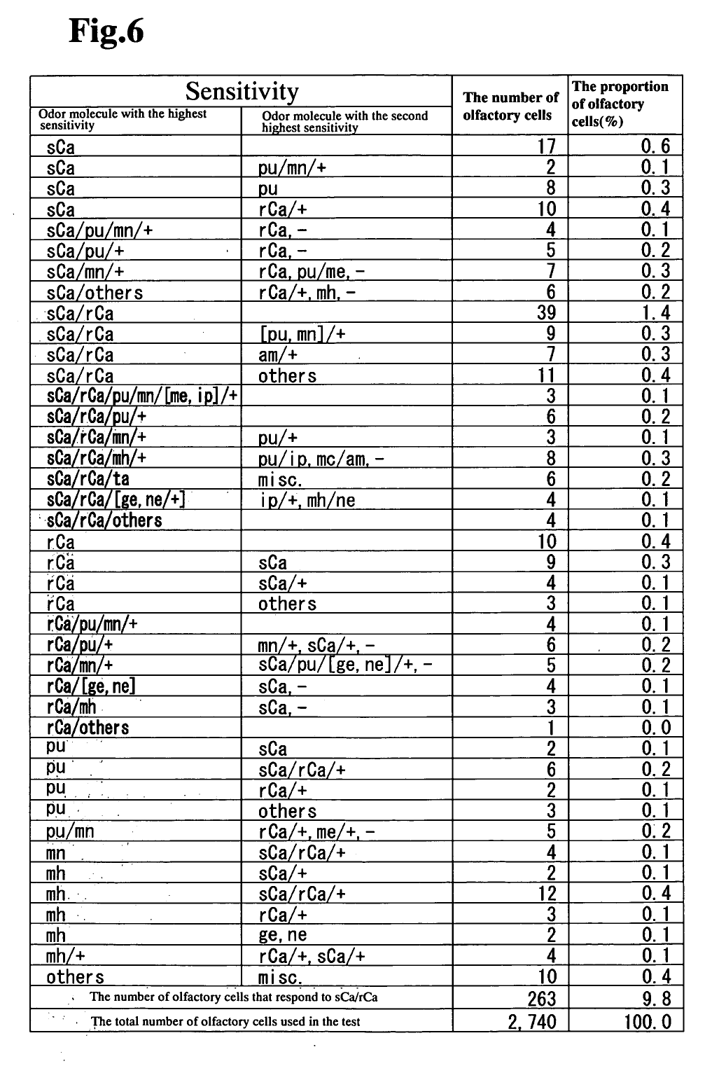

In the present example, it is described in detail that the sensation-evaluation system described above as an embodiment of the present invention is consistent with the olfactory system for identifying biological odor referring to FIGS. 6-8.

The odor molecules corresponding to the abbreviations used in FIGS. 6-8 are as follows:
sCa(S(+)-carvone):S(+)-5-isopropenyl-2-methyl-2-cyclohexenon
rCa(R(−)-carvone):R(−)-5-isopropenyl-2-methyl-2-cyclohexenon
mn ((−)menthone):(2S,5R)-2-isopropyl-5-methylcyclohexanone
pu(R(+)-pulegone):(R)-p-menth-4(8)-en-3-one
ip(isopulegol):2-isopropenyl-5-methylcyclohexanol
me(menthol):2-isopropyl-5-methylcyclohexanol
lim(R(+)-limonene):(R)-1-methyl-4-(1-methylethyl)-cyclohexene
am(isoamyl acetate):3-methylbutylacetate
va(vanillin):4-hydroxy-3-methoxybenzaldehyde
ova(o-vanillin):3-methyoxysalicylaldehyde
ge(geraniol):3,7-dimethyl-(E)-2,6-octadiene-1-ol
ne(nerol):3,7-dimethyl-(Z)-2,6-octadiene-1-ol
mc6:hexanoic acid
mc7:heptanoic acid
mc8:octanoic acid
mc9:nonanoic acid
mh6:1-hexanol
mh7:1-heptanol
mh8:1-octanol
mh9:1-nonanol
in(indole):1-H-indole
ta:triethylamine
iv(isovaleric acid):3-methylbuthylic acid

FIG. 6, for 2,740 biological olfactory cells, shows the measurement results of responding olfactory cells and their response intensities, which are classified according to two kinds of odor substances, S(+)carvone and R(−)carvone, individually referring to multiple odor molecules containing S(+)carvone and R(−)carvone.

In FIG. 6, the olfactory receptors responding to two kinds of odor substances of S(+)carvone (mainly causing caraway odor) and R(−)carvone (mainly causing spearmint odor) are comprehensively searched, the members near to almost all the receptors are detected, and their identification properties for the specific odor molecules are clarified. Each line in FIG. 6 shows the type of olfactory cell, i.e. olfactory receptor, using the abbreviations of reacting odor molecules. The line of “odor molecule with the highest sensitivity” shows the odor molecule or the odor molecule group to which the olfactory cell having each receptor showed the highest sensitivity and the line of “odor molecule with the second highest sensitivity” shows the odor molecule or the odor molecule group to which the olfactory cell having each receptor showed the second highest sensitivity. The number of olfactory cells responding to carvone (hereinafter, “carvone” indicates both S(+)carvone and R(−)carvone) was 263, corresponding to 9.6% of the total.

As an experimental result, there were about 70-100 kinds of olfactory receptors (corresponding to about 9.6% of about 1,000 kinds of all the olfactory cells) functioning to identify carvone, wherein about ⅕ of them responded to S(+)carvone with relatively high sensitivity, about ⅕ of them responded to R(−)carvone with relatively high sensitivity, about ⅖ of them responded to both carvones equally with relatively high sensitivity, and about ⅕ of them responds to other odor substances with relatively high sensitivity.

This result is consistent with the sensation-evaluation system described as an embodiment of the present invention. Especially, the appropriate setting of indicated coefficient aj(i) at selected portion SAi in pretreated portion P1 in FIG. 2 permits the invention's consistency with this experimental result. Conversely, the determination of coefficient aj(i) to be consistent with the experimental result allows the sensitive identification the odor substance at evaluated portion EV.

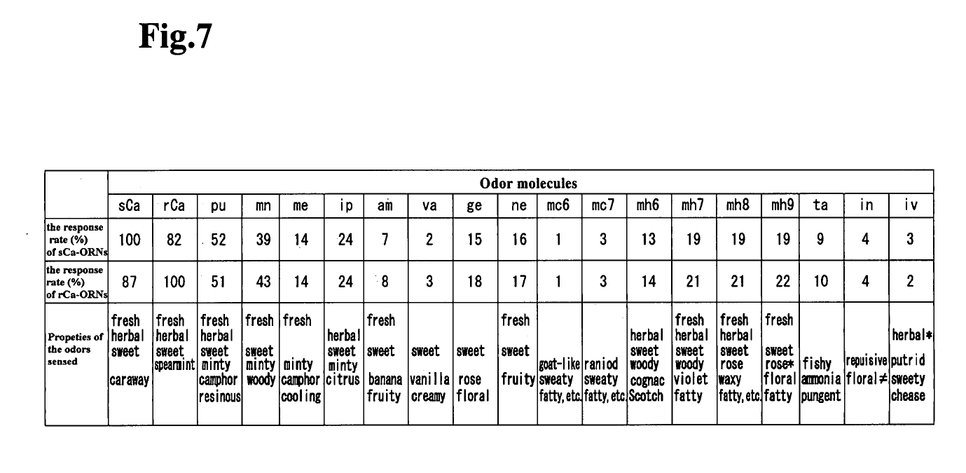

FIG. 7 shows the results of the response rates of the olfactory receptors shown in FIG. 6 to odor molecules other than carvone, which are calculated separately for the olfactory receptor group responding to S(+)carvone and the olfactory receptor group responding to R(−)carvone. In FIG. 7, the response rate (%) is the ratio of the number of olfactory cells responding to each odor molecule to the number of all the target olfactory cells, showing the ratio for the olfactory receptor group responding to S(+)carvone in the first line and the olfactory receptor group responding to R(−)carvone in the second line. Therefore, it can be considered that the response rates (%) in FIG. 7 indicate that the odor qualities which S(+)carvone and R(−)carvone have (fresh, herbal, sweet, caraway and spearmint) are shared in common to an odor molecule with higher intensity. The rates are the reference values of the qualitative/quantitative evaluation of stimulus (odor substance) and the sense amount (odor) actually perceived by living entity is close to the amount obtained by treating the output obtained by multiplying these values by the coefficients dependent on the relative sensor sensitivity to S(+)carvone and R(−)carvone.

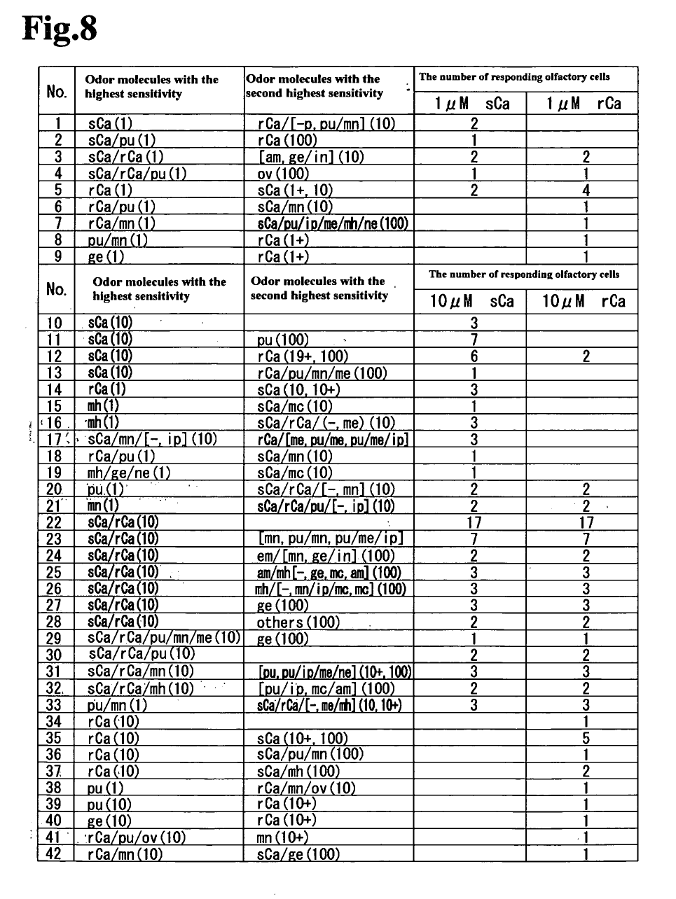

FIG. 8 shows the numbers of responsing cells determined by the rate of presence of the receptors with high sensitivity to S(+)carvone and R(−)carvone. The comparison among the receptors with high sensitivity allows to explain the differences in odor quality and sensitivity between S(+)carvone and R(−)carvone. Only two S(+)carvone-selective cells responded to S(+)carvone at 1 μM, which is considered to be insufficient for detection as a significant signal within the brain. At 10 μM, 10-hold higher concentration than the above, seventeen S(+)carvone-selective cells responded, allowing formation of the signal characteristic to S(+)carvone in the brain. On the other hand, four R(−)carvone-selective cells responded to R(−)carvone at 1 μM and it is expected that this would result in signals significant to some degree would be transmitted to the brain. It was also observed that three cells responded equally to both carvones at 1 μM. It can be considered that these cells are core for forming “sweet” or “herbal” odor qualities of the odor common to both. It is considered that the signals from these cells (olfactory receptors) being the core, by inhibiting the signals from other cells (olfactory receptors), emphasize the odor quality characteristic of an individual odor molecule and decrease the shared odor quality. Furthermore, these findings may explain human show higher sensitivity to R(−)carvone than to S(+)carvone and perceive that R(−)carvone has a mint odor but S(+)carvone does not have a definite mint odor.

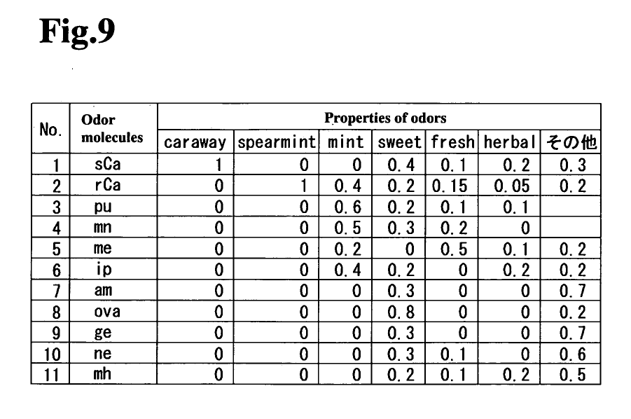

Specifically, regarding the data in FIG. 8, the case wherein the output of olfactory receptors is calculated as the number of responses is described. Here, the stimulation during conditions where the response amplitude is ½ as large as the usual one (e.g. olfactory receptor in line 8) is treated with further decrease in the response amplitude by two-fold. FIG. 9 shows an example of the relative intensity of odor quality, which humans perceive for each odor molecule. Considering the relevance of odor qualities induced by odor molecules, shown in FIG. 7, the values of sCa (the first line) and rCa (the second line), regarding the relative intensity of the odor quality with each highest intensity as a reference (i.e. 1), indicates the relative intensities of the other odor qualities. Regarding the odor molecules in the third line and below, the relative intensity of each odor qualities is determined for the sum of relative intensities of odor qualities in each line to be 1. The following calculations use the values in FIG. 9. The following values of each calculation result include the errors caused by rounding-up in calculation, which have no influence on the explanation of the method of calculating the output of olfactory receptors as the number of responses. According to the regulations described above, 1 micromole of rCa is calculated. As shown in FIG. 8, the olfactory receptors responding to rCa are described in the extreme right line, i.e. two in the third line to one in the ninth line respond. Therefore, by multiplying the number of olfactory receptors shown on the extreme right in each line by the relative intensity in corresponding line in FIG. 9, the contribution to each odor quality is calculated. For the olfactory receptors responding only to single odor molecule, the relative intensities in FIG. 9 are directly used, and, for the olfactory receptors responding to multiple kinds of odor molecules, the minimum values are used. For example, regarding the sixth line in FIG. 8, since the receptors there respond to rCa and pu, for each odor quality, the lower value of the value in the second line and the value in the third line in FIG. 9 is used. In such a case, caraway=0, spearmint=0, mint=0.4, sweet=0.2, fresh=0.1, and herbal=0.1. Here, as the variables indicating the values on each odor quality, the names of odor qualities are used. The figures are properly rounded up in calculation.

In this way, the calculation is performed for each line from the third line to the ninth line in FIG. 8 and the result is added by odor quality, resulting in caraway=0, spearmint=4, mint=2.65, sweet=2.05, fresh=1.2, and herbal=0.4. Furthermore, from these values, the ratio of specific values to the sum total excluding the specific value is calculated. For example, in spearmint, 4/(0+2.65+2.05+1.2+0.4)=0.635, and, in mint, 2.65/(0+4+2.05+1.2+0.4)=0.35. As a result, it is obtained that caraway=0, spearmint=0.635, mint=0.35, sweet=0.25, fresh=0.13, and herbal=0.04. If normalized by the highest value among these, i.e. the value in spearmint, 0.635, it is obtained that caraway=0, spearmint=1, mint=0.55, sweet=0.39, fresh=0.21, and herbal=0.06. The following calculation is performed regarding 0 as 0.1 because it is considered that infinitely significant inhibition cannot be applied and thus about 1/10 is appropriate.

The coefficient obtained as described above is multiplied by the calculated value for 10 micromoles as a coefficient. Thus, the values (numbers) on the extreme right in lines 20-42 in FIG. 8 are multiplied by caraway=0.1, spearmint=1, mint=0.55, sweet=0.39, fresh=0.21, and herbal=0.06. By odor quality, the calculated result for 10 micromoles in each line is added to the calculated value for 1 micromole, resulting that caraway=0.1, spearmint=13, mint=6.9, sweet=7.4, fresh=2.7, and herbal=0.6. These values are largely consistent with the sensual evaluation results of the relative intensity of odor quality actually perceived by humans.

Regarding sCa, since the output of 1 micromole of olfactory receptor does not result in the output of sense element large enough to be recognized, the calculation is performed using the experimental values for 1 micromole and the experimental values for 10 micromoles. The results are caraway=19, spearmint=4, mint=4.4, sweet=21, fresh=8.7, and herbal=7.3. Inhibitory effects are expected to appear corresponding to the outputs and the values obtained by multiplying the effects by these relative intensities (0.86, 0.14, 0.22, 1, 0.32 and 0.26) as coefficients are caraway=16.3, spearmint=0.54, mint=0.95, sweet=21.1, fresh=2.81, and herbal=1.9. The sum total is 43.6, while the corresponding value of rCa is 30.7, and these values are reversed in magnitude, in comparison with the sense intensity actually perceived by humans. Therefore, for sCa, the values obtained by multiplying by 0.7 as an adjustment coefficient are caraway=11, spearmint=0.4, mint=0.7, sweet=15, fresh=2, and herbal=1.3. It is considered that the composition of the odor quality can be explained to some degree by these values.

As described above, it can be considered that the fact that the calculation results obtained from the experimental data for rCa and sCa are largely consistent with the sensory evaluation results of the relative intensity of the odor quality actually perceived by humans at the prior stage of parameter optimization indicates that the treatment using the sensation-evaluation system defined by the present invention is consistent with actual odor processing in humans.

In the present invention, the output signals from the sensors responding to sensory stimulus of the senses perceived by humans, such as olfaction, gustation and taction, allow the qualitative/quantitative evaluation of the amounts of these senses and the blending of stimulants capable of reproducing the qualities of optional sense amounts.

The present invention allows the development of a sensor system for measuring the odor quality, quality components and those intensities similar to the odor quality and intensity perceived by humans, animals, and the like, a sensor system for estimating the causal components or the compositions of stimulant elements of an arising odor, or the system for automatically blending odor solutions/gases to have a desired odor. Also, the present invention can be used, for other senses related to chemical substances, such as gustation and taction, using similar devices and systems.

Furthermore, it is expected that the development of sensory function alternative devices, control/manufacturing apparatuses using sensation-evaluation technology, robots having senses and judgment, and the like, will be accelerated. The emergence of human-friendly technology beyond our imagination is expected, which will be directed to things which have previously been thought difficult to realize, specifically, odor sensors, recording/reproducing device of odor information, food manufacturing process control system, and medical diagnostic apparatus using odor.

### Example 3

### Manufacture of Chips for Sensors

Formulations below were prepared in the present Example.

(Cellular Adhesion Factors)

As candidates for a cellular adhesion molecule, various extracellular matrix proteins and variants or fragments thereof were prepared. The materials prepared in the present Example are as follows. Cellular adhesion factors used were commercially available.
1) ProNectin F (Sanyo Chemical Industries, Kyoto, Japan);
2) ProNectin L (Sanyo Chemical Industries);
3) ProNectin Plus (Sanyo Chemical Industries);
4) gelatin.

Plasmids were prepared as DNA for transfection. Plasmids, pEGFP-N1 and pDsRed2-N1 (both from BD Biosciences, Clontech, CA, USA) were used. In these plasmids, gene expression was under the control of cytomegalovirus (CMV) promoters. The plasmid DNA was amplified in E. coli (XL1 blue, Stratgene, TX, USA) and the amplified plasmid DNA was used as a complex partner. The DNA was dissolved in distilled water free from DNase and RNase.

The following transfection reagents were used: Effectene Transfection Reagent (cat. no. 301425, Qiagen, CA), TransFast™ Transfection Reagent (E2431, Promega, WI), Tfx™-20 Reagent (E2391, Promega, WI), SuperFectTransfection Reagent (301305, Qiagen, CA), PolyFect Transfection Reagent (301105, Qiagen, CA), LipofectAMINE 2000 Reagent (11668-019, Invitrogen corporation, CA), JetPEI (x4) conc. (101-30, Polyplus-transfection, France), and ExGen 500 (RO511, Fermentas Inc., MD). These transfection reagents were added to the above-described DNA and cellular adhesion molecules in advance or complexes thereof with the DNA were produced in advance.

The thus-obtained solution was used in assays using transfection arrays described below.

### Example 4

### Transfection Array—Demonstration using Mesenchymal Stem Cells)

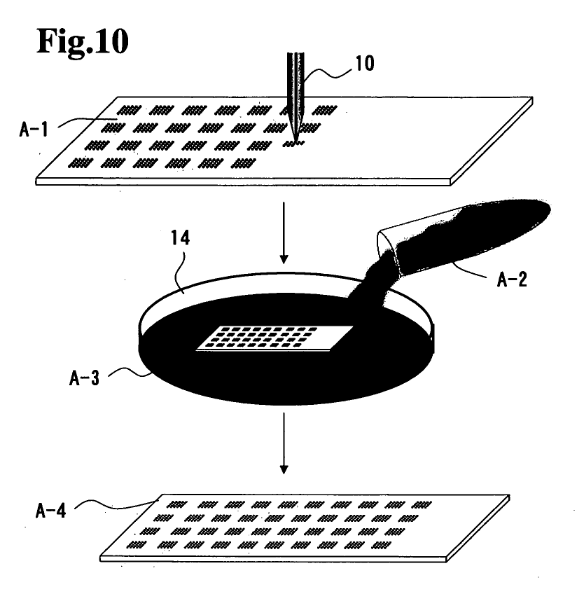

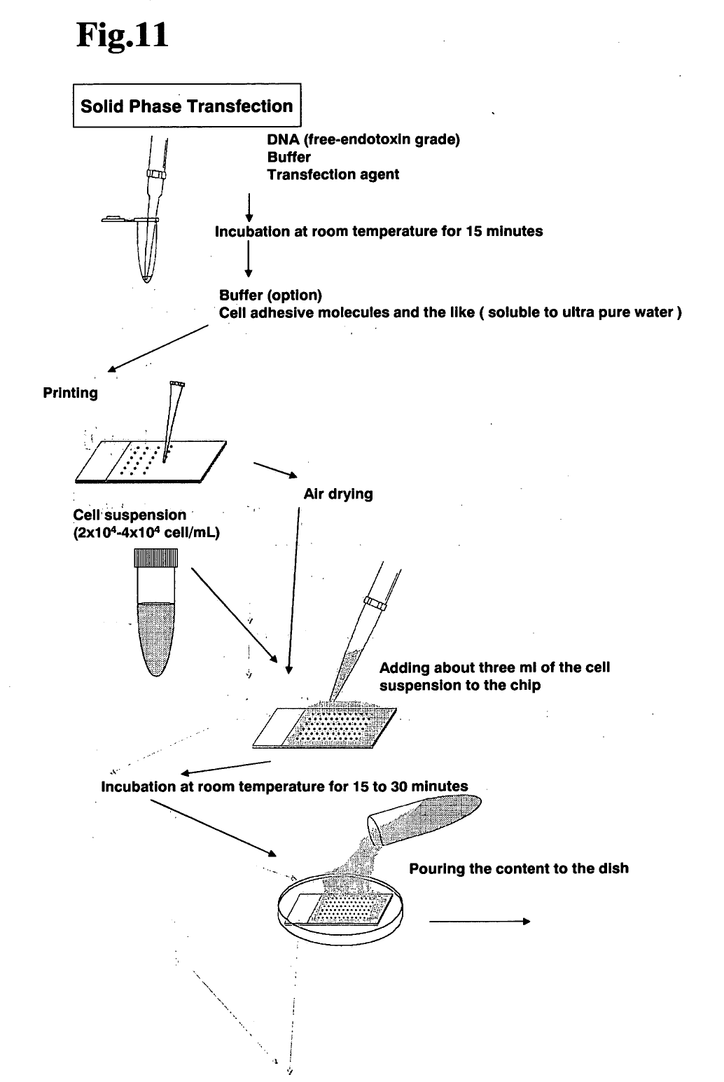

In the present Example, an effect of the transfection efficiency of solid phase was observed. The protocol used will be described below (see FIGS. 10 and 11).

(Protocol)

The final concentration of DNA was adjusted to 1 μg/μL. An actin acting substance was preserved as a stock having a concentration of 10 μg/μL in ddH₂O. All dilutions were made using PBS, ddH₂O, or Dulbecco's MEM. A series of dilutions, for example, 0.2 μg/μL, 0.27 μg/μL, 0.4 μg/μL, 0.53 μg/μL, 0.6 μg/μL, 0.8 μg/μL, 1.0 μg/μL, 1.07 μg/μL, 1.33 μg/μL, and the like, were formulated.

Transfection reagents were used in accordance with instructions provided by each manufacturer.

E. coli transformed with plasmid DNA was removed from a glycerol stock and amplified in 100 mL L-amp overnight. Qiaprep Miniprep or Qiagen Plasmid Purification Maxi kits were used to purify DNA in accordance with a standard protocol provided by the manufacturer.

In the present Example, the following 5 cells were used to confirm an effect: human mesenchymal stem cell (hMSCs, PT-2501, Cambrex BioScience Walkersville, Inc., MD); human embryonic renal cell (HEK293, RCB1637, RIKEN Cell Bank, JPN); NIH3T3-3 cell (RCB0150, RIKEN Cell Bank, JPN); HeLa cell (RCB0007, RIKEN Cell Bank, JPN); and HepG2 (RCB1648, RIKEN Cell Bank, JPN). These cells were cultured in DMEM/10% IFS containing L-glut and pen/strep.

(Dilution and DNA Spots)

Transfection reagents and DNA were mixed to form a DNA-transfection reagent complex. The complex formation requires a certain period of time. Therefore, the mixture was spotted onto a solid phase support (e.g., a poly-L-lysine slide) using an arrayer. In the present Example, as a solid phase support, an APS slide, a MAS slide, and an uncoated slide were used as well as a poly-L-lysine slide. These slides are available from Matsunami Glass (Kishiwada, Japan) or the like.

For complex formation and spot fixation, the slides were dried overnight in a vacuum dryer. Drying was performed in the range of 2 hours to 1 week.

Although the actin acting substance might be used during the complex formation, it was also used immediately before spotting in the present Example.

(Formulation of Mixed Solution and Application to Solid Phase Supports)

300 μL of DNA concentrated buffer (EC buffer)+16 μL of an enhancer were mixed in an Eppendorf tube. The mixture was mixed with a Vortex, followed by incubation for 5 minutes. 50 μL of a transfection reagent (Effectene, etc.) was added to the mixture, followed by mixing by pipetting. To apply a transfection reagent, an annular wax barrier was formed around the spots on the slide. 366 μL of the mixture was added to the spot region surrounded by the wax, followed by incubation at room temperature for 10 to 20 minutes. Thereby, the fixation to the support was manually achieved.

(Distribution of Cells)

Next, a protocol for adding cells will be described. Cells were distributed for transfection. The distribution was typically performed by reduced-pressure suction in a hood. A slide was placed on a dish, and a solution containing cells was added to the dish for transfection. The cells were distributed as follows.

The growing cells were distributed to a concentration of 10⁷ cells/25 mL. The cells were plated on the slide in a 100×100×15 mm squared Petri dish or a 100 mm (radius)×15 mm circular dish. Transfection was conducted for about 40 hours. This period of time corresponded to about 2 cell cycles. The slide was treated for immunofluorescence.

(Evaluation of Gene Introduction)

Gene introduction was evaluated by detection using, for example, immunofluorescence, fluorescence microscope examination, laser scanning, radioactive labels, and sensitive films, or emulsion.

When an expressed protein to be visualized is a fluorescent protein, such a protein can be observed with a fluorescence microscope and a photograph thereof can be taken. For large-sized expression arrays, slides may be scanned using a laser scanner for storage of data. If an expressed protein can be detected using fluorescence antibodies, an immunofluorescence protocol can be successively performed. If detection is based on radioactivity, the slide may be adhered as described above, and autoradiography using film or emulsion can be performed to detect radioactivity.

(Laser Scanning and Quantification of Fluorescence Intensity)

To quantify transfection efficiency, the present inventors used a DNA microarray scanner (GeneTAC UC4×4, Genomic Solutions Inc., MI). Total fluorescence intensity (arbitrary unit) was measured, and thereafter, fluorescence intensity per unit surface area was calculated.

(Cross-Sectional Observation by Confocal Scanning Microscope)

Cells were seeded on tissue culture dishes at a final concentration of 1×10⁵ cells/well and cultured in appropriate medium (Human Mesenchymal Cell Basal Medium (MSCGM BulletKit PT-3001, Cambrex BioScience Walkersville, Inc., MD). After fixation of the cell layer with 4% paraformaldehyde solution, SYTO and Texas Red-X phalloidin (Molecular Probes Inc., OR, USA) was added to the cell layer for observation of nuclei and F-actin. The samples emitting light due to gene products and the stained samples were observed with a Confocal laser microscope (LSM510: Carl Zeus Co., Ltd., pin hole size=Ch1=123 μm, Ch2=108 μm, image interval=0.4) to obtain cross sectional views.

### Example 5

### Odor Sensor

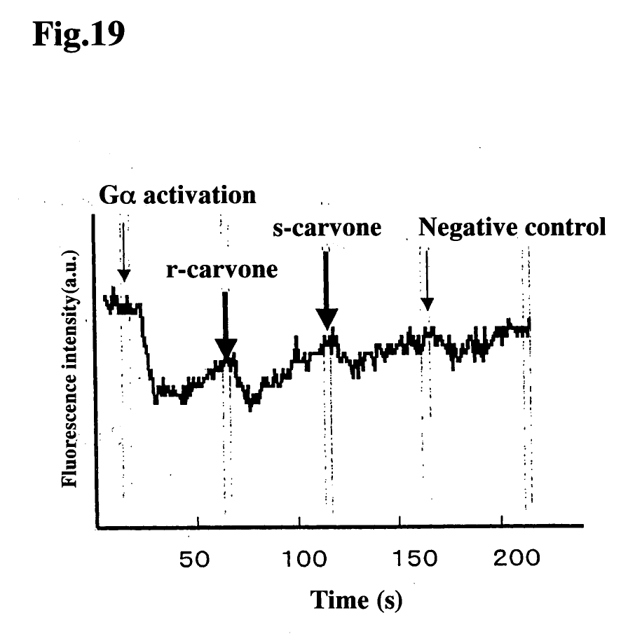

Next, the Example, to which a sensor of the present invention is applied to, is described wherein an olfactory receptor is set as a typical example of a chemical substance receptor. When a preliminary example was implemented, it was proved that transfection arrays can also be used in an olfactory receptor (FIG. 19).

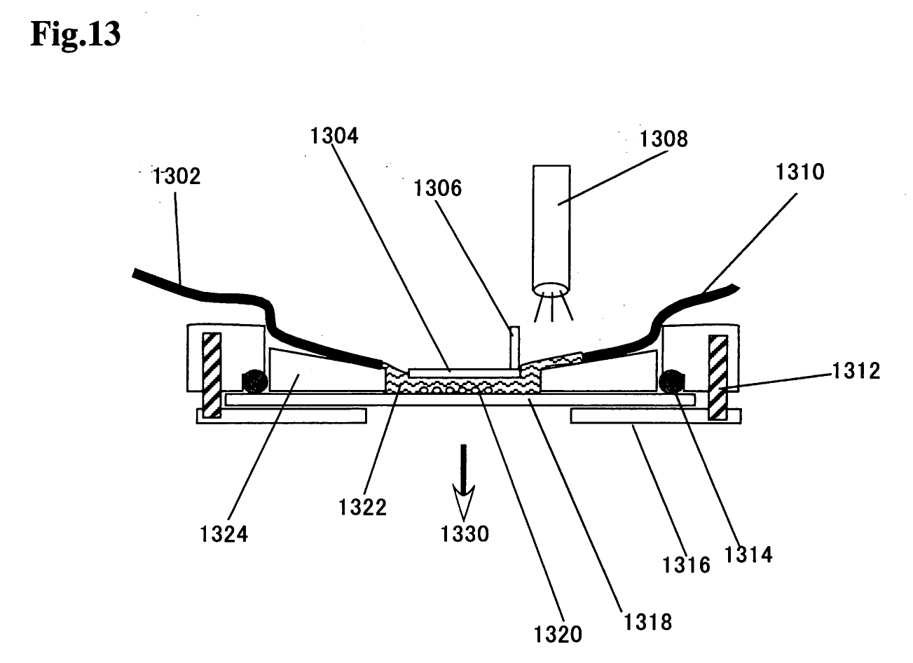

The olfactory receptor expression vector group was spotted per every kind of receptor, on a cover glass, which was made like an array, was secured with screws and the like in a chamber for signal measurement, and cells having almost homogeneous nature, were cultured thereon (FIG. 13). Regarding a chamber for signal measurement, sample gas was introduced in a known structure (Proc. Natl. Acad. Sci. USA, 96(1999): 4040-4045 and the like). Other devised chambers are also intended. As one example of chambers used in the present Examples, a central cross section of a chamber for signal measurement is shown in FIG. 13. During response measurement, culture medium was flowed at a constant speed. Culture was supplied to the chamber for measurement from the opening of a culture medium supplying tube, and a sample gas supplying tube was secured at the position preferably near the liquid level, which is the upper portion of an interval whose boundary is decided by reaching a wall which prevents approach of culture over a cover glass for a ceiling of the measument member, so that sample gas can be supplied to culture medium flowing acorss the interval. This sample gas supplying tube was preferably made of materials to which lipophilic odor substances such as Teflon and peak, and dust are not readily adsorbed. The higher effect was obtained in the situation wherein, at the time other than introducing sample gas, sample gas remaining in a tube was removed, and to preferably keep the interior clean, the tube (preferably with a broad opening) could be washed with odorless air by setting a three-way valve in the mid course, or by setting a check valve at a joint of an odorless air supplying tube. However, it was not necessary. The example could also be implemented in the situation wherein, at the time of other than introducing sample gas from outside for an appropriate time such as 0.5-4 seconds, odorless air was introduced from mid course of a sample gas supplying tube near a opening for collecting gas from outside, the interior of the tube was washed therewith, and at the same time, odorless gas was supplied to the culture medium as sample gas to promote the removal of remaining gas in a measurement chamber. A supporting base for the upper-glass cover slip is made of water repellent opaque plastic such as Teflon. A width of flow channel, where culture medium flows, is about 2-fold of a width of an array, and the array is disposed in the center of the flow channel. Regarding a culture medium supplying tube and an overflow culture medium sucking tube, a part of several millimeters from the opening at the side of the measurement chamber is made using materials, which has high hydrophilicity and is difficult to deform, such as stainless steel. The upper portion of the supporting base the upper glass cover-slip where culture medium flows, from the openings of both tubes to an array, was coated, or covered with a pieces of lens paper and the like in order to provide sufficient hydrophilicity. Negative pressure for suction was adjusted at the grade such that measurements were not affected by vibration from sound generated by aspiration culture.

Generally, response measurement could be implemented 2 days after the gene introduced by the vector expressed. Since an upper glass cover-slip was required only at the time of measurement, it was not required to install it during culture until the gene was expressed. Therefore, the Example could be implemented, adding an upper glass cover slip which is integrated with a wall which prevents leakage of culture medium, and a supporting base for the upper glass cover slip, to a chamber for measurement, when setting a chamber for measurement of change in fluorescence measured by an apparatus after the gene expressed. The Example could also be implemented in the situation wherein culture medium was exchanged without using a culture medium supply tube and an overflow culture sucking tube during culture until the gene was expressed. An amount of about 10 ml of culture medium was supplied and exchanged at the frequency of about 1 time per several hours-1 day, during the time tissue culture only was performed.

Size of odor response could be optically measured using a two-dimentional image sensor such as a sensitive video camera, with a calcium ion sensitive fluorescent dye fura-2 and the like absorbed into the cell. Measurement interval preferably has time resolution which can evaluate time constants of build-up and recovery of response of about ⅓-1 second. However, if average response time curve or its theoretical formula had been obtained, actual change was estimated from measurement results at 5 points with 5-second-interval of 5, 10, 15, 20, and 25 seconds after stimulation. The obtained estimates of time constant of response starting time, response build-up time, and response recovery time was set as an index, and evaluation could be made as to whether a signal was induced by odor, or generated by spontaneous activity of a cell or other abnormalities.

### Example 6

### Measurement of Calcium Concentration in Olfactory Receptor Neuron

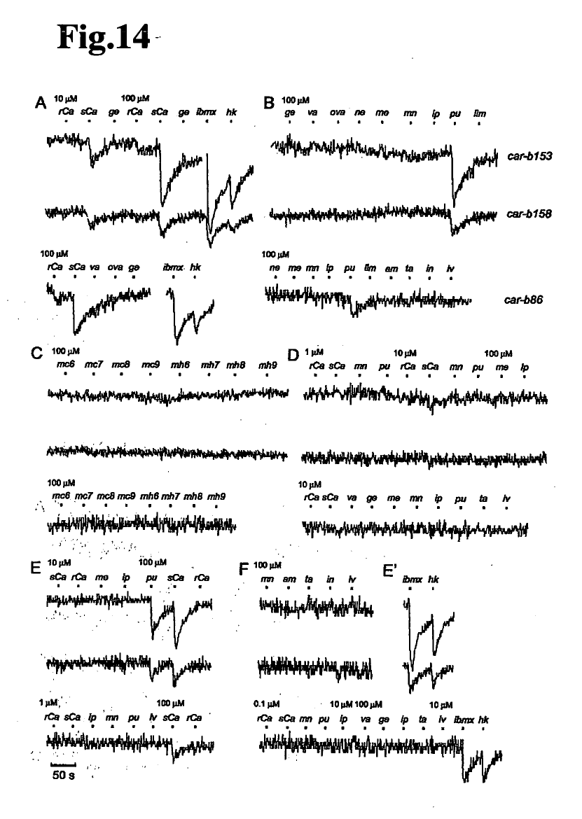

In this Example, response of an expressed olfactory receptor in olfactory receptor neuron was studied by measuring the change of fluorescence intensity of calcium sensitive fluorescent dye (FIG. 14). Decrease of fluorescence intensity (downward change) corresponds to response of an olfactory receptor. Odor molecules, which are indicated by abbreviation marks in the Figure, were added to culture at the concentration indicated above them as stimulation source, and administered to a cell during the time indicated by a bar (4 or 2 seconds). 3 olfactory receptors are identical and express in different cells. car-b153 and car-b158 were signals obtained by simultaneous measurement using cells which were simultaneously adjusted. car-b86 was the response measured in a different cell which was adjusted at different times. As undersood from this example, responses measured simultaneously in a simultaneously adjusted cell have high intercommunity in response time characteristics, response threshold concentration corresponding to different stimulation per cell, and relative value of response amplitude. However, cells adjusted at a different times show some differences. These results show that the highest measurement reliability can be obtained by measuring odor response using a sensor arrayed to a size that allows a homogeneous administration of sample gas, providing the same adjustment conditions.

Gene sequences of these 3 kinds of olfactory receptors used in the Example were the same as far as the study. Therefore, it can be considered that affinity for odor molecules is shared at the same level between 3 types of olfactory receptors expressed in these 3 olfactory receptor neurons. In fact, it is understood that the response signal change of car-b153 expressing cell and car-b158 expressing cell, which were measured simultaneously, shows good conformity in such as increase, decrease, relative amplitude compared to control response and the like, but it is difficult to judge that car-b86 expressing cell, which was not measured simultaneously, has the same conformity of responsiveness. It can be considered that these results depend upon the whether or not there is homogeneity in cell sample and administered stimulation.

In FIG. 14, odor molecules correspond to abbreviation marks as follows: S(+)-carvone (sCa); R(−)-carvone (rCa); (−)menthone (mn); R(+)-pulegone (pu); isopulegol (ip); menthol (me); R(+)-limonene (lim); isoamyl acetate (am); vanillin (va); o-vanillin (ova); geraniol (ge); nerol (ne); hexanoic acid (mc6); heptanoic acid (mc7); octanoic acid (mc8); nonanoic acid (mc9); 1-hexanol (mh6); 1-heptanol (mh7); 1-octanol (mh8); 1-nonanol (mh9); indole (in); triethylamine (ta); isovaleric acid (iv); KCl (hk), potassium chloride; 3-isobutyl-1-methylxanthine (ibmx).

### Example 7

### Response Threshold of Olfactory Receptor

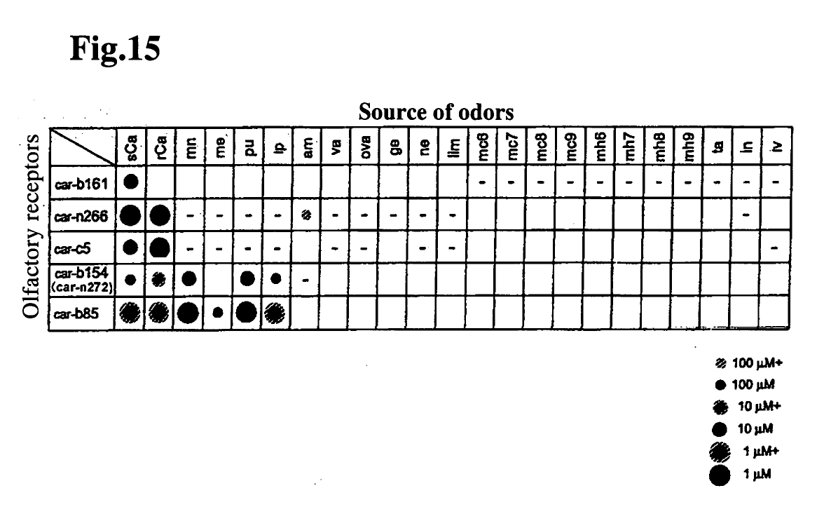

Next, in this Example, response thresholds, corresponding to specific types of odor molecule, of 5 kinds of olfactory receptors measured in olfactory receptor cells, were studied. By making an array sensor using whole or a part of these receptors, the following evaluation can be implemented, comparing responses of each receptor, using relative sensitivity against each odor molecule which those receptor have as index: which of sCa and rCa is the primary component in targeted odor stimulation; and whether or not mn is included as a secondary component. As a result, it was proved that the following evaluations are apparent: it has spearmint odor (in which rCa is primary component with a proportional share of 70%, and mn is included at about 1/70 of rCa), or caraway odor (in which sCa is primary component with a proportional share of 50-60%, and few mn is included), or peppermint odor or mint odor (in which me is primary component with a proportional share of about 40%, and mn is included at a proportional share of 20%) (FIG. 15). Sequences of receptors used in FIG. 15 are as follows:
car-b161 SEQ ID NO: 21
car-b154 (car-n272) SEQ ID NO: 15
car-n266 SEQ ID NO: 13
car-c5 SEQ ID NO: 19
car-b85 SEQ ID NO: 17.

As seen from FIG. 15, it was proved that each receptor has a specific directivity. If such an example is implemented using other receptors such as an olfactory receptor having a sequence indicated in SEQ ID NO: 34, 36, 38, 40, 42, 44, 46, 48, 50, 52, 54, 56, 58, 76, 78, 80, 82, 84, 86, 88, 90, 92, 94, and 96, it becomes clear that each has a specific sensitivity.

### Example 8

### Response Characteristics of Olfactory Receptor C257

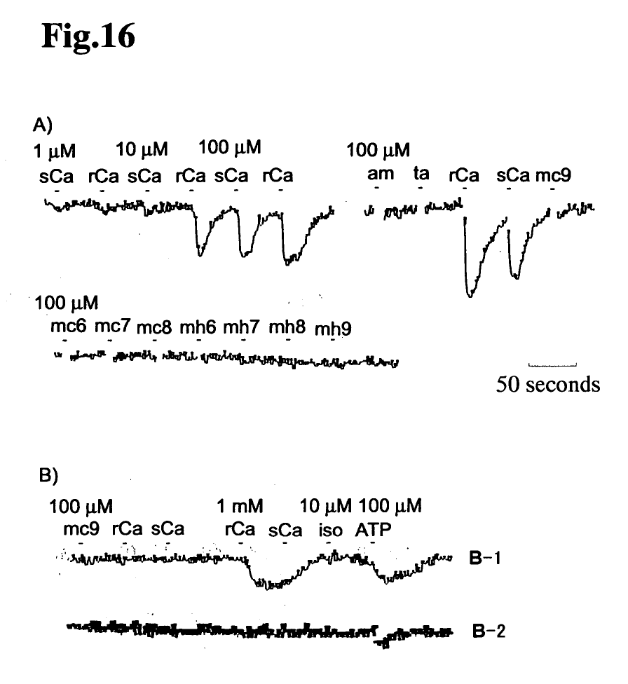

Next, in this Example, response characteristics of olfactory receptor C257 were studied. A) is the case wherein it is expressed in olfactory receptor cell, and B) is the case wherein it is expressed in cell established and cultured cell CHO. When expressed in CHO, sensitivity was decreased by one order of magnitude difference from the previous one, and therefore, high sensitivity could be confirmed by rCa. However, response to sCa was not apparent (FIG. 16). Accordingly, it was proved that it is preferable to structure sensor array using all the same cell systems. Regarding discrimination, characteristics of the olfactory receptor can be considered to be expressed, independent of the cell type. However, regarding sensitivity and response starting time, they are change with regards to all odor molecules depending on the cell type. Therefore, it is necessary to evaluate the stimulation quality, paying attention to this point.

### Example 9

### A Sensor using Modified Olfactory Receptor

Next, the demonstration was implemented as to whether or not the purpose of the present invention can also be accomplished using a modified olfactory receptor. A chimeric receptor cassette, IHS, used in the present Example was prepared by substituting the tag domain of rhodopsin (Rho) of that reported in Krautwurst D. et al., Cell 95: 917-926 (1998), with IHS sequence domain amongst the sequences added to the receptors used for expression of an olfactory receptor, to one attached by a rhodopsin tag reported in Gaillard I., et al., European J. Neurosci., 15: 409-418 (2002).

Transmembrane domain II-VII sequence of a targeted olfactory receptor was inserted in the place of a dummy sequence: CGCTGGTGC. For that reason, regarding the terminii of transmembrane domain II-VII sequence of a targeted olfactory receptor, at II, sequence including a sequence CTGCAG sectioned by restriction enzyme Pst I, for example, ACCGAACACCGCCTGCAG (SEQ ID NO: 102), was added, and at VII, sequence digested by Bsp EI, for example, sequence including TCCGGA such as TCCGGAACAAGGAATTGA (SEQ ID NO: 103) and the like were inserted. Then the sequence of an olfactory receptor requied for insertion after digestion of the dummy sequence was digested by Pst I and Bsp EI, and inserted. As an exemplary chimeric receptor cassette (including dummy sequence) used in the Example, Rho-M4 chimeric cassette with a rhodopsin tag attached, the IHS-M4 chimeric cassette of IHS sequence and the like were used. The sequence of Rho-M4 chimera cassette for pBK-CMV vector without the dummy is shown in SEQ ID NO: 104 and 105. Sequence of Rho-M4 chimeric cassette for pBK-CMV vector with the dummy sequence is shown in SEQ ID NO: 106 and 107. Sequence of IHS-M4 chimeric cassette for pBK-CMV vector without dummy is shown in SEQ ID NO: 108 and 109. Sequence of the IHS-M4 chimeric cassette for pBK-CMV vector with the dummy sequence is shown in SEQ ID NO: 110 and 111.

Using this cassette as an olfactory receptor, a nucleic acid molecule, connecting a nucleic acid molecule having a sequence shown in SEQ ID NO: 1, 3, 5, 7, 9, 11, 13, 15, 17, 19 and 21, was prepared for expression in a CHO cell. When an olfactory receptor sensor is prepared using such a cell, the sensor is found to have modified discrimination.

### Example 10

### Odor Sensor using a Partial Sequence

The present example demonstrated that partial sequences can be used to produce functional odor sensors. In order to express a nucleic acid molecule, a cassette for chimeric receptors was used wherein the cassette comprises chimeric receptor including, the region from the N-terminus to the first amino acid of the transmembrane domain II of olfactory receptor M4, as N region from 5′ non-translated region to first 20 amino acids including the translation initiation signal methionine, the region of the amino acid next to the transmembrane domain II at C-terminus of olfactory receptor M4 to the C-terminus, as the C-terminus, and transmembrane domains II to VII of a desired olfactory receptors inserted therebetween. It is possible to express the chimeric olfactory receptors by inserting transmembrane domains II to VII of a desired olfactory receptor in a cultured cell to obtain response property of the cell. In the present Example, sequences set forth in SEQ ID NO: 1, 3, 5 and 7 can be used to confirm that such partial sequences can function as a sensor. Alternatively, each transmembrane domain among the sequences set forth in SEQ ID NO: 34, 36, 38, 40, 42, 44, 46, 48, 50, 52, 54, 56, 58, 76, 78, 80, 82, 84, 86, 88, 90, 92, 94 and 96 may be selected for incorporation into the cassette to express the same for evaluation. It can be understood that such cassettes are used to prepare a sensor of the present invention in an enabling manner.

### Example 11

### Experiments using Epidermal Growth Factor Receptors (EGF))

Similar experiments were conducted in the present Example by using EGF receptor, the sequence of which is set forth in SEQ ID NO: 74 as descried in Example 5. In the case of EGF receptors, phosphorylation was determined by observing phosphate fluorescent labeled in order to detect phosphorylation.

Next, a variety of cytokines such as HGF, FGF, in addition to EGF, as well as albumin as a control, were used as stimulants to ensure that EGF was well recognized. Next, a number of systems in which a variety of chemical substances were used, were also prepared to determine whether the system functioned. A similar method can be used when using HGF receptor and FGF receptor, to determine high specificity to the HGF and FGF, respectively.

### Example 12

### Experiments using Mercury Pathway Profiling Systems

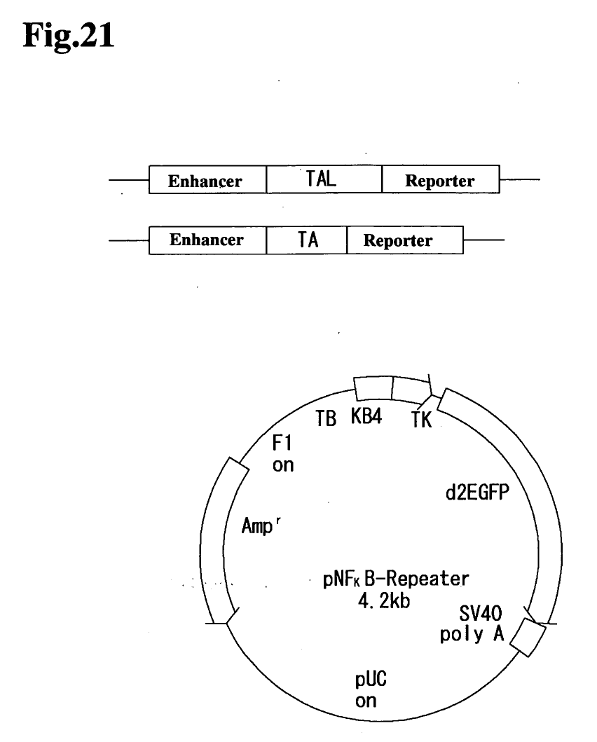

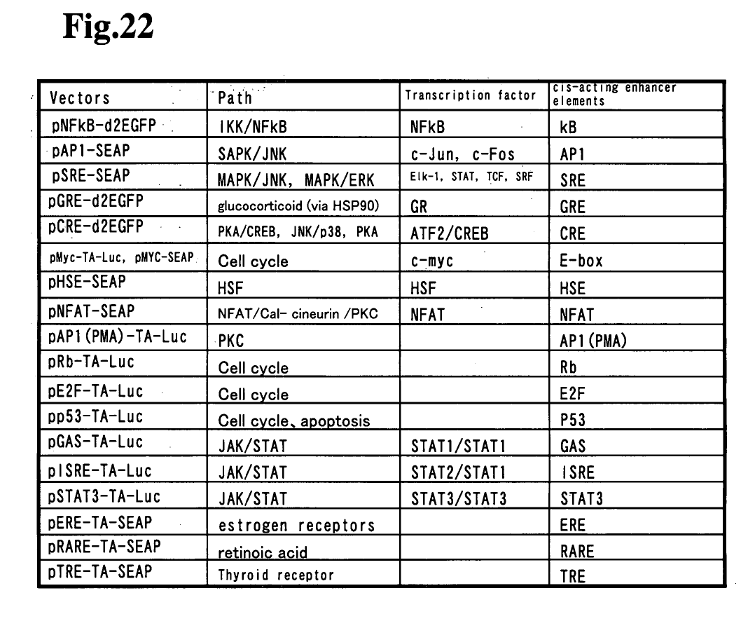

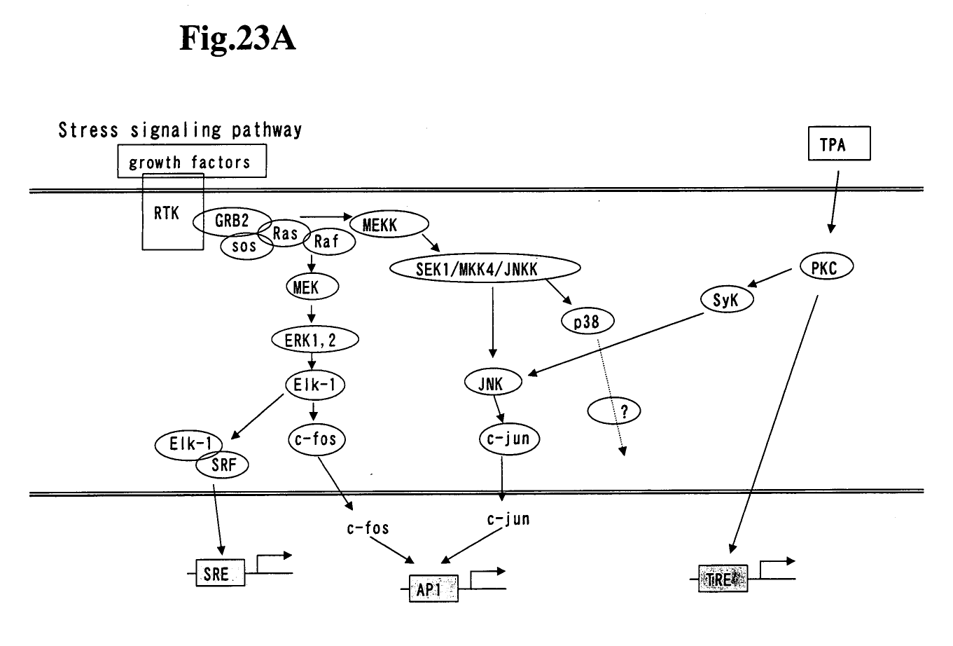

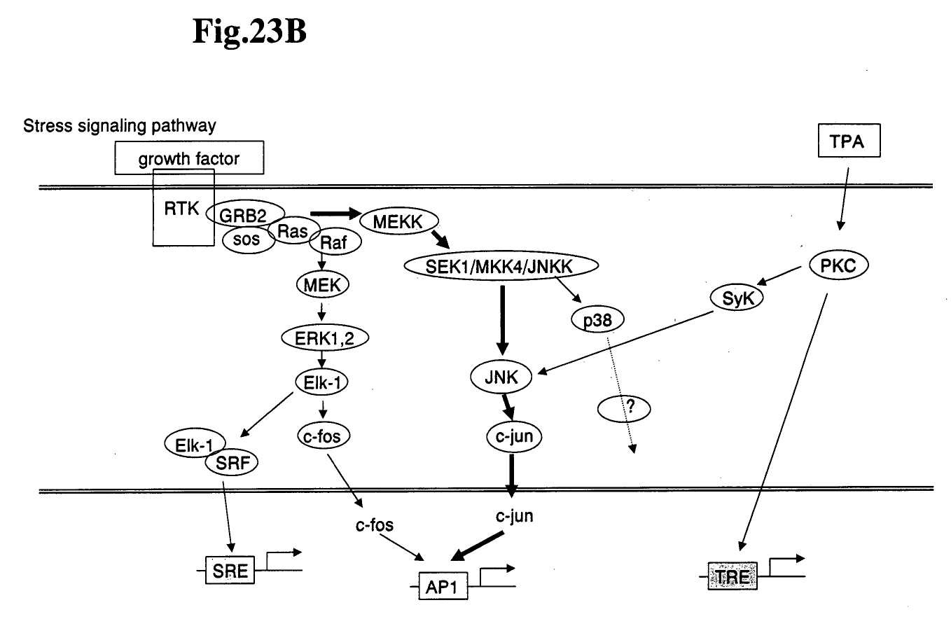

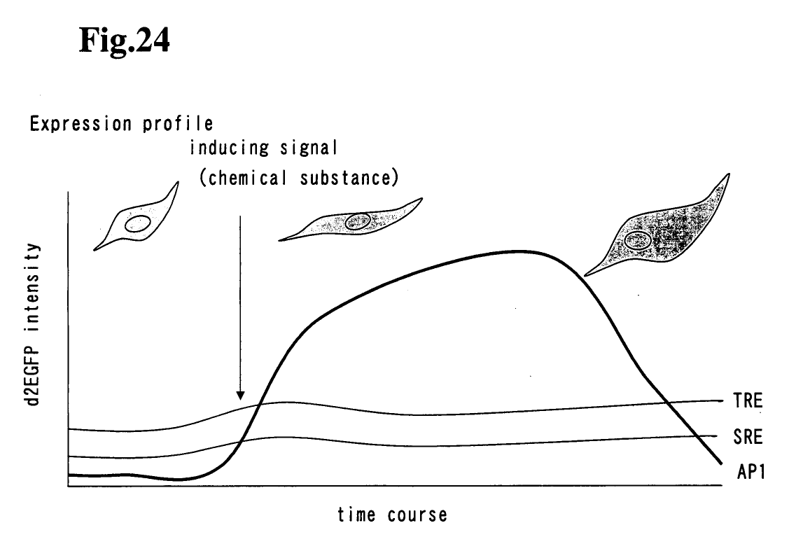

Next, similar experiments as in Example 9 were conducted to investigate response property of a variety of chemicals using the Mercury Pathway Profiling System. Vectors used are shown in FIGS. 21 and 22. The signal transduction pathway of the system is shown in in FIG. 23, and as can be seen, a number of kinases are mediators of the pathway. In this case, stimuli such as chemicals may activate a specific transcriptional agent as shown in FIG. 24. As such, activation is invested to allow specification of the stimulant in issue.

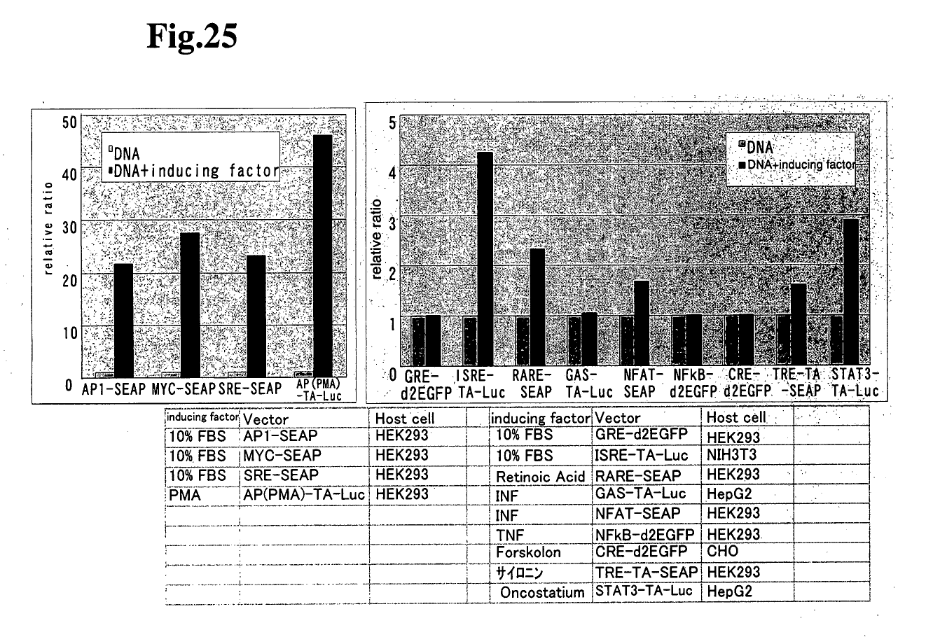

In order to carry out the reaction on a chip as described above in the examples, whether or not each gene could transduce the response of interest under control conditions of each gene was determined. Comparision between conditions with and without stimulus is shown in FIG. 25. As such, it was confirmed that the cells which contain an element of the system react in a proper manner with respect to the stimulus of interest.

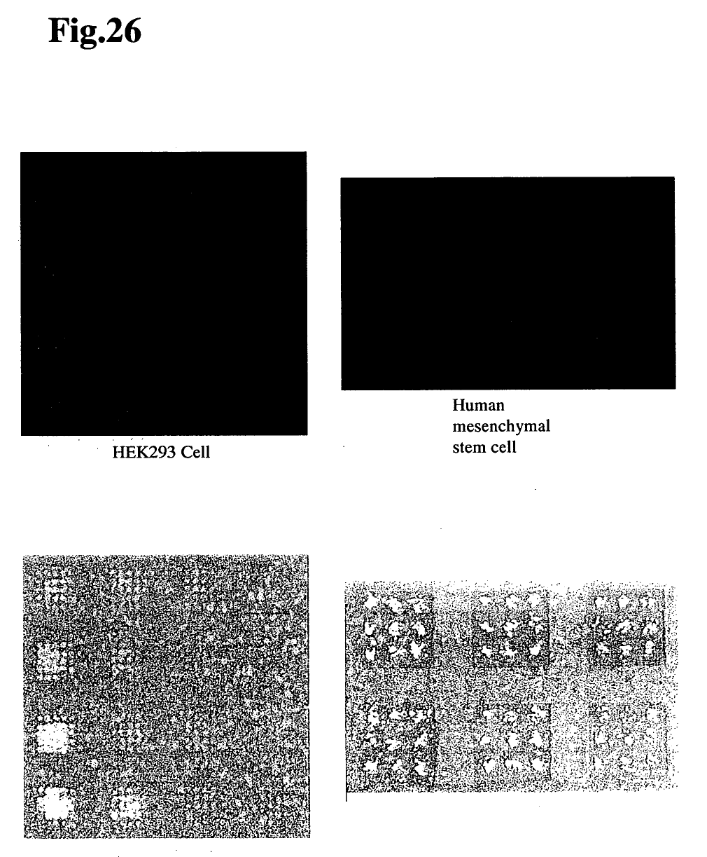

It now is possible to introduce a plurality of genes onto a solid phase for a plurality of cells, and to configurate a sensor for capturing information where the cell is in situ. An array in which cells have been printed/arrayed and gene expression was established, is shown in FIG. 26. Left shows HEK293 cells, and right shows human mesenchymal stem cells. As such, even a tiny spot may be separately recognized. Accordingly, such chip allows production of chemical sensor using.

### EXPERIMENTAL PROTOCOLS

(Cell Sources, Culture Media, and Culture Conditions)

In this example, two different cell lines were used: human mesenchymal stem cells (hMSCs, PT-2501, Cambrex BioScience Walkersville, Inc., MD), and human embryonic kidney cell HEK293 (RCB1637, RIKEN Cell Bank, JPN). In the case of human MSCs, cells were maintained in commercialized Human Mesenchymal Cell Basal Medium (MSCGM Bullet Kit PT-3001, Cambrex BioScience Walkersville, Inc., MD). In case of HEK293, cells were maintained in Dulbecco's Modified Eagle's Medium (DMEM, high glucose 4.5 g/L with L-Glutamine and sodium pyruvate; 14246-25, Nakalai Tesque, JPN) with 10% fetal bovine serum (FBS, 29-167-54, Lot No. 2025F, Dainippon Pharmaceutical CO., LTD., JPN). All cells were cultivated in a controlled incubator at 37° C. in 5% CO₂. In experiments involving hMSCs, we used hMSCs of less than five passages, in order to avoid phenotypic changes.

(Plasmids and Transfection Reagents)

To evaluate the efficiency of transfection, the pEGFP-N1 and pDsRed2-N1 vectors (cat. no. 6085-1, 6973-1, BD Biosciences Clontech, CA) were used. Expression of both genes was under the control of the cytomegalovirus (CMV) promoter. Transfected cells continuously expressed EGFP or DsRed2, respectively. Plasmid DNAs were amplified using Escherichia coli, XL1-blue strain (200249, Stratagene, TX), and purified by EndoFree Plasmid Kit (EndoFree Plasmid Maxi Kit 12362, QIAGEN, CA). In all cases, plasmid DNA was dissolved in DNase and RNase free water. Transfection reagents were obtained as below: Effectene Transfection Reagent (cat. no. 301425, Qiagen, CA), TransFast™ Transfection Reagent (E2431, Promega, WI), Tfx™-20 Reagent (E2391, Promega, WI), SuperFectTransfectionReagent (301305, Qiagen, CA), PolyFect Transfection Reagent (301105, Qiagen, CA), LipofectAMINE 2000 Reagent (11668-019, Invitrogen corporation, CA), JetPEI (×4) conc. (101-30, Polyplus-transfection, France), and ExGen 500 (RO511, Fermentas Inc., MD).

(Solid-Phase Transfection Array (SPTA) Production)

The detail of protocols for ‘reverse transfection’ was described in the web site, ‘Reverse Transfection Homepage’ (http://staffa.wi.mit.edu/sabatini_public/reverse_transfection.htm) or J. Ziauddin, D. M. Sabatini, Nature, 411, 2001, 107; and R. W. Zu, S. N. Bailey, D. M. Sabatini, Trends in Cell Biology, Vol. 12, No. 10, 485. In our solid phase transfection (SPTA method), three types of glass slides were studied (silanized glass slides; APS slides, and poly-L-lysine coated glass slides; PLL slides, and MAS coated slides; Matsunami Glass, JPN) with a 48 square pattern (3 mm×3 mm) separated by a hydrophobic fluoride resin coating.

(Plasmid DNA Printing Solution Preparation)

Two different ways to produce a SPTA were developed. The main differences reside in the preparation of the plasmid DNA printing solution.

(Method A)

In the case of using Effectene Transfection Reagent, the printing solution contained plasmid DNA and cell adhesion molecules (bovine plasma fibronectin (cat. no. 16042-41, Nakalai Tesque, JPN), dissolved in ultra-pure water at a concentration of 4 mg/mL). The above solution was applied on the surface of the slide using an inkjet printer (synQUAD™, Cartesian Technologies, Inc., CA) or manually, using a 0.5 to 10 μL tip. This printed slide was dried up over 15 minutes at room temperature in a safety-cabinet. Before transfection, total Effectene reagent was gently poured on the DNA-printed glass slide and incubated for 15 minutes at room temperature. The excess Effectene solution was removed from the glass slide using a vacuum aspirator and dried at room temperature for 15 minutes in a safety-cabinet. The DNA-printed glass slide obtained was set in the bottom of a 100-mm culture dish and approximately 25 mL of cell suspension (2 to 4×10⁴ cells/mL) was gently poured into the dish. Then, the dish was transferred to an incubator at 37° C. in 5% CO₂ and incubated for 2 or 3 days.

(Method B)

In the case of other transfection reagents (TransFast™, Tfx™-20, SuperFect, PolyFect, LipofectAMINE 2000, JetPEI (×4) conc., orExGen), plasmidDNA, fibronectin, and the transfection reagent were mixed homogeneously in a 1.5-mL micro-tube according to the ratios indicated in the manufacturer's instructions and incubated at room temperature for 15 minutes before printing onto a chip. The printing solution was applied onto the surface of the glass-slide using an inkjet printer or a 0.5- to 10-μL tip. The printed glass-slide was completely dried up at room temperature over 10 minutes in a safety-cabinet. The printed glass-slide was placed in the bottom of a 100-mm culture dish and approximately 3 mL of cell suspension (2 to 4×10⁴ cells/mL) was added and incubated at room temperature for 15 minutes in a safety-cabinet. After incubation, fresh medium was poured gently into the dish. Then, the dish was transferred to an incubator at 37° C. in 5% CO₂ and incubated for 2 to 3 days. After incubation, using fluorescence microscopy (IX-71, Olympus PROMARKERING, INC., JPN), we observed the transfectants, based on their expression of enhanced fluorescent proteins (EFP, EGFP and DsRed2). Phase contrast images were taken with the same microscope. In both protocols, cells were fixed by using a paraformaldehyde (PFA) fixation method (4% PFA in PBS, treatment time was 10 minutes at room temperature).

(Laser Scanning and Fluorescence Intensity Quantification)

In order to quantify the transfection efficiency, we used a DNA micro-array scanner (GeneTAC UC4×4, Genomic Solutions Inc., MI). The total fluorescence intensity (arbitrary units) was measured, and thereafter, the fluorescence intensity per surface area was calculated.

Although certain preferred embodiments have been described herein, it is not intended that such embodiments be construed as limitations on the scope of the invention except as set forth in the appended claims. Various other modifications and equivalents will be apparent to and can be readily made by those skilled in the art, after reading the description herein, without departing from the scope and spirit of this invention. All patents, published patent applications and publications cited herein are incorporated by reference as if set forth fully herein.

### INDUSTRIAL APPLICABILITY

The present invention allows determination of chemicals as a stimulus in a detailed manner by using a chemical receptor. Such determination allows diagnosis, prevention, therapy, and the range of applications are not limited to medicine, but also to the food industry, cosmetic industry, agriculture, environment industry and the like. It also provides a sensation-evaluation system allowing qualitative/quantitative evaluation of a sense from the signal output of a sensor, a method for evaluation and method for composing a specific stimulant. It also provides a sensation-evaluation system for evaluating sensation arising from a stimulus using the output signal of a sensor comprising a plurality of sensors having different response characteristics from each other against stimuli from outside; B) a signal processing member for using a stimulus species categorizing method based on a stimulus element tuning specificity of a cell having a chemical receptor to add a first signal output by a predetermined plurality of said sensors, to calculate a value of sensory elemental information expressing a sensation, and outputting a calculation result as a second signal; and an evaluation member for effecting qualitative and/or quantitative evaluation using the second signal output by the signal processing member.

The present invention is expected to accelerate development of sense function substitution apparatus, a controlling apparatus/manufacturing apparatus using sense quantity evaluation technology, a robot having sense and determination capabilities, and the like, by providing for the first time a qualitative/quantitative determination of a odor or olfactory sense, which was difficult for processing using an engineering approach. In particular, it is of use in that providing technology for identifying R(−)-carvone and S(+)-carvone, as the identification thereof in a distinguishing manner therebetween is difficult, as optical isomers. The present invention may be used for evaluation of quality of the composed mint odor of a food. Further, the technology of the present invention may be used for applying to a odor sensor for general use, recording and reproduction apparatus for olfactory information, a system for controlling food manufacture process, and a medical apparatus for diagnosis using an olfactory sense, and the like, which previsouly been thought to be difficult to achieve, which are of engineering technology that is friendly to human beings for beyond our expectation.

The present invention identifies odors such as spearmint, caraway, mint and sweet odors, which were believed to be difficult to identify.

Gene sequences of olfactory receptors which were elucidated with respect to response properties and can be used for developing an odor sensor for a purpose of detecting spearmint, caraway, mint and sweet odors and the like, for example, among odors perceived by humans and animals, are provided. The present invention is expected to accelerate the analysis of three dimensional structure of a olfactory molecule binding site controlling odor identification function based on the gene sequence information presented. Further, cells expressing such an olfactory receptor and the like are used as a sensor element to achieve an odor sensor allowing detection of spearmint, caraway, mint and sweet odors. The present invention may be used for evaluating the composition of the mint odor of a food, and for determining a variety of evaluation of separation of for example, R(−)-carvone and S(+)-carvone, optical isomers for which are difficult to distinguish between, and the like.

What is claims is:

1. A chemical sensor comprising:
a) a nucleic acid comprising a sequence encoding a chemical receptor gene;
b) a support with a cell located thereon, wherein the cell has the nucleic acid introduced therein;
c) means for measuring a signal caused by the chemical receptor; and
d) means for providing information relating to a chemical by calculating the extent of activation of the chemical receptor from the intensity of the measured signal.

2. The sensor according to claim 1, wherein said nucleic acid further comprises a sequence encoding a marker gene.

3. The sensor according to claim 1, wherein said chemical receptor comprises a receptor selected from the group consisting of nuclear receptors, cytoplasmic receptors and cellular membrane receptors.

4. The sensor according to claim 1, wherein said chemical receptor is selected from the group consisting of G protein coupled receptors, kinase type receptors, ion-channel type receptors, nuclear receptors, hormone receptors, chemokine receptors, and cytokine receptors.

5. The sensor according to claim 1, wherein said chemical receptor comprises an olfactory receptor.

6. The sensor according to claim 1, wherein said chemical receptor gene is selected from the group consisting of retinoic acid receptors, EGF receptors, interleukin receptors, and CSF receptors.

7. The sensor according to claim 1, wherein said chemical receptor gene comprises a nucleic acid sequence or a variant or fragment thereof, selected from the group consisting of SEQ ID NO: 1, 3, 5, 7, 9, 11, 13, 15, 17, 19, 21, 34, 36, 38, 40, 42, 44, 46, 48, 50, 52, 54, 56, 58, 76, 78, 80, 82, 84, 86, 88, 90, 92, 94 and 96.

8. The sensor according to claim 1, wherein said chemical receptor gene comprises at least about two species.

9. The sensor according to claim 1, wherein said chemical receptor gene comprises at least about ten species.

10. The sensor according to claim 1, wherein said chemical receptor gene comprises at least about twenty species.

11. The sensor according to claim 1, wherein said chemical receptor gene comprises at least about three hundred species.

12. The sensor according to claim 1, wherein said chemical receptor gene comprises substantially all species which are possessed by an animal selected from the group consisting of mice, humans, rats, dogs and cats.

13. The sensor according to claim 1, wherein said chemical receptor gene comprises substantially all species of olfactory receptor genes which are possessed by an animal selected from the group consisting of mice, humans, rats, dogs and cats.

14. The sensor according to claim 2, wherein said marker gene comprises G protein, said chemical receptor itself, or arrestin.

15. The sensor according to claim 2, wherein said marker gene comprises Gα gene.

16. The sensor according to claim 2, wherein said marker gene comprises Gα15, Gαq or Gαolf gene.

17. The sensor according to claim 2, wherein said marker gene comprises Gα gene, Gβ gene and Gγ gene.

18. The sensor according to claim 1, wherein said support comprises solid support.

19. The sensor according to claim 1, wherein the support comprises a material selected from the group consisting of glass, silicon, silica, polystyrene and polymer films, which is coated or non-coated.

20. The sensor according to claim 1, wherein the signal comprises an agent selected from the group consisting of intracellular calcium concentration, inositol triphosphate, cyclicAMP, diacyl glycerol, cyclic GMP and cellular membrane potential.

21. The sensor according to claim 1, wherein the signal is intracellular calcium concentration, and said means for measuring the signal comprises a means for electronically, chemically or biologically measuring calcium concentration.

22. The sensor according to claim 2, wherein the marker gene is different from the genes which originally exist in said cell.

23. The sensor according to claim 1, wherein the cell comprises a cell selected from the group consisting of HEK 293 cell, CHO cell, COS-7 cell, neuroblastoma and NG108-15.

24. The sensor according to claim 1, wherein the cell substantially comprises one type of cell.

25. The sensor according to claim 5, wherein the olfactory receptor gene is different from the genes which originally exist in said cell.

26. The sensor according to claim 1, wherein the support comprises a cellular adhesion molecule.

27. The sensor according to claim 16, wherein the cellular adhesion molecule comprises fibronectin, vitronectin, or laminin, or a fragment or variant thereof.

28. The sensor according to claim 1, wherein the nucleic acid molecule or cell on said support is arranged thereon in an array format.

29. The sensor according to claim 28, wherein the arrayed region has an area of about 200 mm² or less.

30. The sensor according to claim 28, wherein the arrayed region has about 15 mm or less in length in the longitudinal direction.

31. The sensor according to claim 1 further comprising liquid sufficient for covering the cell.

32. The sensor according to claim 1 further comprising a medium for maintaining a cell.

33. The sensor according to claim 32, wherein the medium is a liquid medium.

34. The sensor according to claim 1, wherein said d) means for providing information comprises:
d-1) a signal processing member for using a stimulus species categorizing method based on a stimulus element tuning specificity of a cell having a chemical receptor to add a first signal output by predetermined plurality of said sensors, to calculate a value of sensory elemental information expressing a sensation, and outputting a calculation result as a second signal; and
d-2) an evaluation member for effecting qualitative and/or quantitative evaluation using the second signal output by the signal processing member.

35. The sensor according to claim 34, wherein the subqualities of sensation to the stimulus categorizing method uses classification according to species of the chemical receptor.

36. The sensor according to claim 34, wherein said signal processing member reduces, when one of first signals output by the plurality of sensors exceeds a predetermined value, the first signal output by a sensor different from the sensor and uses the reduced signal for producing the second signal.

37. The sensor according to claim 34, wherein the signal processing member comprises:
a plurality of selection members and addition members corresponding to sensory elemental information;
a plurality of amplification members corresponding to each of the sensors;
a coefficient calculation member for controlling the amplification member, wherein
the selection members multiply a plurality of the first signal with the coefficient designated by each of the sensors to produce a plurality of third signals;
the addition members add the plurality of third signals output by the corresponding selection member to produce a plurality of fourth signal;
the coefficient calculation member detects the maximum value among the plurality of fourth signals and normalizes each of the fourth signals using the maximum value to calculate control signals;
the amplification members use the corresponding control signals to produce the second signals corresponding to the intensity of sensory elemental information.

38. The sensor according to claim 37, wherein when a stimulus is presented, the first signal output by the sensor, is transiently produced directed to a predetermined value corresponding to the intensity or concentration of the stimulus from zero level, wherein the zero level is set as a status where no response is found in response to no stimuli;
the third signal is transiently produced associated therewith directing to a predetermined value corresponding to the intensity or concentration of a stimulus from zero level:
the coefficient calculation member determines a sensor response starting at base time when one of the first signals is determined to be the signal output in response to a stimulus by the sensor for the first time, and
calculates at a predetermined time as an elapsed time from the base time, the control signal for controlling the amplification member using the third signal at the predetermined time;
controls the amplification member using the control signal which was calculated at the last time until a control signal is calculated at the predetermined time.

39. The sensor according to claim 34, wherein when a stimulus is presented, the first signal output by the sensor, is transiently produced directed to a predetermined value corresponding to the intensity or concentration of the stimulus from zero level, wherein the zero level is set as a status where no response is found in response to no stimuli;
the third signal is transiently produced associated therewith directing to a predetermined value corresponding to the intensity or concentration of a stimulus from zero level;
the coefficient calculation member determines a sensor response starting at base time when one of the first signals is determined to be the signal output in response to a stimulus by the sensor for the first time;
during a period of time when the predetermined number of the plurality of third signals changes from augmentation to reduction,
calculates, at each time when the third signal is determined to start occurring significant output as a corresponding sense element, and when the third signal is determined to have achieved a plurality of boundary values which divide the section between the significant output value and the maximum value preset to the third signal into a plurality of segments;
controls the amplification member using the control signal which was calculated for the last time until the control signal is calculated.

40. A chip for use in a chemical sensor comprising:
a) a nucleic acid molecule comprising a base sequence encoding a chemical receptor gene; and
b) a support having a cell thereon arranged with the nucleic acid molecule introduced therein.

41. The chip according to claim 40, wherein the nucleic acid molecule further comprises a sequence encoding a marker gene.

42. The chip according to claim 40, further comprising means for transmitting a signal derived from the chemical receptor gene.

43. The chip according to claim 40, wherein the chemical receptor comprises a receptor selected from the group consisting of intranuclear receptors, cytoplasmic receptors and cellular membrane receptors.

44. The chip according to claim 40, wherein the chemical receptor comprises a receptor selected from the group consisting of G-protein coupled receptors, kinase-type receptors, ion-channel type receptors, intranuclear receptors, hormone receptors, chemokine receptors, and cytokine receptors.

45. The chip according to claim 40 wherein the chemical receptor comprises an olfactory receptor.

46. The chip according to claim 40, wherein the chemical receptor gene comprises a receptor selected from the group consisting of retinoic acid receptors, EGF receptors, interleukin receptors and CSF receptors.

47. The chip according to claim 40, wherein the chemical receptor gene comprises a nucleic acid sequence set forth in SEQ ID NO. selected from the group consisting of SEQ ID NO: 1, 3, 5, 7, 9, 11, 13, 15, 17, 19, 21, 34, 36, 38, 40, 42, 44, 46, 48, 50, 52, 54, 56, 58, 76, 78, 80, 82, 84, 86, 88, 90, 92, 94 and 96, or a variant or fragment thereof.

48. The chip according to claim 40, wherein the chemical receptor gene comprises at least about two species.

49. The chip according to claim 40, wherein the chemical receptor gene comprises at least about ten species.

50. The chip according to claim 40, wherein the chemical receptor gene comprises at least about twenty species.

51. The chip according to claim 40, wherein the chemical receptor gene comprises at least about three hundred species.

52. The chip according to claim 40, wherein the chemical receptor gene comprises all species of chemical receptor genes which are possessed by an animal selected from the group consisting of mice, humans, rats, dogs and cats.

53. The chip according to claim 40, wherein the chemical receptor gene comprises substantially all species of olfactory receptor genes which are possessed by an animal selected from the group consisting of mice, humans, rats, dogs and cats.

54. The chip according to claim 41, wherein the marker gene comprises Gα gene, the chemical receptor itself, or arrestin.

55. The chip according to claim 41, wherein the marker gene comprises a Gα gene.

56. The chip according to claim 41, wherein the marker gene comprises Gα15, Gαq or Gαolf.

57. The chip according to claim 41, wherein the marker gene comprises Gα gene, Gβ gene and Gγ gene.

58. The chip according to claim 40, wherein the support comprises a solid support.

59. The chip according to claim 40, wherein the support comprises material selected from the group consisting of coated or non-coated glass, silicon, silica, polystyrene and polymer film.

60. The chip according to claim 41, wherein a signal produced by the marker gene comprises an agent selected from the group consisting of intracellular calcium concentration, inositol triphosphate, cyclic AMP, diacylglycerol, cyclic GMP and cell membrane potential.

61. The chip according to claim 41, wherein the marker gene is different from any genes already present in the cell.

62. The chip according to claim 40, wherein the cell comprises a cell consisting of HEK293 cell, CHO cell, COS-7 cell, neiuroblastoma and NG108-15 cell.

63. The chip according to claim 41, wherein the support comprises a connecting point for transmitting a signal derived from the marker.

64. The chip according to claim 40, wherein the support comprises a connecting point capable of transmitting at least one signal selected from the group consisting of an electrical signal, chemical signal and biological signal.

65. The chip according to claim 45, wherein the olfactory receptor gene is different from any genes already present in the cell.

66. The chip according to claim 40, wherein the support comprises a cellular adhesion molecule.

67. The chip according to claim 66, wherein the cellular adhesion molecule comprises fibronectin, vitronectin or laminin, or a fragment or a variant thereof.

68. The chip according to claim 40, wherein the nucleic acid molecule or cell on said support is arranged thereon in an array format.

69. The chip according to claim 68, wherein the arrayed region has an area of about 200 mm² or less.

70. The chip according to claim 68, wherein the arrayed region has a length of about 15 mm or less in the longitudinal direction.

71. The chip according to claim 40, further comprising a liquid sufficient for covering the cell.

72. The chip according to claim 40, further comprising a medium for maintaining the cell.

73. The chip according to claim 72, wherein the medium is a liquid medium.

74. A method for obtaining information relating to a chemical in a sample, comprising the steps of:
A) providing a cell having a nucleic acid molecule introduced therein, wherein the nucleic acid molecule comprises a sequence encoding a chemical receptor gene;
B) providing the cell with a sample comprising or suspected to comprise a chemical of interest;
C) measuring a change induced by the chemical in a signal derived from the chemical receptor gene in the cell; and
D) calculating a level of activation of the chemical receptor from the change in intensity of the measured signal to provide information on the chemical.

75. The method according to claim 74, wherein the nucleic acid molecule further comprises a sequence encoding a marker gene.

76. The method according to claim 74, wherein the chemical is a source of olfaction.

77. The method according to claim 74, wherein the chemical receptor gene comprises an olfactory receptor.

78. The method according to claim 74, wherein the chemical receptor gene comprises at least about two species.

79. The method according to claim 74, wherein the chemical receptor gene comprises at least about ten species.

80. The method according to claim 74, wherein the chemical receptor gene comprises at least about twenty species.

81. The method according to claim 74, wherein the chemical receptor gene comprises at least about three hundred species.

82. The method according to claim 74, wherein the chemical receptor gene comprises all species of chemical receptor genes which are possessed by an animal selected from the group consisting of mice, humans, rats, dogs and cats.

83. The method according to claim 75, wherein the marker gene comprises G protein-coupled protein.

84. The method according to claim 75, wherein the marker gene comprises Gα gene, the chemical receptor itself, or arrestin.

85. The method according to claim 75, wherein the marker gene comprises Gα15, Gαq or Gαolf.

86. The method according to claim 75, wherein the marker gene comprises Gα gene, Gβ gene and Gγ gene.

87. The method according of claim 74 wherein the cell is fixed on a support.

88. The method according to claim 87, wherein the support comprises a solid support.

89. The method according to claim 74, wherein the support comprises material selected from the group consisting of coated or non-coated glass, silicon, silica, polystyrene and polymer film.

90. The method according to claim 75, wherein a signal produced by the marker gene comprises an agent selected from the group consisting of intracellular calcium concentration, inositol triphosphate, cyclic AMP, diacylglycerol, cyclic GMP and cell membrane potential.

91. The method according to claim 75, wherein the marker gene is different from any genes already present in the cell.

92. The method according to claim 74, wherein the cell comprises a cell consisting of HEK293 cell, CHO cell, COS-7 cell, neiuroblastoma and NG108-15 cell.

93. The method according to claim 74, wherein the olfactory receptor gene is different from any genes already present in the cell.

94. The method according to claim 74, wherein the support comprises a cellular adhesion molecule.

95. The method according to claim 94, wherein the cellular adhesion molecule comprises fibronectin, vitronectin or laminin, or a fragment or a variant thereof.

96. The method according to claim 74, wherein the information regarding the signal comprises change in level of an agent selected from the group consisting of intracellular calcium concentration, inositol triphosphate, cyclic AMP, diacylglycerol, cyclic GMP and cell membrane potential.

97. The method according to claim96, wherein the information is presented at real time.

98. The method according to claim 74, wherein the step B) comprises a step of providing the cell with the sample at a flow rate of about 1-4 mm/second.

99. The method according to claim 74, wherein the step B) comprises a step of providing the cell with the sample at a flow rate of about 2-3 mm/second.

100. The method according to claim 74, wherein the cell is provided with a liquid sufficient for covering the cell, and the step of B) comprises a step of providing the sample into the liquid.

101. The method according to claim 74, wherein further the cell is provided with a medium for maintaining the cell.

102. The method according to claim 101, wherein said medium comprises a liquid medium.

103. The method according to claim 87, wherein the nucleic acid molecule or cell on said support is arranged thereon in an array format.

104. The method according to claim 74, further comprising the step of correlating the information on the chemical with the information on the sample comprising or suspected to comprise the chemical of interest.

105. A sensation-evaluation system for evaluating sensation arising from a stimulant using output signal of a sensor comprising:
A) a plurality of sensors having different response characteristics from each other against stimuli from outside;
B) a signal processing member for using a stimulus species categorizing method based on a stimulus element tuning specificity of a cell having a chemical receptor to add a first signal output by predetermined plurality of said sensors, to calculate a value of sensory elemental information expressing a sensation, and outputting a calculation result as a second signal; and
C) an evaluation member for effecting qualitative and/or quantitative evaluation using the second signal output by the signal processing member.

106. The sensor evaluation system according to claim 105, wherein the cell is transfected with a nucleic acid molecule comprising a nucleic acid sequence encoding said chemical receptor.

107. The sensation-evaluation system according to claim 105, wherein said signal processing member reduces, when one of first signals output by the plurality of sensors exceeds a predetermined value, the first signal output by a sensor different from the sensor and uses the reduced signal for producing the second signal.

108. The sensation-evaluation system according to claim 105, wherein the signal processing member comprises:
a plurality of selection members and addition members corresponding to sensory elemental information;
a plurality of amplification members corresponding to each of the sensors;
a coefficient calculation member for controlling the amplification member, and wherein
the selection members multiply a plurality of the first signal with the coefficient designated by each of the sensors to produce a plurality of third signals;
the addition members add the plurality of third signals output by the corresponding selection member to produce a plurality of fourth signal;
the coefficient calculation member detects the maximum value among the plurality of fourth signals and normalizes each of the fourth signals using the maximum value to calculate control signals; and
the amplification members use the corresponding control signals to produce the second signals corresponding to the intensity of sensory elemental information.

109. The sensation-evaluation system according to claim 105, wherein when a stimulus is presented, the first signal output by the sensor, is transiently produced directed to a predetermined value corresponding to the intensity or concentration of the stimulus from zero level, wherein the zero level is set as a status where no response is found in response to no stimuli;
the third signal is transiently produced associated therewith directing to a predetermined value corresponding to the intensity or concentration of a stimulus from zero level:
the coefficient calculation member determines a sensor response starting at base time when one of the first signals is determined to be the signal output in response to a stimulus by the sensor for the first time; and
calculates at a predetermined time as an elapsed time from the base time, the control signal for controlling the amplification member using the third signal at the predetermined time;
controls the amplification member using the control signal which was calculated at the last time until a control signal is calculated at the predetermined time.

110. The sensation-evaluation system according to claim 108, wherein when a stimulus is presented, the first signal output by the sensors, is transiently produced directed to a predetermined value corresponding to the intensity or concentration of the stimulus from zero level, wherein the zero level is set as a status where no response is found in response to no stimuli;
the third signal is transiently produced associated therewith directing to a predetermined value corresponding to the intensity or concentration of a stimulus from zero level;
the coefficient calculation member determines a sensor response starting at base time when one of the first signals is determined to be the signal output in response to a stimulus by the sensor for the first time;
during a period of time when the predetermined number of the plurality of third signals change from augmentation to reduction,
calculates, at each time when the third signal is determined to start occurring significant output as a corresponding sense element, and when the third signal is determined to have achieved a plurality of boundary values which divide the section between the significant output value and the maximum value preset to the third signal into a plurality of segments;
controls the amplification member using the control signal which was calculated for the last time until the control signal is calculated, wherein when a stimulus is presented, the first signal output by the sensor, is transiently produced directed to a predetermined value corresponding to the intensity or concentration of the stimulus from zero level, wherein the zero level is set as a status where no response is found in response to no stimuli;
the third signal is transiently produced associated therewith directing to a predetermined value corresponding to the intensity or concentration of a stimulus from zero level;
the coefficient calculation member determines a sensor response starting base time when one of the first signals is determined to be the signal output in response to a stimulus by the sensor for the first time;
during a period of time when the predetermined number of the plurality of third signals change from augmentation to reduction,
calculates, at each time when the third signal is determined to start occurring significant output as a corresponding sense element, and when the third signal is determined to have achieved a plurality of boundary values which divide the section between the significant output value and the maximum value preset to the third signal into a plurality of segments;
controls the amplification member using the control signal which was calculated for the last time until the control signal is calculated.

111. The sensation-evaluation system according to claim 105, wherein the chemical receptor is an olfactory receptor; and
the sensor is a sensor responding to an olfactory stimulus.

112. A method for evaluating a sense in a sensation-evaluation system for evaluating sensation arising from a stimulus using output signals of a sensor comprising a plurality of sensors having different response characteristics from each other against stimuli from outside and a signal processing member for processing an output signal from the sensors, comprising the steps of:
the first step wherein the signal processing member for using a stimulus species categorizing method based on a stimulus element tuning specificity of a cell having a chemical receptor to add a first signal output by predetermined plurality of said sensors, to calculate a value of sensory elemental information expressing a sensation, and outputting a calculation result as a second signal;
the second step wherein an evaluation member for effecting qualitative and/or quantitative evaluation using the second signal output by the signal processing member.

113. The method according to claim 112, wherein the cell is transfected with a nucleic acid molecule comprising a nucleic acid sequence encoding said chemical receptor.

114. The method according to claim 112, further comprising the third step wherein in the first step, said signal processing member reduces, when one of first signals output by the plurality of sensors exceeds a predetermined value, the first signal output by a sensor different from the sensor and uses the reduced signal for producing the second signal.

115. The method according to claim 112, wherein the signal processing member comprises:
a plurality of selection members and addition members corresponding to sensory elemental information;
a plurality of amplification members corresponding to each of the sensor;
coefficient calculation member for controlling the amplification member, the first step further comprising the fourth step wherein the selection members multiplies a plurality of the first signal with the coefficient designated by each of the sensors to produce a plurality of third signals;
the fifth step wherein the addition members add the plurality of third signals output by the corresponding selection member to produce a plurality of fourth signal;
the sixth step wherein the coefficient calculation member detects the maximum value among the plurality of fourth signals and normalizes each of the fourth signals using the maximum value to calculate control signals; and
the seventh step wherein the amplification members use the corresponding control signals to produce the second signals corresponding to the intensity of sensory elemental information.

116. The method according to claim 114, wherein when a stimulus is presented, the first signal output by the sensor, is transiently produced directed to a predetermined value corresponding to the intensity or concentration of the stimulus from zero level, wherein the zero level is set as a status where no response is found in response to no stimuli and the signal produced by the fourth step is transiently produced associated therewith directing to a predetermined value corresponding to the intensity or concentration of a stimulus from zero level;
the method further comprises the eighth step wherein in the sixth step, the coefficient calculation member determines a sensor response starting at base time when one of the first signals is determined to be the signal output in response to a stimulus by the sensor for the first time, and
calculates at a predetermined time as an elapsed time from the base time, the control signal for controlling the amplification member using the signal produced by the fifth step at the predetermined time, and
controls the amplification member using the control signal which was calculated at the last time until a control signal is calculated at the predetermined time.

117. The method according to claim 114, wherein when a stimulus is presented, the first signal output by the sensor, is transiently produced directed to a predetermined value corresponding to the intensity or concentration of the stimulus from zero level, wherein the zero level is set as a status where no response is found in response to no stimuli and the signal produced by the fourth step is transiently produced associated therewith directing to a predetermined value corresponding to the intensity or concentration of a stimulus from zero level;
the method further comprising the eighth step wherein in the sixth step, the coefficient calculation member determines a sensor response starting at base time when one of the first signals is determined to be the signal output in response to a stimulus by the sensor for the first time;
during a period of time when the predetermined number of the plurality of signals produced by the fifth step change from augmentation to reduction,
calculates, at each time when the signal produced by the fifth step is determined to start occurring significant output as a corresponding sense element, and when the signal produced by the fifth step is determined to have achieved a plurality of boundary values which divide the section between the significant output value and the maximum value preset to the signal produced by the fifth step into a plurality of segments;
controls the amplification member using the control signal which was calculated for the last time until the control signal is calculated.

118. The method according to claim 112, wherein
the chemical receptor is an olfactory receptor; and
the sensor is a sensor responding to an olfactory stimulus.

119. A method for formulating a stimulant, comprising the steps of:
the first step of evaluating a predetermined stimulant using a sensation-evaluation system for evaluating sensation arising from a stimulant using output signal of a sensor comprising:
A-1) a plurality of sensors having different response characteristics from each other against stimuli from outside;
A-2) a signal processing member for using a stimulus species categorizing method based on a stimulus element tuning specificity of a cell having a chemical receptor to add a first signal output by predetermined plurality of said sensors, to calculate a value of sensory elemental information expressing a sensation, and outputting a calculation result as a second signal; and
A-3) an evaluation member for effecting qualitative and/or quantitative evaluation using the second signal output by the signal processing member;
B) the second step of determining a ratio of stimulant elements to be mixed corresponding thereto using a result of evaluation corresponding to the stimulant elements obtained by the evaluation result of the first step and the sensation-evaluation system; and
C) the third step of mixing the determined stimulant elements at the determined ratio.

120. The method according to claim 119, further comprising:
the fourth step of evaluating the mixed stimulant in the third step using the sensation-evaluation system; and
the fifth the step of comparing the evaluation step of fourth step and the evaluation result of the first step to determine the ratio to be newly mixed corresponding the stimulant element.

121. A computer readable recording medium having a computer program recorded thereon for implementing a process in a computer in a sensation-evaluation system for evaluating sensation arising from a stimulant using the output signal of a sensor comprising a plurality of sensors having different response characteristics from each other against stimuli from outside and a signal processing member for processing output signals from the sensors, the process comprising the procedures of:
the first procedure wherein the signal processing member using a stimulus species categorizing method based on a stimulus element tuning specificity of a cell having a chemical receptor to add a first signal output by predetermined plurality of said sensors, to calculate a value of sensory elemental information expressing a sensation, and outputting a calculation result as a second signal;
the second procedure wherein an evaluation member for effecting qualitative and/or quantitative evaluation using the second signal output by the signal processing member.

122. A computer program for implementing a process in a computer in a sensation-evaluation system for evaluating sensation arising from a stimulant using output signal of a sensor comprising a plurality of sensors having different response characteristics from each other against stimuli from outside and a signal processing member for processing an output signal from the sensors, the process comprising the procedures of:
the first procedure wherein the signal processing member using a stimulus species categorizing method based on a stimulus element tuning specificity of a cell having a chemical receptor to add a first signal output by predetermined plurality of said sensors, to calculate a value of sensory elemental information expressing a sensation, and outputting a calculation result as a second signal;
the second procedure wherein an evaluation member for effecting qualitative and/or quantitative evaluation using the second signal output by the signal processing member.

123. A nucleic acid molecule comprising:
(a) a polynucleotide having a base sequence set forth in SEQ ID NO. selected from the group consisting of SEQ ID NO: 1, 3, 5, 7, 9, 11, 13, 15, 17, 19and21, or a sequence fragment thereof;
(b) a polynucleotide encoding a polypeptide consisting of an amino acid sequence set forth in SEQ ID NO. selected from the group consisting of SEQ ID NO: 2, 4, 6, 8, 10, 12, 14, 16, 18, 20 and 22, or a fragment thereof;
(c) a polynucleotide encoding a variant polypeptide having an amino acid sequence set forth in SEQ ID NO. selected from the group consisting of SEQ ID NO: 2, 4, 6, 8, 10, 12, 14, 16, 18, 20 and 22, having at least one mutation selected from the group consisting of at least one amino acid substitution, addition and deletion, and having biological activity;
(d) a polynucleotide which is an allelic variant of DNA consisting of a base sequence set forth in SEQ ID NO. selected from the group consisting of SEQ ID NO: 1, 3, 5, 7, 9, 11, 13, 15, 17, 19 and 21;
(e) a polynucleotide encoding a species homolog of a polypeptide consisting of an amino acid sequence set forth in SEQ ID NO. selected from the group consisting of SEQ ID NO: 2, 4, 6, 8, 10, 12, 14, 16, 18, 20 and 22;
(f) a polynucleotide encoding a polypeptide hybridizable to any one of the polynucleotides (a) to (e) under stringent conditions, and having biological activity; or
(g) a polynucleotide consisting of a base sequence having at least 70% identity to any one of the polynucleotides (a) to (e) or a complementary sequence thereof, and having biological activity.

124. The nucleic acid molecule according to claim 123, wherein the biological activity comprises a signal transduction activity of a chemical.

125. A polypeptide comprising:
(a) a polypeptide encoded by polynucleotide of a nucleic acid sequence set forth in SEQ ID NO. selected from the group consisting of SEQ ID NO: 1, 3, 5, 7, 9, 11, 13, 15, 17, 19 and 21, or a fragment thereof
(b) a polypeptide consisting of an amino acid sequence set forth in SEQ ID NO. selected from the group consisting of SEQ ID NO: 2, 4, 6, 8, 10, 12, 14, 16, 18, 20 and 22, or a fragment thereof;
(c) a polypeptide comprising an amino acid sequence set forth in SEQ ID NO. selected from the group consisting of SEQ ID NO: 2, 4, 6, 8, 10, 12, 14, 16, 18, 20 and 22, having at least one mutation selected from at least one amino acid substitution, addition and deletion, and having biological activity;
(d) a polypeptide encoded by an allelic variant of a base sequence set forth in SEQ ID NO. selected from the group consisting of SEQ ID NO: 1, 3, 5, 7, 9, 11, 13, 15, 17, 19 and 21;
(e) a polypeptide which is a species homolog of an amino acid sequence set forth in SEQ ID NO. selected from the group consisting of SEQ ID NO: 2, 4, 6, 8, 10, 12, 14, 16, 18, 20 and 22; or
(f) a polypeptide having an amino acid sequence having at least 70% identity to any one of the polypeptides (a) to (e), and having biological activity.

126. The polypeptide according to claim 125, wherein said biological activity comprises signal transduction activity of a chemical.

127. Use of the nucleic acid molecule according to claim 123 or the polypeptide according to claim 125 for detecting a chemical.
